(19) United States
(12) Patent Application Publication
(10) Pub. No.: US 2016/0108027 A1
(43) Pub. Date: Apr. 21, 2016

(54) SUBSTITUTED BENZOXAZOLES

(71) Applicant: BAYER PHARMA AKTIENGESELLSCHAFT, Berlin (DE)

(72) Inventors: Swen ALLERHEILlGEN, Essen (DE); Anja BUCHMÜLLER, Essen (DE); Karen ENGEL, Rossdorf (DE); Christoph GERDES, Köln (DE); Kersten Matthias GERICKE, Wuppertal (DE); Michael GERISCH, Wuppertal (DE); Stefan HEITMEIER, Wülfrath (DE); Alexander HILLlSCH, Solingen (DE); Tom KINZEL, Düsseldorf (DE); Philip LIENAU, Berlin (DE); Bernd RIEDL, Wuppertal, (DE); Susanne RÖHRIG, Hilden (DE); Martina Victoria SCHMIDT, Köln (DE); Julia STRASSBURGER, Wuppertal (DE); Adrian TERSTEEGEN, Wuppertal (DE)

(21) Appl. No.: 14/895,625

(22) PCT Filed: May 30, 2014
(86) PCT No.: PCT/EP2014/061228

§ 371 Date: Dec. 3, 2015

(30) Foreign Application Priority Data
Jun. 3, 2013 (EP) 13170214.4

(51) Int. Cl. C07D 413/14; C07D 487/04

(52) U.S. Cl. CPC C07D 413/14; C07D 487/04

(57) ABSTRACT

The invention relates to substituted benzoxazoles and to processes for their preparation and to their use for preparing medicaments for the treatment and/or prophylaxis of diseases, in particular of cardiovascular disorders, preferably of thrombotic or thromboembolic disorders.

The invention relates to substituted benzoxazoles and to processes for their preparation and to their use for preparing medicaments for the treatment and/or prophylaxis of diseases, in particular of cardiovascular disorders, preferably of thrombotic or thromboembolic disorders.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a final joint reaction path, are distinguished. Here, factors Xa and IIa (thrombin) play key roles: Factor Xa bundles the signals of the two coagulation paths since it is formed both via factor VIIa/tissue factor (extrinsic path) and via the tenase complex (intrinsic path) by conversion of factor X. The activated serine protease Xa cleaves prothrombin to thrombin which, via a series of reactions, transduces the impulses from the cascade to the coagulation state of the blood: thrombin directly cleaves fibrinogen to fibrin. It activates factor XIII, required for stabilization of the fibrin clot, to factor XIIIa. In addition, thrombin is a potent trigger of platelet aggregation (via PAR-1 activation), which also contributes considerably to haemostasis. By activating TAFI (thrombin-activatable fibrinolysis inhibitor) to TAFIa, thrombin in a complex with thrombomodulin inhibits the dissolution of the clot. Activation of factors V and VIII potentiates the production of thrombin and thus in turn amplifies the coagulation reaction.

In addition to unbound thrombin in the blood, bound forms are also known. During the formation of a fibrin clot, thrombin and prothrombinase (factor Xa in a complex) are bound to the fibrin skeleton. These enzyme molecules are still active and cannot be inhibited by endogenous antithrombin III. Thus, in this manner, clots have a general coagulative potential.

In addition, thrombin, in particular via activation of PAR-1 receptors on endothelial cells, is also involved in inflammatory processes which, in interaction with the coagulation system, accelerates both processes.

Uncontrolled activation of the coagulation system or defect inhibition of the activation processes may lead to the formation of local thromboses or embolisms in vessels (arteries, veins, lymph vessels) or cardiac cavities. In addition, systemic hypercoagulability may lead to system-wide formation of thrombi and finally to consumption coagulopathy in the context of a disseminated intravasal coagulation. Thromboembolic complications are furthermore encountered in microangiopathic haemolytic anaemias, extracorporeal circulatory systems, such as haemodialysis, and also prosthetic heart valves and stents.

In the course of many cardiovascular and metabolic disorders, owing to systemic factors such as hyperlipidaemia, diabetes or smoking, owing to changes in blood flow with stasis, for example in atrial fibrillation, or owing to pathological changes in vessel walls, for example endothelial dysfunctions or atherosclerosis there is an increased tendency for coagulation and platelet activation which, via formation of fibrin- and platelet-rich thrombi, may lead to thromboembolic disorders and thrombotic complications with life-threatening conditions. Accordingly, thromboembolic disorders are still the most frequent cause of morbidity and mortality in most industrialized countries [Heart Disease: A Textbook of Cardiovascular Medicine, Eugene Braunwald, 5th edition, 1997, W.B. Saunders Company, Philadelphia].

The anticoagulants known from the prior art, that is to say substances for inhibiting or preventing blood coagulation, have various disadvantages. In the therapy and prophylaxis of thromboembolic disorders, use is made, firstly, of heparin which is administered parenterally or subcutaneously. Because of more favourable pharmacokinetic properties, preference is these days increasingly given to low-molecular-weight heparin; however, the known disadvantages described hereinbelow encountered in heparin therapy cannot be avoided either in this manner. Thus, heparin is orally ineffective and has only a comparatively short half-life. In addition, there is a high risk of bleeding, there may in particular be cerebral haemorrhages and bleeding in the gastrointestinal tract, and there may be thrombopaenia, alopecia medicomentosa or osteoporosis [Pschyrembel, Klinisches Wtirterbuch [clinical dictionary], 257th edition, 1994, Walter de Gruyter Verlag, page 610, keyword “Heparin”; Römpp Lexikon Chemie, version 1.5, 1998, Georg Thieme Verlag Stuttgart, keyword “Heparin”]. Low-molecular-weight heparins do have a lower probability of leading to the development of heparin-induced thrombocytopaenia; however, they can likewise only be administered subcutaneously. This also applies to fondaparinux, a synthetically produced selective factor Xa inhibitor having a long half-life.

A second class of anticoagulants are the vitamin K antagonists. These include, for example, 1,3-indanediones and in particular compounds such as warfarin, phenprocoumon, dicumarol and other coumarin derivatives which non-selectively inhibit the synthesis of various products of certain vitamin K-dependent coagulation factors in the liver. Owing to the mechanism of action, the onset of action is only very slow (latency to the onset of action 36 to 48 hours). The compounds can be administered orally; however, owing to the high risk of bleeding and the narrow therapeutic index complicated individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683]. In addition, other side-effects such as gastrointestinal problems, hair loss and skin necroses have been described.

More recent approaches for oral anticoagulants are in various phases of clinical evaluation or in clinical use; however, they have also displayed disadvantages such as, for example, highly variable bioavailability, liver damage and bleeding complications, in particular in patients with damaged kidneys.

For antithrombotic medicaments, the therapeutic width is of importance: The distance between the therapeutically active dose for coagulation inhibition and the dose where bleeding may occur should be as big as possible so that maximum therapeutic activity is achieved at a minimum risk profile.

In particular under therapeutic conditions with thrombi already present, it may be advantageous to inhibit also the factor IIa present in the thrombus, and thereby promote a rapid degradation of the thrombus. Using, for example, argatroban or hirudin as FIIa inhibitors, the advantageous effect of FIIa inhibition on an existing thrombus alone or in the presence of tissue plaminogen activator (tPA) has been demonstrated in various in-vitro and in-vivo models.

Accordingly, it is an object of the present invention to provide novel compounds as thrombin inhibitors for the treatment of cardiovascular disorders, in particular of thrombotic or thromboembolic disorders, in humans and animals, which compounds have a broad therapeutic width and good pharmacokinetic properties.

WO 98/37075 describes inter alia benzoxazole derivatives having an amidinobenzylamino substituent as thrombin inhibitors. Amidino-substituted thrombin inhibitors have a short half-life and low oral bioavailability. As such, the compounds are only suitable for parenteral administration and, when administered orally, have to be employed as prodrugs (A. Casimiro-Garcia, D. A. Dudley, R. J. Heemstra, K. J. Filipski, C. F. Bigge, J. J. Edmunds, Expert Opin. Ther. Patents 2006, 16(2), 119-145).

WO 2007/140982 describes the use of benzoxazoles as thrombin inhibitors.

EP-A 0 535 521 describes the use of benzoxazoles as leukotriene biosynthesis inhibitors for the treatment of inflammatory disorders.

The invention provides compounds of the formula

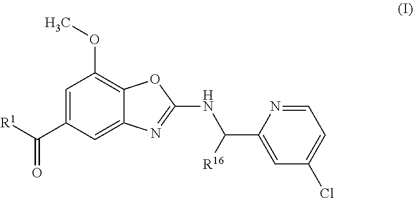

in which
R¹ represents a group of the formula

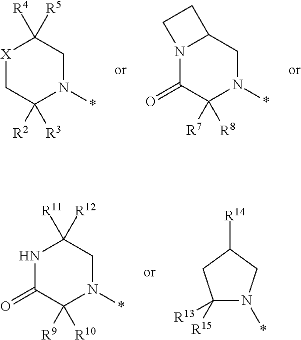

where * is the point of attachment to the carbonyl group,
X represents an oxygen atom, a sulphur atom or CH—R⁶,
   where
   R⁶ represents hydrogen or hydroxy,
R² represents hydrogen, aminocarbonyl, C₁-C₆-alkyl, C₃-C₆-cycloalkyl or phenyl,
   where alkyl and cycloalkyl may be substituted by a substituent selected from the group consisting of hydroxy, methoxy, cyano, hydroxycarbonyl, aminocarbonyl, methylsulphonyl, difluoromethoxy, trifluoromethoxy and cyclopropyl,
      where cyclopropyl for its part may be substituted by a hydroxy substituent,
   or
   where alkyl and cycloalkyl may be substituted by 1 to 3 fluorine substituents,
R³ represents hydrogen or C₁-C₄-alkyl,
or
R² and R³ together with the carbon atom to which they are attached form a cyclopropyl ring, cyclobutyl ring or cyclopentyl ring,
   where the cyclobutyl ring and the cyclopentyl ring may be substituted by a hydroxy substituent,
R⁴ represents hydrogen or C₁-C₆-alkyl,
   where alkyl may be substituted by a hydroxy substituent,
   or
   where alkyl may be substituted by 1 to 3 fluorine substituents,
R⁵ represents C₁-C₄-alkyl,
or
R⁴ and R⁵ together with the carbon atom to which they are attached form a cyclopropyl ring, cyclobutyl ring or cyclopentyl ring,
   where the cyclobutyl ring and the cyclopentyl ring may be substituted by a hydroxy substituent,
R⁷ represents hydrogen or C₁-C₆-alkyl,
   where alkyl may be substituted by one substituent selected from the group consisting of cyano, hydroxy and methoxy,
   or
   where alkyl may be substituted by 1 to 3 fluorine substituents,
R⁸ represents hydrogen,
R⁹ represents hydrogen or C₁-C₆-alkyl,
   where alkyl may be substituted by one substituent selected from the group consisting of hydroxy, cyano and aminocarbonyl,
   or
   where alkyl may be substituted by 1 to 3 fluorine substituents,
R¹⁰ represents hydrogen,
R¹¹ represents C₁-C₄-alkyl,
   where alkyl may be substituted by a hydroxy substituent,
R¹² represents hydrogen or C₁-C₄-alkyl,
or
R¹¹ and R¹² together with the carbon atom to which they are attached form a cyclopropyl ring, cyclobutyl ring or cyclopentyl ring,
   where the cyclobutyl ring and the cyclopentyl ring may be substituted by a hydroxy substituent,
R¹³ represents methyl, ethyl, (3-fluoroazetidin-1-yl)carbonyl, (3,3-difluoroazetidin-1-yl)carbonyl or morpholin-4-ylcarbonyl,
   where methyl and ethyl are substituted by a substituent selected from the group consisting of cyano and hydroxy,
R¹⁴ represents hydrogen, methoxy, ethoxy or cyclopropyloxy,
   where methoxy and ethoxy may be substituted by 1 to 3 substituents selected from the group consisting of deuterium and fluorine,
R¹⁵ represents hydrogen or methyl,
   and
   R¹⁶ represents hydrogen, methyl or fluoromethyl,
   and the salts thereof, the solvates thereof and the solvates of the salts thereof.

Compounds according to the invention are the compounds of the formula (I) and the salts, solvates and solvates of the salts thereof, and also the compounds encompassed by formula (I) and specified hereinafter as working example(s), and the salts, solvates and solvates of the salts thereof, to the extent that the compounds encompassed by formula (I) and specified hereinafter are not already salts, solvates and solvates of the salts.

The compounds according to the invention may, depending on their structure, exist in different stereoisomeric forms, i.e. in the form of configurational isomers or else optionally as conformational isomers (enantiomers and/or diastereomers, including those in the case of atropisomers). The present invention therefore encompasses the enantiomers and diastereomers, and the respective mixtures thereof. The stereoisomerically uniform constituents can be isolated from such mixtures of enantiomers and/or diastereomers in a known manner; chromatography processes are preferably used for this, especially HPLC chromatography on an achiral or chiral phase.

If the compounds according to the invention can occur in tautomeric forms, the present invention encompasses all the tautomeric forms.

The present invention also encompasses all suitable isotopic variants of the inventive compounds. An isotopic variant of an inventive compound is understood here to mean a compound in which at least one atom within the inventive compound has been exchanged for another atom of the same atomic number but with a different atomic mass from the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds according to the invention may therefore in some cases also constitute a preferred embodiment of the present invention. Isotopic variants of the inventive compounds can be prepared by the processes known to those skilled in the art, for example by the methods described below and the procedures described in the working examples, by using corresponding isotopic modifications of the respective reagents and/or starting compounds.

Preferred salts in the context of the present invention are physiologically acceptable salts of the inventive compounds. The invention also encompasses salts which themselves are unsuitable for pharmaceutical applications but which can be used, for example, for the isolation or purification of the inventive compounds.

Physiologically acceptable salts of the inventive compounds include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

Solvates in the context of the invention are described as those forms of the inventive compounds which form a complex in the solid or liquid state by coordination with solvent molecules. Hydrates are a specific form of the solvates in which the coordination is with water.

In addition, the present invention also encompasses prodrugs of the inventive compounds. The term “prodrugs” includes compounds which may themselves be biologically active or inactive but are converted to inventive compounds while resident in the body (for example metabolically or hydrolytically).

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, unless specified otherwise, the substituents are defined as follows:

Alkyl represents a straight-chain or branched alkyl radical having 1 to 6 carbon atoms, preferably 1 to 4 carbon atoms, by way of example and with preference methyl, ethyl, n-propyl, isopropyl, n-butyl, isobutyl, 1-methylpropyl, tert-butyl, n-pentyl, isopentyl, 1-ethylpropyl, 1-methylbutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl and 2-ethylbutyl.

Cycloalkyl represents a monocyclic cycloalkyl group having 3 to 6 carbon atoms, preferred examples of cycloalkyl being cyclopropyl, cyclobutyl, cyclopentyl and cyclohexyl.

In the formulae of the group which may represent R¹, the end point of the line marked by * in each case does not represent a carbon atom or a CH₂ group, but is part of the bond to the atom to which R¹ is attached.

Preference is given to compounds of the formula (I) in which

R¹ represents a group of the formula

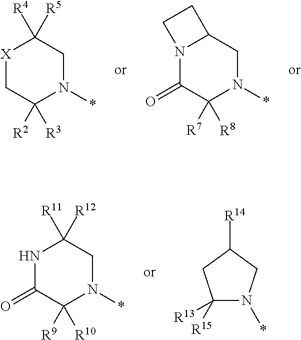

where * is the point of attachment to the carbonyl group,
X represents an oxygen atom or CH—R⁶,
   where
   R⁶ represents hydrogen,
R² represents hydrogen, C₁-C₄-alkyl or C₃-C₆-cycloalkyl,
   where alkyl and cycloalkyl may be substituted by a substituent selected from the group consisting of hydroxy, methoxy, hydroxycarbonyl, difluoromethoxy and cyclopropyl,
      where cyclopropyl for its part may be substituted by a hydroxy substituent,
   or
   where alkyl may be substituted by 1 to 3 fluorine substituents,
R³ represents hydrogen or C₁-C₄-alkyl,
or
R² and R³ together with the carbon atom to which they are attached form a cyclobutyl ring,
   where the cyclobutyl ring may be substituted by a hydroxy substituent,
R⁴ represents hydrogen or C₁-C₄-alkyl,
   where alkyl may be substituted by a hydroxy substituent,
R⁵ represents C₁-C₄-alkyl,
R⁷ represents C₁-C₄-alkyl,
   where alkyl may be substituted by a methoxy substituent,
R⁸ represents hydrogen,
R⁹ represents C₁-C₄-alkyl,
   where alkyl may be substituted by one substituent selected from the group consisting of cyano and aminocarbonyl,
R¹⁰ represents hydrogen,
R¹¹ represents C₁-C₄-alkyl,
R¹² represents hydrogen or C₁-C₄-alkyl,
or
R¹¹ and R¹² together with the carbon atom to which they are bonded form a cyclopropyl ring,
R¹³ represents methyl, ethyl, (3-fluoroazetidin-1-yl)carbonyl, (3,3-difluoroazetidin-1-yl)carbonyl or morpholin-4-ylcarbonyl,
   where methyl and ethyl are substituted by a substituent selected from the group consisting of cyano and hydroxy,
R¹⁴ represents hydrogen, ethoxy or cyclopropyloxy,
   where ethoxy may be substituted by 1 to 3 substituents selected from the group consisting of deuterium and fluorine,
R¹⁵ represents hydrogen or methyl,
   and
   R¹⁶ represents hydrogen or methyl,
   and the salts thereof, the solvates thereof and the solvates of the salts thereof.

Preference is also given to compounds of the formula (I) in which

R¹ represents a group of the formula

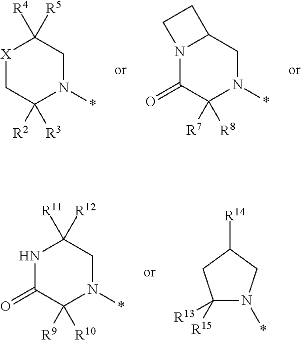

where * is the point of attachment to the carbonyl group,
X represents an oxygen atom,
R² represents C₁-C₄-alkyl or cyclobutyl,
   where alkyl may be substituted by one substituent selected from the group consisting of hydroxy and methoxy,
   or
   where alkyl may be substituted by 1 to 3 fluorine substituents,
   and
   where cyclobutyl is substituted by a hydroxy substituent,
R³ represents hydrogen or methyl,
R⁴ represents hydrogen or methyl,
and
R⁵ represents methyl,
or
R² represents methyl or ethyl,
   where methyl or ethyl may be substituted by 1 to 3 fluorine substituents,
R³ represents hydrogen or methyl,
R⁴ represents C₁-C₄-alkyl,
   where alkyl is substituted by a hydroxy substituent,
and
R⁵ represents methyl,
or
R² and R³ together with the carbon atom to which they are attached form a cyclobutyl ring,
   where the cyclobutyl ring is substituted by a hydroxy substituent,
R⁴ represents hydrogen or methyl,
and
R⁵ represents methyl,
R⁷ represents methyl or ethyl,
   where methyl and ethyl may be substituted by a methoxy substituent,
R⁸ represents hydrogen,
R⁹ represents methyl or ethyl,
   where methyl and ethyl may be substituted by a substituent selected from the group consisting of cyano and aminocarbonyl,
R¹⁰ represents hydrogen,
R¹¹ represents methyl,
R¹² represents hydrogen,
or
R¹¹ and R¹² together with the carbon atom to which they are bonded form a cyclopropyl ring,
R¹³ represents methyl,
   where methyl is substituted by a hydroxy substituent,
R¹⁴ represents ethoxy or cyclopropyloxy,
   where ethoxy may be substituted by 1 to 3 substituents selected from the group consisting of deuterium and fluorine,
R¹⁵ represents hydrogen,
   and
   R¹⁶ represents hydrogen or methyl,
   and the salts thereof, the solvates thereof and the solvates of the salts thereof.

Preference is also given to compounds of the formula (I) in which

R¹ represents a group of the formula

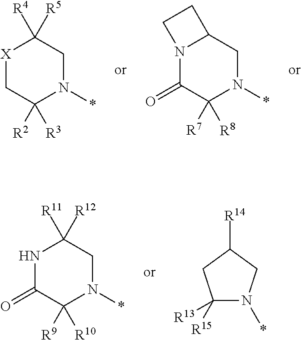

where * is the point of attachment to the carbonyl group,
X represents an oxygen atom,
R² represents C₁-C₄-alkyl or cyclobutyl,
   where alkyl is substituted by a hydroxy substituent,
   and
   where cyclobutyl is substituted by a hydroxy substituent,
R³ represents hydrogen,
R⁴ represents hydrogen or methyl,
and
R⁵ represents methyl,
or
R² represents methyl,
   where methyl may be substituted by 1 to 2 fluorine substituents,
R³ represents hydrogen or methyl,
R⁴ represents C₁-C₄-alkyl,
   where alkyl is substituted by a hydroxy substituent,
and
R⁵ represents methyl,
or
R² and R³ together with the carbon atom to which they are attached form a cyclobutyl ring,
   where the cyclobutyl ring is substituted by a hydroxy substituent,
R⁴ represents hydrogen,
and
R⁵ represents methyl,
R⁷ represents methyl,
R⁸ represents hydrogen,
R⁹ represents methyl or ethyl,
   where methyl may be substituted by a cyano substituent,
R¹⁰ represents hydrogen,
R¹¹ represents methyl,
R¹² represents hydrogen,
or
R¹¹ and R¹² together with the carbon atom to which they are bonded form a cyclopropyl ring,
R¹³ represents methyl,
   where methyl is substituted by a hydroxy substituent,
R¹⁴ represents ethoxy or cyclopropyloxy,
   where ethoxy may be substituted by 1 to 3 substituents selected from the group consisting of deuterium and fluorine,
R¹⁵ represents hydrogen,
   and
   R¹⁶ represents hydrogen or methyl,
   and the salts thereof, the solvates thereof and the solvates of the salts thereof.

Preference is also given to compounds of the formula (I) in which

R¹ represents a group of the formula

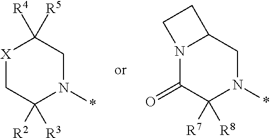

where * is the point of attachment to the carbonyl group,
X represents an oxygen atom, a sulphur atom or CH—R⁶,
   where
   R⁶ represents hydrogen or hydroxy,
R² represents hydrogen, aminocarbonyl, C₁-C₆-alkyl, C₃-C₆-cycloalkyl or phenyl,
   where alkyl and cycloalkyl may be substituted by a substituent selected from the group consisting of hydroxy, methoxy, cyano, hydroxycarbonyl, aminocarbonyl, methylsulphonyl, difluoromethoxy, trifluoromethoxy and cyclopropyl,
      where cyclopropyl for its part may be substituted by a hydroxy substituent,
   or
   where alkyl and cycloalkyl may be substituted by 1 to 3 fluorine substituents,
R³ represents hydrogen or C₁-C₄-alkyl,
or
R² and R³ together with the carbon atom to which they are attached form a cyclopropyl ring, cyclobutyl ring or cyclopentyl ring,
   where the cyclobutyl ring and the cyclopentyl ring may be substituted by a hydroxy substituent,
R⁴ represents hydrogen or C₁-C₆-alkyl,
   where alkyl may be substituted by a hydroxy substituent,
   or
   where alkyl may be substituted by 1 to 3 fluorine substituents,
R⁵ represents C₁-C₄-alkyl,
or
R⁴ and R⁵ together with the carbon atom to which they are attached form a cyclopropyl ring, cyclobutyl ring or cyclopentyl ring,
   where the cyclobutyl ring and the cyclopentyl ring may be substituted by a hydroxy substituent,
R⁷ represents hydrogen or C₁-C₆-alkyl,
   where alkyl may be substituted by one substituent selected from the group consisting of cyano, hydroxy and methoxy,
   or
   where alkyl may be substituted by 1 to 3 fluorine substituents,
R⁸ represents hydrogen,
   and
   R¹⁶ represents hydrogen, methyl or fluoromethyl,
   and the salts thereof, the solvates thereof and the solvates of the salts thereof.

Preference is also given to compounds of the formula (I) in which

R¹ represents a group of the formula

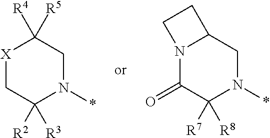

where * is the point of attachment to the carbonyl group,
X represents an oxygen atom or CH—R⁶,
   where
   R⁶ represents hydrogen,
R² represents hydrogen, C₁-C₄-alkyl or C₃-C₆-cycloalkyl,
   where alkyl and cycloalkyl may be substituted by a substituent selected from the group consisting of hydroxy, methoxy, hydroxycarbonyl, difluoromethoxy and cyclopropyl,
      where cyclopropyl for its part may be substituted by a hydroxy substituent,
   or
   where alkyl may be substituted by 1 to 3 fluorine substituents,
R³ represents hydrogen or C₁-C₄-alkyl,
or
R² and R³ together with the carbon atom to which they are attached form a cyclobutyl ring,
   where the cyclobutyl ring may be substituted by a hydroxy substituent,
R⁴ represents hydrogen or C₁-C₄-alkyl,
   where alkyl may be substituted by a hydroxy substituent,
R⁵ represents C₁-C₄-alkyl,
R⁷ represents C₁-C₄-alkyl,
   where alkyl may be substituted by a methoxy substituent,
R⁸ represents hydrogen,
   and
   R¹⁶ represents hydrogen or methyl,
   and the salts thereof, the solvates thereof and the solvates of the salts thereof.

Preference is also given to compounds of the formula (I) in which

R¹ represents a group of the formula

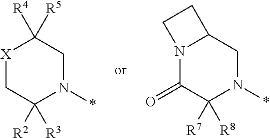

where * is the point of attachment to the carbonyl group,
X represents an oxygen atom,
R² represents C₁-C₄-alkyl or cyclobutyl,
   where alkyl may be substituted by one substituent selected from the group consisting of hydroxy and methoxy,
   or
   where alkyl may be substituted by 1 to 3 fluorine substituents,
   and
   where cyclobutyl is substituted by a hydroxy substituent,
R³ represents hydrogen or methyl,
R⁴ represents hydrogen or methyl,
and
R⁵ represents methyl,
or
R² represents methyl or ethyl,
   where methyl or ethyl may be substituted by 1 to 3 fluorine substituents,
R³ represents hydrogen or methyl,
R⁴ represents C₁-C₄-alkyl,
   where alkyl is substituted by a hydroxy substituent,
and
R⁵ represents methyl,
or
R² and R³ together with the carbon atom to which they are attached form a cyclobutyl ring,
   where the cyclobutyl ring is substituted by a hydroxy substituent,
R⁴ represents hydrogen or methyl,
and
R⁵ represents methyl,
R⁷ represents methyl or ethyl,
   where methyl and ethyl may be substituted by a methoxy substituent,
R⁸ represents hydrogen,
   and
   R¹⁶ represents hydrogen or methyl,
   and the salts thereof, the solvates thereof and the solvates of the salts thereof.

Preference is also given to compounds of the formula (I) in which

R¹ represents a group of the formula

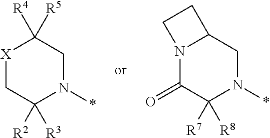

where * is the point of attachment to the carbonyl group,
X represents an oxygen atom,
R² represents C₁-C₄-alkyl or cyclobutyl,
   where alkyl is substituted by a hydroxy substituent,
   and
   where cyclobutyl is substituted by a hydroxy substituent,
R³ represents hydrogen,
R⁴ represents hydrogen or methyl,
and
R⁵ represents methyl,
or
R² represents methyl,
   where methyl may be substituted by 1 to 2 fluorine substituents,
R³ represents hydrogen or methyl,
R⁴ represents C₁-C₄-alkyl,
   where alkyl is substituted by a hydroxy substituent,
and
R⁵ represents methyl,
or
R² and R³ together with the carbon atom to which they are attached form a cyclobutyl ring,
   where the cyclobutyl ring is substituted by a hydroxy substituent,
R⁴ represents hydrogen,
and
R⁵ represents methyl,
R⁷ represents methyl,
R⁸ represents hydrogen,
   and
   R¹⁶ represents hydrogen or methyl,
   and the salts thereof, the solvates thereof and the solvates of the salts thereof.

Preference is also given to compounds of the formula (I) in which

R¹ represents a group of the formula

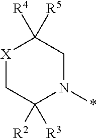

where * is the point of attachment to the carbonyl group,
X represents an oxygen atom, a sulphur atom or CH—R⁶,
where
   R⁶ represents hydrogen or hydroxy,
R² represents hydrogen, aminocarbonyl, C₁-C₆-alkyl, C₃-C₆-cycloalkyl or phenyl,
   where alkyl and cycloalkyl may be substituted by a substituent selected from the group consisting of hydroxy, methoxy, cyano, hydroxycarbonyl, aminocarbonyl, methylsulphonyl, difluoromethoxy, trifluoromethoxy and cyclopropyl,
      where cyclopropyl for its part may be substituted by a hydroxy substituent,
   or
   where alkyl and cycloalkyl may be substituted by 1 to 3 fluorine substituents,
R³ represents hydrogen or C₁-C₄-alkyl,
or
R² and R³ together with the carbon atom to which they are attached form a cyclobutyl ring, cyclobutyl ring or cyclopentyl ring,
   where the cyclobutyl ring and the cyclopentyl ring may be substituted by a hydroxy substituent,
R⁴ represents hydrogen or C₁-C₆-alkyl,
   where alkyl may be substituted by a hydroxy substituent,
   or
   where alkyl may be substituted by 1 to 3 fluorine substituents,
R⁵ represents C₁-C₄-alkyl,
or
R⁴ and R⁵ together with the carbon atom to which they are attached form a cyclopropyl ring, cyclobutyl ring or cyclopentyl ring,
   where the cyclobutyl ring and the cyclopentyl ring may be substituted by a hydroxy substituent,
      and
      R¹⁶ represents hydrogen, methyl or fluoromethyl,
      and the salts thereof, the solvates thereof and the solvates of the salts thereof.

Preference is also given to compounds of the formula (I) in which

R¹ represents a group of the formula

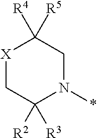

where * is the point of attachment to the carbonyl group,
X represents an oxygen atom or CH—R⁶,
   where
   R⁶ represents hydrogen,
R² represents hydrogen, C₁-C₄-alkyl or C₃-C₆-cycloalkyl,
   where alkyl and cycloalkyl may be substituted by a substituent selected from the group consisting of hydroxy, methoxy, hydroxycarbonyl, difluoromethoxy and cyclopropyl,
      where cyclopropyl for its part may be substituted by a hydroxy substituent,
   or
   where alkyl may be substituted by 1 to 3 fluorine substituents,
R³ represents hydrogen or C₁-C₄-alkyl,
or
R² and R³ together with the carbon atom to which they are attached form a cyclobutyl ring,
   where the cyclobutyl ring may be substituted by a hydroxy substituent,
R⁴ represents hydrogen or C₁-C₄-alkyl,
   where alkyl may be substituted by a hydroxy substituent,
R⁵ represents C₁-C₄-alkyl,
   and
   R¹⁶ represents hydrogen or methyl,
   and the salts thereof, the solvates thereof and the solvates of the salts thereof.

Preference is also given to compounds of the formula (I) in which

R¹ represents a group of the formula

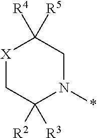

where * is the point of attachment to the carbonyl group,
X represents an oxygen atom,
R² represents C₁-C₄-alkyl or cyclobutyl,
   where alkyl may be substituted by one substituent selected from the group consisting of hydroxy and methoxy,
   or
   where alkyl may be substituted by 1 to 3 fluorine substituents,
   and
   where cyclobutyl is substituted by a hydroxy substituent,
R³ represents hydrogen or methyl,
R⁴ represents hydrogen or methyl,
and
R⁵ represents methyl,
or
R² represents methyl or ethyl,
   where methyl or ethyl may be substituted by 1 to 3 fluorine substituents,
R³ represents hydrogen or methyl,
R⁴ represents C₁-C₄-alkyl,
   where alkyl is substituted by a hydroxy substituent,
and
R⁵ represents methyl,
or
R² and R³ together with the carbon atom to which they are attached form a cyclobutyl ring,
   where the cyclobutyl ring is substituted by a hydroxy substituent,
R⁴ represents hydrogen or methyl,
and
R⁵ represents methyl,
   and
   R¹⁶ represents hydrogen or methyl,
   and the salts thereof, the solvates thereof and the solvates of the salts thereof.

Preference is also given to compounds of the formula (I) in which

R¹ represents a group of the formula

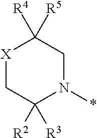

where * is the point of attachment to the carbonyl group,
X represents an oxygen atom,
R² represents C₁-C₄-alkyl or cyclobutyl,
   where alkyl is substituted by a hydroxy substituent,
   and
   where cyclobutyl is substituted by a hydroxy substituent,
R³ represents hydrogen,
R⁴ represents hydrogen or methyl,
and
R⁵ represents methyl,
or
R² represents methyl,
   where methyl may be substituted by 1 to 2 fluorine substituents,
R³ represents hydrogen or methyl,
R⁴ represents C₁-C₄-alkyl,
   where alkyl is substituted by a hydroxy substituent,
and
R⁵ represents methyl,
or
R² and R³ together with the carbon atom to which they are attached form a cyclobutyl ring,
   where the cyclobutyl ring is substituted by a hydroxy substituent,
R⁴ represents hydrogen,
and
R⁵ represents methyl,
   and
   R¹⁶ represents hydrogen or methyl,
   and the salts thereof, the solvates thereof and the solvates of the salts thereof.

Preference is also given to compounds of the formula (I) in which

R¹ represents a group of the formula

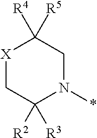

where * is the point of attachment to the carbonyl group,
X represents an oxygen atom,
R² represents C₁-C₄-alkyl or cyclobutyl,
   where alkyl is substituted by a hydroxy substituent,
   and
   where cyclobutyl is substituted by a hydroxy substituent,
R³ represents hydrogen,
R⁴ represents hydrogen or methyl,
and
R⁵ represents methyl,
   and
   R¹⁶ represents hydrogen or methyl,
   and the salts thereof, the solvates thereof and the solvates of the salts thereof.

Preference is also given to compounds of the formula (I) in which

R¹ represents a group of the formula

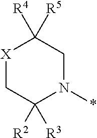

where * is the point of attachment to the carbonyl group,
X represents an oxygen atom,
R² represents methyl,
   where methyl may be substituted by 1 to 2 fluorine substituents,
R³ represents hydrogen or methyl,
R⁴ represents C₁-C₄-alkyl,
   where alkyl is substituted by a hydroxy substituent,
and
R⁵ represents methyl,
   and
   R¹⁶ represents hydrogen or methyl,
   and the salts thereof, the solvates thereof and the solvates of the salts thereof.

Preference is also given to compounds of the formula (I) in which

R¹ represents a group of the formula

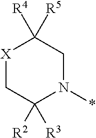

where * is the point of attachment to the carbonyl group,
X represents an oxygen atom,
R² and R³ together with the carbon atom to which they are attached form a cyclobutyl ring,
   where the cyclobutyl ring is substituted by a hydroxy substituent,
R⁴ represents hydrogen,
and
R⁵ represents methyl,
   and
   R¹⁶ represents hydrogen or methyl,
   and the salts thereof, the solvates thereof and the solvates of the salts thereof.

Preference is also given to compounds of the formula (I) in which

R¹ represents a group of the formula

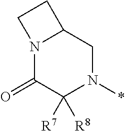

where * is the point of attachment to the carbonyl group,
R⁷ represents hydrogen or C₁-C₆-alkyl,
   where alkyl may be substituted by one substituent selected from the group consisting of cyano, hydroxy and methoxy,
   or
   where alkyl may be substituted by 1 to 3 fluorine substituents,
R⁸ represents hydrogen,
   and
   R¹⁶ represents hydrogen, methyl or fluoromethyl,
   and the salts thereof, the solvates thereof and the solvates of the salts thereof.

Preference is also given to compounds of the formula (I) in which

R¹ represents a group of the formula

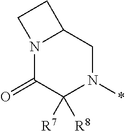

where * is the point of attachment to the carbonyl group,
R⁷ represents C₁-C₄-alkyl,
   where alkyl may be substituted by a methoxy substituent,
R⁸ represents hydrogen,
   and
   R¹⁶ represents hydrogen or methyl,
   and the salts thereof, the solvates thereof and the solvates of the salts thereof.

Preference is also given to compounds of the formula (I) in which

R¹ represents a group of the formula

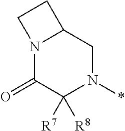

where * is the point of attachment to the carbonyl group,
X represents an oxygen atom,
R⁷ represents methyl or ethyl,
   where methyl and ethyl may be substituted by a methoxy substituent,
R⁸ represents hydrogen,
   and
   R¹⁶ represents hydrogen or methyl,
   and the salts thereof, the solvates thereof and the solvates of the salts thereof.

Preference is also given to compounds of the formula (I) in which

R¹ represents a group of the formula

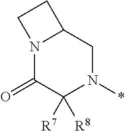

where * is the point of attachment to the carbonyl group,
R⁷ represents methyl,
R⁸ represents hydrogen,
   and
   R¹⁶ represents hydrogen or methyl,
   and the salts thereof, the solvates thereof and the solvates of the salts thereof.

Preference is also given to compounds of the formula (I) in which

R¹ represents a group of the formula

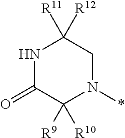

where * is the point of attachment to the carbonyl group,
R⁹ represents hydrogen or C₁-C₆-alkyl,
   where alkyl may be substituted by one substituent selected from the group consisting of hydroxy, cyano and aminocarbonyl,
   or
   where alkyl may be substituted by 1 to 3 fluorine substituents,
R¹⁰ represents hydrogen,
R¹ represents C₁-C₄-alkyl,
   where alkyl may be substituted by a hydroxy substituent,
R¹² represents hydrogen or C₁-C₄-alkyl,
or
R¹¹ and R¹² together with the carbon atom to which they are attached form a cyclopropyl ring, cyclobutyl ring or cyclopentyl ring,
   where the cyclobutyl ring and the cyclopentyl ring may be substituted by a hydroxy substituent,
      and
      R¹⁶ represents hydrogen, methyl or fluoromethyl,
      and the salts thereof, the solvates thereof and the solvates of the salts thereof.

Preference is also given to compounds of the formula (I) in which

R¹ represents a group of the formula

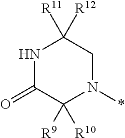

where * is the point of attachment to the carbonyl group,
R⁹ represents C₁-C₄-alkyl,
   where alkyl may be substituted by one substituent selected from the group consisting of cyano and aminocarbonyl,
R¹⁰ represents hydrogen,
R¹¹ represents C₁-C₄-alkyl,
R¹² represents hydrogen or C₁-C₄-alkyl,
or
R¹¹ and R¹² together with the carbon atom to which they are bonded form a cyclopropyl ring,
   and
   R¹⁶ represents hydrogen or methyl,
   and the salts thereof, the solvates thereof and the solvates of the salts thereof.

Preference is also given to compounds of the formula (I) in which

R¹ represents a group of the formula

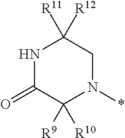

where * is the point of attachment to the carbonyl group,
R⁹ represents methyl or ethyl,
   where methyl and ethyl may be substituted by a substituent selected from the group consisting of cyano and aminocarbonyl,
R¹⁰ represents hydrogen,
R¹¹ represents methyl,
R¹² represents hydrogen,
or
R¹¹ and R¹² together with the carbon atom to which they are bonded form a cyclopropyl ring,
   and
   R¹⁶ represents hydrogen or methyl,
   and the salts thereof, the solvates thereof and the solvates of the salts thereof.

Preference is also given to compounds of the formula (I) in which

R¹ represents a group of the formula

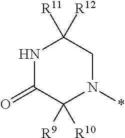

where * is the point of attachment to the carbonyl group,
R⁹ represents methyl or ethyl,
   where methyl may be substituted by a cyano substituent,
R¹⁰ represents hydrogen,
R¹¹ represents methyl,
R¹² represents hydrogen,
or
R¹¹ and R¹² together with the carbon atom to which they are bonded form a cyclopropyl ring,
   and
   R¹⁶ represents hydrogen or methyl,
   and the salts thereof, the solvates thereof and the solvates of the salts thereof.

Preference is also given to compounds of the formula (I) in which

R¹ represents a group of the formula

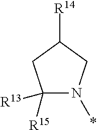

where * is the point of attachment to the carbonyl group,
R¹³ represents methyl, ethyl, (3-fluoroazetidin-1-yl)carbonyl, (3,3-difluoroazetidin-1-yl)carbonyl or morpholin-4-ylcarbonyl,
   where methyl and ethyl are substituted by a substituent selected from the group consisting of cyano and hydroxy,
R¹⁴ represents hydrogen, methoxy, ethoxy or cyclopropyloxy,
   where methoxy and ethoxy may be substituted by 1 to 3 substituents selected from the group consisting of deuterium and fluorine,
R¹⁵ represents hydrogen or methyl,
   and
   R¹⁶ represents hydrogen, methyl or fluoromethyl,
   and the salts thereof, the solvates thereof and the solvates of the salts thereof.

Preference is also given to compounds of the formula (I) in which

R¹ represents a group of the formula

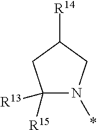

where * is the point of attachment to the carbonyl group,
R¹³ represents methyl, ethyl, (3-fluoroazetidin-1-yl)carbonyl, (3,3-difluoroazetidin-1-yl)carbonyl or morpholin-4-ylcarbonyl,
   where methyl and ethyl are substituted by a substituent selected from the group consisting of cyano and hydroxy,
R¹⁴ represents hydrogen, ethoxy or cyclopropyloxy,
   where ethoxy may be substituted by 1 to 3 substituents selected from the group consisting of deuterium and fluorine,
R¹⁵ represents hydrogen or methyl,
   and
   R¹⁶ represents hydrogen or methyl,
   and the salts thereof, the solvates thereof and the solvates of the salts thereof.

Preference is also given to compounds of the formula (I) in which

R¹ represents a group of the formula

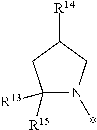

where * is the point of attachment to the carbonyl group,
R¹³ represents methyl,
   where methyl is substituted by a hydroxy substituent,
R¹⁴ represents ethoxy or cyclopropyloxy,
   where ethoxy may be substituted by 1 to 3 substituents selected from the group consisting of deuterium and fluorine,
R¹⁵ represents hydrogen,
   and
   R¹⁶ represents hydrogen or methyl,
   and the salts thereof, the solvates thereof and the solvates of the salts thereof.

Preference is also given to compounds of the formula (I) in which R¹⁶ represents hydrogen.

Preference is also given to compounds of the formula (I) in which R¹⁶ represents methyl.

Preference is also given to compounds having the formula (Ia)

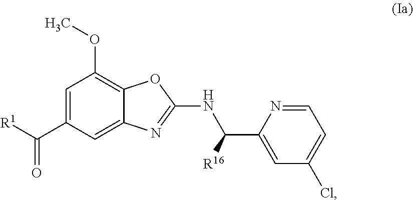

where R¹ and R¹⁵ are as defined above.

Preference is also given to
(2-{[(4-chloropyridin-2-yl)methyl]amino}-7-methoxy-1,3-benzoxazol-5-yl)[(5R)-2-(2-hydroxyethyl)-2,5-dimethylmorpholin-4-yl]methanone [enantiomerically pure isomer]
   or
(2-{[(4-chloropyridin-2-yl)methyl]amino}-7-methoxy-1,3-benzoxazol-5-yl) [5-(2-hydroxypropan-2-yl)-2-methylmorpholin-4-yl]methanone [enantiomerically pure isomer 2]
   or
7-[(2-{[(4-chloropyridin-2-yl)methyl]amino}-7-methoxy-1,3-benzoxazol-5-yl)carbonyl]-6-ethyl-4,7-diazaspiro[2.5]octan-5-one [enantiomerically pure isomer 1]
   or
{1-[(2-{[(4-chloropyridin-2-yl)methyl]amino}-7-methoxy-1,3-benzoxazol-5-yl)carbonyl]-5,5-dimethyl-3-oxopiperazin-2-yl}acetonitrile [enantiomerically pure isomer 2]
   or
(2-{[(4-chloropyridin-2-yl)methyl]amino}-7-methoxy-1,3-benzoxazol-5-yl)[5-(1-hydroxyethyl)-2,2-dimethylmorpholin-4-yl]methanone [enantiomerically pure isomer 1]
   or one of the salts, the solvates or the solvates of the salts of these compounds.

The invention further provides a process for preparing the compounds of the formula (I), or the salts thereof, solvates thereof and the solvates of the salts thereof, wherein the compounds of the formula

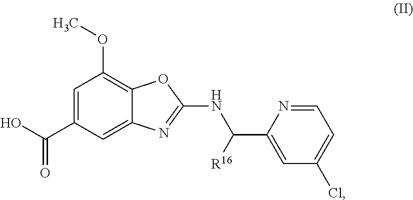

in which
R¹⁶ is as defined above,
are reacted with compounds of the formula

R¹—H (III),

in which
R¹ is as defined above,
and dehydrating agents.

The reaction is generally carried out in inert solvents, if appropriate in the presence of a base, preferably in a temperature range from 0° C. to room temperature at atmospheric pressure.

Suitable dehydrating agents here are, for example, carbodiimides such as N,N′-diethyl-, N,N′-dipropyl-, N,N′-diisopropyl-, N,N′-dicyclohexylcarbodiimide, N-(3-dimethylaminoisopropyl)-N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC) (optionally in the presence of pentafluorophenol (PFP)), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulphate or 2-tert-butyl-5-methyl-isoxazolium perchlorate, or acylamino compounds such as 2-ethoxy-1-ethoxycarbonyl-1,2-dihydroquinoline, or propanephosphonic anhydride, or isobutyl chloroformate, or bis-(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), (benzotriazol-1-yloxy)bisdimethylaminomethylium fluoroborate (TBTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or mixtures of these, with bases. Preferably, the condensation is carried out using HATU or N-(3-dimethylaminoisopropyl)-N′-ethylcarbodiimide hydrochloride (EDC).

Bases are, for example, alkali metal carbonates such as sodium carbonate or potassium carbonate, or sodium bicarbonate or potassium bicarbonate, or organic bases such as trialkylamines, for example triethylamine, N-methylmorpholine, N-methylpiperidine, 4-dimethylaminopyridine or diisopropylethylamine, preference being given to diisopropylethylamine or 4-dimethylaminopyridine.

Preference is given to the combination of HATU and diisopropylethylamine or N-(3-dimethylaminoisopropyl)-N′-ethylcarbodiimide hydrochloride (EDC) and 4-dimethylaminopyridine.

Inert solvents are, for example, halogenated hydrocarbons such as dichloromethane or trichloromethane, hydrocarbons such as benzene, or other solvents such as nitromethane, dioxane, dimethylformamide, dimethyl sulphoxide or acetonitrile, or mixtures of the solvents; preference is given to dimethylformamide.

The compounds of the formula (III) are known, can be synthesized from the corresponding starting compounds by known processes or can be prepared analogously to the processes described in the Examples section.

The compound of the formula (II) is known or can be prepared by reacting the compounds of the formula

in which

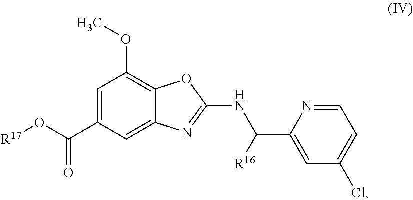

R¹⁶ has the meaning given above and
R¹⁷ represents methyl or ethyl
with a base.

The reaction is generally carried out in inert solvents, preferably in a temperature range of from 0° C. to room temperature at atmospheric pressure.

Bases are, for example, alkali metal hydroxides such as sodium hydroxide, lithium hydroxide or potassium hydroxide, or alkali metal carbonates such as caesium carbonate, sodium carbonate or potassium carbonate; preference is given to sodium hydroxide.

Inert solvents are, for example, halogenated hydrocarbons such as dichloromethane, trichloromethane, carbon tetrachloride, trichloroethane, tetrachloroethane, 1,2-dichloroethane or trichloroethylene, ethers such as diethyl ether, methyl tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or mineral oil fractions, or other solvents such as dimethylformamide, dimethylacetamide, dimethyl sulphoxide, acetonitrile or pyridine, or mixtures of solvents; preference is given to dioxane.

The compounds of the formula (IV) are known or can be prepared by reacting compounds of the formula

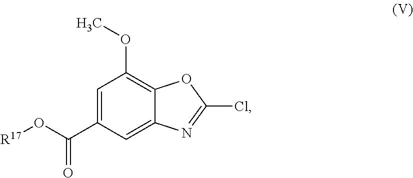

in which
R¹⁷ represents methyl or ethyl
with compounds of the formula

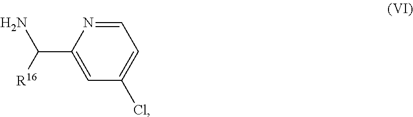

in which
R¹⁶ has the meaning given above,
in the presence of a base.

The reaction is generally carried out in inert solvents, preferably in a temperature range of from room temperature to reflux of the solvents at atmospheric pressure.

The compounds of the formulae (V) and (VI) are known, can be synthesized from the corresponding starting compounds by known processes or can be prepared analogously to the processes described in the Examples section.

The preparation of the starting compounds and of the compounds of the formula (I) can be illustrated by the synthesis scheme below.

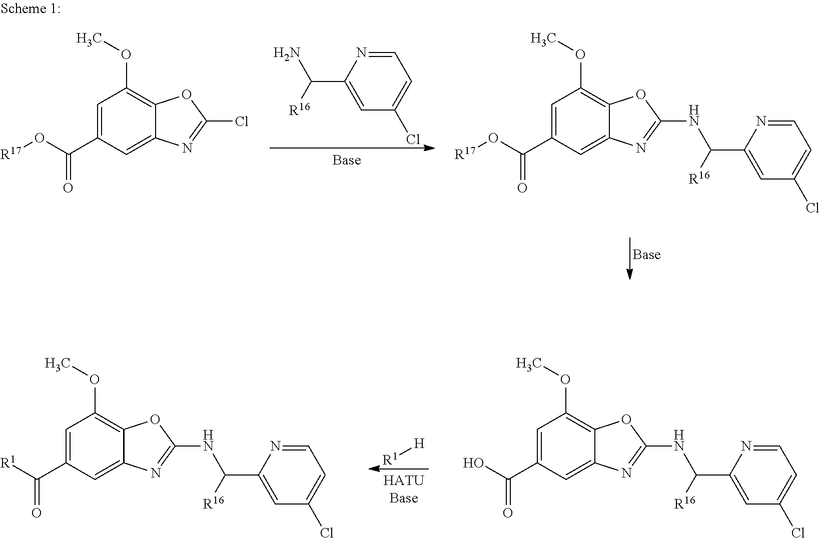

The compounds according to the invention have an unforeseeable useful pharmacological activity spectrum and good pharmacokinetic behaviour. They are compounds modulating the proteolytic activity of the serine protease thrombin. The compounds according to the invention inhibit the thrombin-catalysed enzymatic cleavage of substrates which play an essential role in the activation of blood coagulation, platelet aggregation (via PAR-1 activation of the platelets) and thrombin-induced inflammation, fibrosis and angiogenesis processes.

They are therefore suitable for use as medicaments for the treatment and/or prophylaxis of diseases in humans and animals.

The present invention further provides for the use of the compounds according to the invention for the treatment and/or prophylaxis of disorders, in particular cardiovascular disorders, preferably thrombotic or thromboembolic disorders and/or thrombotic or thromboembolic complications.

As a key enzyme at the end of the coagulation cascade, thrombin translates, via a series of conversions, the impulses of the cascade into the coagulation state of the blood. By conversion of fibrinogen into insoluble fibrin, fibrin clots are formed, which are stabilized by factor XIIIa likewise activated by thrombin. By activating TAFI (thrombin-activatable fibrinolysis inhibitor) to TAFIa, thrombin in a complex with thrombomodulin inhibits the dissolution of the clot. Activation of factors V and VIII potentiates the production of thrombin and thus in turn amplifies the coagulation reaction. In addition, thrombin is a potent trigger of platelet aggregation (via PAR-1 activation), which also contributes considerably to haemostasis.

Accordingly, the compounds according to the invention are suitable for the treatment and/or prophylaxis of disorders or complications which arise or may arise from the formation of clots.

For the purpose of the present invention, the “thrombotic or thromboembolic disorders” include disorders which occur both in the arterial and in the venous vasculature and which can be treated with the compounds according to the invention, in particular disorders in the coronary arteries of the heart, such as acute coronary syndrome (ACS), myocardial infarction with ST segment elevation (STEMI) and without ST segment elevation (non-STEMI), stable angina pectoris, unstable angina pectoris, reocclusions and restenoses after coronary interventions such as angioplasty, stent implantation or aortocoronary bypass, but also thrombotic or thromboembolic disorders in further vessels leading to peripheral arterial occlusive disorders, pulmonary embolisms, venous thromboembolisms, venous thromboses, in particular in deep leg veins and kidney veins, transitory ischaemic attacks and also thrombotic stroke and thromboembolic stroke.

Stimulation of the coagulation system may occur by various causes or associated disorders. In the context of surgical interventions, immobility, confinement to bed, infections or cancer or cancer therapy, inter alia, the coagulation system can be highly activated, and there may be thrombotic complications, in particular venous thromboses. The compounds according to the invention are therefore suitable for the prophylaxis of thromboses in the context of surgical interventions in patients suffering from cancer. The compounds according to the invention are therefore also suitable for the prophylaxis of thromboses in patients having an activated coagulation system, for example in the stimulation situations described.

The inventive compounds are therefore also suitable for the prevention and treatment of cardiogenic thromboembolisms, for example brain ischaemias, stroke and systemic thromboembolisms and ischaemias, in patients with acute, intermittent or persistent cardial arrhythmias, for example atrial fibrillation, and those undergoing cardioversion, and also in patients with heart valve disorders or with artificial heart valves.

Thromboembolic complications are also encountered in microangiopathic haemolytic anaemias, extracorporeal circulatory systems, such as haemodialysis, and also prosthetic heart valves.

Moreover, the compounds according to the invention are particularly suitable for the treatment of disorders where a clot is already present, since in particular thrombin incorporated in the clot helps to stabilize the clot. Since the inhibition of these thrombin molecules accelerates the degradation of the clot, the compounds according to the invention can be used for the treatment of existing clots. These clots may be formed in the entire vascular system and may cause grave complications in various organs, in particular via ischaemia, inflammatory reactions or formation of embolisms, for example myocardial infarction or stroke, but also pulmonary embolism or post-thrombotic syndrome in particular after deep vein thromboses in the leg. Accordingly, the compounds according to the invention are also suitable for the treatment of venous and arterial occlusions of the ocular blood vessels caused by clots, for example age-related macular degeneration.

By virtue of the synergistic effects observed with lytic therapeutic principles such as the tissue plasminogen activator (tPA), the compounds are suitable for adjunctive use in the context of thrombolysis therapy.

Moreover, the compounds according to the invention are suitable for the treatment and/or prophylaxis of disorders involving microclot formation or fibrin deposits in cerebral blood vessels which may lead to dementia disorders such as vascular dementia or Alzheimer's disease. Here, the clot may contribute to the disorder both via occlusions and by binding further disease-relevant factors.

Moreover, the compounds according to the invention are suitable in particular for the treatment and/or prophylaxis of disorders where, in addition to the pro-coagulant component, the pro-inflammatory component of thrombin action plays an essential role. Mutual enhancement of coagulation and inflammation in particular can be prevented by the compounds according to the invention, thus decisively lowering the probability of thrombotic complications. Here, the treatment and/or prophylaxis in the context of atherosclerotic vascular disorders, inflammations in the context of rheumatic disorders of the locomotor system, inflammatory disorders of the lung, such as pulmonary fibroses, inflammatory disorders of the kidney, such as glomerulonephritides, inflammatory disorders of the intestine, such as Crohn's disease or ulcerative colitis, or disorders which may be present in the context of a diabetic underlying disease, such as diabetic retinopathy or nephropathy, may be considered, inter alia.

Moreover, the compounds according to the invention can be used for inhibiting tumour growth and the formation of metastases, and also for the prophylaxis and/or treatment of thromboembolic complications, such as, for example, venous thromboembolisms, for tumour patients, in particular those undergoing major surgical interventions or chemo- or radiotherapy.

In addition, the inventive compounds are also suitable for the prophylaxis and/or treatment of pulmonary hypertension.

In the context of the present invention, the term “pulmonary hypertension” includes pulmonary arterial hypertension, pulmonary hypertension associated with disorders of the left heart, pulmonary hypertension associated with pulmonary disorders and/or hypoxia and pulmonary hypertension owing to chronic thromboembolisms (CTEPH).

“Pulmonary arterial hypertension” includes idiopathic pulmonary arterial hypertension (IPAH, formerly also referred to as primary pulmonary hypertension), familial pulmonary arterial hypertension (FPAH) and associated pulmonary-arterial hypertension (APAH), which is associated with collagenoses, congenital systemic-pulmonary shunt vitia, portal hypertension, HIV infections, the ingestion of certain drugs and medicaments, with other disorders (thyroid disorders, glycogen storage disorders, Morbus Gaucher, hereditary teleangiectasia, haemoglobinopathies, myeloproliferative disorders, splenectomy), with disorders having a significant venous/capillary contribution, such as pulmonary-venoocclusive disorder and pulmonary-capillary haemangiomatosis, and also persisting pulmonary hypertension of neonatants.

Pulmonary hypertension associated with disorders of the left heart includes a diseased left atrium or ventricle and mitral or aorta valve defects.

Pulmonary hypertension associated with pulmonary disorders and/or hypoxia includes chronic obstructive pulmonary disorders, interstitial pulmonary disorder, sleep apnoea syndrome, alveolar hypoventilation, chronic high-altitude sickness and inherent defects.

Pulmonary hypertension owing to chronic thromboembolisms (CTEPH) comprises the thromboembolic occlusion of proximal pulmonary arteries, the thromboembolic occlusion of distal pulmonary arteries and non-thrombotic pulmonary embolisms (tumour, parasites, foreign bodies).

The present invention further provides for the use of the inventive compounds for production of medicaments for the treatment and/or prophylaxis of pulmonary hypertension associated with sarcoidosis, histiocytosis X and lymphangiomatosis.

In addition, the inventive substances may also be useful for the treatment of pulmonary and hepatic fibroses.

In addition, the inventive compounds may also be suitable for the treatment and/or prophylaxis of disseminated intravascular coagulation in the context of an infectious disease, and/or of systemic inflammatory syndrome (SIRS), septic organ dysfunction, septic organ failure and multiorgan failure, acute respiratory distress syndrome (ARDS), acute lung injury (ALI), septic shock and/or septic organ failure.

In the course of an infection, there may be a generalized activation of the coagulation system (disseminated intravascular coagulation or consumption coagulopathy, hereinbelow referred to as “DIC”) with microthrombosis in various organs and secondary haemorrhagic complications. Moreover, there may be endothelial damage with increased permeability of the vessels and seeping of fluids and proteins into the extravasal lumen. As the infection progresses, there may be failure of an organ (for example kidney failure, liver failure, respiratory failure, central-nervous deficits and cardiovascular failure) or multiorgan failure.

In the case of DIC, there is a massive activation of the coagulation system at the surface of damaged endothelial cells, the surfaces of foreign bodies or injured extravascular tissue. As a consequence, there is coagulation in small vessels of various organs with hypoxia and subsequent organ dysfunction. A secondary effect is the consumption of coagulation factors (for example factor X, prothrombin and fibrinogen) and platelets, which reduces the coagulability of the blood and may result in heavy bleeding.

The compounds according to the invention are very particularly suitable for the treatment and/or prophylaxis of acute coronary syndrome (ACS), venous thromboembolisms, venous thromboses, in particular in deep leg veins and kidney veins, pulmonary embolisms, stroke and/or thrombosis prophylaxis in the context of surgical interventions, in particular in the context of surgical interventions in patients suffering from cancer.

The present invention further provides for the use of the compounds according to the invention for the treatment and/or prophylaxis of disorders, especially the disorders mentioned above.

The present invention further provides for the use of the compounds according to the invention for production of a medicament for the treatment and/or prophylaxis of disorders, especially the disorders mentioned above.

The present invention further provides a method for the treatment and/or prophylaxis of disorders, especially the disorders mentioned above, using a therapeutically effective amount of a compound according to the invention.

The present invention further provides the compounds according to the invention for use in a method for the treatment and/or prophylaxis of disorders, especially the disorders mentioned above, using a therapeutically effective amount of a compound according to the invention.

The present invention further provides medicaments comprising a compound according to the invention and one or more further active compounds.

In addition, the compounds according to the invention can also be used for preventing coagulation ex vivo, for example for the protection of organs to be transplanted against organ damage caused by formation of clots and for protecting the organ recipient against thromboemboli from the transplanted organ, for preserving blood and plasma products, for cleaning/pretreating catheters and other medical auxiliaries and instruments, for coating synthetic surfaces of medical auxiliaries and instruments used in vivo or ex vivo or for biological samples which may comprise factor IIa.

The present invention further provides a method for preventing the coagulation of blood in vitro, in particular in banked blood or biological samples which may contain factor IIa, which method is characterized in that an anticoagulatory amount of the compound according to the invention is added.

The present invention further provides medicaments comprising a compound according to the invention and one or more further active compounds, in particular for the treatment and/or prophylaxis of the disorders mentioned above. Preferred examples of active compounds suitable for combinations include:
lipid-lowering substances, especially HMG-CoA (3-hydroxy-3-methylglutaryl-coenzyme A) reductase inhibitors, for example lovastatin (Mevacor), simvastatin (Zocor), pravastatin (Pravachol), fluvastatin (Lescol) and atorvastatin (Lipitor);
coronary therapeutics/vasodilators, especially ACE (angiotensin converting enzyme) inhibitors, for example captopril, lisinopril, enalapril, ramipril, cilazapril, benazepril, fosinopril, quinapril and perindopril, or AII (angiotensin II) receptor antagonists, for example embusartan, losartan, valsartan, irbesartan, candesartan, eprosartan and temisartan, or β-adrenoceptor antagonists, for example carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, for example prazosine, bunazosine, doxazosine and terazosine, or diuretics, for example hydrochlorothiazide, furosemide, bumetanide, piretanide, torasemide, amiloride and dihydralazine, or calcium channel blockers, for example verapamil and diltiazem, or dihydropyridine derivatives, for example nifedipin (Adalat) and nitrendipine (Bayotensin), or nitro preparations, for example isosorbide 5-mononitrate, isosorbide dinitrate and glycerol trinitrate, or substances causing an increase in cyclic guanosine monophosphate (cGMP), for example stimulators of soluble guanylate cyclase, for example riociguat;
plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors) such as, for example, tissue plasminogen activator (t-PA, for example Actilyse®), streptokinase, reteplase and urokinase;
anticoagulatory substances (anticoagulants), for example heparin (UFH), low-molecular-weight heparins (LMWH), for example tinzaparin, certoparin, parnaparin, nadroparin, ardeparin, enoxaparin, reviparin, dalteparin, danaparoid, semuloparin (AVE 5026), adomiparin (M118) and EP-42675/ORG42675;
direct thrombin inhibitors (DTI) such as, for example, Pradaxa (dabigatran), atecegatran (AZD-0837), DP-4088, SSR-182289A, argatroban, bivalirudin and tanogitran (BIBT-986 and prodrug BIBT-1011), hirudin;
direct factor Xa inhibitors such as, for example, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, idraparinux and fondaparinux;
platelet aggregation-inhibiting substances (platelet aggregation inhibitors, thrombocyte aggregation inhibitors), for example acetylsalicylic acid (for example Aspirin), ticlopidine (Ticlid), clopidogrel (Plavix), prasugrel, ticagrelor, cangrelor, elinogrel, vorapaxar;
fibrinogen receptor antagonists (glycoprotein-IIb/IIIa antagonists), for example abciximab, eptifibatide, tirofiban, lamifiban, lefradafiban and fradafiban;
recombinant human activated protein C such as, for example, Xigris;
and also antiarrhythmics.

The present invention furthermore provides the combination of a compound according to the invention and 5-chloro-N-({(5S)-2-oxo-3-[4-(3-oxo-4-morpholinyl)-phenyl]-1,3-oxazolidin-5-yl}methyl)-2-thiophenecarboxamide (rivaroxaban) [WO 01/47919] having the structural formula

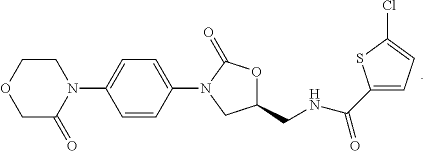 [SMILES: O=C(NC[C@H]1CN(c2ccc(N3CCOCC3=O)cc2)C(=O)O1)c1ccc(Cl)s1]

The compounds according to the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds according to the invention can be administered in suitable administration forms for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay, which control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

Oral administration is preferred.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

The inventive compounds can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctants.

The present invention further provides medicaments comprising at least one inventive compound, preferably together with one or more inert nontoxic pharmaceutically suitable excipients, and the use thereof for the purposes mentioned above.

In the case of parenteral administration, it has generally been found to be advantageous to administer amounts of about 5 to 250 mg every 24 hours to achieve effective results. In the case of oral administration, the amount is about 5 to 500 mg every 24 hours.

It may nevertheless be necessary in some cases to deviate from the stated amounts, specifically as a function of the body weight, route of administration, individual response to the active ingredient, nature of the preparation and time or interval over which administration takes place.

Unless stated otherwise, the percentages in the tests and examples which follow are percentages by weight; parts are parts by weight. Solvent ratios, dilution ratios and concentration data for the liquid/liquid solutions are based in each case on volume. “w/v” means “weight/volume”. For example, “10% w/v” means: 100 ml of solution or suspension comprise 10 g of substance.

### A) EXAMPLES

### Abbreviations

br. d. broad doublet (in NMR)
br. m. broad multiplet (in NMR)
br. s. broad singlet (in NMR)
d day(s), doublet (in NMR)
TLC thin-layer chromatography
DCI direct chemical ionization (in MS)
dd doublet of doublets (in NMR)
DMAP 4-dimethylaminopyridine
DMF N,N-dimethylformamide
DMSO dimethyl sulphoxide
dt doublet of triplets (in NMR)
EDCI N-[3-(dimethylamino)propyl]-N′-ethylcarbodiimide
ESI electrospray ionization (in MS)
GC-MS gas chromatography-coupled mass spectroscopy
h hour(s)
HATU O-(7-azabenzotriazol-1-yl)-N,N,N′,N′-tetramethyluronium-hexafluorophosphate
HPLC high-pressure, high-performance liquid chromatography
LC-MS liquid chromatography-coupled mass spectroscopy
m multiplet (in NMR)
M molar
m_{c} centred multiplet (in NMR)
min minute(s)
MS mass spectroscopy
prep. preparative (in HPLC)
N normal
NMR nuclear magnetic resonance spectroscopy
q quartet (in NMR)
quant. quantitative
quin quintet (in NMR)
RP reversed phase (in HPLC)
RT room temperature
Rₜ retention time (in HPLC)
s singlet (in NMR)
t triplet (in NMR)
TFA trifluoroacetic acid
THF tetrahydrofuran
UV ultraviolet
UPLC ultra high pressure, ultra high performance liquid chromatography

### LC-MS Methods:

Method 1A: Instrument: Waters ACQUITY SQD UPLC system; column: Waters Acquity UPLC HSS T3 1.8μ 50×1 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 208-400 nm.

Method 2A: Instrument: Waters ACQUITY SQD UPLC system; column: Waters Acquity UPLC HSS T3 1.8μ 30×2 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; oven: 50° C.; flow rate: 0.60 ml/min; UV detection: 208-400 nm.

Method 3A: Instrument: Micromass Quattro Premier with Waters UPLC Acquity; column: Thermo Hypersil GOLD 1.9μ 50×1 mm; mobile phase A: 1 l of water+0.5 ml of 50% strength formic acid, mobile phase B: 1 l of acetonitrile+0.5 ml of 50% strength formic acid; gradient: 0.0 min 97% A→0.5 min 97% A→3.2 min 5% A→4.0 min 5% A; oven: 50° C.; flow rate: 0.3 ml/min; UV detection: 210 nm.

Method 4A: MS instrument: Waters (Micromass) Quattro Micro; HPLC instrument: Agilent 1100 series; column: YMC-Triart C18 3μ 50×3 mm; mobile phase A: 1 l of water+0.01 mol of ammonium carbonate, mobile phase B: 1 l of acetonitrile; gradient: 0.0 min 100% A→2.75 min 5% A→4.5 min 5% A; oven: 40° C.; flow rate: 1.25 ml/min; UV detection: 210 nm.

Method 5A: MS instrument: Waters (Micromass) QM; HPLC instrument: Agilent 1100 series; column: Agient ZORBAX Extend-C18 3.0×50 mm 3.5-Micron; mobile phase A: 1 l of water+0.01 mol of ammonium carbonate, mobile phase B: 1 l of acetonitrile; gradient: 0.0 min 98% A→0.2 min 98% A→3.0 min 5% A→4.5 min 5% A; oven: 40° C.; flow rate: 1.75 ml/min; UV detection: 210 nm.

Method 6A: MS instrument: Waters (Micromass) ZQ; HPLC instrument: Agilent 1100 series; column: Agient ZORBAX Extend-C18 3.0×50 mm 3.5-Micron; mobile phase A: 1 l of water+0.01 mol of ammonium carbonate, mobile phase B: 1 l of acetonitrile; gradient: 0.0 min 98% A→0.2 min 98% A→3.0 min 5% A→4.5 min 5% A; oven: 40° C.; flow rate: 1.75 ml/min; UV detection: 210 nm.

Method 7A: Instrument: Waters ACQUITY SQD UPLC system; column: Waters Acquity UPLC HSS T3 1.8μ 50×1 mm; mobile phase A: 1 l of water+0.25 ml of 99% strength formic acid, mobile phase B: 1 l of acetonitrile+0.25 ml of 99% strength formic acid; gradient: 0.0 min 95% A→6.0 min 5% A→7.5 min 5% A; oven: 50° C.; flow rate: 0.35 ml/min; UV detection: 210-400 nm.

### GC-MS Methods:

Method 1B: Instrument: Thermo DFS, Trace GC Ultra; column: Restek RTX-35, 15 m×200 μm×0.33 μm; constant helium flow rate: 1.20 ml/min; oven: 60° C.; inlet: 220° C.; gradient: 60° C., 30° C./min→300° C. (maintain for 3.33 min).

Method 2B: Instrument: Micromass GCT, GC6890; column: Restek RTX-35, 15 m×200 μm×0.33 μm; constant helium flow rate: 0.88 ml/min; oven: 70° C.; inlet: 250° C.; gradient: 70° C., 30° C./min→310° C. (maintain for 3 min).

### MS Methods:

Method 1C: Instrument: Thermo Fisher-Scientific DSQ; chemical ionization; reactant gas NH₃; source temperature: 200° C.; ionization energy 70 eV.

Method 2C: Instrument: Waters ZQ 2000; electrospray ionization; mobile phase A: 1 l of water+0.25 ml of 99% strength formic acid, mobile phase B: 1 l of acetonitrile+0.25 ml of 99% strength formic acid; 25% A, 75% B; flow rate: 0.25 ml/min.

### Preparative Enantiomer/Diastereomer Separation on a Chiral Phase:

Method 1D: phase: Daicel Chiralpak AZ-H, 5 μm 250 mm×30 mm, mobile phase: isohexane/ethanol 50:50; flow rate: 40 ml/min; temperature: 20° C.; UV detection: 220 nm.

Method 2D: phase: Daicel Chiralpak AZ-H, 5 μm 250 mm×30 mm, mobile phase: isohexane/ethanol 50:50; flow rate: 40 ml/min, temperature: 25° C.; UV detection: 220 nm.

Method 3D: phase: Daicel Chiralpak AD-H SFC, 10 μm 250 mm×20 mm, mobile phase: carbon dioxide/ethanol 70:30; flow rate: 100 ml/min, makeup flow rate: 30 ml/min, back pressure: 80 bar; temperature: 40° C.; UV detection: 220 nm.

Method 4D: phase: Daicel Chiralpak AD-H, 5 μm 250 mm×20 mm, mobile phase: isohexane/isopropanol 70:30; flow rate: 20 ml/min; temperature: 25° C.; UV detection: 230 nm.

Method 5D: phase: Daicel Chiralpak AZ-H, 5 μm 250 mm×30 mm, mobile phase: isohexane/ethanol 90:10; flow rate: 40 ml/min; temperature: 25° C.; UV detection: 220 nm.

Method 6D: phase: Daicel Chiralpak AY-H, 5 μm 250 mm×20 mm, mobile phase: isohexane/ethanol 90:10; flow rate: 40 ml/min; temperature: 40° C.; UV detection: 220 nm.

Method 7D: phase: Daicel Chiralpak AS-H, 5 μm 250 mm×20 mm, mobile phase: isohexane/ethanol 70:30; flow rate: 20 ml/min; temperature: 25° C.; UV detection: 230 nm.

Method 8D: phase: Daicel Chiralpak AZ-H, 5 μm 250 mm×30 mm, mobile phase: isohexane/ethanol 50:50; flow rate: 20 ml/min; temperature: 25° C.; UV detection: 220 nm.

Method 9D: phase: Daicel Chiralpak OZ-H, 5 μm 250 mm×20 mm, mobile phase: isohexane/ethanol 50:50; flow rate: 15 ml/min; temperature: 30° C.; UV detection: 220 nm.

Method 10D: phase: Daicel Chiralpak OD-H, 5 μm 250 mm×20 mm, mobile phase: isohexane/ethanol 60:40; flow rate: 20 ml/min; temperature: 22° C.; UV detection: 230 nm.

Method 11D: phase: Daicel Chiralpak AD-H SFC, 10 μm 250 mm×20 mm, mobile phase: carbon dioxide/methanol 70:30; flow rate: 100 ml/min, makeup flow rate: 30 ml/min, back pressure: 80 bar; temperature: 40° C.; UV detection: 210 nm.

Method 12D: phase: Daicel Chiralcel AD-H, 5 μm 250 mm×20 mm; mobile phase: isohexane/ethanol 50:50+0.2% diethylamine; flow rate: 20 ml/min; temperature: 20° C.; UV detection: 220 nm.

Method 13D: phase: Daicel Chiralcel AD-H, 5 μm 250 mm×20 mm; mobile phase: isohexane/isopropanol 50:50+0.2% diethylamine; flow rate: 20 ml/min; temperature: 20° C.; UV detection: 230 nm.

Method 14D: phase: Daicel Chiralpak OD-H, 5 μm 250 mm×20 mm, mobile phase: isohexane/isopropanol 50:50; flow rate: 20 ml/min; temperature: 25° C.; UV detection: 230 nm.

Method 15D: phase: Daicel Chiralpak IC, 5 μm 250 mm×20 mm, mobile phase: tert-butyl methyl ether/methanol 50:50; flow rate: 20 ml/min; temperature: 25° C.; UV detection: 220 nm.

Method 16D: phase: Daicel Chiralpak AY-H, 5 μm 250 mm×20 mm, mobile phase: isohexane/ethanol 50:50; flow rate: 20 ml/min; temperature: 20° C.; UV detection: 230 nm.

Method 17D: phase: Daicel Chiralpak AS-H, 5 μm 250 mm×20 mm, mobile phase: isohexane/ethanol 90:10; flow rate: 20 ml/min; temperature: 25° C.; UV detection: 220 nm.

Method 18D: phase: Daicel Chiralcel AZ-H, 5 μm 250 mm×40 mm; mobile phase: isohexane/ethanol 90:10+0.2% diethylamine; flow rate: 35 ml/min; temperature: 25° C.; UV detection: 230 nm.

Method 19D: phase: Daicel IA, 5 μm 250 mm×40 mm; mobile phase: tert-butyl methyl ether/methanol 50:50; flow rate: 20 ml/min; temperature: 25° C.; UV detection: 230 nm.

Method 20D: phase: Daicel Chiralcel AD-H, 5 μm 250 mm×20 mm; mobile phase: isohexane/isopropanol 60:40+0.2% diethylamine; flow rate: 20 ml/min; temperature: 20° C.; UV detection: 220 nm.

Method 21D: phase: Daicel Chiralpak IC, 5 μm 250 mm×20 mm, mobile phase: tert-butyl methyl ether/methanol/acetonitrile 50:25:25; flow rate: 15 ml/min; temperature: 35° C.; UV detection: 220 nm.

Method 22D: phase: Daicel Chiralcel AZ-H, 5 μm 250 mm×40 mm; mobile phase: isohexane/ethanol 90:10+0.2% diethylamine; flow rate: 15 ml/min; temperature: 30° C.; UV detection: 220 nm.

Method 23D: phase: Daicel Chiralcel AD-H, 5 μm 250 mm×20 mm; mobile phase: isohexane/isopropanol 50:50; flow rate: 20 ml/min; temperature: 40° C.; UV detection: 210 nm.

Method 24D: phase: Daicel Chiralpak IC, 5 μm 250 mm×20 mm, mobile phase: acetonitrile/methanol 30:70; flow rate: 30 ml/min; temperature: 25° C.; UV detection: 220 nm.

Method 25D: phase: Daicel Chiralpak OD-H, 5 μm 250 mm×20 mm, mobile phase: isohexane/ethanol 50:50; flow rate: 20 ml/min, temperature: 20° C.; UV detection: 220 nm.

Method 26D: phase: Daicel Chiralpak AS-H, 5 μm 250 mm×20 mm, mobile phase: isohexane/ethanol 70:30+0.2% diethylamine; flow rate: 20 ml/min; temperature: 20° C.; UV detection: 220 nm.

Method 27D: phase: Daicel Chiralpak AD-H SFC, 5 μm 250 mm×30 mm, mobile phase: carbon dioxide/methanol 80:20; flow rate: 100 ml/min, stepped gradient after 3 min for 1.5 min carbon dioxide/methanol 70:30; makeup flow rate: 30 ml/min, back pressure: 120 bar; temperature: 40° C.; UV detection: 210 nm.

Method 28D: phase: Daicel Chiralpak AD-H, 5 μm 250 mm×20 mm, mobile phase: isohexane/ethanol 30:70; flow rate: 20 ml/min, temperature: 40° C.; UV detection: 210 nm.

Method 29D: phase: Daicel Chiralcel AD-H, 5 μm 250 mm×20 mm; mobile phase: isohexane/ethanol 50:50; flow rate: 20 ml/min; temperature: 40° C.; UV detection: 210 nm.

Method 30D: phase: Daicel Chiralpak AS-H, 5 μm 250 mm×20 mm, mobile phase: isohexane/ethanol 50:50; flow rate: 20 ml/min, temperature: 35° C.; UV detection: 230 nm.

Method 31D: phase: Daicel Chiralcel AD-H, 5 μm 250 mm×20 mm; mobile phase: isohexane/isopropanol 50:50; flow rate: 20 ml/min; temperature: 25° C.; UV detection: 230 nm.

Method 32D: phase: Daicel Chiralpak OD-H, 5 μm 250 mm×20 mm, mobile phase: isohexane/isopropanol 80:20; flow rate: 20 ml/min, temperature: 25° C.; UV detection: 220 nm.

Method 33D: phase: Daicel Chiralpak AY-H, 5 μm 250 mm×20 mm, mobile phase: isohexane/isopropanol 50:50+0.2% diethylamine; flow rate: 15 ml/min, temperature: 40° C.; UV detection: 220 nm.

Method 34D: phase: Daicel Chiralcel AD-H, 5 μm 250 mm×20 mm; mobile phase: isohexane/isopropanol 50:50; flow rate: 20 ml/min; temperature: 20° C.; UV detection: 220 nm.

Method 35D: phase: Daicel Chiralpak OZ-H, 5 μm 250 mm×20 mm, mobile phase: isohexane/ethanol 50:50; flow rate: 20 ml/min, temperature: 25° C.; UV detection: 220 nm.

Method 36D: phase: Daicel Chiralcel AD-H, 5 μm, 250 mm×20 mm; mobile phase: ethanol+0.2% acetic acid/acetonitrile+0.2% acetic acid 90:10; flow rate: 20 ml/min; temperature: 25° C.; UV detection: 230 nm.

Method 37D: phase: Daicel Chiralpak ID, 5 μm 250 mm×20 mm, mobile phase: tert-butyl methyl ether/methanol 70:30; flow rate: 20 ml/min, temperature: 25° C.; UV detection: 230 nm.

Method 38D: phase: Daicel Chiralcel AZ-H, 5 μm 250 mm×20 mm; mobile phase: isohexane/isopropanol 50:50+0.2% diethylamine; flow rate: 20 ml/min; temperature: 45° C.; UV detection: 220 nm.

Method 39D: phase: Daicel Chiralpak ID, 5 μm 250 mm×20 mm, mobile phase: tert-butyl methyl ether/methanol 70:30; flow rate: 20 ml/min, temperature: 20° C.; UV detection: 230 nm.

Method 40D: phase: Daicel Chiralcel AZ-H, 5 μm 250 mm×40 mm; mobile phase: ethanol; flow rate: 13 ml/min; temperature: 45° C.; UV detection: 220 nm.

Method 41D: phase: Daicel Chiralcel AD-H, 5 μm 250 mm×20 mm; mobile phase: isohexane/isopropanol 50:50+0.2% diethylamine; flow rate: 20 ml/min; temperature: 25° C.; UV detection: 220 nm.

Method 42D: phase: Daicel Chiralpak OZ-H, 5 μm 250 mm×20 mm, mobile phase: isohexane/ethanol 30:70+0.2% diethylamine; flow rate: 15 ml/min, temperature: 40° C.; UV detection: 220 nm.

Method 43D: phase: Daicel Chiralcel OD-H, 5 μm 250 mm×40 mm; mobile phase: isohexane/isopropanol 90:10+0.2% diethylamine; flow rate: 20 ml/min; temperature: 25° C.; UV detection: 220 nm.

Method 44D: phase: Daicel Chiralcel AZ-H, 5 μm 250 mm×40 mm; mobile phase: isohexane/ethanol 80:20+0.2% diethylamine; flow rate: 20 ml/min; temperature: 25° C.; UV detection: 220 nm.

Method 45D: phase: Daicel Chiralpak AZ-H, 5 μm 250 mm×20 mm, mobile phase: isohexane/ethanol 70:30; flow rate: 20 ml/min, temperature: 40° C.; UV detection: 220 nm.

Method 46D: phase: Daicel Chiralpak OZ-H, 5 μm 250 mm×20 mm, mobile phase: isohexane/ethanol 25:75; flow rate: 15 ml/min, temperature: 40° C.; UV detection: 220 nm.

Method 47D: phase: Daicel Chiralpak IC, 5 μm 250 mm×20 mm, mobile phase: tert-butyl methyl ether/methanol 50:50; flow rate: 20 ml/min, temperature: 30° C.; UV detection: 220 nm.

Method 48D: phase: Daicel IA, 5 μm 250 mm×40 mm; mobile phase: tert-butyl methyl ether/methanol 50:50; flow rate: 20 ml/min; temperature: 30° C.; UV detection: 220 nm.

Method 49D: phase: Daicel Chiralpak AS-H, 5 μm, 250 mm×20 mm, mobile phase: 50% isohexane, 50% ethanol; flow rate: 20 ml/min; temperature: 25° C.; detection: 220 nm.

Method 50D: phase: Daicel Chiralpak IC, 5 μm, 250 mm×20 mm, mobile phase: 50% isohexane, 50% ethanol; flow rate: 15 ml/min; temperature: 40° C.; detection: 220 nm.

Method 51D: phase: Daicel Chiralpak OD-H, 5 μm, 250 mm×4 mm, mobile phase: 95% isohexane, 5% ethanol+1% diethylamine; flow rate: 20 ml/min; temperature: 40° C.; detection: 220 nm.

Method 52D: phase: Daicel Chiralpak AZ-H, 5 μm, 250 mm×30 mm, mobile phase: 10% isohexane, 90% ethanol+0.2% diethylamine; flow rate: 40 ml/min; temperature: 20° C.; detection: 220 nm.

Method 53D: phase: Daicel Chiralpak OD-H, 5 μm, 250 mm×20 mm, mobile phase: 95% isohexane, 5% ethanol; flow rate: 20 ml/min; temperature: 40° C.; detection: 220 nm.

Method 54D: phase: Daicel Chiralpak IC, 5 μm, 250 mm×20 mm, mobile phase: 70% acetonitrile, 30% methanol with 0.2% diethylamine; flow rate: 15 ml/min; temperature: 45° C.; detection: 220 nm.

Method 55D: phase: Daicel Chiralpak OD-H, 5 μm, 250 mm×20 mm, mobile phase: 70% isohexane, 30% ethanol with 2% diethylamine; flow rate: 20 ml/min; temperature: 25° C.; detection: 220 nm.

Method 56D: phase: Daicel Chiralpak OD-H, 5 μm, 250 mm×20 mm, mobile phase: 50% isohexane, 50% ethanol with 0.2% diethylamine; flow rate: 15 ml/min; temperature: 40° C.; detection: 220 nm.

Method 57D: phase: Daicel Chiralpak OD-H, 5 μm, 250 mm×20 mm, mobile phase: 50% isohexane, 50% ethanol; flow rate: 20 ml/min; temperature: 25° C.; detection: 220 nm.

Method 58D: phase: Daicel Chiralcel OZ-H, 5 μm, 250 mm×20 mm, mobile phase: 50% isohexane, 50% iso-ethanol; flow rate: 15 ml/min; temperature: 40° C.; detection: 220 nm.

Method 59D: phase: Daicel Chiralpak IC-H, 5 μm, 250 mm×20 mm, mobile phase: 50% tert-butyl methyl ether, 50% methanol; flow rate: 20 ml/min; temperature: 25° C.; detection: 220 nm.

Method 60D: phase: Daicel Chiracel OD-H, 5 μm, 250 mm×20 mm, mobile phase: 70% isohexane, 30% isopropanol; flow rate: 20 ml/min, temperature: 25° C.; detection: 220 nm.

### Analytical Enantiomer/Diastereomer Separation on a Chiral Phase:

Method 1E: phase: Daicel Chiralcel OZ-H, 5 μm 250 mm×4.6 mm; mobile phase: isohexane/ethanol 50:50; flow rate: 1 ml/min; temperature: 30° C.; UV detection: 220 nm.

Method 2E: phase: Daicel Chiralcel AZ-H, 5 μm 250 mm×4.6 mm; mobile phase: isohexane/ethanol 50:50; flow rate: 1 ml/min; temperature: 30° C.; UV detection: 220 nm.

Method 3E: phase: Daicel Chiralpak AD-H SFC, 5 μm 250 mm×4.6 mm; mobile phase: carbon dioxide/ethanol 70:30; flow rate: 3 ml/min; temperature: 30° C.; UV detection: 220 nm.

Method 4E: phase: Daicel Chiralpak AD-H, 5 μm 250 mm×4.6 mm, mobile phase: isohexane/isopropanol 50:50; flow rate: 1 ml/min; temperature: 30° C.; UV detection: 220 nm.

Method 5E: phase: LUX Amylose-2, 5 μm 250 mm×4.6 mm; mobile phase: isohexane/ethanol 90:10; flow rate: 1 ml/min; temperature: 30° C.; UV detection: 220 nm.

Method 6E: phase: Daicel Chiralpak AS-H, 5 μm 250 mm×4.6 mm, mobile phase: isohexane/isopropanol 50:50; flow rate: 1 ml/min; temperature: 30° C.; UV detection: 220 nm.

Method 7E: phase: Daicel Chiralcel OD-H, 5 μm 250 mm×4.6 mm; mobile phase: isohexane/ethanol 80:20+0.2% diethylamine; flow rate: 1 ml/min; temperature: 40° C.; UV detection: 220 nm.

Method 8E: phase: Daicel Chiralpak AD-H, 5 μm 250 mm×4.6 mm, mobile phase: isohexane/ethanol 50:50; flow rate: 1 ml/min; temperature: 30° C.; UV detection: 220 nm.

Method 9E: phase: Daicel Chiralcel OZ-H, 5 μm 250 mm×4.6 mm; mobile phase: isohexane/ethanol 50:50; flow rate: 1 ml/min; temperature: 40° C.; UV detection: 220 nm.

Method 10E: phase: Daicel Chiralcel OD-H, 5 μm 250 mm×4.6 mm; mobile phase: isohexane/ethanol 50:50; flow rate: 1 ml/min; temperature: 30° C.; UV detection: 220 nm.

Method 11E: phase: Daicel Chiralpak AD-H SFC, 5 μm 250 mm×4.6 mm; mobile phase: carbon dioxide/ethanol 70:30; flow rate: 4 ml/min; temperature: 30° C.; UV detection: 220 nm.

Method 12E: phase: Daicel Chiralcel AD-H, 5 μm 250 mm×4.6 mm; mobile phase: isohexane/ethanol 50:50+0.2% diethylamine; flow rate: 1 ml/min; temperature: 40° C.; UV detection: 220 nm.

Method 13E: phase: Daicel Chiralpak OD-H, 5 μm 250 mm×4.6 mm, mobile phase: isohexane/isopropanol 50:50; flow rate: 1 ml/min; temperature: 25° C.; UV detection: 220 nm.

Method 14E: phase: Daicel Chiralpak IC, 5 μm 250 mm×4.6 mm, mobile phase: tert-butyl methyl ether/methanol 50:50; flow rate: 1 ml/min; temperature: 30° C.; UV detection: 220 nm.

Method 15E: phase: Daicel Chiralpak AY-H, 5 μm 250 mm×4.6 mm, mobile phase: isohexane/ethanol 50:50; flow rate: 1 ml/min; temperature: 45° C.; UV detection: 220 nm.

Method 16E: phase: Daicel Chiralcel AZ-H, 5 μm 250 mm×4.6 mm; mobile phase: isohexane/ethanol 90:10; flow rate: 1 ml/min; temperature: 30° C.; UV detection: 220 nm.

Method 17E: phase: Daicel Chiralpak AS-H, 5 μm 250 mm×4.6 mm, mobile phase: isohexane/ethanol 90:10; flow rate: 1 ml/min; temperature: 30° C.; UV detection: 220 nm.

Method 18E: phase: Daicel Chiralcel AZ-H, 5 μm 250 mm×4.6 mm; mobile phase: isohexane/ethanol 90:10+0.2% diethylamine; flow rate: 1 ml/min; temperature: 40° C.; UV detection: 230 nm.

Method 19E: phase: Daicel IA, 5 μm 250 mm×4.6 mm; mobile phase: tert-butyl methyl ether/methanol 50:50; flow rate: 1 ml/min; temperature: 30° C.; UV detection: 220 nm.

Method 20E: phase: Daicel Chiralcel AD-H, 5 μm 250 mm×4.6 mm; mobile phase: isohexane/isopropanol 50:50+0.2% diethylamine; flow rate: 1 ml/min; temperature: 40° C.; UV detection: 220 nm.

Method 21E: phase: Daicel Chiralpak IC, 5 μm 250 mm×4.6 mm, mobile phase: tert-butyl methyl ether/methanol 50:50; flow rate: 1 ml/min; temperature: 40° C.; UV detection: 220 nm.

Method 22E: phase: Daicel Chiralpak IC, 5 μm 250 mm×4.6 mm, mobile phase: acetonitrile/methanol 30:70; flow rate: 1 ml/min; temperature: 30° C.; UV detection: 220 nm.

Method 23E: phase: Daicel Chiralcel OD-H, 5 μm 250 mm×4.6 mm; mobile phase: isohexane/ethanol 80:20; flow rate: 1 ml/min; temperature: 40° C.; UV detection: 220 nm.

Method 24E: phase: Daicel Chiralcel OZ-H, 5 μm 250 mm×4.6 mm; mobile phase: isohexane/ethanol 50:50; flow rate: 1 ml/min; temperature: 30° C.; UV detection: 220 nm.

Method 25E: phase: Daicel Chiralpak AD-H SFC, 5 μm 250 mm×4.6 mm; mobile phase: carbon dioxide/methanol 70:30; flow rate: 3 ml/min; temperature: 30° C.; UV detection: 220 nm.

Method 26E: phase: Daicel Chiralpak AD-H, 5 μm 250 mm×4.6 mm, mobile phase: isohexane/ethanol 30:70; flow rate: 1 ml/min, temperature: 40° C.; UV detection: 220 nm.

Method 27E: phase: Daicel Chiralpak AD-H, 5 μm 250 mm×4.6 mm, mobile phase: isohexane/ethanol 50:50; flow rate: 1 ml/min, temperature: 40° C.; UV detection: 220 nm.

Method 28E: phase: Daicel Chiralpak AS-H, 5 μm 250 mm×4.6 mm, mobile phase: isohexane/isopropanol 50:50; flow rate: 1 ml/min, temperature: 40° C.; UV detection: 220 nm.

Method 29E: phase: Daicel Chiralpak OD-H, 5 μm 250 mm×4.6 mm, mobile phase: isohexane/isopropanol 80:20; flow rate: 1 ml/min, temperature: 30° C.; UV detection: 220 nm.

Method 30E: phase: Daicel Chiralpak AY-H, 5 μm 250 mm×4.6 mm, mobile phase: isohexane/isopropanol 50:50; flow rate: 1 ml/min, temperature: 40° C.; UV detection: 220 nm.

Method 31E: phase: Daicel Chiralpak AD-H, 5 μm 250 mm×4.6 mm, mobile phase: isohexane/isopropanol 50:50; flow rate: 1 ml/min, temperature: 25° C.; UV detection: 230 nm.

Method 32E: phase: Daicel Chiralcel AD-H, 5 μm, 250 mm×4.6 mm; mobile phase: ethanol+0.2% acetic acid/acetonitrile+0.2% acetic acid 90:10; flow rate: 1 ml/min, temperature: 25° C.; UV detection: 230 nm.

Method 33E: phase: Daicel Chiralpak ID, 5 μm 250 mm×4 mm, mobile phase: tert-butyl methyl ether/methanol 70:30; flow rate: 1 ml/min, temperature: 40° C.; UV detection: 220 nm.

Method 34E: phase: Daicel Chiralcel AZ-H, 5 μm 250 mm×4.6 mm; mobile phase: isohexane/isopropanol 50:50+0.2% diethylamine; flow rate: 1 ml/min; temperature: 45° C.; UV detection: 220 nm.

Method 35E: phase: Daicel Chiralpak ID, 5 μm 250 mm×4 mm, mobile phase: tert-butyl methyl ether/methanol 70:30; flow rate: 1 ml/min, temperature: 30° C.; UV detection: 220 nm.

Method 36E: phase: Daicel Chiralcel AZ-H, 5 μm 250 mm×4.6 mm; mobile phase: ethanol; flow rate: 1 ml/min; temperature: 45° C.; UV detection: 220 nm.

Method 37E: phase: Daicel Chiralpak OZ-H, 5 μm 250 mm×4.6 mm, mobile phase: isohexane/ethanol 30:70+0.2% diethylamine; flow rate: 1 ml/min, temperature: 40° C.; UV detection: 230 nm.

Method 38E: phase: Daicel Chiralcel OD-H, 5 μm 250 mm×4.6 mm; mobile phase: isohexane/isopropanol 90:10+0.2% diethylamine; flow rate: 1 ml/min; temperature: 25° C.; UV detection: 220 nm.

Method 39E: phase: Daicel Chiralpak AZ-H, 5 μm 250 mm×4.6 mm, mobile phase: isohexane/ethanol 70:30; flow rate: 1 ml/min, temperature: 30° C.; UV detection: 220 nm.

Method 40E: phase: Daicel Chiralpak OZ-H, 5 μm 250 mm×4.6 mm, mobile phase: isohexane/ethanol 25:75; flow rate: 1 ml/min, temperature: 40° C.; UV detection: 220 nm.

Method 41E: phase: Daicel Chiralpak OZ-H, 5 μm 250 mm×4.6 mm, mobile phase: isohexane/ethanol 20:80; flow rate: 1 ml/min, temperature: 40° C.; UV detection: 220 nm.

Method 42E: phase: Daicel Chiralpak AS-H, 5 μm, 250 mm×4.6 mm, mobile phase: 50% isohexane, 50% ethanol; flow rate: 1 ml/min; temperature: 25° C.; detection: 220 nm.

Method 43E: phase: Daicel Chiralpak IC, 5 μm, 250 mm×4.6 mm, mobile phase: 50% isohexane, 50% ethanol; flow rate: 1 ml/min; temperature: 40° C.; detection: 220 nm.

Method 44E: phase: Daicel Chiralpak AS-H, 5 μm, 250 mm×4.6 mm, mobile phase: 30% isohexane, 70% ethanol; flow rate: 1 ml/min; temperature: 30° C.; detection: 220 nm.

Method 45E: phase: Daicel Chiralpak OD-H, 5 μm, 250 mm×4.6 mm, mobile phase: 95% isohexane, 5% ethanol; flow rate: 1 ml/min; temperature: 30° C.; detection: 220 nm.

Method 46E: phase: Daicel Chiralpak IC, 5 μm, 250 mm×4.6 mm, mobile phase: ethanol+0.2% diethylamine; flow rate: 1 ml/min; temperature: 45° C.; detection: 235 nm.

Method 47E: phase: Daicel Chiralpak OD-H, 5 μm, 250 mm×4 mm, mobile phase: 50% isohexane, 50% ethanol+0.2% diethylamine; flow rate: 1 ml/min; temperature: 40° C., detection: 220 nm.

Method 48E: phase: Daicel Chiralpak OD-H, 5 m, 250 mm×4 mm, mobile phase: 50% isohexane, 50% ethanol+0.2% diethylamine; flow rate: 1 ml/min; temperature: 40° C.; detection: 220 nm.

Method 49E: phase: Daicel Chiralpak OD-H, 5 m, 250 mm×4.6 mm, mobile phase: 50% isohexane, 50% ethanol; flow rate: 1 ml/min; temperature: 30° C.; detection: 220 nm.

Method 50E: phase: Daicel Chiralpak OZ-H, 5 m, 250 mm×4.6 mm, mobile phase: 60% isohexane, 40% ethanol; flow rate: 1 ml/min; temperature: 30° C.; detection: 220 nm.

Method 51E: phase: Daicel Chiralpak IC-H, 5 m, 250 mm×4.6 mm, mobile phase: 50% tert-butyl methyl ether, 50% methanol; flow rate: 1 ml/min; temperature: 30° C.; detection: 220 nm.

Method 52E: phase: Daicel Chiralpak OD-H, 5 m, 250 mm×4.6 mm, mobile phase: 70% isohexane, 30% isopropanol, flow rate: 1 ml/min; temperature: 30° C.; detection: 220 nm.

### Preparative Purification:

Method 1F: phase: Sunfire C-18, 5 μm 250 mm×20 mm, mobile phase: water/acetonitrile gradient 80:20→5:95, flow rate: 23.75 ml/min+constant addition of 2% strength formic acid; flow rate: 1.25 ml/min; UV detection: 210 nm.

Method 2F: phase: Sunfire C-18, 5 μm 250 mm×20 mm, mobile phase: water/acetonitrile 30:70; flow rate: 25 ml/min; temperature: 24° C.; UV detection: 210 nm.

Method 3F: phase: Sunfire C-18, 5 μm 250 mm×20 mm, mobile phase: water/methanol/1% ammonia in water 32:60:8; flow rate: 25 ml/min; temperature: 25° C.; UV detection: 210 nm.

Method 4F: phase: Shield C-18, 5 μm 100 mm×190 mm, water/methanol/1% trifluoroacetic acid in water 48:40:12; flow rate: 23.8 ml/min; temperature: 40° C.; UV detection: 210 nm.

Method 5F: phase: Shield C-18, 5 μm 100 mm×190 mm, water/acetonitrile gradient 90:10→5:95; flow rate: 25 ml/min; temperature: 23° C.; UV detection: 210 nm.

Method 6F: phase: X-Bridge C-18, 5 μm 150 mm×19 mm, mobile phase: water/acetonitrile gradient 95:5→5:95, flow rate: 23.75 ml/min+constant addition of 2% strength ammonia in water; flow rate: 1.25 ml/min; temperature: 23° C.; UV detection: 210 nm.

Method 7F: phase: Daiso C-18 Bio, 10 μm 300 mm×100 mm, mobile phase: water, 0.1% TFA/acetonitrile isocratic 20:80; flow rate: 250 ml/min; temperature: 20° C.; UV detection: 210 nm.

Method 8F: phase: Kromasil 100 C18, 5 μm, 250 mm×20 mm, mobile phase: 24% of water, 70% of methanol; flow rate: 23.75 ml/min, addition of 6% 1% strength trifluoroacetic acid at a flow rate of 1.25 ml/min; temperature: 40° C.; detection: 210 nm.

### Preparative Diastereomer Separation on an Achiral Phase:

Method 1G: phase: Sunfire C-18, 5 μm 250 mm×20 mm, mobile phase: water/methanol 60:40, flow rate: 60 ml/min, temperature: 23° C., UV detection: 210 nm.

Method 2G: phase: Sunfire C18, 5 μm, 150 mm×19 mm, mobile phase: 60% water, 40% methanol; from 10.00 min onwards 23% water, 77% methanol, from 10.10 min onwards 60% water, 40% methanol; flow rate: 23.75 ml/min, addition of 0.1% 2% strength formic acid at a flow rate of 1.25 ml/min; temperature: 25° C.; detection: 220 nm.

Method 3G: phase: Sunfire C18, 5 μm, 250 mm×20 mm, mobile phase: 65% of water, 35% of acetonitrile; flow rate: 23.75 ml/min, addition of 0.1% 2% strength trifluoroacetic acid at a flow rate of 1.25 ml/min; temperature: 23° C.; detection: 210 nm.

### Microwave

The microwave reactor used was a single-mode instrument of the Biotage Initiator Microwave Synthesizer type.

When inventive compounds are purified by preparative HPLC by the above-described methods in which the eluents contain additives, for example trifluoroacetic acid, formic acid or ammonia, the inventive compounds may be obtained in salt form, for example as trifluoroacetate, formate or ammonium salt, if the inventive compounds contain a sufficiently basic or acidic functionality. Such a salt can be converted to the corresponding free base or acid by various methods known to the person skilled in the art.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF3COOH”, “x Na+″₃COOH”, “x Na+″⁺” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

### Starting Materials

### Example 1A

### Methyl 7-methoxy-2-thioxo-2,3-dihydro-1,3-benzoxazole-5-carboxylate

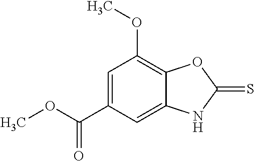 [SMILES: COC(=O)c1cc(OC)c2oc(=S)[nH]c2c1]

20.0 g (101 mmol) of methyl 3-amino-4-hydroxy-5-methoxybenzoate and 17.9 g (112 mmol) of potassium O-ethyl dithiocarbonate were dissolved in pyridine (400 ml), and the solution was stirred under reflux for 3 h (analogously to lit.: R. Lok et al., J. Org. Chem. 1996, 61, 3289-3297). The reaction mixture was then cooled and poured onto a mixture of ice (600 g) and concentrated hydrochloric acid (60 ml). The solid formed was filtered off under reduced pressure and washed with water (5×200 ml). The solid was dried initially at 50° C./40 mbar and then under high vacuum. Yield: 23.3 g (96% of theory).

LC-MS (Method 1A): Rₜ=0.79 min; MS (ESIpos): m/z=240 [M+H]⁺;

¹H-NMR (400 MHz, DMSO-d₆): δ[ppm]=14.1 (br. s., 1H), 7.45 (d, 1H), 7.32 (d, 1H), 4.00 (s, 3H), 3.88 (s, 3H).

### Example 2A

### Methyl 2-chloro-7-methoxy-1,3-benzoxazole-5-carboxylate

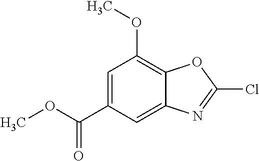 [SMILES: COC(=O)c1cc(OC)c2oc(Cl)nc2c1]

150 g (627 mmol) of methyl 7-methoxy-2-thioxo-2,3-dihydro-1,3-benzoxazole-5-carboxylate were suspended in thionyl chloride (450 ml), catalytic amounts of N,N-dimethylformamide (1.0 ml) were added and the mixture was then stirred for 3 h (analogously to lit.: R. Lok et al., J. Org. Chem. 1996, 61, 3289-3297). More N,N-dimethylformamide (1.0 ml) was added, and the mixture was stirred at 70° C. until the evolution of gas had ceased (about 4 h). The reaction solution was concentrated under reduced pressure and the residue was coevaporated with dichloromethane (3×200 ml) to completely remove the thionyl chloride. The solid was dried under high vacuum and then purified by column chromatography on silica gel (dichloromethane). Alternatively, the crude product can also be used further directly. Yield: 125.6 g (82% of theory).

LC-MS (Method 1A): Rₜ=1.00 min; MS (ESIpos): m/z=242 [M+H]⁺;

¹H-NMR (400 MHz, CDCl₃): δ [ppm]=7.99 (d, 1H), 7.62 (d, 1H), 4.07 (s, 3H), 3.96 (s, 3H).

### Example 3A

### Methyl 2-{[(4-chloropyridin-2-yl)methyl]amino}-7-methoxy-1,3-benzoxazole-5-carboxylate

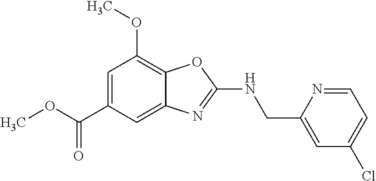 [SMILES: COC(=O)c1cc(OC)c2oc(NCc3cc(Cl)ccn3)nc2c1]

At RT, 72.8 g (98.2 ml, 563 mmol) of N,N-diisopropylethylamine were added to 25.5 g (93.9 mmol, purity: 90%) of methyl 2-chloro-7-methoxy-1,3-benzoxazole-5-carboxylate and 20.2 g (93.9 mmol) of 1-(4-chloropyridin-2-yl)methanamine dihydrochloride in 1,4-dioxane (700 ml), and the mixture was then stirred at 80° C. for 8 h. The reaction solution was concentrated under reduced pressure and the residue was taken up in ethyl acetate and washed with 0.5M aqueous hydrogen chloride solution. The organic phases were dried over sodium sulphate, filtered and concentrated under reduced pressure. The solid obtained was triturated with a little acetonitrile, filtered under reduced pressure and dried under high vacuum. Yield: 24.8 g (75% of theory).

LC-MS (Method 1A): Rₜ=0.94 min; MS (ESIpos): m/z=348 [M+H]⁺.

### Example 4A

### 2-{[(4-Chloropyridin-2-yl)methyl]amino}-7-methoxy-1,3-benzoxazole-5-carboxylic acid

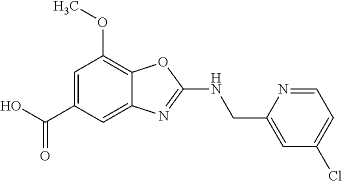 [SMILES: COc1cc(C(=O)O)cc2nc(NCc3cc(Cl)ccn3)oc12]

3.47 g (9.98 mmol) of methyl 2-{[(4-chloropyridin-2-yl)methyl]amino}-7-methoxy-1,3-benzoxazole-5-carboxylate were initially charged in 1,4-dioxane (200 ml), and 2N aqueous sodium hydroxide solution (100 ml) was then added. The mixture was stirred at RT for 16 h and most of the 1,4-dioxane was then removed under reduced pressure. The residue was diluted with water (200 ml) and then adjusted to pH=5 using concentrated hydrogen chloride solution. The precipitated solid was filtered off under reduced pressure, washed with water and dried under high vacuum.

Yield: 2.47 g (75% of theory).

LC-MS (Method 3A): Rₜ=1.72 min; MS (ESIpos): m/z=334 [M+H]⁺.

### Example 5A

### Methyl 2-{[1-(4-chloropyridin-2-yl)ethyl]amino}-7-methoxy-1,3-benzoxazole-5-carboxylate [racemate]

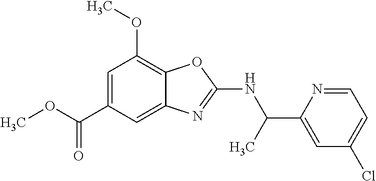 [SMILES: COC(=O)c1cc(OC)c2oc(NC(C)c3cc(Cl)ccn3)nc2c1]

At RT, 18.6 g (25.0 ml, 129 mmol) of N,N-diisopropylethylamine were added to 13.3 g (47.9 mmol, purity: 87%) of methyl 2-chloro-7-methoxy-1,3-benzoxazole-5-carboxylate and 7.50 g (157 mmol) of 1-(4-chloropyridin-2-yl)ethanamine [racemate, Lit.: V. Gotor et al, Adv. Synth. Catal. 2007, 349, 1481-1488] in tetrahydrofuran (650 ml), and the mixture was then stirred overnight. The reaction solution was concentrated under reduced pressure and the residue was taken up in ethyl acetate and the resulting solid was filtered off under reduced pressure. The filtrate was washed with 0.5M aqueous hydrogen chloride solution and the organic phases were dried over sodium sulphate, filtered and concentrated under reduced pressure. The solids were combined and dried under high vacuum. Yield: 20.5 g (100% of theory, purity: 90%).

LC-MS (Method 1A): Rₜ=0.94 min; MS (ESIpos): m/z=362 [M+H]⁺.

### Example 6A

### 2-{[1-(4-Chloropyridin-2-yl)ethyl]amino}-7-methoxy-1,3-benzoxazole-5-carboxylic acid [racemate]

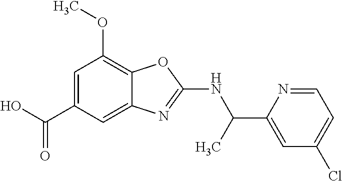 [SMILES: COc1cc(C(=O)O)cc2nc(NC(C)c3cc(Cl)ccn3)oc12]

20.5 g (51.0 mmol, purity: 90%) of methyl 2-{[1-(4-chloropyridin-2-yl)ethyl]amino}-7-methoxy-1,3-benzoxazole-5-carboxylate [racemate] were initially charged in 1,4-dioxane (550 ml), and 2N aqueous sodium hydroxide solution (275 ml) was then added. The mixture was stirred at 55° C. for 5 h and most of the 1,4-dioxane was then removed under reduced pressure. The residue was adjusted to pH=5 using 2N hydrogen chloride solution and the precipitated solid was filtered off under reduced pressure and dried under high vacuum. Yield: 14.1 g (62% of theory, purity: 79%).

LC-MS (Method 3A): Rₜ=1.85 min; MS (ESIpos): m/z=348 [M+H]⁺.

### Example 7A

### 1-Benzyl 2-ethyl (4R)-4-ethoxypyrrolidine-1,2-dicarboxylate [diastereomer mixture, 2 isomers]

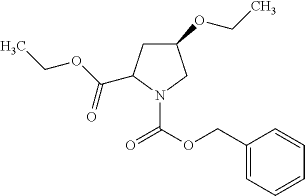 [SMILES: CCOC(=O)C1C[C@@H](OCC)CN1C(=O)OCc1ccccc1]

Under argon, 10.0 g (37.7 mmol) of (4R)-1-[(benzyloxy)carbonyl]-4-hydroxy-L-proline were initially charged in N,N-dimethylformamide (110 ml), and 1.96 g (49.0 mmol, 60% suspension in paraffin oil) of sodium hydride were added at 0° C. The reaction mixture was stirred for 30 min, and 7.54 ml (14.7 g, 94.2 mmol) of iodoethane were then added. The mixture was warmed to RT and then once more cooled to 0° C., a further 1.96 g (49.0 mmol, 60% suspension in paraffin oil) of sodium hydride were added and the mixture was stirred for 30 min. A further 7.54 ml (14.7 g, 94.2 mmol) of iodoethane were added, and the mixture was warmed to RT and stirred overnight. Water was added carefully, and the reaction mixture was then extracted with ethyl acetate. The organic phase was dried over sodium sulphate, filtered and concentrated under reduced pressure. The crude product was used without further purification in the next step. Yield: 14.8 g (94% of theory, purity: 77%).

LC-MS (Method 1A): Rₜ=1.06 min; MS (ESIpos): m/z=322 [M+H]⁺.

### Example 8A

### Benzyl (4R)-4-ethoxy-2-(hydroxymethyl)pyrrolidine-1-carboxylate [diastereomer mixture, 2 isomers]

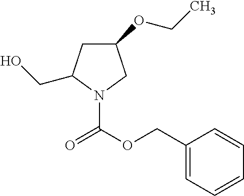 [SMILES: CCO[C@@H]1CC(CO)N(C(=O)OCc2ccccc2)C1]

13.5 g (32.6 mmol, purity: 77%) of 1-benzyl 2-ethyl (4R)-4-ethoxypyrrolidine-1,2-dicarboxylate [diastereomer mixture, 2 isomers] were initially charged in tetrahydrofuran (150 ml) under argon, and 817 mg (37.5 mmol) of lithium borohydride were added at 0° C. The reaction mixture was warmed to RT and then stirred overnight. Water (100 ml) was added carefully to the reaction mixture, the pH was adjusted to pH=1 using an aqueous 2N hydrogen chloride solution and the mixture was then extracted with ethyl acetate. The organic phases were washed with saturated sodium chloride solution, dried over sodium sulphate, filtered and concentrated under reduced pressure. The crude product was purified by preparative RP-HPLC (acetonitrile/water). Yield: 4.78 g (52% of theory, diastereomer ratio: about 2:1).

LC-MS (Method 1A): Rₜ=0.81 min (diastereomer 1), Rₜ=0.83 min (diastereomer 2);

MS (ESIpos): m/z=280 [M+H]⁺.

### Example 9A

### [(4R)-4-Ethoxypyrrolidin-2-yl]methanol [diastereomer mixture, 2 isomers]

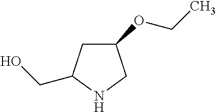

2.00 g (7.16 mmol) of benzyl (4R)-4-ethoxy-2-(hydroxymethyl)pyrrolidine-1-carboxylate [diastereomer mixture, 2 isomers] were initially charged in methanol (46.3 ml), 221 mg of palladium on carbon (10%) and 111 mg of platinum(IV) oxide were added under argon and the mixture was then stirred under a hydrogen atmosphere at standard pressure until the hydrogen uptake had ended. The reaction solution was filtered through kieselguhr, the filter cake was washed with methanol and the filtrate was concentrated under reduced pressure. Yield: 1.17 g (quant.).

MS (Method 2C): m/z=146 [M+H]⁺;

¹H-NMR (400 MHz, DMSO-d₆): δ[ppm]=4.04-2.67 (m, 10H), 2.10-1.30 (m, 2H), 1.22-0.97 (m, 3H).

### Example 10A

### 1-Benzyl 2-methyl (4R)-4-(2,2-difluoroethoxy)pyrrolidine-1,2-dicarboxylate [diastereomer mixture, 2 isomers]

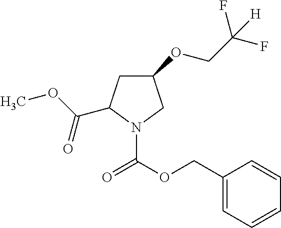 [SMILES: COC(=O)C1C[C@@H](OCC(F)F)CN1C(=O)OCc1ccccc1]

8.20 g (24.7 mmol, purity: 84%) of 1-benzyl 2-methyl (4R)-4-hydroxypyrrolidine-1,2-dicarboxylate and 5.81 g (27.1 mmol) of 2,2-difluoroethyl trifluoromethanesulphonate were initially charged in N,N-dimethylformamide (200 ml) under argon, and 1.28 g (32.1 mmol, 60% suspension in paraffin oil) of sodium hydride were added at 0° C. The mixture was allowed to warm to RT and stirred for 1 h. The reaction mixture was concentrated under reduced pressure, and the residue was taken up in dichloromethane and then washed with water and saturated aqueous sodium chloride solution. The organic phase was dried over sodium sulphate, filtered and concentrated under reduced pressure. The crude product was purified by preparative RP-HPLC (acetonitrile/water). Yield: 6.55 g (69% of theory, purity: 90%, diastereomer ratio: about 5:4).

LC-MS (Method 2A): Rₜ=0.98 min (enantiomerically pure isomer 1), Rₜ=0.99 min (enantiomerically pure isomer 2);

MS (ESIpos): m/z=344 [M+H]⁺.

### Example 11A

### Benzyl (4R)-4-(2,2-difluoroethoxy)-2-(hydroxymethyl)pyrrolidine-1-carboxylate [diastereomer mixture, 2 isomers]

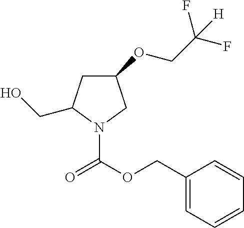 [SMILES: O=C(OCc1ccccc1)N1C[C@H](OCC(F)F)CC1CO]

6.50 g (17.0 mmol, purity: 90%) of 1-benzyl 2-methyl (4R)-4-(2,2-difluoroethoxy)pyrrolidine-1,2-dicarboxylate [diastereomer mixture, 2 isomers] were initially charged in tetrahydrofuran (200 ml), and 1.11 g (51.1 mmol) of lithium borohydride were added at 0° C. The reaction mixture was warmed to RT and then stirred overnight. Water (50 ml) was added carefully to the reaction mixture and the tetrahydrofuran was removed under reduced pressure. The residue was taken up in dichloromethane and washed with 1M sodium carbonate solution. The organic phase was dried over sodium sulphate, filtered and concentrated under reduced pressure. The crude product was purified by preparative RP-HPLC (acetonitrile/water). Yield: 4.25 g (79% of theory, diastereomer ratio: about 1:1).

LC-MS (Method 2A): Rₜ=0.87 min (enantiomerically pure isomer 1), Rₜ=0.88 min (enantiomerically pure isomer 2);

MS (ESIpos): m/z=316 [M+H]⁺.

### Example 12A

### Benzyl (4R)-4-(2,2-difluoroethoxy)-2-(hydroxymethyl)pyrrolidine-1-carboxylate [enantiomerically pure isomer 1]

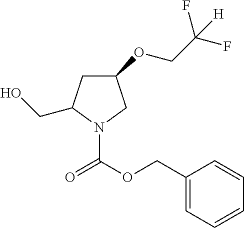 [SMILES: O=C(OCc1ccccc1)N1C[C@H](OCC(F)F)CC1CO]

Diastereomer separation on a chiral phase of 1.06 g of the compound from Example 11A according to Method 27D gave 447 mg of Example 12A (enantiomerically pure isomer 1) and 510 mg of Example 13A (enantiomerically pure isomer 2).

HPLC (Method 25E): Rₜ=1.90 min, >99.0% de;

LC-MS (Method 2A): Rₜ=0.88 min; MS (ESIpos): m/z=316 [M+H]⁺.

### Example 13A

### Benzyl (4R)-4-(2,2-difluoroethoxy)-2-(hydroxymethyl)pyrrolidine-1-carboxylate [enantiomerically pure isomer 2]

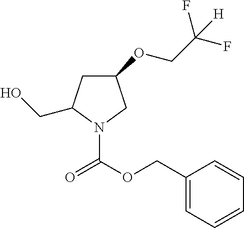 [SMILES: O=C(OCc1ccccc1)N1C[C@H](OCC(F)F)CC1CO]

Diastereomer separation on a chiral phase of 1.06 g of the compound from Example 11A according to Method 27D gave 447 mg of Example 12A (enantiomerically pure isomer 1) and 510 mg of Example 13A (enantiomerically pure isomer 2).

HPLC (Method 25E): Rₜ=2.97 min, >99.0% de;

LC-MS (Method 2A): Rₜ=0.87 min; MS (ESIpos): m/z=316 [M+H]⁺.

### Example 14A

### [(4R)-4-(2,2-Difluoroethoxy)pyrrolidin-2-yl]methanol [enantiomerically pure isomer 2]

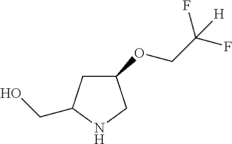 [SMILES: OCC1C[C@@H](OCC(F)F)CN1]

510 mg (1.62 mmol) of benzyl (4R)-4-(2,2-difluoroethoxy)-2-(hydroxymethyl)pyrrolidine-1-carboxylate [enantiomerically pure isomer 2, Example 13A] were initially charged in methanol (10.4 ml), 52.0 mg of palladium on carbon (10%) were added under argon and the mixture was stirred under a hydrogen atmosphere at standard pressure until the hydrogen uptake had ended. The reaction solution was filtered through kieselguhr, the filter cake was washed with methanol and the filtrate was concentrated under reduced pressure. Yield: 299 mg (quant.).

MS (Method 2C): m/z=182 [M+H]⁺.

### Example 15A

### 1-Benzyl 2-methyl (4R)-4-[(1,1-²H₂)ethyloxy]pyrrolidine-1,2-dicarboxylate [diastereomer mixture, 2 isomers]

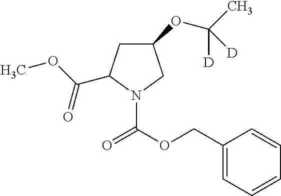 [SMILES: [2H]C([2H])(C)O[C@@H]1CC(C(=O)OC)N(C(=O)OCc2ccccc2)C1]

Under argon, 1.35 g (33.8 mmol, 60% suspension in paraffin oil) of sodium hydride were added to 6.29 g (22.5 mmol) of 1-benzyl 2-methyl (4R)-4-hydroxypyrrolidine-1,2-dicarboxylate in N,N-dimethylformamide (100 ml) at 0° C. The mixture was stirred for 30 min, 5.00 g (9.37 ml, 45.1 mmol) of 1-bromo(2,2-²H₂)propane and 832 mg (2.25 mmol) of tetra-n-butylammonium iodide were added and the mixture was warmed to RT and stirred for 3 h. Water was added carefully, and the reaction mixture was then concentrated under reduced pressure. The residue was taken up in ethyl acetate and washed with water and saturated aqueous sodium chloride solution. The organic phase was dried over sodium sulphate, filtered and concentrated under reduced pressure. The crude product was used without further purification in the next step. Yield: 5.07 g (60% of theory, purity: 83%, diastereomer ratio: about 4:5).

LC-MS (Method 1A): Rₜ=0.98 min (enantiomerically pure isomer 1), Rₜ=0.99 min (enantiomerically pure isomer 2);

MS (ESIpos): m/z=310 [M+H]⁺.

### Example 16A

### Benzyl (4R)-4-[(1,1-²H₂)ethyloxy]-2-(hydroxymethyl)pyrrolidine-1-carboxylate [diastereomer mixture, 2 isomers]

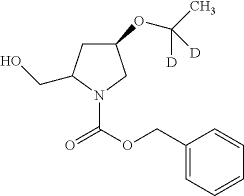 [SMILES: [2H]C([2H])(C)O[C@@H]1CC(CO)N(C(=O)OCc2ccccc2)C1]

4.00 g (10.8 mmol, purity: 83%) of 1-benzyl 2-methyl (4R)-4-[(1,1-²H₂)ethyloxy]pyrrolidine-1,2-dicarboxylate [diastereomer mixture, 2 isomers] were initially charged in tetrahydrofuran (26.0 ml), and 270 mg (12.4 mmol) of lithium borohydride were added at 0° C. The reaction mixture was warmed to RT and then stirred overnight. Water was then added carefully and the mixture was subsequently acidified with 2N aqueous hydrogen chloride solution. The aqueous phase was extracted repeatedly with ethyl acetate and the collected organic phases were washed with saturated aqueous sodium chloride solution, dried over sodium sulphate, filtered and concentrated under reduced pressure. The crude product was used without further purification in the next step. Yield: 1.01 g (29% of theory, purity: 88%, diastereomer ratio: about 1:1).

LC-MS (Method 1A): Rₜ=0.81 min (enantiomerically pure isomer 1), Rₜ=0.84 min (enantiomerically pure isomer 2);

MS (ESIpos): m/z=282 [M+H]⁺.

### Example 17A

### {(4R)-4-[(1,1-²H₂)Ethyloxy]pyrrolidin-2-yl}methanol [diastereomer mixture, 2 isomers]

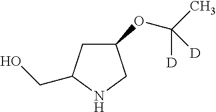

1.00 g (3.55 mmol) of benzyl (4R)-4-[(1,1-²H₂)ethyloxy]-2-(hydroxymethyl)pyrrolidine-1-carboxylate [diastereomer mixture, 2 isomers] were initially charged in methanol (23.0 ml), 110 mg of palladium on carbon (10%) and 55.0 mg of platinum(IV) oxide were added under argon and the mixture was then stirred under a hydrogen atmosphere at standard pressure until the hydrogen uptake had ended. The reaction solution was filtered through kieselguhr, the filter cake was washed with methanol and the filtrate was concentrated under reduced pressure. Yield: 576 mg (quant.).

MS (Method 2C): m/z=148 [M+H]⁺.

### Example 19A

### Benzyl (4R)-2-(cyanomethyl)-4-[(1,1-²H₂)ethyloxy]pyrrolidine-1-carboxylate [diastereomer mixture, 2 isomers]

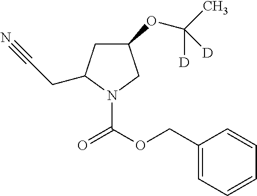 [SMILES: [2H]C([2H])(C)O[C@@H]1CC(CC#N)N(C(=O)OCc2ccccc2)C1]

Under argon, 2.90 g (10.3 mmol) of benzyl (4R)-4-[(1,1-²H₂)ethyloxy]-2-(hydroxymethyl)pyrrolidine-1-carboxylate [diastereomer mixture, 2 isomers] were initially charged in dichloromethane (100 ml), and 3.54 g (2.39 ml, 30.9 mmol) of methanesulphonyl chloride and 3.13 g (4.31 ml, 30.9 mmol) of triethylamine were added at 0° C. The reaction mixture was warmed to RT and then stirred for 2 h. Water was added carefully to the reaction mixture, the phases were separated and the aqueous phase was extracted with dichloromethane. The collected organic phases were washed with saturated aqueous sodium chloride solution, dried over sodium sulphate, filtered and concentrated under reduced pressure. The crude product (2.40 g) was dissolved in N,N-dimethylformamide (10 ml) and, under argon and at RT, added to a 0.5N solution of lithium cyanide in N,N-dimethylformamide (20 ml, 10 mmol). The reaction mixture was initially stirred at RT overnight, then at 60° C. for a further night and then at 80° C. for 2 d. The reaction solution was concentrated under reduced pressure and the residue was taken up in ethyl acetate and water. After separation of the phases, the aqueous phase was extracted twice with ethyl acetate. The collected organic phases were washed with saturated aqueous sodium chloride solution, dried over sodium sulphate, filtered and concentrated under reduced pressure. The crude product was purified by means of two preparative RP-HPLCs (acetonitrile/water+0.1% formic acid). Yield: 430 mg (14% of theory over two steps).

LC-MS (Method 1A): Rₜ=0.96 min; MS (ESIpos): m/z=291 [M+H]⁺.

### Example 20A

### {(4R)-4-[(1,1-²H₂)Ethyloxy]pyrrolidin-2-yl}acetonitrile [diastereomer mixture, 2 isomers]

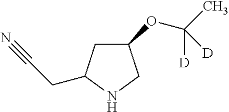

430 mg (1.48 mmol) of benzyl (4R)-2-(cyanomethyl)-4-[(1,1-²H₂)ethyloxy]pyrrolidine-1-carboxylate [diastereomer mixture, 2 isomers] were initially charged in ethanol (50 ml), 26 mg of palladium on carbon (10%) were added under argon and the mixture was then stirred under an atmosphere of hydrogen at standard pressure overnight. The reaction solution was filtered, the filter cake was washed with ethanol and the filtrate was concentrated under reduced pressure. The crude product was used directly in Example 9. Yield: 234 mg (quant.).

### Example 21A

### 1-Benzyl 2-methyl (2S,4R)-4-(1-ethoxyethoxy)pyrrolidine-1,2-dicarboxylate [diastereomer mixture, 2 isomers]

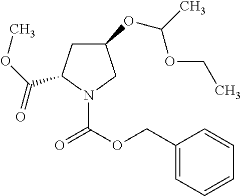 [SMILES: CCOC(C)O[C@@H]1C[C@@H](C(=O)OC)N(C(=O)OCc2ccccc2)C1]

5.00 g (17.9 mmol) of 1-benzyl 2-methyl (2S,4R)-4-hydroxypyrrolidine-1,2-dicarboxylate [enantiomerically pure isomer] were initially charged in ethyl vinyl ether (32.4 ml), and 12.0 mg (8.1 μl, 0.11 mmol) of trifluoroacetic acid were added at 0° C. The reaction mixture was warmed to RT and stirred for 4 d. Solid sodium bicarbonate (800 mg) was then added, and the reaction mixture was stirred for 1 h. The reaction mixture was then filtered and concentrated under reduced pressure at 40° C. to 50° C. The crude product was used without further purification in the next step.

Yield: 6.20 g (93% of theory).

LC-MS (Method 2A): Rₜ=1.05 min; MS (ESIpos): m/z=352 [M+H]⁺;

¹H-NMR (400 MHz, DMSO-d₆): δ[ppm]=7.42-7.22 (m, 5H), 5.17-4.90 (m, 2H), 4.79-4.69 (m, 1H), 4.40-4.23 (m, 2H), 3.69-3.30 (m, 7H), 2.30 (m_{c}, 1H), 2.04 (m_{c}, 1H), 1.18 (m_{c}, 3H), 1.12-1.05 (m, 3H).

### Example 22A

### 1-Benzyl 2-methyl (2S,4R)-4-(vinyloxy)pyrrolidine-1,2-dicarboxylate [enantiomerically pure isomer]

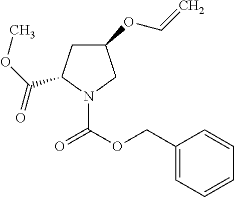 [SMILES: C=CO[C@@H]1C[C@@H](C(=O)OC)N(C(=O)OCc2ccccc2)C1]

20 g (17.3 mmol, purity: 30%) of 1-benzyl 2-methyl (2S,4R)-4-(1-ethoxyethoxy)pyrrolidine-1,2-dicarboxylate [diastereomer mixture, 2 isomers] were initially charged in dichloromethane (60 ml) under argon, and 1.82 g (2.51 ml, 18.0 mmol) of triethylamine and 3.93 g (3.21 ml, 17.7 mmol) of trimethylsilyl trifluoromethanesulphonate were added dropwise at 0° C. The reaction mixture was warmed to RT and stirred for 3.5 h. The mixture was then once more cooled to 0° C. and another 1.82 g (2.51 ml, 18.0 mmol) of triethylamine and 3.93 g (3.21 ml, 17.7 mmol) of trimethylsilyl trifluoromethanesulphonate were added. The reaction solution was warmed to RT and stirred for 2 d. The mixture was then once more cooled to 0° C. and another 1.82 g (2.51 ml, 18.0 mmol) of triethylamine and 3.93 g (3.21 ml, 17.7 mmol) of trimethylsilyl trifluoromethanesulphonate were added. The reaction solution was warmed to RT and stirred overnight. 1N aqueous sodium hydroxide solution (60 ml) was then added, and the reaction mixture was diluted with water and extracted. The organic phase was dried over sodium sulphate, filtered and concentrated under reduced pressure. The crude product was purified by means of flash chromatography on silica gel (cyclohexane/ethyl acetate 3:1). Yield: 2.48 g (42% of theory, purity: 90%).

LC-MS (Method 1A): Rₜ=0.98 min; MS (ESIpos): m/z=306 [M+H]⁺.

### Example 23A

### 1-Benzyl 2-methyl (2S,4R)-4-(cyclopropyloxy)pyrrolidine-1,2-dicarboxylate [enantiomerically pure isomer]

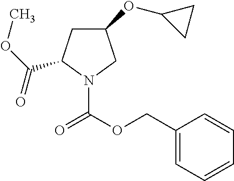 [SMILES: COC(=O)[C@@H]1C[C@@H](OC2CC2)CN1C(=O)OCc1ccccc1]

Under argon, 13.4 ml (13.4 mmol) of a 1M diethylzinc solution in hexane were initially charged in dichloromethane (70 ml), and 3.53 g (1.06 ml, 13.2 mmol) of diiodomethane in dichloromethane (14 ml) were then added dropwise at 0° C. After 30 min, 1.88 g (5.36 mmol, purity: 87%) of 1-benzyl 2-methyl (2S,4R)-4-(vinyloxy)pyrrolidine-1,2-dicarboxylate [enantiomerically pure isomer] in dichloromethane (21 ml) were added dropwise and the reaction mixture was warmed to RT and stirred overnight. The reaction was terminated by addition of saturated aqueous ammonium chloride solution, the phases were separated and the aqueous phase was extracted with ethyl acetate. The collected organic phases were washed with saturated aqueous sodium carbonate solution, dried over sodium sulphate, filtered and concentrated under reduced pressure. The crude product was purified by preparative RP-HPLC (acetonitrile/water). Yield: 1.09 g (59% of theory).

LC-MS (Method 2A): Rₜ=1.03 min; MS (ESIpos): m/z=320 [M+H]⁺.

### Example 24A

### Methyl (4R)-4-(cyclopropyloxy)-L-prolinate [enantiomerically pure isomer]

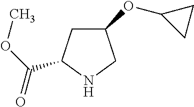

1.09 g (3.21 mmol) of 1-benzyl 2-methyl (2S,4R)-4-(cyclopropyloxy)pyrrolidine-1,2-dicarboxylate [enantiomerically pure isomer] were initially charged in methanol (20.6 ml), 103 mg of palladium on carbon (10%) were added under argon and the mixture was then stirred under an atmosphere of hydrogen at standard pressure for 4 h. The reaction solution was filtered through kieselguhr, the filter cake was washed with methanol and the filtrate was concentrated under reduced pressure.

Yield: 630 mg (100% of theory).

MS (Method 2C): m/z=186 [M+H]⁺.

### Example 25A

### Methyl (4R)-1-[(2-{[(4-chloropyridin-2-yl)methyl]amino}-7-methoxy-1,3-benzoxazol-5-yl)carbonyl]-4-(cyclopropyloxy)-L-prolinate [enantiomerically pure isomer]

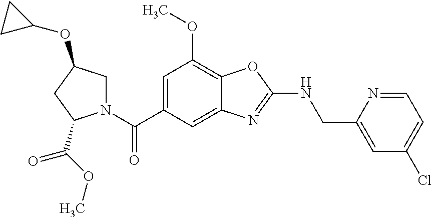 [SMILES: COC(=O)[C@@H]1C[C@@H](OC2CC2)CN1C(=O)c1cc(OC)c2oc(NCc3cc(Cl)ccn3)nc2c1]

1.20 g (2.91 mmol, purity: 81%) of 2-{[(4-chloropyridin-2-yl)methyl]amino}-7-methoxy-1,3-benzoxazole-5-carboxylic acid and 630 mg (3.20 mmol) of methyl (4R)-4-(cyclopropyloxy)-L-prolinate [enantiomerically pure isomer] were initially charged in N,N-dimethylformamide (24.2 ml), and 1.51 g (2.03 ml, 11.6 mmol) of N,N-diisopropylethylamine were added. 1.33 g (3.49 mmol) of HATU were then added at RT, and the mixture was stirred for 2 h. The reaction solution was concentrated under reduced pressure and then, without further work-up, purified directly by preparative RP-HPLC (acetonitrile/water). Yield: 748 mg (12% of theory, purity: 24%).

LC-MS (Method 1A): Rₜ=0.92 min; MS (ESIpos): m/z=501 [M+H]⁺.

### Example 26A

### (4R)-1-[(2-{[(4-Chloropyridin-2-yl)methyl]amino}-7-methoxy-1,3-benzoxazol-5-yl)carbonyl]-4-(cyclopropyloxy)-L-proline [enantiomerically pure isomer]

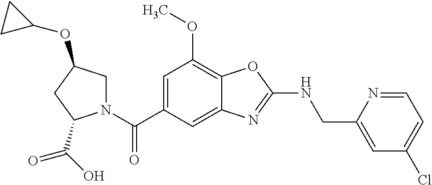 [SMILES: COc1cc(C(=O)N2C[C@H](OC3CC3)C[C@H]2C(=O)O)cc2nc(NCc3cc(Cl)ccn3)oc12]

749 mg (0.366 mmol, purity: 24%) of methyl (4R)-1-[(2-{[(4-chloropyridin-2-yl)methyl]amino}-7-methoxy-1,3-benzoxazol-5-yl)carbonyl]-4-(cyclopropyloxy)-L-prolinate [enantiomerically pure isomer] were dissolved in tetrahydrofuran/water (3:1, 12.0 ml), 35.1 mg (1.46 mmol) of lithium hydroxide were added and the mixture was stirred at RT for 1 h. The reaction solution was then acidified with 1N aqueous hydrogen chloride solution and extracted with ethyl acetate. The collected organic phases were washed with saturated aqueous sodium chloride solution, dried over sodium sulphate, filtered and concentrated under reduced pressure. The crude product was used without further purification in the next step. Yield: 878 mg (quant., purity: 26%).

LC-MS (Method 2A): Rₜ=0.79 min; MS (ESIpos): m/z=487 [M+H]⁺.

### Example 27A

### Benzyl (2S,4R)-4-(cyclopropyloxy)-2-(hydroxymethyl)pyrrolidine-1-carboxylate [enantiomerically pure isomer]

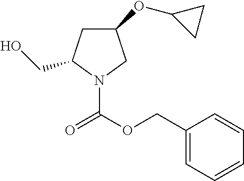 [SMILES: O=C(OCc1ccccc1)N1C[C@H](OC2CC2)C[C@H]1CO]

1.35 g (4.23 mmol) of 1-benzyl 2-methyl (2S,4R)-4-(cyclopropyloxy)pyrrolidine-1,2-dicarboxylate [enantiomerically pure isomer] were initially charged in tetrahydrofuran (20.0 ml), and 106 mg (4.87 mmol) of lithium borohydride were added at 0° C. The reaction mixture was warmed to RT and then stirred overnight. Water (200 ml) was added carefully and the reaction mixture was subsequently acidified with 2N aqueous hydrogen chloride solution. The aqueous phase was extracted repeatedly with ethyl acetate and the collected organic phases were washed with saturated aqueous sodium chloride solution, dried over sodium sulphate, filtered and concentrated under reduced pressure. The crude product was purified by preparative RP-HPLC (acetonitrile/water).

Yield: 1.09 g (59% of theory).

LC-MS (Method 2A): Rₜ=0.89 min; MS (ESIpos): m/z=292 [M+H]⁺;

¹H-NMR (400 MHz, DMSO-d₆): δ[ppm]=7.42-7.27 (m, 5H), 5.10 (d, 1H), 5.03 (d, 1H), 4.76 (br. s., 1H), 4.16 (br. s., 1H), 3.81 (br. s., 1H), 3.61-3.36 (m, 4H), 3.28-3.19 (m, 1H), 2.13-1.92 (m, 2H), 0.51-0.36 (m, 4H).

### Example 28A

### [(2S,4R)-4-(Cyclopropyloxy)pyrrolidin-2-yl]methanol [enantiomerically pure isomer]

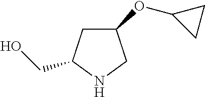 [SMILES: OC[C@@H]1C[C@@H](OC2CC2)CN1]

690 mg (2.37 mmol) of benzyl (2S,4R)-4-(cyclopropyloxy)-2-(hydroxymethyl)pyrrolidine-1-carboxylate [enantiomerically pure isomer] were initially charged in methanol (20.0 ml), 91 mg of palladium on carbon (10%) were added under argon and the mixture was then stirred under an atmosphere of hydrogen at standard pressure overnight. The reaction solution was filtered through kieselguhr, the filter cake was washed with methanol and the filtrate was concentrated under reduced pressure. Yield: 383 mg (98% of theory).

MS (Method 2C): m/z=158 [M+H]⁺.

### Example 29A

### 1-Benzyl 2-methyl 2-methylpyrrolidin-1,2-dicarboxylate [enantiomerically pure isomer 2]

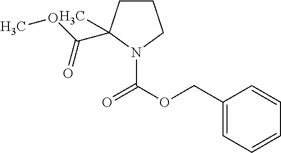 [SMILES: COC(=O)C1(C)CCCN1C(=O)OCc1ccccc1]

2.75 g (10.5 mmol) of 1-benzyl 2-methyl pyrrolidine-1,2-dicarboxylate [racemate] were initially charged in tetrahydrofuran (24 ml), 15.7 ml (15.7 mmol) of 1M lithium hexamethyldisilazide solution in tetrahydrofuran were added under argon and at −78° C. and the reaction mixture was then stirred for 30 min. Subsequently, at −78° C., 2.37 g (1.04 ml, 16.7 mmol) of iodomethane were added, and the reaction mixture was warmed to RT and stirred overnight. The reaction solution was then concentrated under reduced pressure, water was added to the residue and the mixture was extracted with dichloromethane. The organic phase was dried over sodium sulphate, filtered and concentrated under reduced pressure. The crude product was separated into the enantiomers on a chiral phase according to Method 32D. The target compound corresponded to the enantiomerically pure isomer 2, which eluted later. Yield: 942 mg (32% of theory, enantiomerically pure isomer 2).

HPLC (Method 29E): Rₜ=7.26 min, >99.9% ee;

LC-MS (Method 2A): Rₜ=1.04 min; MS (ESIpos): m/z=278 [M+H]⁺.

The target compound is described as a racemate in the patent Zhu, Gui-Dong et al., US 20060229289, 2006, however, the preparation is carried out using sodium hexamethyldisilazide solution.

### Example 30A

### Methyl 2-methylprolinate [enantiomerically pure isomer]

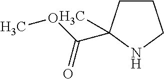

11.6 g (41.8 mmol) of 1-benzyl 2-methyl 2-methylpyrrolidine-1,2-dicarboxylate [enantiomerically pure isomer 2, Example 29A] were initially charged in methanol (270 ml), 1.34 g of palladium on carbon (10%) were added under argon and the mixture was then stirred under a hydrogen atmosphere at standard pressure until the hydrogen uptake had ended. The reaction solution was filtered through kieselguhr, the filter cake was washed with methanol and the filtrate was concentrated under reduced pressure. Yield: 5.05 g (84% of theory).

MS (Method 2C): m/z=144 [M+H]⁺;

optical rotation: [α]_{D}²⁰=2.59° (c=0.45, chloroform).

### Example 31A

### Methyl 1-[(2-{[(4-chloropyridin-2-yl)methyl]amino}-7-methoxy-1,3-benzoxazol-5-yl)carbonyl]-2-methylprolinate [enantiomerically pure isomer]

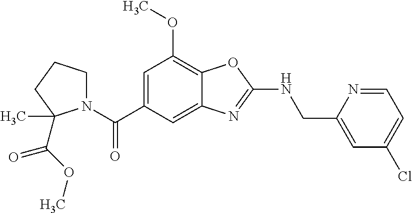 [SMILES: COC(=O)C1(C)CCCN1C(=O)c1cc(OC)c2oc(NCc3cc(Cl)ccn3)nc2c1]

300 mg (0.899 mmol) of 2-{[(4-chloropyridin-2-yl)methyl]amino}-7-methoxy-1,3-benzoxazole-5-carboxylic acid and 193 mg (1.35 mmol) of methyl 2-methylprolinate [enantiomerically pure isomer] were initially charged in N,N-dimethylformamide (4.50 ml), and 349 mg (470 μl, 2.70 mmol) of N,N-diisopropylethylamine were added. 615 mg (1.62 mmol) of HATU were then added at RT, and the mixture was stirred for 14 h. The reaction solution was concentrated under reduced pressure and then, without further work-up, purified directly by preparative RP-HPLC (acetonitrile/water). Yield: 213 mg (51% of theory).

LC-MS (Method 2A): Rₜ=0.90 min; MS (ESIpos): m/z=459 [M+H]⁺;

¹H-NMR (400 MHz, DMSO-d₆): δ[ppm]=8.71 (t, 1H), 8.52 (d, 1H), 7.53 (d, 1H), 7.46 (dd, 1H), 6.94 (d, 1H), 6.76 (s, 1H), 4.63 (d, 2H), 3.92 (s, 3H), 3.67-3.58 (m, 4H), 3.51-3.42 (m, 1H), 2.14-2.05 (m, 1H), 1.97-1.81 (m, 3H), 1.57 (s, 3H).

### Example 32A

### 1-[(2-{[(4-Chloropyridin-2-yl)methyl]amino}-7-methoxy-1,3-benzoxazol-5-yl)carbonyl]-2-methylproline [enantiomerically pure isomer]

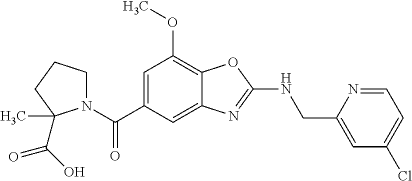 [SMILES: COc1cc(C(=O)N2CCCC2(C)C(=O)O)cc2nc(NCc3cc(Cl)ccn3)oc12]

213 mg (0.464 mmol) of methyl 1-[(2-{[(4-chloropyridin-2-yl)methyl]amino}-7-methoxy-1,3-benzoxazol-5-yl)carbonyl]-2-methylprolinate [enantiomerically pure isomer] were dissolved in tetrahydrofuran/water (3:1, 12 ml), 13.3 mg (0.557 mmol) of lithium hydroxide were added and the mixture was stirred at 70° C. overnight. The reaction solution was then concentrated under reduced pressure, water was added and the mixture was neutralized with 1N aqueous hydrogen chloride solution. The reaction mixture was extracted with ethyl acetate and the aqueous phase was then concentrated under reduced pressure. The crude product (which contains salt) was used without further purification in the next step. Yield: 288 mg (100% of theory, purity: 71%).

LC-MS (Method 3A): Rₜ=1.84 min; MS (ESIpos): m/z=445 [M+H]⁺.

### Example 33A

### N-Benzyl-2-chloro-N-(1,3-dihydroxy-2-methylpropan-2-yl)propanamide [racemate]

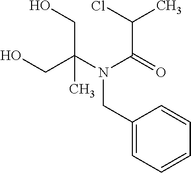 [SMILES: CC(Cl)C(=O)N(Cc1ccccc1)C(C)(CO)CO]

10.7 g (54.8 mmol) of 2-(benzylamino)-2-methylpropane-1,3-diol [lit.: J. Cossy et al., J. Org. Chem. 2012, 77, 6087-6099] were initially charged in dichloromethane (400 ml), the mixture was cooled to 0° C. and 8.32 g (11.5 ml, 82.2 mmol) of triethylamine were added. 8.35 g (6.52 ml, 65.8 mmol) of 2-chloropropionyl chloride [racemate] were then added dropwise. After 30 min of stirring, a further 5.57 g (3.70 ml, 37.3 mmol) of 2-chloropropionyl chloride [racemate] were added dropwise, and the reaction solution was then warmed to RT. The reaction solution was concentrated under reduced pressure, and the residue was taken up in 1N aqueous hydrogen chloride solution and extracted repeatedly with dichloromethane. The organic phases were dried over sodium sulphate, filtered and concentrated under reduced pressure. The crude product was used without further purification in the next step. Yield: 15.6 g (99% of theory).

LC-MS (Method 1A): Rₜ=0.58 min; MS (ESIpos): m/z=286 [M+H]⁺.

### Example 34A

### 4-Benzyl-5-(hydroxymethyl)-2,5-dimethylmorpholin-3-one [diastereomer mixture, 4 isomers]

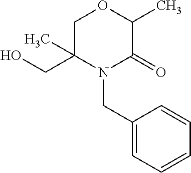 [SMILES: CC1OCC(C)(CO)N(Cc2ccccc2)C1=O]

15.6 g (54.8 mmol) of N-benzyl-2-chloro-N-(1,3-dihydroxy-2-methylpropan-2-yl)propanamide [racemate] were initially charged in isopropanol (300 ml), the mixture was cooled to 0° C. and 24.6 g (219 mmol) of potassium tert-butoxide were added in one portion. The reaction was stirred overnight and allowed to warm to RT during this time. Most of the isopropanol was removed under reduced pressure, and the residue was taken up in 2N aqueous hydrogen chloride solution (300 ml) and extracted repeatedly with dichloromethane. The organic phases were dried over sodium sulphate, filtered and concentrated under reduced pressure. The crude product was used without further purification in the next step. Yield: 15.2 g (96% of theory, purity: 86%, diastereomer ratio about 1:1).

LC-MS (Method 1A): Rₜ=0.72 min (diastereomer 1, 2 isomers), Rₜ=0.74 min (diastereomer 2, 2 isomers); MS (ESIpos): m/z=250 [M+H]⁺.

### Example 35A

### (4-Benzyl-3,6-dimethylmorpholin-3-yl)methanol [diastereomer mixture, 4 isomers]

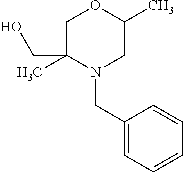 [SMILES: CC1CN(Cc2ccccc2)C(C)(CO)CO1]

15.6 g (62.6 mmol) of 4-benzyl-5-(hydroxymethyl)-2,5-dimethylmorpholin-3-one [diastereomer mixture, 4 isomers] were initially charged in tetrahydrofuran (300 ml), 93.9 ml (188 mmol) of 2M borane/dimethyl sulphide complex solution in tetrahydrofuran were added under argon and the reaction mixture was then stirred under reflux for 3 h. The mixture was subsequently cooled to RT, methanol (150 ml) was added carefully and the mixture was then stirred under reflux for 4 h. The mixture was then concentrated completely under reduced pressure and the residue was purified by preparative RP-HPLC (acetonitrile/water). Yield: 6.53 g (44% of theory).

LC-MS (Method 4A): Rₜ=0.28 min; MS (ESIpos): m/z=236 [M+H]⁺.

### Example 36A

### (4-Benzyl-3,6-dimethylmorpholin-3-yl)methanol [enantiomerically pure isomer 1]

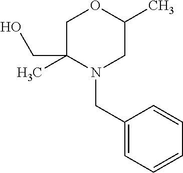 [SMILES: CC1CN(Cc2ccccc2)C(C)(CO)CO1]

Enantiomer separation on a chiral phase of 6.53 g of the compound from Example 35A according to Method 18D and re-purification by preparative RP-HPLC (acetonitrile/water) gave 1.12 g of Example 36A (enantiomerically pure isomer 1), 1.23 g of Example 37A (enantiomerically pure isomer 2), 441 mg of Example 38A (enantiomerically pure isomer 3) and 457 mg of Example 39A (enantiomerically pure isomer 4).

HPLC (Method 18E): Rₜ=5.13 min, >99.0% ee;

LC-MS (Method 4A): Rₜ=2.56 min; MS (ESIpos): m/z=236 [M+H]⁺.

### Example 37A

### (4-Benzyl-3,6-dimethylmorpholin-3-yl)methanol [enantiomerically pure isomer 2]

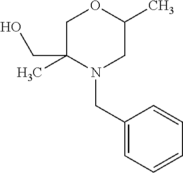 [SMILES: CC1CN(Cc2ccccc2)C(C)(CO)CO1]

Enantiomer separation on a chiral phase of 6.53 g of the compound from Example 35A according to Method 18D and re-purification by preparative RP-HPLC (acetonitrile/water) gave 1.12 g of Example 36A (enantiomerically pure isomer 1), 1.23 g of Example 37A (enantiomerically pure isomer 2), 441 mg of Example 38A (enantiomerically pure isomer 3) and 457 mg of Example 39A (enantiomerically pure isomer 4).

HPLC (Method 18E): Rₜ=5.73 min, >99.0% ee;

LC-MS (Method 4A): Rₜ=2.52 min; MS (ESIpos): m/z=236 [M+H]⁺.

### Example 38A

### (4-Benzyl-3,6-dimethylmorpholin-3-yl)methanol [enantiomerically pure isomer 3]

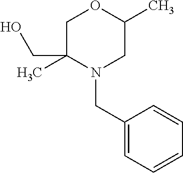 [SMILES: CC1CN(Cc2ccccc2)C(C)(CO)CO1]

Enantiomer separation on a chiral phase of 6.53 g of the compound from Example 35A according to Method 18D and re-purification by preparative RP-HPLC (acetonitrile/water) gave 1.12 g of Example 36A (enantiomerically pure isomer 1), 1.23 g of Example 37A (enantiomerically pure isomer 2), 441 mg of Example 38A (enantiomerically pure isomer 3) and 457 mg of Example 39A (enantiomerically pure isomer 4).

HPLC (Method 18E): Rₜ=6.57 min, >99.0% ee;

LC-MS (Method 4A): Rₜ=2.60 min; MS (ESIpos): m/z=236 [M+H]⁺.

### Example 39A

### (4-Benzyl-3,6-dimethylmorpholin-3-yl)methanol [enantiomerically pure isomer 4]

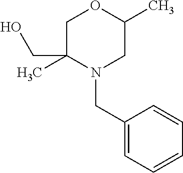 [SMILES: CC1CN(Cc2ccccc2)C(C)(CO)CO1]

Enantiomer separation on a chiral phase of 6.53 g of the compound from Example 35A according to Method 18D and re-purification by preparative RP-HPLC (acetonitrile/water) gave 1.12 g of Example 36A (enantiomerically pure isomer 1), 1.23 g of Example 37A (enantiomerically pure isomer 2), 441 mg of Example 38A (enantiomerically pure isomer 3) and 457 mg of Example 39A (enantiomerically pure isomer 4).

HPLC (Method 18E): Rₜ=6.92 min, >99.0% ee;

LC-MS (Method 4A): Rₜ=2.61 min; MS (ESIpos): m/z=236 [M+H]⁺.

### Example 40A

### (3,6-Dimethylmorpholin-3-yl)methanol [enantiomerically pure isomer 1]

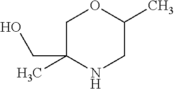

1.12 g (4.76 mmol) of (4-benzyl-3,6-dimethylmorpholin-3-yl)methanol [enantiomerically pure isomer 1, Example 36A] were initially charged in ethanol (120 ml), 112 mg of palladium on carbon (10%) and 112 mg of palladium hydroxide on carbon (20%) were added under argon and the mixture was then stirred under an atmosphere of hydrogen at standard pressure overnight. The reaction solution was filtered through kieselguhr and the filter residue was washed with methanol. The filtrate was concentrated under reduced pressure and the product was dried under high vacuum. Yield: 651 mg (94% of theory).

¹H-NMR (400 MHz, DMSO-d₆): δ[ppm]=4.44 (br. s., 1H), 3.56 (d, 1H), 3.40 (d, 2H), 3.36-3.22 (m, 2H), 3.06 (d, 1H), 1.98 (br. s., 1H), 0.99 (d, 3H), 0.77 (s, 3H), One proton not visible.

### Example 41A

### (3,6-Dimethylmorpholin-3-yl)methanol [enantiomerically pure isomer 4]

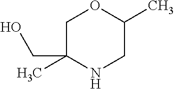

457 mg (1.94 mmol) of (4-benzyl-3,6-dimethylmorpholin-3-yl)methanol [enantiomerically pure isomer 4, Example 39A] were initially charged in ethanol (50 ml), 46 mg of palladium on carbon (10%) and 46 mg of palladium hydroxide on carbon (20%) were added under argon and the mixture was then stirred under an atmosphere of hydrogen at standard pressure overnight. The reaction solution was filtered through kieselguhr and the filter residue was washed with methanol. The filtrate was concentrated under reduced pressure and the product was dried under high vacuum. Yield: 280 mg (99% of theory).

¹H-NMR (400 MHz, DMSO-d₆): δ[ppm]=4.62 (br. s., 1H), 3.40 (d, 1H), 3.30-3.18 (m, 2H), 3.11 (br. s., 2H), 2.58-2.55 (m, 1H), 1.81 (m_{c}, 1H), 1.05-0.96 (m, 6H), One proton not visible.

### Example 42A

### N-Benzyl-2-chloro-N-(1,3-dihydroxypropan-2-yl)propanamide [racemate]

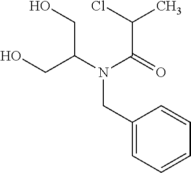 [SMILES: CC(Cl)C(=O)N(Cc1ccccc1)C(CO)CO]

60.5 g (334 mmol) of 2-(benzylamino)propane-1,3-diol [lit.: W. Lacôte et al., Org. Lett. 2011, 13, 5990-5993] were initially charged in isopropanol (0.93 l), the mixture was cooled to 0° C. and 50.7 g (69.8 ml, 501 mmol) of triethylamine were added. 50.9 g (38.9 ml, 401 mmol) of 2-chloropropionyl chloride [racemate] were then added dropwise. The reaction solution was allowed to warm to RT and then concentrated under reduced pressure. 0.5N aqueous hydrogen chloride solution was added to the residue, and the mixture was extracted with dichloromethane. The organic phases were dried over sodium sulphate, filtered and concentrated under reduced pressure. The crude product was used without further purification in the next step. Yield: 91.7 g (94% of theory).

LC-MS (Method 1A): Rₜ=0.71 min; MS (ESIpos): m/z=272 [M+H]⁺.

### Example 43A

### 4-Benzyl-5-(hydroxymethyl)-2-methylmorpholin-3-one [diastereomer mixture, 4 isomers]

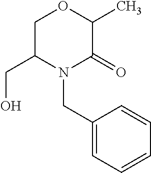 [SMILES: CC1OCC(CO)N(Cc2ccccc2)C1=O]

81.3 g (272 mmol, purity: 91%) of N-benzyl-2-chloro-N-(1,3-dihydroxypropan-2-yl)propanamide [racemate] were initially charged in isopropanol (600 ml), the mixture was cooled to 0° C. and 91.6 g (817 mmol) of potassium tert-butoxide were added. The reaction solution was allowed to warm to RT and was stirred at RT overnight. Most of the isopropanol was removed under reduced pressure and the residue was taken up in dichloromethane. The mixture was washed with water and the organic phase was dried over magnesium sulphate, filtered and concentrated under reduced pressure. The crude product was used without further purification in the next step. Yield: 61.7 g (96% of theory, diastereomer ratio about 7:3).

LC-MS (Method 2A): Rₜ=0.61 min (diastereomer 1, 2 isomers), Rₜ=0.62 min (diastereomer 2, 2 isomers);

MS (ESIpos): m/z=236 [M+H]⁺.

### Example 44A

### 4-Benzyl-5-({[tert-butyl(dimethyl)silyl]oxy}methyl)-2-methylmorpholin-3-one [diastereomer mixture, 4 isomers]

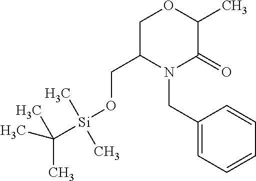 [SMILES: CC1OCC(CO[Si](C)(C)C(C)(C)C)N(Cc2ccccc2)C1=O]

21.5 g (91.4 mmol) of 4-benzyl-5-(hydroxymethyl)-2-methylmorpholin-3-one [diastereomer mixture, 4 isomers] were initially charged in N,N-dimethylformamide (126 ml), and 12.4 g (183 mmol) of imidazole and then 14.5 g (96.0 mmol) of tert-butyldimethylsilyl chloride were added at RT. The mixture was stirred for 2 h, and most of the solvent was then removed under reduced pressure. The residue was taken up in ethyl acetate/water and the organic phase was washed with water, 0.4N aqueous hydrogen chloride solution, saturated aqueous sodium bicarbonate solution and water. The organic phase was dried over sodium sulphate, filtered and concentrated under reduced pressure. The crude product was used without further purification in the next step. Yield: 31.2 g (97% of theory, diastereomer ratio about 7:3).

LC-MS (Method 1A): Rₜ=1.41 min; MS (ESIpos): m/z=350 [M+H]⁺;

¹H-NMR (400 MHz, DMSO-d₆): δ[ppm]=7.38-7.18 (m, 5H), 5.00 (d, 0.3H), 4.95 (d, 0.7H), 4.32-4.19 (m, 2H), 3.92-3.85 (m, 1H), 3.75-3.62 (m, 3H), 3.32-3.26 (m, 0.3H), 3.19-3.13 (m, 0.7H), 1.35 (d, 0.9H), 1.32 (d, 2.1H), 0.84-0.80 (m, 9H), 0.04-0.03 (m, 6H).

### Example 45A

### 2-Allyl-4-benzyl-5-({[tert-butyl(dimethyl)silyl]oxy}methyl)-2-methylmorpholin-3-one [diastereomer mixture, 4 isomers]

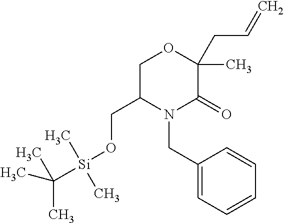 [SMILES: C=CCC1(C)OCC(CO[Si](C)(C)C(C)(C)C)N(Cc2ccccc2)C1=O]

30.6 g (87.5 mmol) of 4-benzyl-5-({[tert-butyl(dimethyl)silyl]oxy}methyl)-2-methylmorpholin-3-one [diastereomer mixture, 4 isomers] were initially charged in tetrahydrofuran (1.13 l), 123 ml (123 mmol) of 1M lithium hexamethyldisilazide solution in tetrahydrofuran were added under argon and at −78° C. and the reaction mixture was then stirred for 15 min. Subsequently, at −78° C., 17.6 g (9.61 ml, 105 mmol) of allyl iodide were added, and the reaction mixture was warmed to RT and stirred overnight. The reaction was terminated by addition of saturated aqueous ammonium chloride solution and the reaction mixture was then extracted with ethyl acetate. The organic phase was washed with saturated aqueous sodium chloride solution, dried over sodium sulphate, filtered and concentrated under reduced pressure. The crude product was used without further purification in the next step. Yield: 36.6 g (100% of theory).

LC-MS (Method 1A): Rₜ=1.53 min; MS (ESIpos): m/z=390 [M+H]⁺.

### Example 46A

### [4-Benzyl-5-({[tert-butyl(dimethyl)silyl]oxy}methyl)-2-methyl-3-oxomorpholin-2-yl]acetaldehyde [diastereomer mixture, 4 isomers]

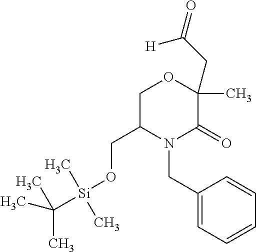 [SMILES: CC1(CC=O)OCC(CO[Si](C)(C)C(C)(C)C)N(Cc2ccccc2)C1=O]

1.80 g (4.62 mmol) of 2-allyl-4-benzyl-5-({[tert-butyl(dimethyl)silyl]oxy}methyl)-2-methylmorpholin-3-one [diastereomer mixture, 4 isomers] were initially charged in tetrahydrofuran (100 ml) and water (60 ml), and 1.16 ml (0.427 mmol) of a 2.5% solution of osmium tetroxide in tert-butanol and 2.96 g (13.9 mmol) of sodium periodate were added at 0° C. The mixture was then warmed to RT and stirred for 20 h. The reaction solution was filtered through kieselguhr and the filter residue was washed with ethyl acetate. After separation of the phases, the organic phase was dried over sodium sulphate, filtered and concentrated under reduced pressure. The crude product was used without further purification in the next step. Yield: 2.02 g (87% of theory, purity: 78%).

LC-MS (Method 2A): Rₜ=1.42 min; MS (ESIpos): m/z=392 [M+H]⁺.

### Example 47A

### 4-Benzyl-5-({[tert-butyl(dimethyl)silyl]oxy}methyl)-2-(2-hydroxyethyl)-2-methylmorpholin-3-one [diastereomer mixture, 4 isomers]

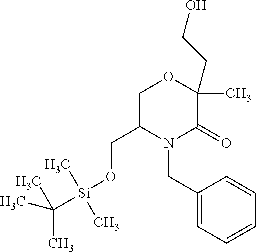 [SMILES: CC1(CCO)OCC(CO[Si](C)(C)C(C)(C)C)N(Cc2ccccc2)C1=O]

22.2 g (32.5 mmol, purity: 57%) of [4-benzyl-5-({[tert-butyl(dimethyl)silyl]oxy}-methyl)-2-methyl-3-oxomorpholin-2-yl]acetaldehyde [diastereomer mixture, 4 isomers] were initially charged in methanol (242 ml), and 1.84 g (48.7 mmol) of sodium borohydride were added at 0° C. The mixture was then warmed to RT and stirred for 30 min. Water was added to the reaction solution, most of the methanol was removed under reduced pressure and the residue was extracted with ethyl acetate. The organic phase was dried over sodium sulphate, filtered and concentrated under reduced pressure. The crude product was used without further purification in the next step. Yield: 22.8 g (quant., purity: 62%).

LC-MS (Method 1A): Rₜ=1.28 min; MS (ESIpos): m/z=394 [M+H]⁺.

### Example 48A

### 4-Benzyl-5-({[tert-butyl(dimethyl)silyl]oxy}methyl)-2-(2-{[tert-butyl(diphenyl)silyl]oxy}ethyl)-2-methylmorpholin-3-one [diastereomer mixture, 4 isomers]

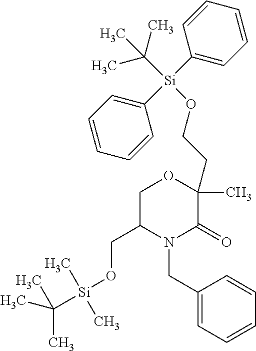 [SMILES: CC1(CCO[Si](c2ccccc2)(c2ccccc2)C(C)(C)C)OCC(CO[Si](C)(C)C(C)(C)C)N(Cc2ccccc2)C1=O]

15.0 g (38.1 mmol) of 4-benzyl-5-({[tert-butyl(dimethyl)silyl]oxy}methyl)-2-(2-hydroxyethyl)-2-methylmorpholin-3-one [diastereomer mixture, 4 isomers], 7.73 g (114 mmol) of imidazole and 4-(dimethylamino)pyridine (6 mg) were initially charged in N,N-dimethylformamide (52.8 ml), and 15.7 g (57.2 mmol) of tert-butyldiphenylsilyl chloride were added at 0° C. The mixture was stirred for 60 h and warmed to RT during this time. Subsequently, most of the solvent was removed under reduced pressure, the residue was taken up in ethyl acetate/water and the organic phase was washed with water and saturated aqueous sodium chloride solution. The organic phase was dried over magnesium sulphate, filtered and concentrated under reduced pressure. The crude product obtained was purified by means of flash chromatography on silica gel (cyclohexane/ethyl acetate 9:1). Yield: 15.6 g (49% of theory, purity: 50%).

LC-MS (Method 7A): Rₜ=6.96 min; MS (ESIpos): m/z=633 [M+H]⁺.

### Example 49A

### 4-Benzyl-2-(2-{[tert-butyl(diphenyl)silyl]oxy}ethyl)-5-(hydroxymethyl)-2-methylmorpholin-3-one [diastereomer mixture, 4 isomers]

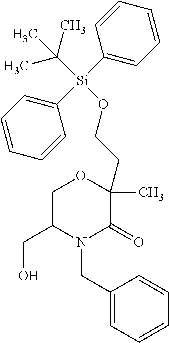 [SMILES: CC1(CCO[Si](c2ccccc2)(c2ccccc2)C(C)(C)C)OCC(CO)N(Cc2ccccc2)C1=O]

15.6 g (12.3 mmol, crude product) of 4-benzyl-5-({[tert-butyl(dimethyl)silyl]oxy}methyl)-2-(2-{[tert-butyl(diphenyl)silyl]oxy}ethyl)-2-methylmorpholin-3-one [diastereomer mixture, 4 isomers] were dissolved in concentrated acetic acid, tetrahydrofuran and water (250 ml, 3:1:1), and the mixture was stirred at RT overnight. The reaction solution was then diluted with ethyl acetate and washed three times with water, once with saturated aqueous sodium bicarbonate solution and then with saturated aqueous sodium chloride solution. The organic phase was dried over sodium sulphate, filtered and concentrated under reduced pressure. The crude product obtained was purified by means of flash chromatography on silica gel (cyclohexane/ethyl acetate 7:3-1:1).

Yield: 2.55 g (40% of theory).

LC-MS (Method 1A): Rₜ=1.43 min; MS (ESIpos): m/z=518 [M+H]⁺.

### Example 50A

### 4-Benzyl-5-(fluoromethyl)-2-(2-hydroxyethyl)-2-methylmorpholin-3-one [diastereomer mixture, 4 isomers]

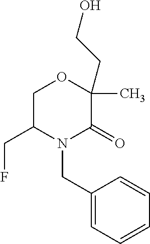 [SMILES: CC1(CCO)OCC(CF)N(Cc2ccccc2)C1=O]

2.00 g (3.86 mmol) of 4-benzyl-2-(2-{[tert-butyl(diphenyl)silyl]oxy}ethyl)-5-(hydroxymethyl)-2-methylmorpholin-3-one [diastereomer mixture, 4 isomers] were initially charged in tetrahydrofuran (40.0 ml), and 20.0 ml (46.5 mmol) of bis(2-methoxyethyl)aminosulphur trifluoride (Deoxofluor, 50% strength solution in tetrahydrofuran) were added slowly at RT. 1 drop of methanol was then added and the mixture was subsequently stirred at RT for 2 h and then under reflux for 2 h. The reaction solution was subsequently carefully added dropwise to saturated aqueous sodium bicarbonate solution, the phases were separated and the aqueous phase was extracted with dichloromethane. The combined organic phases were dried over sodium sulphate, filtered and concentrated under reduced pressure. The crude product was taken up in tetrahydrofuran (40.0 ml), and 15.5 ml (15.5 mmol) of tetra-n-butylammonium fluoride solution (1.0M in tetrahydrofuran) were added. The reaction solution was stirred at RT overnight and then concentrated under reduced pressure. The residue was taken up in dichloromethane and washed with water and the organic phase was then dried over sodium sulphate, filtered and concentrated under reduced pressure. The crude product was used without further purification in the next step. Yield: 2.74 g (74% of theory, purity: 29%).

LC-MS (Method 1A): Rₜ=0.76 min; MS (ESIpos): m/z=282 [M+H]⁺.

### Example 51A

### 2-[4-Benzyl-5-(fluoromethyl)-2-methylmorpholin-2-yl]ethanol [diastereomer mixture, 4 isomers]

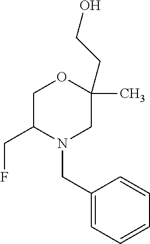 [SMILES: CC1(CCO)CN(Cc2ccccc2)C(CF)CO1]

2.74 g (2.87 mmol, purity: 29%) of 4-benzyl-5-(fluoromethyl)-2-(2-hydroxyethyl)-2-methylmorpholin-3-one [diastereomer mixture, 4 isomers] were initially charged in tetrahydrofuran (29.1 ml), 5.74 ml (11.5 mmol) of 2M borane/dimethyl sulphide complex solution in tetrahydrofuran were added under argon and the reaction mixture was then stirred under reflux for 3 h. The mixture was subsequently cooled to 0° C., methanol (7.5 ml) was added carefully and the mixture was then stirred under reflux for 30 min. The mixture was then concentrated completely under reduced pressure and the residue was purified directly by preparative RP-HPLC (acetonitrile/water, isocratic). Yield: 769 mg (97% of theory, diastereomer ratio: about 3:2).

LC-MS (Method 1A): Rₜ=0.63 min (diastereomer 1, 2 isomers), Rₜ=0.65 min (diastereomer 2, 2 isomers).

MS (ESIpos): m/z=268 [M+H]⁺.

### Example 52A

### 2-[5-(Fluoromethyl)-2-methylmorpholin-2-yl]ethanol [diastereomer mixture, 4 isomers]

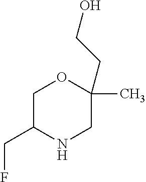

769 mg (2.80 mmol) of 2-[4-benzyl-5-(fluoromethyl)-2-methylmorpholin-2-yl)ethanol [diastereomer mixture, 4 isomers] were initially charged in ethanol (41.8 ml), 154 mg of palladium on carbon (10%) and 77.0 mg of palladium hydroxide on carbon (20%) were added under argon, and the mixture was then stirred under an atmosphere of hydrogen at standard pressure for 4 h. The reaction solution was filtered through kieselguhr and the filter residue was washed with ethanol. The filtrate was concentrated under reduced pressure and repeatedly co-evaporated with dichloromethane, and the product was then dried under high vacuum. Yield: 521 mg (99% of theory).

MS (Method 1C): m/z=178 [M+H]⁺.

### Example 53A

### 4-Benzyl-5-({[tert-butyl(dimethyl)silyl]oxy}methyl)-2-(2-hydroxypropyl)-2-methylmorpholin-3-one [diastereomer mixture, 8 isomers]

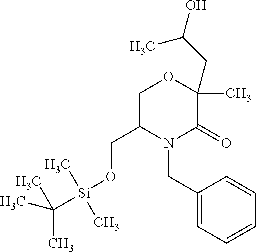 [SMILES: CC(O)CC1(C)OCC(CO[Si](C)(C)C(C)(C)C)N(Cc2ccccc2)C1=O]

10.0 g (17.9 mmol, purity: 70%) of [4-benzyl-5-({[tert-butyl(dimethyl)silyl]oxy}methyl)-2-methyl-3-oxomorpholin-2-yl]acetaldehyde [diastereomer mixture, 4 isomers] were initially charged in tetrahydrofuran (75.4 ml), and 26.8 ml (26.8 mmol) of a 1M solution of methylmagnesium bromide in tetrahydrofuran were added at −78° C. The mixture was stirred at −78° C. for 15 min and then warmed to RT. Saturated aqueous ammonium chloride solution was then added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The crude product was used without further purification in the next step. Yield: 9.65 g of crude product.

LC-MS (Method 1A): Rₜ=1.25 min (diastereomer 1, 2 isomers), Rₜ=1.27 min (diastereomer 2, 2 isomers), 1.39 (diastereomer 3, 2 isomers), one diastereoisomer, 2 isomers obscured.

MS (ESIpos): m/z=408 [M+H]⁺.

### Example 54A

### 4-Benzyl-5-({[tert-butyl(dimethyl)silyl]oxy}methyl)-2-(2-{[tert-butyl(diphenyl)silyl]oxy}propyl)-2-methylmorpholin-3-one [diastereomer mixture, 8 isomers]

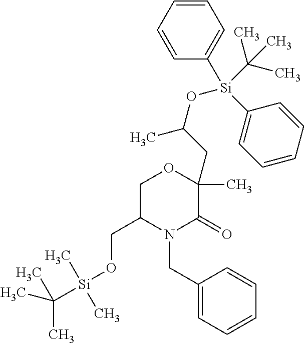 [SMILES: CC(CC1(C)OCC(CO[Si](C)(C)C(C)(C)C)N(Cc2ccccc2)C1=O)O[Si](c1ccccc1)(c1ccccc1)C(C)(C)C]

9.65 g (22.0 mmol) of 4-benzyl-5-({[tert-butyl(dimethyl)silyl]oxy}methyl)-2-(2-hydroxypropyl)-2-methylmorpholin-3-one [diastereomer mixture, 8 isomers], 3.00 g (44.0 mmol) of imidazole and 134 mg (1.10 mmol) of 4-(dimethylamino)pyridine were initially charged in dichloromethane (500 ml), and 6.66 g (6.30 ml, 24.2 mmol) of tert-butyldiphenylsilyl chloride were added at 0° C. The mixture was stirred for 48 h and warmed to RT during this time. The mixture was warmed to 40° C. and stirred for a further 24 h. After addition of a further 1.06 g (1.00 ml, 4.06 mmol) of tert-butyldiphenylsilyl chloride, the mixture was stirred at 40° C. until the reaction had gone to completion. Subsequently, the reaction solution was diluted with dichloromethane and the organic phase was washed with water. The organic phase was dried over magnesium sulphate, filtered and concentrated under reduced pressure. The crude product was used without further purification in the next step. Yield: 17.2 g of crude product.

LC-MS (Method 1A): Rₜ=1.82 min (diastereomer 1, 2 isomers+diastereomer 2, 2 isomers), Rₜ=1.87 min (diastereomer 3, 2 isomers+diastereomer 4, 2 isomers);

MS (ESIpos): m/z=647 [M+H]⁺.

### Example 55A

### 4-Benzyl-2-(2-{[tert-butyl(diphenyl)silyl]oxy}propyl)-5-(hydroxymethyl)-2-methylmorpholin-3-one [diastereomer mixture, 8 isomers]

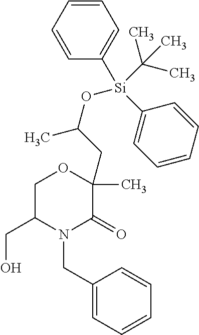 [SMILES: CC(CC1(C)OCC(CO)N(Cc2ccccc2)C1=O)O[Si](c1ccccc1)(c1ccccc1)C(C)(C)C]

17.2 g (6.12 mmol, crude product) of 4-benzyl-5-({[tert-butyl(dimethyl)silyl]oxy}methyl)-2-(2-{[tert-butyl(diphenyl)silyl]oxy}propyl)-2-methylmorpholin-3-one [diastereomer mixture, 8 isomers] were dissolved in concentrated acetic acid (111 ml), tetrahydrofuran (36.0 ml) and water (36.0 ml), and the mixture was stirred at RT for 5 d. The reaction solution was then diluted with ethyl acetate and washed once with water, three times with saturated aqueous sodium bicarbonate solution and then with saturated aqueous sodium chloride solution. The organic phase was dried over sodium sulphate, filtered and concentrated under reduced pressure. The crude product obtained was purified by means of flash chromatography on silica gel (cyclohexane/ethyl acetate 20:1-1:1). Yield: 2.22 g (68% of theory).

LC-MS (Method 1A): Rₜ=1.50 min; MS (ESIpos): m/z=532 [M+H]⁺.

### Example 56A

### 4-Benzyl-2-(2-{[tert-butyl(diphenyl)silyl]oxy}propyl)-5-(fluoromethyl)-2-methylmorpholin-3-one [diastereomer mixture, 8 isomers]

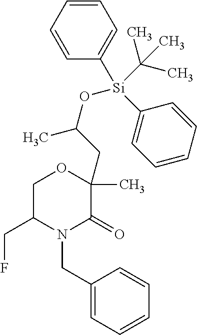 [SMILES: CC(CC1(C)OCC(CF)N(Cc2ccccc2)C1=O)O[Si](c1ccccc1)(c1ccccc1)C(C)(C)C]

2.10 g (3.95 mmol) of 4-benzyl-2-(2-{[tert-butyl(diphenyl)silyl]oxy}propyl)-5-(hydroxymethyl)-2-methylmorpholin-3-one [diastereomer mixture, 8 isomers] were initially charged in tetrahydrofuran (105 ml), and 11.7 ml (27.1 mmol) of bis(2-methoxyethyl)aminosulphur trifluoride (Deoxofluor, 50% strength solution in tetrahydrofuran) were added slowly at RT. 2 drops of ethanol were then added and the mixture was subsequently stirred under reflux for 5 h. The reaction solution was subsequently carefully added dropwise to saturated aqueous sodium bicarbonate solution, the phases were separated and the aqueous phase was extracted with dichloromethane. The combined organic phases were dried over sodium sulphate, filtered and concentrated under reduced pressure. The crude product was used without further purification in the next step. Yield: 3.73 g (quant., purity: 82%).

LC-MS (Method 1A): Rₜ=1.63 min; MS (ESIpos): m/z=534 [M+H]⁺.

### Example 57A

### 4-Benzyl-5-(fluoromethyl)-2-(2-hydroxypropyl)-2-methylmorpholin-3-one [diastereomer mixture, 8 isomers]

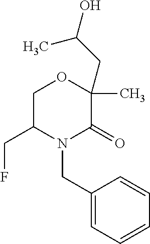 [SMILES: CC(O)CC1(C)OCC(CF)N(Cc2ccccc2)C1=O]

3.70 g (5.68 mmol, purity: 82%) of 4-benzyl-2-(2-{[tert-butyl(diphenyl)silyl]oxy}propyl)-5-(fluoromethyl)-2-methylmorpholin-3-one [diastereomer mixture, 8 isomers] were initially charged in tetrahydrofuran (124 ml), and 19.7 ml (19.7 mmol) of tetra-n-butylammonium fluoride solution (1.0M in tetrahydrofuran) were added at RT. The reaction solution was stirred at RT overnight and then concentrated under reduced pressure. The residue was taken up in dichloromethane and washed with water and the organic phase was then dried over sodium sulphate, filtered and concentrated under reduced pressure. The crude product was then purified by preparative RP-HPLC (acetonitrile/water). Yield: 728 mg (43% of theory).

LC-MS (Method 1A): Rₜ=0.85 min (diastereomer 1+diastereomer 2), Rₜ=0.86 min (diastereomer 3+diastereomer 4);

MS (ESIpos): m/z=296 [M+H]⁺.

### Example 58A

### 1-[4-Benzyl-5-(fluoromethyl)-2-methylmorpholin-2-yl]propan-2-ol trifluoroacetate [diastereomer 1, 2 isomers+diastereomer 2, 2 isomers+diastereomer 3, 2 isomers+diastereomer 4, 2 isomers]

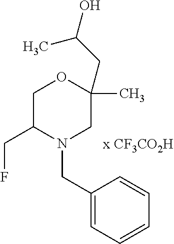 [SMILES: CC(O)CC1(C)CN(Cc2ccccc2)C(CF)CO1]

728 mg (2.46 mmol) of 4-benzyl-5-(fluoromethyl)-2-(2-hydroxypropyl)-2-methylmorpholin-3-one [diastereomer mixture, 8 isomers] were initially charged in tetrahydrofuran (24.2 ml), 4.93 ml (9.86 mmol) of 2M borane/dimethyl sulphide complex solution in tetrahydrofuran were added under argon and the reaction mixture was then stirred under reflux for 2 h. At 0° C., methanol (10 ml) was subsequently added carefully and the mixture was stirred under reflux for 30 min. The mixture was then concentrated completely under reduced pressure and the residue was purified by preparative RP-HPLC (acetonitrile/water). The isomer mixture (262 mg) was then separated on an achiral phase according to Method 3G into the four diastereomers. Yield: 15.2 mg (2% of theory, diastereomer 1, 2 isomers); 166 mg (23% of theory, diastereomer 2, 2 isomers), 44.7 mg (6% of theory, diastereomer 3, 2 isomers), 92.5 mg (13% of theory, diastereomer 4, 2 isomers).

LC-MS (Method 1A): Rₜ=0.68 min (diastereomer 1, 2 isomers), Rₜ=0.69 min (diastereomer 2, 2 isomers), Rₜ=0.73 min (diastereomer 3, 2 isomers), Rₜ=0.68 min (diastereomer 4, 2 isomers).

MS (ESIpos): m/z=282 [M+H-TFA]⁺.

### Example 59A

### 1-[5-(Fluoromethyl)-2-methylmorpholin-2-yl]propan-2-ol trifluoroacetate [diastereomer 2, 2 isomers]

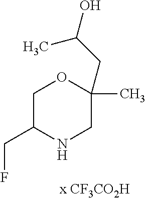

166 mg (0.421 mmol) of 1-[4-benzyl-5-(fluoromethyl)-2-methylmorpholin-2-yl]propan-2-ol trifluoroacetate [diastereomer 2, 2 isomers, Example 58A] were initially charged in ethanol (4.23 ml), 17.0 mg of palladium on carbon (10%) and 8.0 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was then dried under high vacuum. Yield: 126 mg (98% of theory).

GC-MS (Method 2B): Rₜ=3.97 min.

### Example 60A

### N-Benzyl-2-chloro-N-[(2R)-1-hydroxypropan-2-yl]propanamide [diastereomer mixture, 2 isomers]

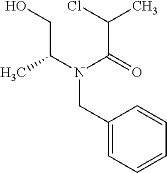 [SMILES: CC(Cl)C(=O)N(Cc1ccccc1)[C@H](C)CO]

16.4 g (99.3 mmol) of (2R)-2-(benzylamino)propan-1-ol [lit.: T. J. Tewson et al., Synthesis 2002, 6, 766-770] were initially charged in isopropanol (500 ml), the mixture was cooled to 0° C. and 20.1 g (27.7 ml, 199 mmol) of triethylamine were added. 13.9 g (10.8 ml, 109 mmol) of 2-chloropropionyl chloride [racemate] were then added dropwise, and the reaction solution was allowed to warm to RT, stirred overnight and then concentrated under reduced pressure. 0.5N aqueous hydrogen chloride solution was added to the residue, and the mixture was extracted with ethyl acetate. The organic phases were dried over sodium sulphate, filtered and concentrated under reduced pressure. The crude product was used without further purification in the next step. Yield: 24.3 g (88% of theory, purity: 92%, diastereomer ratio about 1:1).

LC-MS (Method 1A): Rₜ=0.80 min (diastereomer 1), Rₜ=0.84 min (diastereomer 2);

MS (ESIpos): m/z=256 [M+H]⁺.

### Example 61A

### (5R)-4-Benzyl-2,5-dimethylmorpholin-3-one [diastereomer mixture, 2 isomers]

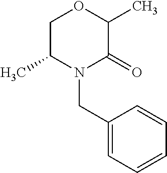 [SMILES: CC1OC[C@@H](C)N(Cc2ccccc2)C1=O]

30.0 g (109 mmol, purity: 93%) of N-benzyl-2-chloro-N-[(2R)-1-hydroxypropan-2-yl]propanamide [diastereomer mixture, 2 isomers] were initially charged in isopropanol (588 ml), the mixture was cooled to 0° C. and 49.0 g (436 mmol) of potassium tert-butoxide were added. The reaction solution was allowed to warm to RT and was stirred overnight. Most of the isopropanol was removed under reduced pressure, and the residue was taken up in water. The mixture was extracted with ethyl acetate, and the organic phases were dried over magnesium sulphate, filtered and concentrated under reduced pressure. The crude product was used without further purification in the next step.

Yield: 22.8 g (93% of theory).

LC-MS (Method 1A): Rₜ=0.85 min; MS (ESIpos): m/z=220 [M+H]⁺.

### Example 62A

### (5R)-2-Allyl-4-benzyl-2,5-dimethylmorpholin-3-one [diastereomer mixture, 2 isomers]

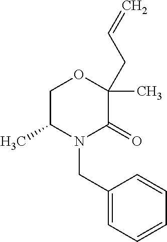 [SMILES: C=CCC1(C)OC[C@@H](C)N(Cc2ccccc2)C1=O]

22.8 g (104 mmol) of (5R)-4-benzyl-2,5-dimethylmorpholin-3-one [diastereomer mixture, 2 isomers] were initially charged in tetrahydrofuran (1.34 l), 146 ml (146 mmol) of 1M lithium hexamethyldisilazide solution in tetrahydrofuran were added under argon and at −78° C. and the mixture was stirred for 15 min. At −78° C., 21.0 g (11.4 ml, 125 mmol) of allyl iodide were then added, and the reaction mixture was allowed to warm to RT and stirred for 3 h. The reaction was terminated by addition of saturated aqueous ammonium chloride solution, and the mixture was then extracted with ethyl acetate. The organic phase was washed with saturated aqueous sodium chloride solution, dried over sodium sulphate, filtered and concentrated under reduced pressure. The crude product was used without further purification in the next step. Yield: 27.5 g (77% of theory, purity: 75%).

LC-MS (Method 1A): Rₜ=0.99 min; MS (ESIpos): m/z=260 [M+H]⁺.

### Example 63A

### [(5R)-4-Benzyl-2,5-dimethyl-3-oxomorpholin-2-yl]acetaldehyde [diastereomer mixture, 2 isomers]

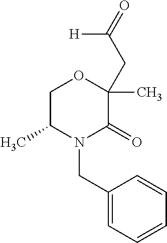 [SMILES: C[C@@H]1COC(C)(CC=O)C(=O)N1Cc1ccccc1]

27.4 g (79.9 mmol, purity: 75%) of (5R)-2-allyl-4-benzyl-2,5-dimethylmorpholin-3-one [diastereomer mixture, 2 isomers] were initially charged in tetrahydrofuran (620 ml) and water (370 ml), and 4.35 ml (1.60 mmol) of a 2.5% solution of osmium tetroxide in tert-butanol and 51.2 g (240 mmol) of sodium periodate were added at 0° C. The reaction solution was allowed to warm to RT and was stirred overnight. The reaction solution was filtered through kieselguhr and the filter residue was washed with tetrahydrofuran. The reaction solution was taken up in ethyl acetate and diluted with water. After separation of the phases, the organic phase was washed with 1N aqueous sodium sulphite solution (2×400 ml), dried over sodium sulphate, filtered and concentrated under reduced pressure. The crude product was used without further purification in the next step. Yield: 23.6 g of crude product.

LC-MS (Method 1A): Rₜ=0.81 min (enantiomerically pure isomer 1), Rₜ=0.84 min (enantiomerically pure isomer 2);

MS (ESIpos): m/z=262 [M+H]⁺.

### Example 64A

### (5R)-4-Benzyl-2-(2-hydroxyethyl)-2,5-dimethylmorpholin-3-one [diastereomer mixture, 2 isomers]

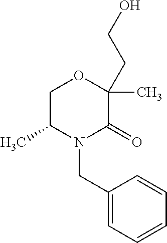 [SMILES: C[C@@H]1COC(C)(CCO)C(=O)N1Cc1ccccc1]

7.00 g (about 26.8 mmol, crude product) of [(5R)-4-benzyl-2,5-dimethyl-3-oxomorpholin-2-yl]acetaldehyde [diastereomer mixture, 2 isomers] were initially charged in methanol (200 ml), and 3.04 g (80.4 mmol) of sodium borohydride were added at 0° C. The reaction solution was allowed to warm to RT and stirred for 30 min. Water was added to the reaction solution, most of the methanol was removed under reduced pressure and the residue was extracted with ethyl acetate. The organic phase was dried over sodium sulphate, filtered and concentrated under reduced pressure. The crude product was purified by preparative RP-HPLC (acetonitrile/water). Yield: 6.82 g (70% of theory, purity: 73%).

LC-MS (Method 1A): Rₜ=0.71 min; MS (ESIpos): m/z=264 [M+H]⁺.

### Example 65A

### 2-[(5R)-4-Benzyl-2,5-dimethylmorpholin-2-yl]ethanol [enantiomerically pure isomer 1+enantiomerically pure isomer 2]

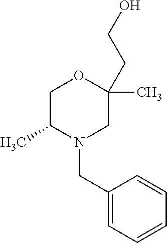 [SMILES: C[C@@H]1COC(C)(CCO)CN1Cc1ccccc1]

6.80 g (18.9 mmol, purity: 73%) of (5R)-4-benzyl-2-(2-hydroxyethyl)-2,5-dimethylmorpholin-3-one [diastereomer mixture, 2 isomers] were initially charged in tetrahydrofuran (191 ml), 37.7 ml (75.4 mmol) of 2M borane/dimethyl sulphide complex solution in tetrahydrofuran were added under argon and the mixture was stirred under reflux for 2 h. The mixture was subsequently cooled to 0° C., methanol (37 ml) was added carefully and the mixture was stirred under reflux for 30 min. The mixture was concentrated completely under reduced pressure, and the residue was taken up in acetonitrile and subjected directly to purification and diastereomer separation by preparative RP-HPLC (acetonitrile/water, isocratic). Enantiomerically pure isomer 1 was the first compound eluted. Yield: 1.34 g (28% of theory, enantiomerically pure isomer 1). Enantiomerically pure isomer 2 was the second compound eluted. Yield: 2.28 g (47% of theory, enantiomerically pure isomer 2).

### Enantiomerically Pure Isomer 1:

LC-MS (Method 4A): Rₜ=2.55 min; MS (ESIpos): m/z=250 [M+H]⁺;

### Enantiomerically Pure Isomer 2:

LC-MS (Method 4A): Rₜ=2.64 min; MS (ESIpos): m/z=250 [M+H]⁺.

### Example 66A

### 2-[(5R)-2,5-Dimethylmorpholin-2-yl]ethanol [enantiomerically pure isomer]

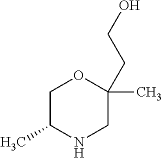

2.25 g (9.02 mmol) of 2-[(5R)-4-benzyl-2,5-dimethylmorpholin-2-yl]ethanol [enantiomerically pure isomer 2, Example 65A] were initially charged in ethanol (90.7 ml), 227 mg of palladium on carbon (10%) and 113 mg of palladium hydroxide on carbon (20%) were added under argon and the mixture was stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 1.46 g (quant.).

MS (Method 1C): m/z=160 [M+H]⁺;

¹H-NMR (400 MHz, DMSO-d₆): δ[ppm]=4.21 (t, 1H), 3.53-3.44 (d, 2H), 3.34 (dd, 1H), 3.14 (t, 1H), 2.65-2.52 (m, 3H), 2.07 (br. s., 1H), 1.52 (td, 2H), 1.18 (s, 3H), 0.85 (d, 3H).

### Example 67A

### (5R)-4-Benzyl-2-(2-hydroxypropyl)-2,5-dimethylmorpholin-3-one [diastereomer mixture, 4 isomers]

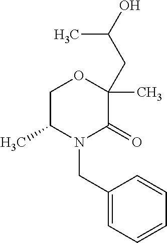 [SMILES: CC(O)CC1(C)OC[C@@H](C)N(Cc2ccccc2)C1=O]

16.8 g (about 64.3 mmol, crude product) of [(5R)-4-benzyl-2,5-dimethyl-3-oxomorpholin-2-yl]acetaldehyde [Example 63A, diastereomer mixture, 2 isomers] were initially charged in tetrahydrofuran (275 ml), and 77.2 ml (77.2 mmol) of a 1M solution of methylmagnesium bromide in tetrahydrofuran were added at −78° C. The reaction solution was stirred at −78° C. for 15 min and then allowed to warm to RT. Saturated aqueous ammonium chloride solution (400 ml) was then added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The crude product was used without further purification in the next step. Yield: 16.2 g of crude product.

LC-MS (Method 1A): Rₜ=0.78, 0.80 min; MS (ESIpos): m/z=278 [M+H]⁺.

### Example 68A

### 1-[(5R)-4-Benzyl-2,5-dimethylmorpholin-2-yl]propan-2-ol [enantiomerically pure isomer 1+enantiomerically pure isomer 2+enantiomerically pure isomer 3+enantiomerically pure isomer 4]

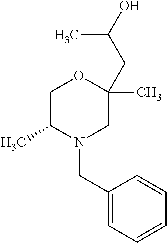 [SMILES: CC(O)CC1(C)CN(Cc2ccccc2)[C@H](C)CO1]

16.2 g (about 39.1 mmol, crude product) of (5R)-4-benzyl-2-(2-hydroxypropyl)-2,5-dimethylmorpholin-3-one [diastereomer mixture, 4 isomers] were initially charged in tetrahydrofuran (397 ml), 78.3 ml (157 mmol) of 2M borane/dimethyl sulphide complex solution in tetrahydrofuran were added under argon and the mixture was stirred under reflux for 2 h. The mixture was subsequently cooled to 0° C., methanol (80 ml) was added carefully and the mixture was stirred under reflux for 30 min. The mixture was subsequently concentrated completely under reduced pressure, and the residue was taken up in acetonitrile and subjected directly to purification and diastereomer separation by preparative RP-HPLC (acetonitrile/water, isocratic). Here, the target compound eluted as third component. Yield: Target compound: 3.11 g (29% of theory; enantiomerically pure isomer 3); enantiomerically pure isomer 1: 2.12 g (20% of theory); enantiomerically pure isomer 2: 506 mg (5% of theory); enantiomerically pure isomer 4: 1.72 g (16% of theory).

### Enantiomerically Pure Isomer 3:

LC-MS (Method 1A): Rₜ=0.39 min; MS (ESIpos): m/z=264 [M+H]⁺;

¹H-NMR (400 MHz, DMSO-d₆): δ[ppm]=7.34-7.18 (m, 5H), 4.10 (d, 1H), 3.96 (d, 1H), 3.79 (m_{c}, 1H), 3.48 (dd, 1H), 3.36 (m_{c}, 1H), 3.04 (d, 1H), 2.46 (d, 1H), 2.28 (m_{c}, 1H), 1.88 (d, 1H), 1.44 (dd, 1H), 1.36 (dd, 1H), 1.23 (s, 3H), 1.01 (d, 3H), 0.98 (d, 3H).

### Enantiomerically Pure Isomer 1:

LC-MS (Method 1A): Rₜ=0.43 min; MS (ESIpos): m/z=264 [M+H]⁺;

¹H-NMR (400 MHz, DMSO-d₆): δ[ppm]=7.33-7.18 (m, 5H), 4.16 (d, 1H), 3.90 (d, 1H), 3.76 (m_{c}, 1H), 3.50 (dd, 1H), 3.26 (dd, 1H), 3.10 (d, 1H), 2.43 (d, 1H), 2.32 (m_{c}, 1H), 2.10 (dd, 1H), 1.84 (d, 1H), 1.27 (dd, 1H), 1.09-1.06 (m, 6H), 0.98 (d, 3H).

### Enantiomerically Pure Isomer 2:

LC-MS (Method 1A): Rₜ=0.45 min; MS (ESIpos): m/z=264 [M+H]⁺;

¹H-NMR (400 MHz, DMSO-d₆): δ[ppm]=7.32-7.20 (m, 5H), 4.11 (d, 1H), 3.92 (d, 1H), 3.57 (m_{c}, 1H), 3.51 (dd, 1H), 3.41 (dd, 1H), 3.06 (d, 1H), 2.47 (d, 1H), 2.34 (m_{c}, 1H), 1.85-1.74 (m, 2H), 1.59 (dd, 1H), 1.06 (s, 3H), 1.03-0.97 (t, 6H).

### Enantiomerically Pure Isomer 4:

LC-MS (Method 1A): Rₜ=0.44 min; MS (ESIpos): m/z=264 [M+H]⁺;

¹H-NMR (400 MHz, DMSO-d₆): δ[ppm]=7.49 (s, 5H), 4.69 (d, 1H), 4.28-4.15 (m, 2H), 3.86-3.73 (m, 3H), 3.63 (t, 1H), 3.32 (t, 1H), 3.21 (br. s., 1H), 2.84 (d, 1H), 1.52-1.38 (m, 4H), 1.28 (s, 3H), 1.01 (d, 3H).

### Example 69A

### 1-[(5R)-2,5-Dimethylmorpholin-2-yl]propan-2-ol [enantiomerically pure isomer]

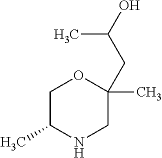

3.10 g (11.8 mmol) of 1-[(5R)-4-benzyl-2,5-dimethylmorpholin-2-yl]propan-2-ol [Example 68A, enantiomerically pure isomer 3] were initially charged in ethanol (118 ml), 296 mg of palladium on carbon (10%) and 148 mg of palladium hydroxide on carbon (20%) were added under argon and the mixture was then stirred under an atmosphere of hydrogen at standard pressure overnight. The reaction solution was filtered through kieselguhr and the filter residue was washed with hot ethanol (100 ml). The filtrate was concentrated under reduced pressure and the product was dried under high vacuum. Yield: 2.06 g (quant.).

GC-MS (Method 1B): Rₜ=3.86 min; MS (EIpos): m/z=173 [M]⁺;

¹H-NMR (400 MHz, DMSO-d₆): δ[ppm]=4.20 (d, 1H), 3.87 (br. s., 1H), 3.35 (dd, 1H), 3.16 (t, 1H), 2.67-2.53 (m, 3H), 2.05 (br. s., 1H), 1.44 (dd, 1H), 1.36 (dd, 1H), 1.23 (s, 3H), 1.04 (d, 3H), 0.85 (d, 3H).

### Example 70A

### N-Benzyl-2-chloro-N-[(2R)-1-hydroxybutan-2-yl]propanamide [diastereomer mixture, 2 isomers]

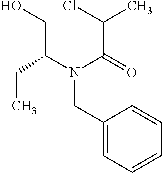 [SMILES: CC[C@H](CO)N(Cc1ccccc1)C(=O)C(C)Cl]

39.24 g (219 mmol) of (2R)-2-(benzylamino)butan-1-ol [lit.: P. Deniz et al., Tetrahedron 2011, 67, 6227-6232] were initially charged in isopropanol (500 ml), and 46.5 g (64.0 ml, 459 mmol) of triethylamine were added. 30.5 g (23.4 ml, 109 mmol) of 2-chloropropionyl chloride [racemate] were then added dropwise and the reaction solution was stirred at RT for 4 h. The mixture was then concentrated under reduced pressure, water was added to the residue and the mixture was extracted with dichloromethane. The organic phases were dried over magnesium sulphate, filtered and concentrated under reduced pressure. The crude product was used without further purification in the next step. Yield: 49.0 g (83% of theory, diastereomer ratio about 1:1).

LC-MS (Method 1A): Rₜ=0.86 min (enantiomerically pure isomer 1), Rₜ=0.88 min (enantiomerically pure isomer 2);

MS (ESIpos): m/z=270 [M+H]⁺.

### Example 71A

### (5R)-4-Benzyl-5-ethyl-2-methylmorpholin-3-one [diastereomer mixture, 2 isomers]

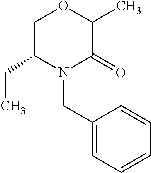 [SMILES: CC[C@@H]1COC(C)C(=O)N1Cc1ccccc1]

25.0 g (92.7 mmol) of N-benzyl-2-chloro-N-[(2R)-1-hydroxybutan-2-yl]propanamide [diastereomer mixture, 2 isomers] were initially charged in isopropanol (400 ml), the mixture was cooled to 0° C. and 34.3 g (306 mmol) of potassium tert-butoxide were then added in one portion. The mixture was slowly warmed to RT and stirred overnight. Most of the isopropanol was removed under reduced pressure and the residue was taken up in ethyl acetate. The organic phase was washed twice with water, once with 1N aqueous hydrogen chloride solution and once with saturated aqueous sodium chloride solution. The organic phase was dried over magnesium sulphate, filtered and concentrated under reduced pressure. The crude product was used without further purification in the next step.

Yield: 19.5 g (90% of theory).

LC-MS (Method 1A): Rₜ=0.93 min; MS (ESIpos): m/z=234 [M+H]⁺.

### Example 72A

### (5R)-2-Allyl-4-benzyl-5-ethyl-2-methylmorpholin-3-one [diastereomer mixture, 2 isomers]

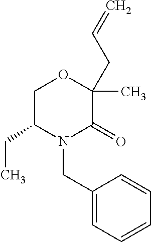 [SMILES: C=CCC1(C)OC[C@@H](CC)N(Cc2ccccc2)C1=O]

17.5 g (75.0 mmol) of (5R)-4-benzyl-5-ethyl-2-methylmorpholin-3-one [diastereomer mixture, 2 isomers] were initially charged in tetrahydrofuran (320 ml), 82.5 ml (82.5 mmol) of 1M lithium hexamethyldisilazide solution in tetrahydrofuran were added under argon and at −78° C. and the mixture was stirred for 30 min. Subsequently, at −78° C., 10.9 g (7.79 ml, 90.0 mmol) of allyl bromide in tetrahydrofuran (20 ml) were added dropwise. The reaction mixture was allowed to warm to RT and stirred overnight. The reaction was terminated by addition of water and the mixture was then extracted with ethyl acetate. The organic phase was washed with saturated aqueous sodium chloride solution, dried over magnesium sulphate, filtered and concentrated under reduced pressure. The crude product was purified by chromatography on silica gel (cyclohexane/ethyl acetate gradient). Yield: 13.5 g (65% of theory).

LC-MS (Method 1A): Rₜ=1.11 min; MS (ESIpos): m/z=274 [M+H]⁺.

### Example 73A

### [(5R)-4-Benzyl-5-ethyl-2-methyl-3-oxomorpholin-2-yl]acetaldehyde [diastereomer mixture, 2 isomers]

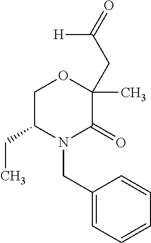 [SMILES: CC[C@@H]1COC(C)(CC=O)C(=O)N1Cc1ccccc1]

10.5 g (38.4 mmol) of (5R)-2-allyl-4-benzyl-5-ethyl-2-methylmorpholin-3-one [diastereomer mixture, 2 isomers] were initially charged in methanol (300 ml), and ozone-containing oxygen was then introduced into the solution at −78° C. for 30 min (ozone generator LAB2B from Triogen/Degrdmont Technologies Ltd., ozone concentration: about 30-50 mg/1). To remove excess ozone, pure oxygen was then introduced into the reaction solution for a few minutes. At −78° C., 23.9 g (28.2 ml, 384 mmol) of dimethyl sulphide were added and the reaction solution was stirred overnight while being allowed to warm to RT. The reaction solution was concentrated under reduced pressure and the residue was taken up in ethyl acetate and washed with water, 1N aqueous hydrogen chloride solution and saturated aqueous sodium chloride solution. The organic phase was dried over magnesium sulphate, filtered and concentrated under reduced pressure. The crude product was used without further purification in the next step. Yield: 10.1 g of crude product.

LC-MS (Method 1A): Rₜ=0.90 min; MS (ESIpos): m/z=276 [M+H]⁺.

### Example 74A

### (5R)-4-Benzyl-5-ethyl-2-(2-hydroxyethyl)-2-methylmorpholin-3-one [diastereomer mixture, 2 isomers]

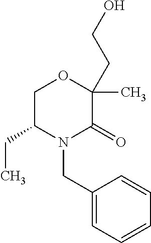 [SMILES: CC[C@@H]1COC(C)(CCO)C(=O)N1Cc1ccccc1]

9.80 g (about 35.6 mmol, crude product) of [(5R)-4-benzyl-5-ethyl-2-methyl-3-oxomorpholin-2-yl]acetaldehyde [diastereomer mixture, 2 isomers] were initially charged in methanol (100 ml), and 1.41 g (37.4 mmol) of sodium borohydride were added at 0° C. The mixture was then allowed to warm to RT and stirred for 30 min. Water was added and the reaction solution was extracted with ethyl acetate. The organic phase was dried over magnesium sulphate, filtered and concentrated under reduced pressure. The crude product was purified by chromatography on silica gel (cyclohexane/ethyl acetate gradient). Yield: 9.79 g (99% of theory).

LC-MS (Method 1A): Rₜ=0.79 min; MS (ESIpos): m/z=278 [M+H]⁺.

### Example 75A

### 2-[(5R)-4-Benzyl-5-ethyl-2-methylmorpholin-2-yl]ethanol [diastereomer mixture, 2 isomers]

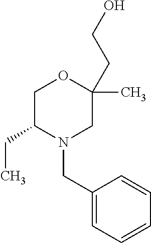 [SMILES: CC[C@@H]1COC(C)(CCO)CN1Cc1ccccc1]

9.79 g (35.3 mmol) of (5R)-4-benzyl-5-ethyl-2-(2-hydroxyethyl)-2-methylmorpholin-3-one [diastereomer mixture, 2 isomers] were initially charged in tetrahydrofuran (180 l), 106 ml (212 mmol) of 2M borane/dimethyl sulphide complex solution in tetrahydrofuran were added under argon and the mixture was stirred at RT overnight. Ethanol was added until the evolution of gas had ended and the mixture was stirred under reflux for 30 min. The mixture was then concentrated completely under reduced pressure, and the residue was taken up in acetonitrile and purified directly by flash chromatography on silica gel (cyclohexane/ethyl acetate gradient). Yield: 8.60 g (92% of theory, diastereomer ratio about 2:1).

LC-MS (Method 2A): Rₜ=0.46 min (enantiomerically pure isomer 1), Rₜ=0.49 min (enantiomerically pure isomer 2);

MS (ESIpos): m/z=274 [M+H]⁺.

### Example 76A

### 2-[(5R)-5-Ethyl-2-methylmorpholin-2-yl]ethanol [diastereomer mixture, 2 isomers]

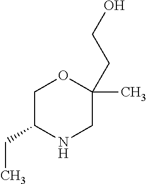

4.00 g (15.2 mmol) of 2-[(5R)-4-benzyl-5-ethyl-2-methylmorpholin-2-yl]ethanol [diastereomer mixture, 2 isomers] were initially charged in ethanol (150 ml), 400 mg of palladium on carbon (10%) and 200 mg of palladium hydroxide on carbon (20%) were added under argon, and the mixture was stirred under an atmosphere of hydrogen at standard pressure for 40 h. The reaction solution was filtered through kieselguhr and the filter residue was washed with ethanol. The filtrate was concentrated under reduced pressure and the product was dried under high vacuum. Yield: 2.55 g (90% of theory).

MS (Method 1C): m/z=174 [M+H]⁺.

### Example 77A

### 4-Benzyl-5-(methoxymethyl)-2-methylmorpholin-3-one [diastereomer mixture, 4 isomers]

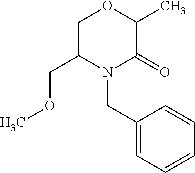 [SMILES: COCC1COC(C)C(=O)N1Cc1ccccc1]

15.0 g (63.8 mmol) of 4-benzyl-5-(hydroxymethyl)-2-methylmorpholin-3-one [diastereomer mixture, 4 isomers] were initially charged in N,N-dimethylformamide (600 ml), and 5.10 g (128 mmol, 60% suspension in paraffin oil) of sodium hydride and 22.6 g (9.92 ml, 159 mmol) of iodomethane were added. The mixture was stirred for 2 h and the reaction was then terminated by slowly adding water (30 ml). The mixture was concentrated under reduced pressure, and the residue was taken up in water and extracted repeatedly with ethyl acetate. The organic phase was dried over sodium sulphate, filtered and concentrated under reduced pressure. The residue was taken up in toluene and washed with water and saturated aqueous sodium chloride solution. The organic phase was dried over sodium sulphate, filtered and concentrated under reduced pressure. The crude product was purified by preparative RP-HPLC (acetonitrile/water). Yield: 16.7 g (96% of theory).

LC-MS (Method 1A): Rₜ=0.82 min; MS (ESIpos): m/z=250 [M+H]⁺.

### Example 78A

### 2-Allyl-4-benzyl-5-(methoxymethyl)-2-methylmorpholin-3-one [diastereomer mixture, 4 isomers]

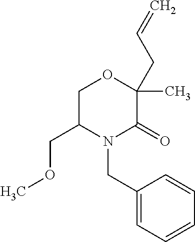 [SMILES: C=CCC1(C)OCC(COC)N(Cc2ccccc2)C1=O]

4.00 g (15.5 mmol) of 4-benzyl-5-(methoxymethyl)-2-methylmorpholin-3-one [diastereomer mixture, 4 isomers] were initially charged in tetrahydrofuran (200 ml), 21.7 ml (21.7 mmol) of 1M lithium hexamethyldisilazide solution in tetrahydrofuran were added under argon and at −78° C. and the reaction mixture was then stirred for 15 min. Subsequently, at −78° C., 3.12 g (1.70 ml, 18.6 mmol) of allyl iodide were added, and the reaction mixture was warmed to RT and stirred overnight. The reaction was terminated by addition of saturated aqueous ammonium chloride solution and the mixture was extracted with ethyl acetate. The organic phase was washed with saturated aqueous sodium chloride solution, dried over magnesium sulphate, filtered and concentrated under reduced pressure. The crude product was used without further purification in the next step. Yield: 5.26 g (99% of theory, purity: 84%).

LC-MS (Method 1A): Rₜ=1.04 min; MS (ESIpos): m/z=290 [M+H]⁺.

### Example 79A

### [4-Benzyl-5-(methoxymethyl)-2-methyl-3-oxomorpholin-2-yl]acetaldehyde [diastereomer mixture, 4 isomers]

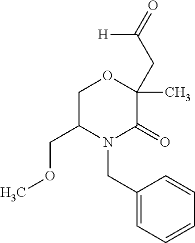 [SMILES: COCC1COC(C)(CC=O)C(=O)N1Cc1ccccc1]

5.26 g (15.4 mmol, purity: 84%) of 2-allyl-4-benzyl-5-(methoxymethyl)-2-methylmorpholin-3-one [diastereomer mixture, 4 isomers] were initially charged in tetrahydrofuran (333 ml) and water (199 ml), and 3.87 ml (1.42 mmol) of a 2.5% solution of osmium tetroxide in tert-butanol and 9.88 g (46.2 mmol) of sodium periodate were added at 0° C. The mixture was then warmed to RT and stirred for 20 h. The reaction solution was filtered through kieselguhr and the tetrahydrofuran was removed under reduced pressure. The aqueous phase was extracted with ethyl acetate and the organic phase was washed with saturated aqueous sodium chloride solution. The organic phase was dried over sodium sulphate, filtered and concentrated under reduced pressure. The crude product was used without further purification in the next step. Yield: 5.06 g (91% of theory, purity: 81%).

LC-MS (Method 1A): Rₜ=0.85 min (diastereomer 1, 2 isomers), Rₜ=0.88 min (diastereomer 2, 2 isomers);

MS (ESIpos): m/z=292 [M+H]⁺.

### Example 80A

### 4-Benzyl-2-(2-hydroxyethyl)-5-(methoxymethyl)-2-methylmorpholin-3-one [diastereomer mixture, 4 isomers]

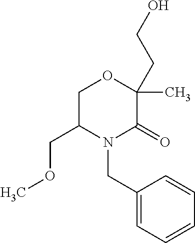 [SMILES: COCC1COC(C)(CCO)C(=O)N1Cc1ccccc1]

5.00 g (13.7 mmol, purity: 80%) of [4-benzyl-5-(methoxymethyl)-2-methyl-3-oxomorpholin-2-yl]acetaldehyde [diastereomer mixture, 4 isomers] were initially charged in methanol (102 ml), and 1.56 g (41.2 mmol) of sodium borohydride were added at 0° C. The mixture was then warmed to RT and stirred for 30 min. Water was added, and the reaction solution was extracted with ethyl acetate. The organic phase was dried over sodium sulphate, filtered and concentrated under reduced pressure. The crude product was used without further purification in the next step. Yield: 4.84 g (65% of theory, purity: 54%).

LC-MS (Method 2A): Rₜ=0.74 min (diastereomer 1, 2 isomers), Rₜ=0.75 min (diastereomer 2, 2 isomers);

MS (ESIpos): m/z=294 [M+H]⁺.

### Example 81A

### 2-[4-Benzyl-5-(methoxymethyl)-2-methylmorpholin-2-yl]ethanol [diastereomer mixture, 4 isomers]

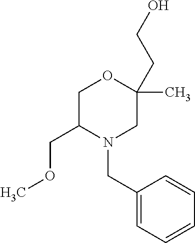 [SMILES: COCC1COC(C)(CCO)CN1Cc1ccccc1]

4.84 g (18.9 mmol, purity: 54%) of 4-benzyl-2-(2-hydroxyethyl)-5-(methoxymethyl)-2-methylmorpholin-3-one [diastereomer mixture, 4 isomers] were initially charged in tetrahydrofuran (88 ml), 44.6 ml (89.2 mmol) of 2M borane/dimethyl sulphide complex solution in tetrahydrofuran were added under argon and the mixture was stirred under reflux for 1 h. The mixture was subsequently cooled to RT, ethanol (40 ml) was added carefully and the mixture was stirred under reflux for 1 h. The mixture was then concentrated completely under reduced pressure, and the residue was taken up in acetonitrile and purified by preparative RP-HPLC (acetonitrile/water, isocratic). Yield: 2.65 g (quant., diastereomer ratio about 3:2).

LC-MS (Method 1A): Rₜ=0.49 min (diastereomer 1, 2 isomers), Rₜ=0.53 min (diastereomer 2, 2 isomers);

MS (ESIpos): m/z=280 [M+H]⁺.

### Example 82A

### 2-[5-(Methoxymethyl)-2-methylmorpholin-2-yl]ethanol [diastereomer mixture, 4 isomers]

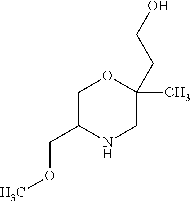

2.65 g (9.49 mmol) of 2-[4-benzyl-5-(methoxymethyl)-2-methylmorpholin-2-yl]ethanol [diastereomer mixture, 4 isomers] were initially charged in ethanol (37.9 ml), 250 mg of palladium on carbon (10%) and 125 mg of palladium hydroxide on carbon (20%) were added under argon, and the mixture was stirred under an atmosphere of hydrogen at standard pressure for 20 h. The reaction solution was filtered through kieselguhr and the filter residue was washed with methanol. The filtrate was concentrated under reduced pressure and the product was dried under high vacuum. Yield: 1.75 g (92% of theory).

MS (Method 2C): m/z=190 [M+H]⁺.

### Example 83A

### N-Benzyl-2-chloro-N-(1-hydroxy-2-methylpropan-2-yl)propanamide [racemate]

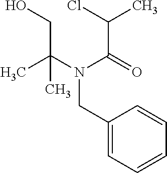 [SMILES: CC(Cl)C(=O)N(Cc1ccccc1)C(C)(C)CO]

20.0 g (106 mmol) of 2-(benzylamino)-2-methylpropan-1-ol [lit.: M. Le Hyaric et al., Chem. Biol. Drug Des. 2011, 78 876-880] were initially charged in isopropanol (350 ml), the mixture was cooled to 0° C. and 22.6 g (31.1 ml, 223 mmol) of triethylamine were added. 15.6 g (12.2 ml, 123 mmol) of 2-chloropropionyl chloride [racemate] were then added dropwise. After 30 min of stirring, a further 7.09 g (5.55 ml, 55.9 mmol) of 2-chloropropionyl chloride [racemate] were added dropwise, and the reaction solution was allowed to warm to RT. The reaction solution was concentrated under reduced pressure and the residue was taken up in ethyl acetate (700 ml) and washed with water (400 ml). The organic phases were dried over sodium sulphate, filtered and concentrated under reduced pressure. The crude product was used without further purification in the next step. Yield: 37.1 g (quant.).

LC-MS (Method 1A): Rₜ=0.95 min; MS (ESIpos): m/z=270 [M+H]⁺.

### Example 84A

### 4-Benzyl-2,5,5-trimethylmorpholin-3-one [racemate]

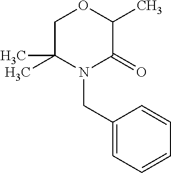 [SMILES: CC1OCC(C)(C)N(Cc2ccccc2)C1=O]

37.1 g (72.9 mmol, purity: 53%) of N-benzyl-2-chloro-N-(1-hydroxy-2-methylpropan-2-yl)propanamide [racemate] in isopropanol (500 ml) were cooled to 0° C., and 24.5 g (219 mmol) of potassium tert-butoxide were added in one portion. The mixture was stirred at 0° C. for 1 h and most of the isopropanol was then removed under reduced pressure. The residue was taken up in dichloromethane and washed with a 1N aqueous hydrogen chloride solution (400 ml). The organic phase was dried over sodium sulphate, filtered and concentrated under reduced pressure. The crude product was used without further purification in the next step. Yield: 23.2 g (quant.).

LC-MS (Method 1A): Rₜ=0.91 min; MS (ESIpos): m/z=234 [M+H]⁺.

### Example 85A

### 2-Allyl-4-benzyl-2,5,5-trimethylmorpholin-3-one [racemate]

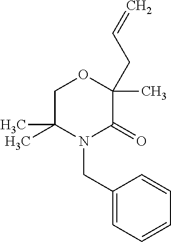 [SMILES: C=CCC1(C)OCC(C)(C)N(Cc2ccccc2)C1=O]

21.58 g (92.1 mmol) of 4-benzyl-2,5,5-trimethylmorpholin-3-one [racemate] were initially charged in tetrahydrofuran (1.19 l), 129 ml (129 mmol) of 1M lithium hexamethyldisilazide solution in tetrahydrofuran were added under argon and at −78° C. and the mixture was stirred for 15 min. Subsequently, at −78° C., 23.2 g (12.6 ml, 138 mmol) of allyl iodide were added, and the reaction mixture was warmed to RT and stirred overnight. The mixture was once more cooled to −78° C., 92.1 ml (92.1 mmol) of a 1M solution of lithiumhexamethyldisilazide in tetrahydrofuran were added and the reaction mixture was then stirred for 30 min. A further 15.5 g (8.42 ml, 92.1 mmol) of allyl iodide were then added and the mixture was warmed to RT. The reaction was terminated by addition of saturated aqueous ammonium chloride solution and the mixture was then extracted with ethyl acetate. The organic phase was washed with saturated aqueous sodium chloride solution, dried over sodium sulphate, filtered and concentrated under reduced pressure. The crude product was used without further purification in the next step. Yield: 23.4 g (93% of theory).

LC-MS (Method 1A): Rₜ=1.09 min; MS (ESIpos): m/z=274 [M+H]⁺.

### Example 86A

### (4-Benzyl-2,5,5-trimethyl-3-oxomorpholin-2-yl)acetaldehyde [racemate]

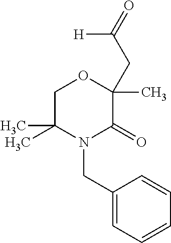 [SMILES: CC1(CC=O)OCC(C)(C)N(Cc2ccccc2)C1=O]

At 0° C., 4.00 ml (1.47 mmol) of a 2.5% strength solution of osmium tetroxide in tert-butanol and 47.1 g (220 mmol) of sodium periodate were added to 23.6 g (73.4 mmol) of (2-allyl-4-benzyl-2,5,5-trimethylmorpholin-3-one [racemate] in tetrahydrofuran (570 ml) and water (340 ml). The mixture was then allowed to warm to RT and stirred overnight. The reaction solution was filtered through kieselguhr and the filter residue was washed with tetrahydrofuran. The mixture was taken up in ethyl acetate and diluted with water. After separation of the phases, the organic phase was washed with 1N aqueous sodium sulphite solution (2×800 ml), dried over magnesium sulphate, filtered and concentrated under reduced pressure. The crude product was used without further purification in the next step. Yield: 19.8 g of crude product.

LC-MS (Method 1A): Rₜ=0.93 min; MS (ESIpos): m/z=276 [M+H]⁺.

### Example 87A

### 4-Benzyl-2-(2-hydroxyethyl)-2,5,5-trimethylmorpholin-3-one [racemate]

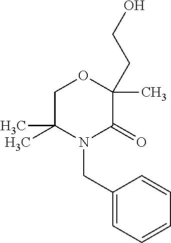 [SMILES: CC1(CCO)OCC(C)(C)N(Cc2ccccc2)C1=O]

At 0° C., 2.68 g (70.8 mmol) of sodium borohydride were added to 6.50 g (about 23.6 mmol, crude product) of (4-benzyl-2,5,5-trimethyl-3-oxomorpholin-2-yl)acetaldehyde [racemate] in methanol (176 ml). The mixture was then allowed to warm to RT and stirred for 30 min. Water was added to the reaction solution, most of the methanol was removed under reduced pressure and the residue was extracted with ethyl acetate. The organic phase was dried over sodium sulphate, filtered and concentrated under reduced pressure. The crude product was purified by preparative RP-HPLC (acetonitrile/water). Yield: 5.89 g (62% of theory, purity: 69%).

LC-MS (Method 4A): Rₜ=2.31 min; MS (ESIpos): m/z=278 [M+H]⁺.

### Example 88A

### 2-(4-Benzyl-2,5,5-trimethylmorpholin-2-yl)ethanol [racemate]

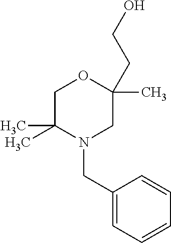 [SMILES: CC1(CCO)CN(Cc2ccccc2)C(C)(C)CO1]

5.85 g (21.1 mmol) of 4-benzyl-2-(2-hydroxyethyl)-2,5,5-trimethylmorpholin-3-one [racemate] were initially charged in tetrahydrofuran (210 ml), 42.2 ml (84.4 mmol) of 2M borane/dimethyl sulphide complex solution in tetrahydrofuran were added under argon and the mixture was stirred under reflux for 2 h. The mixture was subsequently cooled to 0° C., methanol (45 ml) was added carefully and the mixture was stirred under reflux for 30 min to destroy any boron complexes. The mixture was then concentrated completely under reduced pressure, and the residue was taken up in acetonitrile and purified directly by preparative RP-HPLC (acetonitrile/water). Yield: 3.10 g (55% of theory).

LC-MS (Method 4A): Rₜ=2.82 min; MS (ESIpos): m/z=264 [M+H]⁺.

### Example 89A

### 2-(2,5,5-Trimethylmorpholin-2-yl)ethanol [racemate]

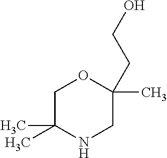

3.00 g (11.4 mmol) of 2-(4-benzyl-2,5,5-trimethylmorpholin-2-yl)ethanol [racemate] were initially charged in ethanol (115 ml), 286 mg of palladium on carbon (10%) and 143 mg of palladium hydroxide on carbon (20%) were added under argon and the mixture was stirred under an atmosphere of hydrogen at standard pressure overnight. Subsequently, a further 286 mg of palladium on carbon (10%) and 143 mg of palladium hydroxide on carbon (20%) were added, and the mixture was once more stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 2.06 g (quant.).

MS (Method 1C): m/z=174 [M+H]⁺.

### Example 90A

### 4-Benzyl-2-(2-hydroxypropyl)-2,5,5-trimethylmorpholin-3-one [diastereomer mixture, 4 isomers]

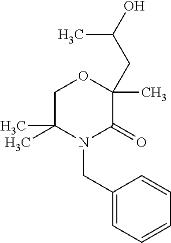 [SMILES: CC(O)CC1(C)OCC(C)(C)N(Cc2ccccc2)C1=O]

6.50 g (about 23.6 mmol, crude product) of (4-benzyl-2,5,5-trimethyl-3-oxomorpholin-2-yl)acetaldehyde [racemate] were initially charged in tetrahydrofuran (101 ml), and 28.3 ml (28.3 mmol) of methylmagnesium bromide (2M solution in tetrahydrofuran) were added slowly at −78° C. The mixture was stirred at −78° C. for 15 min and then allowed to warm to RT. The reaction was terminated by addition of saturated aqueous ammonium chloride solution (about 70 ml) and the tetrahydrofuran was removed under reduced pressure. The residue was taken up in dichloromethane and washed with water. The organic phase was dried over sodium sulphate, filtered and concentrated under reduced pressure. The crude product was used without further purification in the next step. Yield: 6.17 g (54% of theory).

LC-MS (Method 4A): Rₜ=2.46 min; MS (ESIpos): m/z=292 [M+H]⁺.

### Example 91A

### 2-(4-Benzyl-2,5,5-trimethylmorpholin-2-yl)propan-2-ol [diastereomer 1, 2 isomers+diastereomer 2, 2 isomers]

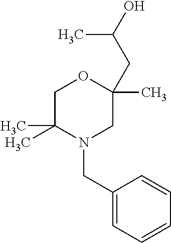 [SMILES: CC(O)CC1(C)CN(Cc2ccccc2)C(C)(C)CO1]

6.15 g (21.1 mmol) of 4-benzyl-2-(2-hydroxypropyl)-2,5,5-trimethylmorpholin-3-one [diastereomer mixture, 4 isomers] were initially charged in tetrahydrofuran (210 ml), 42.2 ml (84.4 mmol) of 2M borane/dimethyl sulphide complex solution in tetrahydrofuran were added under argon and the mixture was stirred under reflux for 2 h. The mixture was subsequently cooled to 0° C., methanol (45 ml) was added carefully and the mixture was stirred under reflux for 30 min. The mixture was subsequently concentrated completely under reduced pressure, and the residue was taken up in acetonitrile and subjected directly to purification and diastereomer separation by preparative RP-HPLC (acetonitrile/water, isocratic). A mixed fraction (1.10 g) was re-purified on an achiral phase according to Method 3F. Yield: 1.74 g (29% of theory, diastereomer 1, 2 isomers) and 434 mg (26% of theory, diastereomer 2, 2 isomers).

### Diastereomer 1, 2 Isomers:

LC-MS (Method 4A): Rₜ=3.06 min; MS (ESIpos): m/z=279 [M+H]⁺.

### Diastereomer 2, 2 Isomers:

LC-MS (Method 4A): Rₜ=3.18 min; MS (ESIpos): m/z=279 [M+H]⁺.

### Example 92A

### 1-(2,5,5-Trimethylmorpholin-2-yl)propan-2-ol [diastereomer 1, 2 isomers]

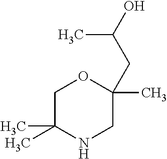

3.00 g (11.4 mmol) of 2-(4-benzyl-2,5,5-trimethylmorpholin-2-yl)propan-2-ol [diastereomer 1, 2 isomers, Example 91A] were initially charged in ethanol (54.0 ml), 135 mg of palladium on carbon (10%) and 67.0 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 1.09 g (quant.).

MS (Method 1C): m/z=188 [M+H]⁺.

### Example 93A

### 4-Benzyl-6-methyl-5-oxomorpholine-3-carboxylic acid [diastereomer mixture, 4 isomers]

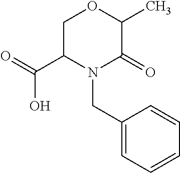 [SMILES: CC1OCC(C(=O)O)N(Cc2ccccc2)C1=O]

20.0 g (85.0 mmol) of 4-benzyl-5-(hydroxymethyl)-2-methylmorpholin-3-one [diastereomer mixture, 4 isomers] were initially charged in acetonitrile (1.50 l), 42.6 g (187 mmol) of periodic acid were added at RT and the mixture was stirred for 15 min. The mixture was then cooled to 0° C., and 733 mg (3.40 mmol) of pyridinium chlorochromate in acetonitrile (30 ml) were added. The mixture was stirred at 0° C. for 2 h and the reaction solution was then concentrated under reduced pressure to about 50 ml. Water (1.00 l) was added and the mixture was extracted with ethyl acetate. The organic phase was dried over sodium sulphate, filtered and concentrated under reduced pressure. The crude product was used without further purification in the next step. Yield: 21.4 g (60% of theory, purity: 60%).

LC-MS (Method 1A): Rₜ=0.65 min; MS (ESIpos): m/z=250 [M+H]⁺.

### Example 94A

### Methyl 4-benzyl-6-methyl-5-oxomorpholin-3-carboxylate [diastereomer mixture, 4 isomers]

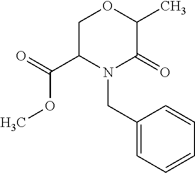 [SMILES: COC(=O)C1COC(C)C(=O)N1Cc1ccccc1]

21.3 g (crude product) of 4-benzyl-6-methyl-5-oxomorpholine-3-carboxylic acid [diastereomer mixture, 4 isomers] in methanol (500 ml) were cooled to 0° C., and 12.5 ml (171 mmol) of thionyl chloride were added slowly. The reaction mixture was stirred under reflux for 2 h and then concentrated completely under reduced pressure. The crude product was used without further purification in the next step. Yield: 19.8 g (88% of theory, crude product).

LC-MS (Method 1A): Rₜ=0.80 min; MS (ESIpos): m/z=264 [M+H]⁺.

### Example 95A

### 4-Benzyl-5-(2-hydroxypropan-2-yl)-2-methylmorpholin-3-one [racemate]

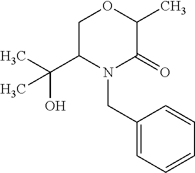 [SMILES: CC1OCC(C(C)(C)O)N(Cc2ccccc2)C1=O]

4.00 g (about 15.2 mmol, crude product) of methyl 4-benzyl-6-methyl-5-oxomorpholin-3-carboxylate [diastereomer mixture, 4 isomers] were initially charged in tetrahydrofuran (55.8 ml), and 53.2 ml (53.2 mmol) of a 1M solution of methylmagnesium bromide in tetrahydrofuran were added at −78° C. The mixture was stirred at −78° C. for 15 min and then allowed to warm to RT. Saturated aqueous ammonium chloride solution (70 ml) was then added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The crude product was purified by preparative RP-HPLC (acetonitrile/water). Yield: 968 mg (24% of theory); at this or any of the preceding stages, there was complete epimerization to one of the two possible diastereomers.

LC-MS (Method 4A): Rₜ=0.79 min; MS (ESIpos): m/z=264 [M+H]⁺.

### Example 96A

### 2-(4-Benzyl-6-methylmorpholin-3-yl)propan-2-ol [racemate]

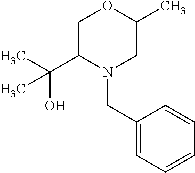 [SMILES: CC1CN(Cc2ccccc2)C(C(C)(C)O)CO1]

967 mg (3.67 mmol) of 4-benzyl-5-(2-hydroxypropan-2-yl)-2-methylmorpholin-3-one [racemate] were initially charged in tetrahydrofuran (36.1 ml), 7.34 ml (14.7 mmol) of 2M borane/dimethyl sulphide complex solution in tetrahydrofuran were added under argon and the mixture was stirred under reflux for 2 h. The mixture was subsequently cooled to 0° C., methanol (10 ml) was added carefully and the mixture was stirred under reflux for 30 min. The mixture was then concentrated completely under reduced pressure, and the residue was taken up in acetonitrile and purified directly by preparative RP-HPLC (acetonitrile/water). Yield: 433 mg (47% of theory).

LC-MS (Method 1A): Rₜ=0.44 min; MS (ESIpos): m/z=250 [M+H]⁺.

### Example 97A

### 2-(4-Benzyl-6-methylmorpholin-3-yl)propan-2-ol [enantiomerically pure isomer 1]

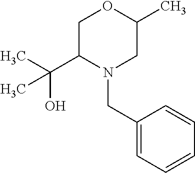 [SMILES: CC1CN(Cc2ccccc2)C(C(C)(C)O)CO1]

Enantiomer separation on a chiral phase of 433 mg of the compound from Example 96A according to Method 22D gave 179 mg of Example 97A (enantiomerically pure isomer 1) and 183 mg of Example 98A (enantiomerically pure isomer 2).

HPLC (Method 18E): Rₜ=5.50 min, 99.0% ee;

LC-MS (Method 1A): Rₜ=0.43 min; MS (ESIpos): m/z=250 [M+H]⁺.

### Example 98A

### 2-(4-Benzyl-6-methylmorpholin-3-yl)propan-2-ol [enantiomerically pure isomer 2]

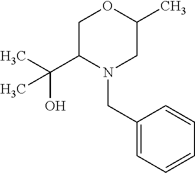 [SMILES: CC1CN(Cc2ccccc2)C(C(C)(C)O)CO1]

Enantiomer separation on a chiral phase of 433 mg of the compound from Example 96A according to Method 22D gave 179 mg of Example 97A (enantiomerically pure isomer 1) and 183 mg of Example 98A (enantiomerically pure isomer 2).

HPLC (Method 18E): Rₜ=6.88 min, 99.0% ee;

LC-MS (Method 1A): Rₜ=0.44 min; MS (ESIpos): m/z=250 [M+H]⁺.

### Example 99A

### 2-(6-Methylmorpholin-3-yl)propan-2-ol [enantiomerically pure isomer 1]

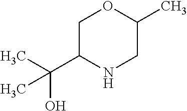

179 mg (0.718 mmol) of 2-(4-benzyl-6-methylmorpholin-3-yl)propan-2-ol [enantiomerically pure isomer 1, Example 97A] were initially charged in ethanol (7.22 ml), 20.9 mg of palladium on carbon (10%) and 10.5 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 94.4 mg (82% of theory).

MS (Method 1C): m/z=160 [M+H]⁺.

### Example 100A

### 2-(6-Methylmorpholin-3-yl)propan-2-ol [enantiomerically pure isomer 2]

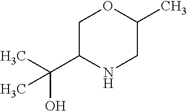

182 mg (0.730 mmol) of 2-(4-benzyl-6-methylmorpholin-3-yl)propan-2-ol [enantiomerically pure isomer 2, Example 98A] were initially charged in ethanol (7.22 ml), 21.3 mg of palladium on carbon (10%) and 10.6 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 118 mg (quant.).

MS (Method 1C): m/z=160 [M+H]⁺.

### Example 101A

### 2-Methyl-2-{[(E)-phenylmethylene]amino}propane-1,3-diol

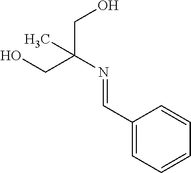

183 g (947 mmol) of 2-amino-2-methylpropane-1,3-diol were suspended in ethyl acetate (200 ml), and 103 g (98.6 ml, 970 mmol) of benzaldehyde were added dropwise with ice cooling. The mixture was then allowed to warm to RT and stirred for 2 h. The mixture was concentrated at 70° C. under reduced pressure and the residue was used in the next step without further purification.

Yield: 183 g (99% of theory).

### Example 102A

### 2-(Benzylamino)-2-methylpropane-1,3-diol

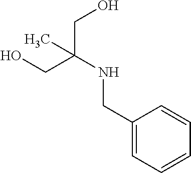

100 g (951 mmol) of 2-methyl-2-{[(E)-phenylmethylene]amino}propane-1,3-diol [lit.: B. Anxionnat et al., J. Org. Chem. 2012, 77, 6087-6099] were initially charged in ethanol (1.00 l), and 71.6 g (1.89 mol) of sodium borohydride were added a little at a time at 0° C. (strong evolution of gas). The mixture was allowed to warm to RT and stirred overnight. The mixture was then concentrated under reduced pressure and the residue was taken up in water (700 ml). The pH was adjusted to about pH=1 with concentrated aqueous hydrogen chloride solution and the mixture was extracted with dichloromethane. The aqueous phase was adjusted to about pH=10 with 50% strength aqueous sodium hydroxide solution and then extracted repeatedly with dichloromethane. The combined organic phases were dried over sodium sulphate, filtered and concentrated under reduced pressure. Yield: 151 g (79% of theory).

LC-MS (Method 4A): Rₜ=1.86 min; MS (ESIpos): m/z=196 [M+H]⁺.

### Example 103A

### N-Benzyl-2-chloro-N-(1,3-dihydroxy-2-methylpropan-2-yl)propanamide [racemate]

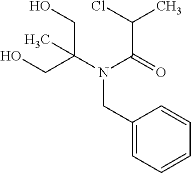 [SMILES: CC(Cl)C(=O)N(Cc1ccccc1)C(C)(CO)CO]

10.7 g (54.8 mmol) of 2-(benzylamino)-2-methylpropane-1,3-diol were initially charged in dichloromethane (400 ml), the mixture was cooled to 0° C. and 8.32 g (11.5 ml, 82.2 mmol) of triethylamine were added. Then first 8.35 g (6.52 ml, 65.8 mmol) of 2-chloropropionyl chloride [racemate] and then a further 5.57 g (4.35 ml, 49.3 mmol) of 2-chloropropionyl chloride [racemate] were added dropwise. After 10 min of stirring, the reaction solution was concentrated under reduced pressure, and the residue was taken up in 1N aqueous hydrogen chloride solution and extracted repeatedly with dichloromethane. The collected organic phases were dried over sodium sulphate, filtered and concentrated under reduced pressure. The crude product was used without further purification in the next step. Yield: 15.6 g (99% of theory).

### Example 104A

### 4-Benzyl-5-(hydroxymethyl)-2,5-dimethylmorpholin-3-one [diastereomer mixture, 4 isomers]

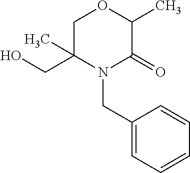 [SMILES: CC1OCC(C)(CO)N(Cc2ccccc2)C1=O]

15.6 g (54.8 mmol) of N-benzyl-2-chloro-N-(1,3-dihydroxy-2-methylpropan-2-yl)propanamide [racemate] were initially charged in isopropanol (300 ml), the mixture was cooled to 0° C. and 24.6 g (219 mmol) of potassium tert-butoxide were added in one portion. The reaction mixture was stirred overnight and allowed to warm to RT during this time. Most of the isopropanol was removed under reduced pressure, and the residue was taken up in 2N aqueous hydrogen chloride solution (300 ml) and extracted repeatedly with dichloromethane. The organic phases were dried over sodium sulphate, filtered and concentrated under reduced pressure. The crude product was used without further purification in the next step. Yield: 15.2 g (96% of theory, purity: 86%, diastereomer ratio about 1:1).

LC-MS (Method 1A): Rₜ=0.72 min (diastereomer 1, 2 isomers), Rₜ=0.74 min (diastereomer 2, 2 isomers);

MS (ESIpos): m/z=250 [M+H]⁺.

### Example 105A

### 4-Benzyl-5-({[tert-butyl(dimethyl)silyl]oxy}methyl)-2,5-dimethylmorpholin-3-one [diastereomer mixture, 4 isomers]

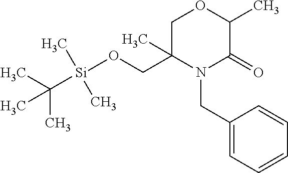 [SMILES: CC1OCC(C)(CO[Si](C)(C)C(C)(C)C)N(Cc2ccccc2)C1=O]

20.0 g (80.2 mmol) of 4-benzyl-5-(hydroxymethyl)-2,5-dimethylmorpholin-3-one [diastereomer mixture, 4 isomers] were initially charged in N,N-dimethylformamide (132 ml), and 10.9 g (160 mmol) of imidazole were added at RT. 12.7 g (84.2 mmol) of tert-butyldimethylsilyl chloride were then added, and the mixture was stirred overnight. The reaction solution was concentrated under reduced pressure, taken up in ethyl acetate and washed repeatedly with water, once with 0.4N aqueous hydrogen chloride solution, once with saturated aqueous sodium bicarbonate solution and again with water. The organic phase was dried over magnesium sulphate, filtered and concentrated under reduced pressure. Yield: 27.9 g (93% of theory, diastereomer ratio: about 1:1).

LC-MS (Method 1A): Rₜ=1.45 min (diastereomer 1, 2 isomers), Rₜ=1.47 min (diastereomer 2, 2 isomers).

MS (ESIpos): m/z=364 [M+H]⁺.

### Example 106A

### 4-Benzyl-5-({[tert-butyl(dimethyl)silyl]oxy}methyl)-2,2,5-trimethylmorpholin-3-one [racemate]

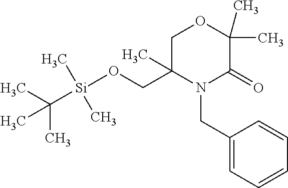 [SMILES: CC1(C)OCC(C)(CO[Si](C)(C)C(C)(C)C)N(Cc2ccccc2)C1=O]

27.9 g (76.7 mmol) of 4-benzyl-5-({[tert-butyl(dimethyl)silyl]oxy}methyl)-2,5-dimethylmorpholin-3-one [diastereomer mixture, 4 isomers] were initially charged in tetrahydrofuran (959 ml), and 59.7 ml (107 mmol) of lithium diisopropylamide solution (2.0M in tetrahydrofuran/n-heptane/ethylbenzene) were added dropwise at −78° C. The mixture was stirred for 15 min, and 13.1 g (5.73 ml, 92.1 mmol) of iodomethane were then added. The mixture was allowed to warm to RT and stirred for 2 h. Saturated aqueous ammonium chloride solution was added and the mixture was extracted with ethyl acetate. The organic phase was washed with saturated aqueous sodium chloride solution, dried over magnesium sulphate, filtered and concentrated under reduced pressure. The crude product was used without further purification in the next step. Yield: 31.9 g (59% of theory, purity: 54%).

LC-MS (Method 1A): Rₜ=1.49 min; MS (ESIpos): m/z=378 [M+H]⁺.

### Example 107A

### 4-Benzyl-5-(hydroxymethyl)-2,2,5-trimethylmorpholin-3-one [racemate]

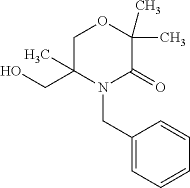 [SMILES: CC1(C)OCC(C)(CO)N(Cc2ccccc2)C1=O]

31.8 g (45.5 mmol, purity: 54%) of 4-benzyl-5-({[tert-butyl(dimethyl)silyl]oxy}methyl)-2,2,5-trimethylmorpholin-3-one [racemate] were initially charged in tetrahydrofuran (991 ml), and 158 ml (158 mmol) of tetra-n-butylammonium fluoride solution (1.0M in tetrahydrofuran) were added at RT. The reaction solution was stirred at RT overnight and then concentrated under reduced pressure. The residue was taken up in dichloromethane and washed with water. The organic phase was dried over sodium sulphate, filtered and concentrated under reduced pressure. The crude product was then purified by preparative RP-HPLC (acetonitrile/water). Yield: 15.2 g (91% of theory, purity: 72%).

LC-MS (Method 1A): Rₜ=0.79 min; MS (ESIpos): m/z=264 [M+H]⁺.

### Example 108A

### (4-Benzyl-3,6,6-trimethylmorpholin-3-yl)methanol [racemate]

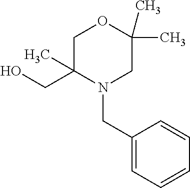 [SMILES: CC1(C)CN(Cc2ccccc2)C(C)(CO)CO1]

3.00 g (11.4 mmol) of 4-benzyl-5-(hydroxymethyl)-2,2,5-trimethylmorpholin-3-one [racemate] were initially charged in tetrahydrofuran (112 ml), 22.8 ml (45.6 mmol) of 2M borane/dimethyl sulphide complex solution in tetrahydrofuran were added under argon and the mixture was stirred under reflux for 2 h. The mixture was subsequently cooled to 0° C. and methanol (26 ml) was added carefully. The mixture was stirred under reflux for 30 min and then concentrated completely under reduced pressure. The crude product was purified by preparative RP-HPLC (acetonitrile/water).

Yield: 1.74 g (58% of theory).

LC-MS (Method 1A): Rₜ=0.46 min; MS (ESIpos): m/z=250 [M+H]⁺.

### Example 109A

### (3,6,6-Trimethylmorpholin-3-yl)methanol [racemate]

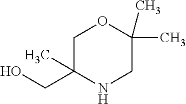

1.74 g (6.98 mmol) of 2-(4-benzyl-3,6,6-trimethylmorpholin-3-yl)methanol [racemate] were initially charged in ethanol (70.1 ml), 200 mg of palladium on carbon (10%) and 100 mg of palladium hydroxide on carbon (20%) were added under argon and the mixture was then stirred under an atmosphere of hydrogen at standard pressure overnight. The reaction solution was filtered through kieselguhr and concentrated under reduced pressure. Yield: 1.16 g (quant.).

MS (Method 1C): m/z=160 [M+H]⁺.

### Example 110A

### 4-Benzyl-5-(methoxymethyl)-2-methylmorpholin-3-one [diastereomer mixture, 4 isomers]

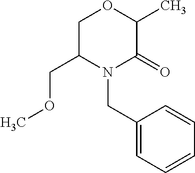 [SMILES: COCC1COC(C)C(=O)N1Cc1ccccc1]

15.0 g (63.8 mmol) of 4-benzyl-5-(hydroxymethyl)-2-methylmorpholin-3-one [diastereomer mixture, 4 isomers] were initially charged in N,N-dimethylformamide (600 ml), and 5.10 g (128 mmol, 60% suspension in paraffin oil) of sodium hydride and 22.6 g (9.92 ml, 159 mmol) of iodomethane were added. The mixture was stirred for 2 h and the reaction was then terminated by slowly adding water (30 ml). The mixture was concentrated under reduced pressure, and the residue was taken up in water and extracted repeatedly with ethyl acetate. The organic phase was dried over sodium sulphate, filtered and concentrated under reduced pressure. The residue was taken up in toluene and washed with water and saturated aqueous sodium chloride solution. The organic phase was dried over sodium sulphate, filtered and concentrated under reduced pressure. The crude product was purified by preparative RP-HPLC (acetonitrile/water). Yield: 16.7 g (96% of theory).

LC-MS (Method 1A): Rₜ=0.82 min; MS (ESIpos): m/z=250 [M+H]⁺.

### Example 111A

### 5-(Methoxymethyl)-2,2-dimethyl-4-(1-phenylethyl)morpholin-3-one [diastereomer mixture, 4 isomers]

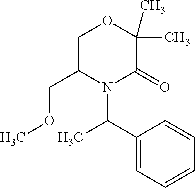 [SMILES: COCC1COC(C)(C)C(=O)N1C(C)c1ccccc1]

8.30 g (30.5 mmol) of 4-benzyl-5-(methoxymethyl)-2-methylmorpholin-3-one [diastereomer mixture, 4 isomers] were initially charged in tetrahydrofuran (381 ml), and 21.3 ml (42.7 mmol) of lithium diisopropylamide solution (1.8M in tetrahydrofuran/n-heptane/ethylbenzene) were added at −78° C. After 15 min, 5.19 g (2.28 ml, 36.6 mmol) of iodomethane were added at −78° C. The mixture was stirred for 2 h and warmed to RT during this time. Saturated aqueous ammonium chloride solution was then added and the reaction mixture was extracted with ethyl acetate. The collected organic phases were washed with saturated aqueous sodium chloride solution, dried over magnesium sulphate, filtered and concentrated under reduced pressure. The crude product was purified by preparative RP-HPLC (acetonitrile/water). Yield: 3.68 g (43% of theory).

LC-MS (Method 1A): Rₜ=0.97 min; MS (ESIpos): m/z=278 [M+H]⁺.

### Example 112A

### 5-(Methoxymethyl)-2,2-dimethyl-4-(1-phenylethyl)morpholine [diastereomer mixture, 4 isomers]

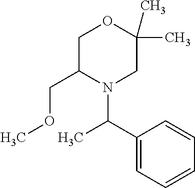 [SMILES: COCC1COC(C)(C)CN1C(C)c1ccccc1]

3.60 g (13.0 mmol) of 5-(methoxymethyl)-2,2-dimethyl-4-(1-phenylethyl)morpholin-3-one [diastereomer mixture, 4 isomers] were initially charged in tetrahydrofuran (128 ml), 26.0 ml (51.9 mmol) of 2M borane/dimethyl sulphide complex solution in tetrahydrofuran were added under argon and the reaction mixture was then stirred under reflux for 2 h. The mixture was subsequently cooled to 0° C., methanol (70 ml) was added carefully and the mixture was then stirred under reflux for 30 min. The mixture was then concentrated completely under reduced pressure, and the residue was taken up in acetonitrile and purified directly by preparative RP-HPLC (acetonitrile/water, isocratic). Yield: 2.65 g (73% of theory).

LC-MS (Method 5A): Rₜ=0.74 min; MS (ESIpos): m/z=264 [M+H]⁺.

### Example 113A

### 5-(Methoxymethyl)-2,2-dimethylmorpholine [racemate]

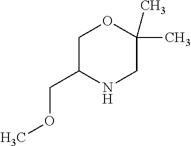

2.65 g (10.1 mmol) of 5-(methoxymethyl)-2,2-dimethyl-4-(1-phenylethyl)morpholine [diastereomer mixture, 4 isomers] were initially charged in ethanol (80.7 ml), 283 mg of palladium on carbon (10%) and 139 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 1.43 g (89% of theory).

GC-MS (Method 2B): Rₜ=2.62 min; MS (EIpos): m/z=160 [M]⁺.

### Example 114A

### 4-Benzyl-5-({[tert-butyl(dimethyl)silyl]oxy}methyl)-2,2-dimethylmorpholin-3-one [racemate]

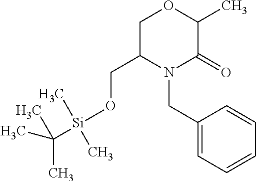 [SMILES: CC1OCC(CO[Si](C)(C)C(C)(C)C)N(Cc2ccccc2)C1=O]

17.0 g (70.7 mmol) of 4-benzyl-5-({[tert-butyl(dimethyl)silyl]oxy}methyl)-2-methylmorpholin-3-one [diastereomer mixture, 4 isomers] were initially charged in tetrahydrofuran (340 ml), and 32.4 ml (58.4 mmol) of lithium diisopropylamide solution (1.8M in tetrahydrofuran/n-heptane/ethylbenzene) were added at −78° C. The mixture was warmed slowly to 0° C., and 8.97 g (3.94 ml, 63.2 mmol) of iodomethane were then added. After 1.5 h, the mixture was again cooled to −78° C., and 5.40 ml (9.73 mmol) of lithium diisopropylamide solution (1.8M in tetrahydrofuran/n-heptane/ethylbenzene) were added. The mixture was then warmed to 0° C., and 2.07 g (0.91 ml, 14.6 mmol) of iodomethane were added. After 1 h, water was added to the reaction solution with cooling, tetrahydrofuran was removed under reduced pressure, the residue was taken up in ethyl acetate and then washed with water and saturated aqueous sodium chloride solution. The organic phase was dried over sodium sulphate, filtered and concentrated under reduced pressure. The crude product was used without further purification in the next step. Yield: 19.8 g (98% of theory, purity: 88%).

LC-MS (Method 1A): Rₜ=1.45 min; MS (ESIpos): m/z=364 [M+H]⁺.

### Example 115A

### 4-Benzyl-5-(hydroxymethyl)-2,2-dimethylmorpholin-3-one [racemate]

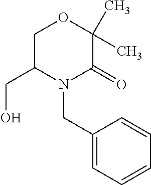 [SMILES: CC1(C)OCC(CO)N(Cc2ccccc2)C1=O]

18.1 g (43.8 mmol, purity: 88%) of 4-benzyl-5-({[tert-butyl(dimethyl)silyl]oxy}methyl)-2,2-dimethylmorpholin-3-one [racemate] were initially charged in tetrahydrofuran (329 ml), 110 ml (110 mmol) of tetra-n-butylammonium fluoride solution (1.0M in tetrahydrofuran) were added at RT and the mixture was stirred overnight. The reaction solution was then concentrated under reduced pressure and the residue was taken up in ethyl acetate and washed with water. The organic phase was dried over sodium sulphate, filtered and concentrated under reduced pressure. The crude product was purified by column chromatography on silica gel (dichloromethane, dichloromethane/methanol 100:3). Yield: 9.99 g (89% of theory).

LC-MS (Method 1A): Rₜ=0.73 min; MS (ESIpos): m/z=250 [M+H]⁺;

¹H-NMR (400 MHz, DMSO-d₆): δ[ppm]=7.43-7.35 (m, 2H), 7.34-7.21 (m, 3H), 5.09-4.98 (m, 2H), 4.23 (d, 1H), 3.90-3.75 (m, 2H), 3.65-3.55 (m, 2H), 3.15 (br. t., 1H), 1.42 (s, 3H), 1.39 (s, 3H).

### Example 116A

### 4-Benzyl-5-(fluoromethyl)-2,2-dimethylmorpholin-3-one [racemate]

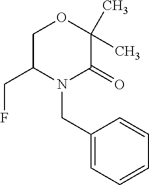 [SMILES: CC1(C)OCC(CF)N(Cc2ccccc2)C1=O]

2.00 g (8.02 mmol) of 4-benzyl-5-(hydroxymethyl)-2,2-dimethylmorpholin-3-one [racemate] were initially charged in tetrahydrofuran (40.1 ml), and 8.09 ml (18.8 mmol) of bis(2-methoxyethyl)aminosulphur trifluoride (Deoxofluor, 50% strength solution in tetrahydrofuran) were added slowly at RT. 2 drops of ethanol were then added and the mixture was subsequently stirred under reflux for 5 h. The reaction solution was carefully added dropwise to saturated aqueous sodium bicarbonate solution, the phases were separated and the aqueous phase was extracted with dichloromethane. The combined organic phases were dried over sodium sulphate, filtered and concentrated under reduced pressure. The crude product was used without further purification in the next step. Yield: 2.15 g (87% of theory, purity: 81%).

LC-MS (Method 1A): Rₜ=0.91 min; MS (ESIpos): m/z=252 [M+H]⁺.

### Example 117A

### 4-Benzyl-5-(fluoromethyl)-2,2-dimethylmorpholine [racemate]

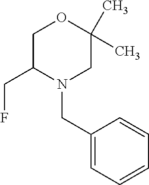 [SMILES: CC1(C)CN(Cc2ccccc2)C(CF)CO1]

2.15 g (8.56 mmol) of 4-benzyl-5-(fluoromethyl)-2,2-dimethylmorpholin-3-one [racemate] were initially charged in tetrahydrofuran (84.2 ml), 17.1 ml (34.2 mmol) of 2M borane/dimethyl sulphide complex solution in tetrahydrofuran were added under argon and the mixture was stirred under reflux for 2 h. The mixture was subsequently cooled to 0° C., methanol (10 ml) was added carefully and the mixture was stirred under reflux for 30 min. The mixture was then concentrated completely under reduced pressure, and the residue was taken up in acetonitrile and purified by preparative RP-HPLC (acetonitrile/water, isocratic). Yield: 1.03 g (43% of theory, purity: 86%).

LC-MS (Method 1A): Rₜ=0.85 min; MS (ESIpos): m/z=238 [M+H]⁺.

### Example 118A

### 5-(Fluoromethyl)-2,2-dimethylmorpholine [racemate]

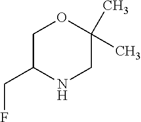

1.00 g (4.21 mmol) of 4-benzyl-5-(fluoromethyl)-2,2-dimethylmorpholine [racemate] was initially charged in ethanol (33.8 ml), 99 mg of palladium on carbon (10%) and 50 mg of palladium hydroxide on carbon (20%) were added under argon and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 192 g (31% of theory).

GC-MS (Method 2B): Rₜ=1.98 min.

### Example 119A

### 4-Benzyl-2,5,5-trimethylmorpholine [racemate]

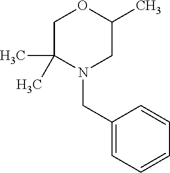 [SMILES: CC1CN(Cc2ccccc2)C(C)(C)CO1]

5.10 g (20.3 mmol) of 4-benzyl-2,5,5-trimethylmorpholin-3-one [racemate] were initially charged in tetrahydrofuran (200 ml), 30.5 ml (61.0 mmol) of 2M borane/dimethyl sulphide complex solution in tetrahydrofuran were added under argon and the mixture was stirred under reflux for 2 h. The mixture was subsequently cooled to 0° C., ethanol (150 ml) was added carefully and the mixture was stirred under reflux for 2 h. The mixture was then concentrated under reduced pressure and the residue was purified by preparative RP-HPLC (acetonitrile/water, isocratic). Yield: 2.42 g (53% of theory).

LC-MS (Method 4A): Rₜ=3.04 min; MS (ESIpos): m/z=236 [M+H]⁺;

¹H-NMR (400 MHz, DMSO-d₆): δ[ppm]=7.34-7.17 (m, 5H), 3.96 (d, 1H), 3.50-3.36 (m, 2H), 3.25 (d, 1H), 2.92 (d, 1H), 2.26 (dd, 1H), 2.04 (m_{c}, 1H), 1.04 (d, 6H), 0.98 (d, 3H).

### Example 120A

### 2,5,5-Trimethylmorpholine [racemate]

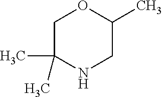

2.40 g (8.29 mmol) of 4-benzyl-2,5,5-trimethylmorpholine [racemate] were initially charged in methanol (80 ml), 240 mg of palladium on carbon (10%) and 120 mg of palladium hydroxide on carbon (20%) were added under argon and the mixture was then stirred under an atmosphere of hydrogen at standard pressure overnight. The reaction solution was filtered through kieselguhr and the filter residue was washed with methanol. The filtrate was concentrated under reduced pressure and the product was dried under high vacuum. Yield: 1.12 g (79% of theory).

¹H-NMR (400 MHz, DMSO-d₆): δ[ppm]=3.37 (d, 1H), 3.27 (m_{c}, 1H), 3.17 (s, 1H), 3.09 (dd, 1H), 1.78 (br. s., 1H), 1.08 (s, 3H), 1.01 (d, 3H), 0.87 (s, 3H), one proton not visible.

### Example 121A

### Methyl[3-(benzyloxy)cyclobutylidene][(tert-butoxycarbonyl)amino]acetate

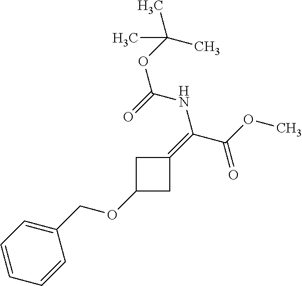 [SMILES: COC(=O)C(NC(=O)OC(C)(C)C)=C1CC(OCc2ccccc2)C1]

928 mg (3.12 mmol) of methyl [(tert-butoxycarbonyl)amino](dimethoxyphosphoryl)acetate [racemate] and 500 mg (2.84 mmol) of 3-(benzyloxy)cyclobutanone [K. Ogura, G. Tsuchihashi et al., Bull. Chem. Soc. Jpn. 1984, 57, 1637-1642] were initially charged in dichloromethane (50 ml), 605 mg (0.590 ml, 3.97) of 1,8-diazabicyclo[5.4.0]undec-7-ene were added at RT and the mixture was then stirred overnight. The reaction solution was concentrated under reduced pressure and the residue was taken up in ethyl acetate. The organic phase was washed with water, 0.5N aqueous hydrogen chloride solution, saturated aqueous sodium bicarbonate solution and saturated aqueous sodium chloride solution, dried over sodium sulphate, filtered and concentrated under reduced pressure. The residue was purified by preparative RP-HPLC (acetonitrile/water). Yield: 651 mg (60% of theory).

LC-MS (Method 1A): Rₜ=1.15 min; MS (ESIpos): m/z=348 [M+H]⁺;

¹H-NMR (400 MHz, DMSO-d₆): δ[ppm]=8.11 (br. s., 1H), 7.41-7.25 (m, 5H), 4.42 (s, 2H), 4.13 (quin, 1H), 3.63 (s, 3H), 3.25 (br. d., 1H), 2.99 (br. d., 1H), 2.85 (br. d., 1H), 2.65 (m, 1H), 1.37 (s, 9H).

### Example 122A

### Methyl[3-(benzyloxy)cyclobutyl][(tert-butoxycarbonyl)amino]acetate [cis and trans isomer mixture, 4 isomers]

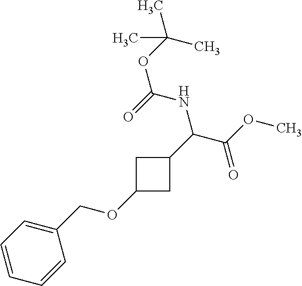 [SMILES: COC(=O)C(NC(=O)OC(C)(C)C)C1CC(OCc2ccccc2)C1]

650 mg (1.87 mmol) of methyl[3-(benzyloxy)cyclobutylidene][(tert-butoxycarbonyl)amino]acetate and 455 mg (18.7 mmol) of magnesium turnings were initially charged in methanol (50 ml) and reacted at RT in an ultrasonic bath [Elma, Transsonic T 780] for 3 h. Semisaturated aqueous ammonium chloride solution was added, and the reaction solution was extracted repeatedly with dichloromethane. The organic phases were dried over sodium sulphate, filtered and concentrated under reduced pressure. The crude product was used without further purification in the next step. Yield: 630 mg (96% of theory).

LC-MS (Method 1A): Rₜ=1.16 min; MS (ESIpos): m/z=350 [M+H]⁺, 250 [M+H-COOC(CH₃)₃];

¹H-NMR (400 MHz, DMSO-d₆): δ[ppm]=7.39-7.20 (m, 6H), 4.34 (s, 2H), 4.07 (quin, 0.3H), 3.99-3.73 (m, 1.7H), 3.60 (s, 3H), 2.34-1.94 (m, 3.5H), 1.74-1.59 (m, 1.5H), 1.45-1.27 (m, 9H).

### Example 123A

### tert-Butyl {1-[3-(benzyloxy)cyclobutyl]-2-hydroxyethyl}carbamate [cis and trans isomer mixture, 4 isomers]

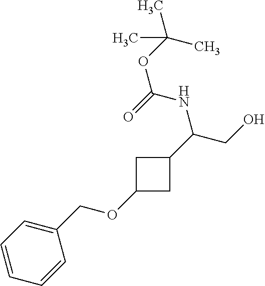 [SMILES: CC(C)(C)OC(=O)NC(CO)C1CC(OCc2ccccc2)C1]

620 mg (1.77 mmol) of methyl[3-(benzyloxy)cyclobutyl][(tert-butoxycarbonyl)amino]acetate [cis and trans isomer mixture, 4 isomers] were initially charged in tetrahydrofuran (6.0 ml), and 4.44 ml (8.87 mmol) of 2M lithium borohydride solution in tetrahydrofuran were added at 0° C. The mixture was then stirred for 4 h and allowed to warm to RT during this time. The reaction was terminated by addition of ethyl acetate (50.0 ml) and the reaction solution was subsequently washed with 0.5N aqueous hydrogen chloride solution. The organic phase was dried over sodium sulphate, filtered and concentrated under reduced pressure. The crude product was used without further purification in the next step. Yield: 560 mg (96% of theory).

LC-MS (Method 1A): Rₜ=0.99 min; MS (ESIpos): m/z=322 [M+H]⁺, 222 [M+H-Boc];

¹H-NMR (400 MHz, DMSO-d₆): δ[ppm]=7.47-7.15 (m, 5H), 6.65-6.41 (m, 1H), 4.46 (br. s., 0.5H), 4.33 (s, 2H), 3.88-3.70 (m, 0.7H), 3.67-3.09 (m, 3.8H), 2.36-1.78 (m, 3.5H), 1.74-1.48 (m, 1.5H), 1.38 (s, 9H).

### Example 124A

### 2-Amino-2-[3-(benzyloxy)cyclobutyl]ethanol trifluoroacetate [cis and trans isomer mixture, 4 isomers]

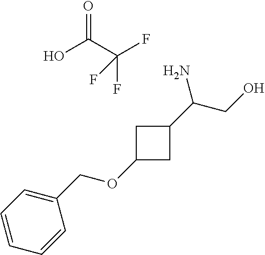

560 mg (1.74 mmol) of tert-butyl {1-[3-(benzyloxy)cyclobutyl]-2-hydroxyethyl}carbamate [cis and trans isomer mixture, 4 isomers] were initially charged in dichloromethane (8.0 ml), 1.0 ml (12.9 mmol) of trifluoroacetic acid was added at RT and the mixture was stirred for 2 h. The reaction solution was then concentrated completely under reduced pressure and excess trifluoroacetic acid was removed by repeated coevaporation with dichloromethane. The crude product was used without further purification in the next step. Yield: 580 mg (95% of theory).

LC-MS (Method 4A): Rₜ=2.10 min; MS (ESIpos): m/z=222 [M+H-TFA]⁺.

### Example 125A

### N-{1-[3-(Benzyloxy)cyclobutyl]-2-hydroxyethyl}-2-chloropropanamide [diastereomer mixture, 8 isomers]

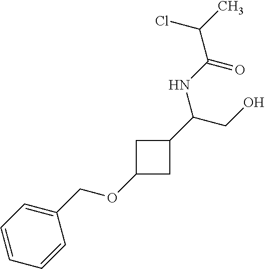 [SMILES: CC(Cl)C(=O)NC(CO)C1CC(OCc2ccccc2)C1]

580 mg (1.73 mmol) of 2-amino-2-[3-(benzyloxy)cyclobutyl]ethanol trifluoroacetate [cis and trans isomer mixture, 4 isomers] were initially charged in isopropanol (15 ml), the mixture was cooled to 0° C. and 700 mg (960 μl, 6.92 mmol) of triethylamine were added. 242 mg (190 μl, 1.90 mmol) of 2-chloropropionyl chloride [racemate] were then added dropwise, and the mixture was stirred at 0° C. for 1 h and then concentrated completely under reduced pressure. 0.5N aqueous hydrogen chloride solution (50 ml) was added to the residue, and the mixture was extracted repeatedly with dichloromethane. The organic phases were dried over sodium sulphate, filtered and concentrated under reduced pressure. The crude product was used without further purification in the next step.

Yield: 638 mg (91% of theory, purity: 77%).

LC-MS (Method 4A): Rₜ=2.36 min; MS (ESIpos): m/z=312 [M+H]⁺.

### Example 126A

### 5-[3-(Benzyloxy)cyclobutyl]-2-methylmorpholin-3-one [diastereomer mixture, 8 isomers]

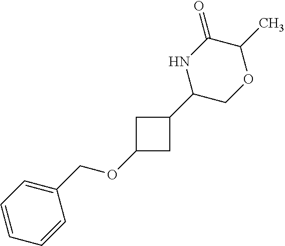 [SMILES: CC1OCC(C2CC(OCc3ccccc3)C2)NC1=O]

1.15 g (3.69 mmol) of N-{1-[3-(benzyloxy)cyclobutyl]-2-hydroxyethyl}-2-chloropropanamide [diastereomer mixture, 8 isomers] were initially charged in isopropanol (30.0 ml), the mixture was cooled to 0° C. and 1.66 g (14.8 mmol) of potassium tert-butoxide were then added in one portion. The mixture was allowed to warm to RT and then stirred at 50° C. for 1 h. Most of the isopropanol was removed under reduced pressure and the residue was taken up in ethyl acetate. The organic phase was washed with 1N aqueous hydrogen chloride solution, dried over sodium sulphate, filtered and concentrated under reduced pressure. The residue was purified by preparative RP-HPLC (acetonitrile/water). Yield: 953 mg (93% of theory).

LC-MS (Method 1A): Rₜ=0.88 min; MS (ESIpos): m/z=276 [M+H]⁺;

¹H-NMR (400 MHz, CDCl3): δ [ppm]=7.43-7.27 (m, 5H), 6.40 (br. s., 0.16H), 6.24 (br. s., 0.38H), 6.12-5.94 (m, 0.46H), 4.41 (s, 2H), 4.24-4.05 (m, 1.25H), 4.03-3.86 (m, 1.25H), 3.82-3.51 (m, 1.5H), 3.31-3.21 (m, 1H), 2.54-1.57 (m, 5H), 1.48-1.41 (m, 3H).

### Example 127A

### 5-[3-(Benzyloxy)cyclobutyl]-2-methylmorpholine [diastereomer mixture, 8 isomers]

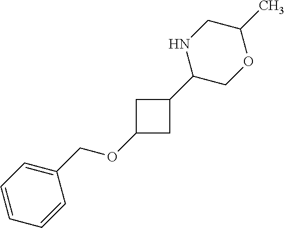 [SMILES: CC1CNC(C2CC(OCc3ccccc3)C2)CO1]

953 mg (3.46 mmol) of 5-[3-(benzyloxy)cyclobutyl]-2-methylmorpholin-3-one [diastereomer mixture, 8 isomers] were initially charged in tetrahydrofuran (10 ml), 6.92 ml (13.8 mmol) of 2M borane/dimethyl sulphide complex solution in tetrahydrofuran were added under argon and the mixture was stirred under reflux for 3 h. The reaction solution was then carefully added dropwise to ethanol (50.0 ml) and stirred under reflux for 8 h. The mixture was then concentrated under reduced pressure, and the residue was taken up in acetonitrile and purified by preparative RP-HPLC (acetonitrile/water). Yield: 780 mg (84% of theory).

LC-MS (Method 1A): Rₜ=0.57, 0.60 min; MS (ESIpos): m/z=262 [M+H]⁺;

¹H-NMR (400 MHz, DMSO-d₆): δ[ppm]=7.39-7.24 (m, 5H), 4.37-4.31 (m, 2H), 4.11-3.98 (m, 0.3H), 3.92-3.78 (m, 0.7H), 3.72-3.54 (m, 0.5H), 3.50-3.40 (m, 1.5H), 2.94-2.70 (m, 1H), 2.61 (td, 0.3H), 2.48-1.82 (m, 5.7H), 1.73-1.40 (m, 2H), 1.06-0.94 (m, 3H), one proton obscured.

### Example 128A

### Benzyl 5-[3-(benzyloxy)cyclobutyl]-2-methylmorpholine-4-carboxylate [diastereomer mixture, 4 isomers]

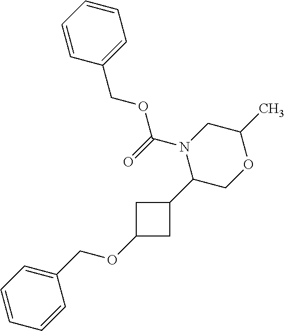 [SMILES: CC1CN(C(=O)OCc2ccccc2)C(C2CC(OCc3ccccc3)C2)CO1]

900 mg (3.44 mmol) of 5-[3-(benzyloxy)cyclobutyl]-2-methylmorpholine [diastereomer mixture, 8 isomers] and 890 mg (1.20 ml, 6.89 mmol) of N,N-diisopropylethylamine were initially charged in dichloromethane (45.0 ml), 881 mg (0.74 ml, 5.17 mmol) of benzyl chloroformate were added dropwise at 0° C. and the mixture was stirred overnight and allowed to warm to RT during this time. The reaction solution was concentrated under reduced pressure and the residue was taken up in acetonitrile. Purification and diastereomer separation by RP-HPLC on an achiral phase (acetonitrile/water) gave 537 mg (36% of theory) of the target compound of Example 128A (diastereomer mixture, 4 isomers) and 588 mg (43% of theory) of the target compound of Example 129A (diastereomer mixture, 4 isomers).

LC-MS (Method 1A): Rₜ=1.26 min; MS (ESIpos): m/z=396 [M+H]⁺;

¹H-NMR (400 MHz, DMSO-d₆): δ[ppm]=7.41-7.24 (m, 10H), 5.22-5.01 (m, 2H), 4.33-4.26 (m, 2H), 4.09-3.66 (m, 4H), 3.51 (d, 1H), 3.29-3.10 (m, 2H), 2.82 (br. s., 0.3H), 2.48-1.79 (m, 3.3H), 1.69-1.52 (m, 1.4H), 1.14-1.07 (m, 3H).

### Example 129A

### Benzyl 5-[3-(benzyloxy)cyclobutyl]-2-methylmorpholine-4-carboxylate [diastereomer mixture, 4 isomers]

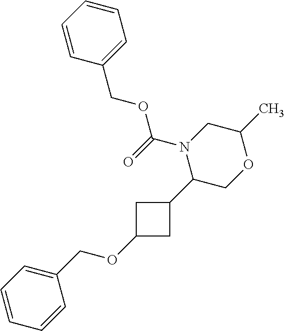 [SMILES: CC1CN(C(=O)OCc2ccccc2)C(C2CC(OCc3ccccc3)C2)CO1]

900 mg (3.44 mmol) of 5-[3-(benzyloxy)cyclobutyl]-2-methylmorpholine [diastereomer mixture, 8 isomers] and 890 mg (1.20 ml, 6.89 mmol) of N,N-diisopropylethylamine were initially charged in dichloromethane (45.0 ml), 881 mg (0.74 ml, 5.17 mmol) of benzyl chloroformate were added dropwise at 0° C. and the mixture was stirred overnight and allowed to warm to RT during this time. The reaction solution was concentrated under reduced pressure and the residue was taken up in acetonitrile. Purification and diastereomer separation by RP-HPLC on an achiral phase (acetonitrile/water) gave 537 mg (36% of theory) of the target compound of Example 128A (diastereomer mixture, 4 isomers) and 588 mg (43% of theory) of the target compound of Example 129A (diastereomer mixture, 4 isomers).

LC-MS (Method 1A): Rₜ=1.29 min; MS (ESIpos): m/z=396 [M+H]⁺;

¹H-NMR (400 MHz, DMSO-d₆): δ[ppm]=7.44-7.22 (m, 10H), 5.20-4.98 (m, 2H), 4.36-4.20 (m, 2H), 4.14-3.34 (m, 6H), 2.88-2.57 (m, 1.5H), 2.44-1.53 (m, 4.5H), 1.10-1.03 (m, 3H).

### Example 130A

### 3-(6-Methylmorpholin-3-yl)cyclobutanol [diastereomer mixture, 4 isomers]

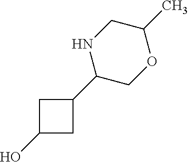

580 mg (1.47 mmol) of benzyl 5-[3-(benzyloxy)cyclobutyl]-2-methylmorpholin-4-carboxylate [Example 129A, diastereomer mixture, 4 isomers] were initially charged in ethanol (100 ml), 58 mg of palladium on carbon (10%) and 58 mg of palladium hydroxide on carbon (20%) were added under argon and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 245 mg (97% of theory).

GC-MS (Method 1B): Rₜ=4.60, 4.67 min; MS (EIpos): m/z=171 [M]⁺;

¹H-NMR (400 MHz, DMSO-d₆): δ[ppm]=4.94-4.84 (m, 1H), 4.16-4.05 (d, 0.6H), 3.93-3.82 (m, 0.7H), 3.55-3.40 (m, 3.3H), 3.19-3.14 (m, 0.7H), 3.17 (d, 1H), 2.47-1.76 (m, 6H), 1.58-1.28 (m, 1.5H), 1.08-0.94 (m, 3.5H).

### Example 131A

### [1-Amino-3-(benzyloxy)cyclobutyl]methanol [diastereomer mixture, 2 isomers, cis/trans about 4:1]

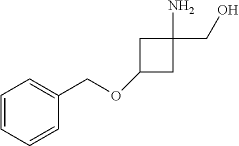 [SMILES: NC1(CO)CC(OCc2ccccc2)C1]

I) 5.00 g (20.3 mmol) of 2-(benzyloxy)-5,7-diazaspiro[3.4]octane-6,8-dione [diastereomer mixture, 2 isomers, cis/trans about 4:1; T. M. Shoup, M. M. Goodman, J. Labelled. Cpd. Radiopharm. 1999, 42, 215-225; US2006/292073 A1] were initially charged in water (100 ml), and 32.0 g (102 mmol) of barium hydroxide octahydrate were added. In seven portions, the suspension was stirred in the microwave (Biotage Synthesizer), in each case for 1.5 h at 140° C. The suspensions were combined and adjusted to a pH of about 4 using a 6N aqueous sulphuric acid solution. The precipitated solid was filtered off under reduced pressure, the filtrate was then concentrated under reduced pressure and the solid obtained was dried under high vacuum. This gave 6.2 g of crude product.

II) 21.3 g (24.9 ml, 196 mmol) of chlorotrimethylsilane were added dropwise to 49.1 ml of a 2M solution of lithium borohydride in tetrahydrofuran (98.2 mmol). The suspension obtained was cooled to 0° C., and 5.43 g of the crude product from I) were then added a little at a time. The mixture was then warmed to RT and then stirred at RT overnight. The reaction was terminated by dropwise addition of methanol (15 ml) and the reaction solution was then concentrated under reduced pressure. The residue was taken up in ethyl acetate and washed with an aqueous 2N sodium hydroxide solution. The aqueous phase was extracted with ethyl acetate, and the combined organic phases were dried over sodium sulphate, filtered and concentrated under reduced pressure. The crude product was used without further purification in the next step. Yield: 3.76 g (crude product).

LC-MS (Method 4A): Rₜ=2.10 min; MS (ESIpos): m/z=208 [M+H]⁺;

¹H-NMR (400 MHz, DMSO-d₆): δ[ppm]=7.39-7.22 (m, 5H), 4.66 (br. s., 1H), 4.32 (s, 2H), 4.15 (quin, 0.2H), 3.70 (quin, 0.8H), 3.22-3.14 (m, 2H), 2.34-2.26 (m, 2H), 1.91-1.74 (m, 2H), 1.72-1.61 (m, 2H).

### Example 132A

### tert-Butyl[3-(benzyloxy)-1-(hydroxymethyl)cyclobutyl]carbamate [enantiomerically pure cis and trans isomer]

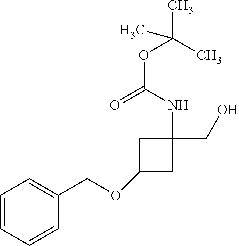 [SMILES: CC(C)(C)OC(=O)NC1(CO)CC(OCc2ccccc2)C1]

3.76 g (18.1 mmol) of [1-amino-3-(benzyloxy)cyclobutyl]methanol [diastereomer mixture, 2 isomers cis/trans about 4:1] were initially charged in dichloromethane (150 ml), 4.36 g (20.0 mmol) of di-tert-butyl dicarbonate and 3.86 g (5.31 ml, 38.1 mmol) of triethylamine were added at RT and the mixture was stirred at RT overnight. The mixture was then washed with 0.5N aqueous hydrogen chloride solution, saturated aqueous sodium bicarbonate solution and water, and the organic phase was dried over sodium sulphate, filtered and concentrated under reduced pressure. The crude product (6.4 g) was purified by preparative RP-HPLC (Method 1G) and separated into the diastereomers. Here, the more rapidly eluting major diastereomer was the cis isomer, and the slower eluting minor diastereomer was the trans isomer. Yield: 3.45 g (61% of theory, enantiomerically pure cis isomer); 690 mg (12% of theory, enantiomerically pure trans isomer).

### Enantiomerically Pure Cis Diastereomer:

LC-MS (Method 1A): Rₜ=2.00 min; MS (ESIpos): m/z=308 [M+H]⁺;

### Enantiomerically Pure Trans Diastereomer:

LC-MS (Method 1A): Rₜ=2.02 min; MS (ESIpos): m/z=308 [M+H]⁺.

### Example 133A

### [cis-1-Amino-3-(benzyloxy)cyclobutyl]methanol hydrochloride [enantiomerically pure cis isomer]

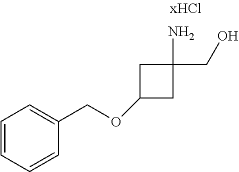 [SMILES: NC1(CO)CC(OCc2ccccc2)C1]

3.45 g (11.2 mmol) of tert-butyl [cis-3-(benzyloxy)-1-(hydroxymethyl)cyclobutyl]carbamate [enantiomerically pure cis isomer from Example 132A] were initially charged in 1,4-dioxane (30 ml), 11.2 ml of a 4N solution of hydrogen chloride in 1,4-dioxane/water were added at RT and the mixture was stirred at RT for 20 h. The mixture was then concentrated under reduced pressure and the residue was dried under high vacuum. The crude product was used without further purification in the next step. Yield: 2.81 g (quant.).

LC-MS (Method 1A): Rₜ=0.40 min; MS (ESIpos): m/z=208 [M+H-HCl]⁺;

¹H-NMR (400 MHz, DMSO-d₆): δ[ppm]=8.24 (br. s., 3H), 7.43-7.25 (m, 5H), 5.54 (br. s., 1H), 4.39 (s, 2H), 3.90 (quin, 1H), 3.46 (br. d., 2H), 2.42 (m_{c}, 2H), 2.12 (m_{c}, 2H).

### Example 134A

### N-[cis-3-(Benzyloxy)-1-(hydroxymethyl)cyclobutyl]-2-chloropropanamide [racemate]

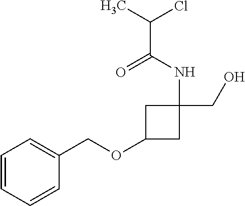 [SMILES: CC(Cl)C(=O)NC1(CO)CC(OCc2ccccc2)C1]

2.81 g (11.5 mmol) of [cis-1-amino-3-(benzyloxy)cyclobutyl]methanol hydrochloride [enantiomerically pure cis isomer] were initially charged in isopropanol (70.0 ml), the mixture was cooled to 0° C. and 4.67 g (6.43 ml, 46.1 mmol) of triethylamine were added. 1.61 g (1.26 ml, 12.7 mmol) of 2-chloropropionyl chloride [racemate] were then added dropwise. The reaction solution was allowed to warm to RT, stirred for 1 h and then concentrated under reduced pressure. The residue was taken up in dichloromethane and washed with an aqueous 1N hydrogen chloride solution. The organic phases were dried over sodium sulphate, filtered and concentrated under reduced pressure. The crude product was used without further purification in the next step. Yield: 3.38 g (97% of theory).

LC-MS (Method 1A): Rₜ=0.85 min; MS (ESIpos): m/z=298 [M+H]⁺.

### Example 135A

### cis-2-(Benzyloxy)-7-methyl-8-oxa-5-azaspiro[3.5]nonan-6-one [racemate]

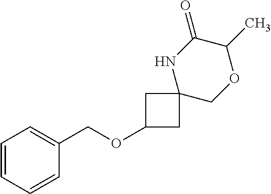 [SMILES: CC1OCC2(CC(OCc3ccccc3)C2)NC1=O]

3.38 g (11.4 mmol) of N-[cis-3-(benzyloxy)-1-(hydroxymethyl)cyclobutyl]-2-chloropropanamide [racemate] were initially charged in isopropanol (250 ml), the mixture was cooled to 0° C. and 3.82 g (34.1 mmol) of potassium tert-butoxide were added in one portion. The mixture was allowed to warm to RT and stirred at 50° C. for 1 h. Most of the isopropanol was then removed under reduced pressure and the residue was taken up in dichloromethane. The organic phase was washed with 1N aqueous hydrogen chloride solution, dried over sodium sulphate, filtered and concentrated under reduced pressure. The residue was purified by preparative RP-HPLC (acetonitrile/water).

Yield: 2.96 g (99% of theory).

LC-MS (Method 1A): Rₜ=0.86 min; MS (ESIpos): m/z=262 [M+H]⁺.

### Example 136A

### cis-2-(Benzyloxy)-7-methyl-8-oxa-5-azaspiro[3.5]nonane [racemate]

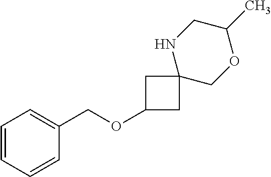 [SMILES: CC1CNC2(CO1)CC(OCc1ccccc1)C2]

2.96 g (11.3 mmol) of cis-2-(benzyloxy)-7-methyl-8-oxa-5-azaspiro[3.5]nonan-6-one [racemate] were initially charged in tetrahydrofuran (200 ml), 22.7 ml (45.3 mmol) of 2M borane/dimethyl sulphide complex solution in tetrahydrofuran were added under argon and the mixture was stirred under reflux for 2 h. The reaction solution was subsequently cooled to 0° C., methanol (100 ml) was added carefully dropwise and the mixture was stirred under reflux for 12 h. The mixture was then concentrated completely under reduced pressure, and the residue was taken up in acetonitrile and purified directly by preparative RP-HPLC (acetonitrile/water). Yield: 2.80 g (91% of theory).

LC-MS (Method 1A): Rₜ=0.61 min; MS (ESIpos): m/z=248 [M+H]⁺.

### Example 137A

### tert-Butyl cis-2-(benzyloxy)-7-methyl-8-oxa-5-azaspiro[3.5]nonane-5-carboxylate [racemate]

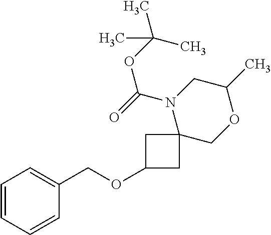 [SMILES: CC1CN(C(=O)OC(C)(C)C)C2(CO1)CC(OCc1ccccc1)C2]

2.80 g (11.3 mmol) of cis-2-(benzyloxy)-7-methyl-8-oxa-5-azaspiro[3.5]nonane [racemate] were initially charged in dichloromethane (150 ml), 3.71 g (17.0 mmol) of di-tert-butyl dicarbonate and 5.73 g (7.89 ml, 56.6 mmol) of triethylamine were added at RT and the mixture was stirred at RT overnight. The reaction solution was washed with an aqueous 0.5N hydrogen chloride solution, saturated aqueous sodium bicarbonate solution and water. The organic phase was dried over sodium sulphate, filtered and concentrated under reduced pressure. Yield: 3.33 g (84% of theory).

LC-MS (Method 1A): Rₜ=1.28 min; MS (ESIpos): m/z=348 [M+H]⁺.

### Example 138A

### tert-Butyl cis-2-(benzyloxy)-7-methyl-8-oxa-5-azaspiro[3.5]nonane-5-carboxylate [enantiomerically pure isomer 1]

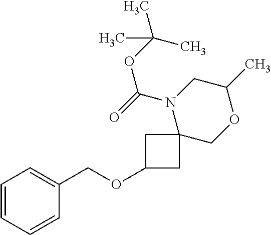 [SMILES: CC1CN(C(=O)OC(C)(C)C)C2(CO1)CC(OCc1ccccc1)C2]

The enantiomer separation of 3.33 g of the compound from Example 137A (Method 5D) gave 1.06 g of the compound from Example 138A (enantiomerically pure isomer 1).

HPLC (Method 11E): Rₜ=5.06 min, 99.9% ee;

LC-MS (Method 1A): Rₜ=1.30 min; MS (ESIpos): m/z=348 [M+H]⁺;

¹H-NMR (400 MHz, DMSO-d₆): δ[ppm]=7.40-7.23 (m, 5H), 4.37 (m_{c}, 2H), 3.79 (quin, 1H), 3.62 (dd, 1H), 3.49-3.33 (m, 3H), 2.69-2.56 (m, 2H), 2.43 (dd, 1H), 2.32-2.23 (m, 1H), 1.78 (m_{c}, 1H), 1.38 (s, 9H), 1.01 (d, 3H).

### Example 139A

### cis-2-(Benzyloxy)-7-methyl-8-oxa-5-azaspiro[3.5]nonane hydrochloride [enantiomerically pure isomer 1]

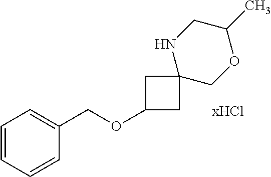 [SMILES: CC1CNC2(CO1)CC(OCc1ccccc1)C2]

1.06 g (3.06 mmol) of tert-butyl cis-2-(benzyloxy)-7-methyl-8-oxa-5-azaspiro[3.5]nonane-5-carboxylate [enantiomerically pure isomer 1 from Example 138A] were initially charged in 1,4-dioxane (30 ml), and 10.0 ml of a 4N solution of hydrogen chloride in 1,4-dioxane were added at RT. The mixture was stirred at RT overnight and then concentrated under reduced pressure, and the product was dried under high vacuum. Yield: 1.04 g (quant.).

LC-MS (Method 1A): Rₜ=0.48 min; MS (ESIpos): m/z=248 [M+H-HCl]⁺.

### Example 140A

### cis-7-Methyl-8-oxa-5-azaspiro[3.5]nonan-2-ol hydrochloride [enantiomerically pure isomer 1]

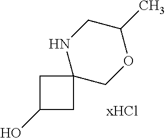

1.03 g (3.66 mmol) of cis-2-(benzyloxy)-7-methyl-8-oxa-5-azaspiro[3.5]nonane hydrochloride [enantiomerically pure isomer 1 from Example 139A] in methanol (36.7 ml) and 3.34 ml of an aqueous 2N hydrogen chloride solution were initially charged, 119 mg of palladium on carbon (10%) and 59.7 mg of palladium hydroxide on carbon (20%) were added under argon and the mixture was then stirred under an atmosphere of hydrogen at standard pressure overnight. The reaction solution was filtered through kieselguhr and the filter residue was washed with methanol. The filtrate was concentrated under reduced pressure and the product was dried under high vacuum. Yield: 785 mg (99% of theory).

MS (Method 1C): m/z=158 [M+H-HCl]⁺;

¹H-NMR (400 MHz, DMSO-d₆): δ[ppm]=9.84 (br. s., 1H), 9.57 (br. s., 1H), 3.78-3.60 (m, 4H), 3.11 (d, 1H), 2.27-2.18 (m, 1H), 2.13-2.00 (m, 2H), 1.09 (d, 3H), three protons obscured.

### Example 141A

### 4-Benzyl-2-[2-(benzyloxy)ethyl]-5-({[tert-butyl(dimethyl)silyl]oxy}methyl)-2-methylmorpholin-3-one [diastereomer mixture, 4 isomers]

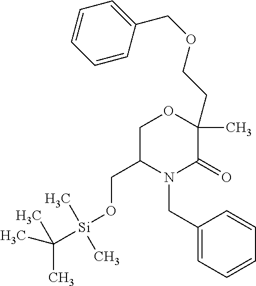 [SMILES: CC1(CCOCc2ccccc2)OCC(CO[Si](C)(C)C(C)(C)C)N(Cc2ccccc2)C1=O]

20.5 g (31.3 mmol, purity: 60%) of 4-benzyl-5-({[tert-butyl(dimethyl)silyl]oxy}methyl)-2-(2-hydroxyethyl)-2-methylmorpholin-3-one [diastereomer mixture, 4 isomers] were initially charged in N,N-dimethylformamide (205 ml) under argon, and 1.12 g (46.9 mmol, 60% suspension in paraffin oil) of sodium hydride were added at 0° C. 4.09 ml (5.88 g, 34.4 mmol) of benzyl bromide were then added dropwise, and the mixture was stirred at RT overnight. A further 560 mg (23.4 mmol, 60% suspension in paraffin oil) of sodium hydride, 2.04 ml (2.96 g, 17.2 mmol) of benzyl bromide and catalytic amounts of tetra-n-butylammonium iodide (about 50 mg) were added and the mixture was stirred at RT for 2 h. The mixture was then stirred at 50° C. for 1 h, another 560 mg (23.4 mmol, 60% suspension in paraffin oil) of sodium hydride were added and the mixture was stirred at 50° C. for 1 h. The reaction mixture was concentrated under reduced pressure and the residue was taken up in ethyl acetate and washed with water. The organic phase was dried over sodium sulphate, filtered and concentrated under reduced pressure. The crude product was purified by preparative RP-HPLC (acetonitrile/water). Yield: 7.19 g (44% of theory).

LC-MS (Method 1A): Rₜ=1.58 min; MS (ESIpos): m/z=484 [M+H]⁺.

### Example 142A

### 4-Benzyl-2-[2-(benzyloxy)ethyl]-5-(hydroxymethyl)-2-methylmorpholin-3-one [diastereomer mixture, 4 isomers]

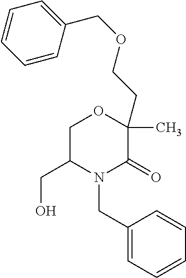 [SMILES: CC1(CCOCc2ccccc2)OCC(CO)N(Cc2ccccc2)C1=O]

7.19 g (13.7 mmol) of 4-benzyl-2-[2-(benzyloxy)ethyl]-5-({[tert-butyl(dimethyl)silyl]oxy}methyl)-2-methylmorpholin-3-one [diastereomer mixture, 4 isomers] were initially charged in tetrahydrofuran (100 ml), and 34.3 ml (34.3 mmol) of tetra-n-butylammonium fluoride solution (1.0M in tetrahydrofuran) were added at RT. The reaction solution was stirred at RT for 4 h and the reaction solution was then concentrated under reduced pressure. The residue was taken up in ethyl acetate and washed with water and the organic phase was dried over sodium sulphate, filtered and concentrated under reduced pressure. The crude product was then purified by preparative RP-HPLC (acetonitrile/water). Yield: 4.83 g (93% of theory).

LC-MS (Method 1A): Rₜ=0.99 min; MS (ESIpos): m/z=370 [M+H]⁺.

### Example 143A

### 4-Benzyl-6-[2-(benzyloxy)ethyl]-6-methyl-5-oxomorpholine-3-carbaldehyde [diastereomer mixture, 4 isomers]

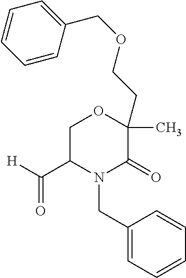 [SMILES: CC1(CCOCc2ccccc2)OCC(C=O)N(Cc2ccccc2)C1=O]

At −78° C., 4.40 g (4.00 ml, 56.3 mmol) of dimethyl sulphoxide in dichloromethane (4.0 ml) were added slowly to 4.09 g (2.81 ml, 32.2 mmol) of oxalyl chloride in dichloromethane (8.0 ml). 3.66 g (9.71 mmol) of 4-benzyl-2-[2-(benzyloxy)ethyl]-5-(hydroxymethyl)-2-methylmorpholin-3-one [diastereomer mixture, 4 isomers] in dichloromethane (8.0 ml) were then added. 10.8 g (14.9 ml, 107 mmol) of triethylamine were slowly added to the cold reaction mixture, and the reaction mixture was allowed to warm to RT and stirred overnight. The reaction mixture was poured into water, and after separation of the phases the organic phase was washed with water, dried over sodium sulphate, filtered and concentrated under reduced pressure. The crude product obtained was purified by silica gel chromatography (cyclohexane/ethyl acetate 2:1). Yield: 3.54 g (86% of theory, purity: 86%).

LC-MS (Method 1A): Rₜ=0.94 min (hydrate), Rₜ=1.11 min (aldehyde)

MS (ESIpos): m/z=368 [M+H]⁺.

### Example 144A

### 4-Benzyl-2-[2-(benzyloxy)ethyl]-5-(difluoromethyl)-2-methylmorpholin-3-one [diastereomer mixture, 4 isomers]

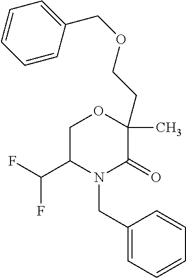 [SMILES: CC1(CCOCc2ccccc2)OCC(C(F)F)N(Cc2ccccc2)C1=O]

3.18 g (8.65 mmol) of 4-benzyl-6-[2-(benzyloxy)ethyl]-6-methyl-5-oxomorpholin-3-carbaldehyde [diastereomer mixture, 4 isomers] were initially charged in dichloromethane (127 ml), and 8.0 ml (18.6 mmol) of bis(2-methoxyethyl)aminosulphur trifluoride (Deoxofluor, 50% strength solution in tetrahydrofuran) were added slowly at RT. 1 drop of methanol was then added and the mixture was subsequently stirred at RT overnight. A further 8.0 ml (18.6 mmol) of bis(2-methoxyethyl)aminosulphur trifluoride (Deoxofluor, 50% strength solution in tetrahydrofuran) were added and the mixture was stirred at RT for 48 h. The reaction solution was carefully added dropwise to saturated aqueous sodium bicarbonate solution, the phases were separated and the aqueous phase was extracted with dichloromethane. The combined organic phases were dried over sodium sulphate, filtered and concentrated under reduced pressure. The crude product was used without further purification in the next step. Yield: 4.29 g (91% of theory, purity: 71%, diastereomer ratio about 2:1).

LC-MS (Method 1A): Rₜ=1.22 min (diastereomer 1, 2 isomers), Rₜ=1.24 min (diastereomer 2, 2 isomers).

MS (ESIpos): m/z=390 [M+H]⁺.

### Example 145A

### 4-Benzyl-2-[2-(benzyloxy)ethyl]-5-(difluoromethyl)-2-methylmorpholine [diastereomer 1, 2 isomers+diastereomer 2, 2 isomers]

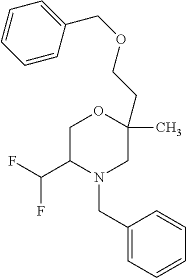 [SMILES: CC1(CCOCc2ccccc2)CN(Cc2ccccc2)C(C(F)F)CO1]

4.29 g (7.90 mmol, purity: 71%) of 4-benzyl-2-[2-(benzyloxy)ethyl]-5-(difluoromethyl)-2-methylmorpholin-3-one [diastereomer mixture, 4 isomers] were initially charged in tetrahydrofuran (80.0 ml), 15.8 ml (31.6 mmol) of 2M borane/dimethyl sulphide complex solution in tetrahydrofuran were added under argon and the mixture was stirred under reflux for 3 h. A further 6.0 ml (12.0 mmol) of 2M borane/dimethyl sulphide complex solution in tetrahydrofuran were then added and the mixture was stirred under reflux for 1 h. Methanol (40 ml) was then added carefully and the mixture was stirred under reflux for 30 min. The mixture was concentrated completely under reduced pressure and the residue was purified on an achiral phase according to Method 2G and separated into the two diastereomers. Yield: 514 mg (17% of theory, diastereomer 1, 2 isomers, minor isomers); 935 mg (31% of theory, diastereomer 2, 2 isomers, major isomers).

LC-MS (Method 1A): Rₜ=1.43 min (diastereomer 1, 2 isomers, minor isomers), Rₜ=1.43 min (diastereomer 2, 2 isomers, major isomers); MS (ESIpos): m/z=376 [M+H]⁺.

¹H-NMR (400 MHz, DMSO-d₆): δ[ppm]=7.38-7.20 (m, 10H), 6.65 (dt, 1H), 4.37 (s, 2H), 3.83 (br. t., 2H), 3.70-3.59 (m, 2H), 3.39 (t, 2H), 2.82 (m_{c}, 1H), 2.19 (d, 1H), 2.03 (dt, 1H), 1.66 (dt, 1H), 1.09 (s, 3H), 1 proton obscured, (minor isomers).

¹H-NMR (400 MHz, DMSO-d₆): δ [ppm]=7.40-7.16 (m, 10H), 6.43 (dt, 1H), 4.37 (s, 2H), 3.90-3.80 (m, 2H), 3.73-3.59 (m, 2H), 3.42 (t, 2H), 2.82 (m_{c}, 1H), 2.60 (d, 1H), 2.11 (d, 1H), 1.89 (dt, 1H), 1.69 (dt, 1H), 1.13 (s, 3H), (major isomers).

### Example 146A

### 2-[5-(Difluoromethyl)-2-methylmorpholin-2-yl]ethanol [diastereomer 1, 2 isomers]

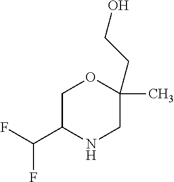

510 mg (1.36 mmol) of 4-benzyl-2-[2-(benzyloxy)ethyl]-5-(difluoromethyl)-2-methylmorpholine [diastereomer 1, 2 isomers, minor isomers, Example 145A] were initially charged in ethanol (63.8 ml), 130 mg of palladium on carbon (10%) and 65.0 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was then dried under high vacuum. Yield: 281 mg (99% of theory).

MS (Method 1C): m/z=196 [M+H]⁺.

¹H-NMR (400 MHz, DMSO-d₆): δ[ppm]=5.91 (dt, 1H), 4.28 (t, 1H), 3.61-3.40 (m, 4H), 2.87 (br. s., 1H), 2.67-2.58 (m, 1H), 1.71-1.61 (m, 1H), 1.60-1.50 (m, 1H), 1.18 (s, 3H), 2 protons obscured.

### Example 147A

### N-Benzyl-2-chloro-N-(1,4-dihydroxybutan-2-yl)propanamide [diastereomer mixture, 4 isomers]

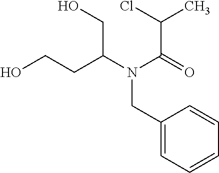 [SMILES: CC(Cl)C(=O)N(Cc1ccccc1)C(CO)CCO]

20.6 g (106 mmol) of 2-(benzylamino)butane-1,4-diol [racemate][lit.: B. L. Feringa, B. de Lange, Heterocycles 1988, 27, 1197-1205] were initially charged in isopropanol (500 ml), the mixture was cooled to 0° C. and 21.4 g (29.4 ml, 211 mmol) of triethylamine were added. 16.1 g (12.6 ml, 127 mmol) of 2-chloropropionyl chloride [racemate] were then added dropwise. After 30 min of stirring, a further 10.4 g (8.37 ml, 84.4 mmol) of 2-chloropropionyl chloride [racemate] were added dropwise, and the reaction solution was allowed to warm to RT and then concentrated under reduced pressure. The residue was taken up in ethyl acetate (500 ml) and washed with 0.5N aqueous hydrogen chloride solution (400 ml). The aqueous phase was extracted repeatedly with ethyl acetate. The organic phases were dried over sodium sulphate, filtered and concentrated under reduced pressure. The crude product was used without further purification in the next step. Yield: 37.5 g (78% of theory, purity: 63%, diastereomer ratio about 2:1).

LC-MS (Method 1A): Rₜ=0.71 min (diastereomer 1, 2 isomers), Rₜ=0.72 min (diastereomer 2, 2 isomers);

MS (ESIpos): m/z=286 [M+H]⁺.

### Example 148A

### 4-Benzyl-5-(2-hydroxyethyl)-2-methylmorpholin-3-one [diastereomer mixture, 4 isomers]

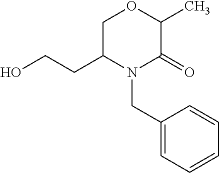 [SMILES: CC1OCC(CCO)N(Cc2ccccc2)C1=O]

37.5 g (82.5 mmol, purity: 63%) of N-benzyl-2-chloro-N-(1,4-dihydroxybutan-2-yl)propanamide [diastereomer mixture, 4 isomers] were initially charged in isopropanol (500 ml), and the mixture was cooled to 0° C. 73.5 g (655 mmol) of potassium tert-butoxide were then added in one portion, and the mixture was stirred at 0° C. for 1 h. Most of the isopropanol was removed under reduced pressure, and the residue was taken up in ethyl acetate and washed with 1N aqueous hydrogen chloride solution (400 ml). The organic phase was dried over sodium sulphate, filtered and concentrated under reduced pressure. The crude product was used without further purification in the next step. Yield: 28.8 g (quant., purity: 82%, diastereomer ratio about 2.5:1).

LC-MS (Method 7A): Rₜ=1.42 min (diastereomer 1, 2 isomers), Rₜ=1.46 min (diastereomer 2, 2 isomers);

MS (ESIpos): m/z=250 [M+H]⁺.

### Example 149A

### 2-(4-Benzyl-6-methylmorpholin-3-yl)ethanol [diastereomer mixture, 4 isomers]

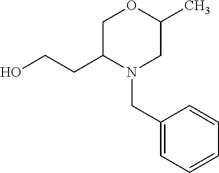 [SMILES: CC1CN(Cc2ccccc2)C(CCO)CO1]

28.8 g (94.7 mmol, purity: 82%) of 4-benzyl-5-(2-hydroxyethyl)-2-methylmorpholin-3-one [diastereomer mixture, 4 isomers] were initially charged in tetrahydrofuran (800 ml), 231 ml (462 mmol) of 2M borane/dimethyl sulphide complex solution in tetrahydrofuran were added under argon and the mixture was then stirred under reflux for 2 h. The mixture was subsequently cooled to 0° C., methanol (220 ml) was added carefully and the mixture was stirred under reflux for 30 min. This was followed by complete concentration under reduced pressure. Yield: 19.2 g (crude product).

LC-MS (Method 1A): Rₜ=0.26 min (diastereomer 1, 2 isomers), Rₜ=0.28 min (diastereomer 2, 2 isomers).

MS (ESIpos): m/z=236 [M+H]⁺.

### Example 150A

### (4-Benzyl-6-methylmorpholin-3-yl)acetaldehyde [diastereomer mixture, 4 isomers]

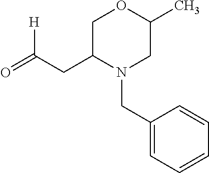 [SMILES: CC1CN(Cc2ccccc2)C(CC=O)CO1]

64.7 g (44.5 ml, 510 mmol) of oxalyl chloride were initially charged in dichloromethane (340 ml), and 79.7 g (72.4 ml, 1.02 mol) of dimethyl sulphoxide in dichloromethane (60 ml) were then added slowly at −78° C. 12.0 g (about 51.0 mmol, crude product) of 2-(4-benzyl-6-methylmorpholin-3-yl)ethanol [diastereomer mixture, 4 isomers] in dichloromethane (60 ml) were then added and the reaction mixture was stirred at −78° C. for 1 h. Over 20 min, 155 g (213 ml, 1.53 mol) of triethylamine were then slowly added to the cold reaction mixture, and the reaction mixture was allowed to warm to RT. The reaction mixture was poured into water, and after separation of the phases the organic phase was washed with water. The organic phase was then dried over sodium sulphate, filtered and concentrated under reduced pressure. The crude product was then purified by flash chromatography on silica gel (cyclohexane/ethyl acetate 10:1-1:1). Yield: 7.14 g (48% of theory, purity: 80%).

LC-MS (Method 4A): Rₜ=2.57 min; MS (ESIpos): m/z=234 [M+H]⁺.

### Example 151A

### 1-(4-Benzyl-6-methylmorpholin-3-yl)propan-2-ol [diastereomer mixture, 8 isomers]

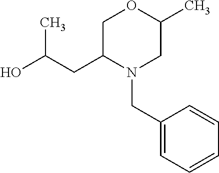 [SMILES: CC(O)CC1COC(C)CN1Cc1ccccc1]

2.30 g (9.86 mmol) of (4-benzyl-6-methylmorpholin-3-yl)acetaldehyde [diastereomer mixture, 4 isomers] were initially charged in tetrahydrofuran (42.2 ml), and 11.8 ml (11.8 mmol) of a 1M solution of methylmagnesium bromide in tetrahydrofuran were added at −78° C. The mixture was stirred at −78° C. for 15 min and then allowed to warm to RT. Saturated aqueous ammonium chloride solution (70 ml) was added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The crude product was used without further purification in the next step. Yield: 1.94 g of crude product.

LC-MS (Method 4A): Rₜ=2.34 min (diastereomer 1, 2 isomers), Rₜ=2.40 min (diastereomer 2, 2 isomers), Rₜ=2.47 min (diastereomer 3, 2 isomers); diastereomer 4, 2 isomers obscured;

MS (ESIpos): m/z=250 [M+H]⁺.

### Example 152A

### 1-(4-Benzyl-6-methylmorpholin-3-yl)acetone [diastereomer mixture, 4 isomers]

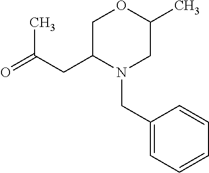 [SMILES: CC(=O)CC1COC(C)CN1Cc1ccccc1]

At −78° C., 11.9 g (10.8 ml, 152 mmol) of dimethyl sulphoxide in dichloromethane (9.0 ml) were added slowly to 9.67 g (6.65 ml, 76.2 mmol) of oxalyl chloride in dichloromethane (50 ml). 1.90 g (about 7.62 mmol, crude product) of 1-(4-benzyl-6-methylmorpholin-3-yl)propan-2-ol [diastereomer mixture, 8 isomers] in dichloromethane (9.0 ml) were then added and the reaction mixture was stirred at −78° C. for 1 h. Over 20 min, 23.1 g (31.9 ml, 229 mmol) of triethylamine were then slowly added to the cold reaction mixture, and the reaction mixture was then allowed to warm to RT. The reaction mixture was poured into water, and after separation of the phases the organic phase was washed with water. The organic phase was dried over sodium sulphate, filtered and concentrated under reduced pressure. The residue was purified by preparative RP-HPLC (acetonitrile/water). Yield: 544 mg (28% of theory, diastereomer ratio: about 1:1).

LC-MS (Method 1A): Rₜ=0.45 min (diastereomer 1, 2 isomers), Rₜ=0.47 min (diastereomer 2, 2 isomers)

MS (ESIpos): m/z=248 [M+H]⁺.

### Example 153A

### 1-(4-Benzyl-6-methylmorpholin-3-yl)-2-methylpropan-2-ol [diastereomer 1, 2 isomers+diastereomer 2, 2 isomers]

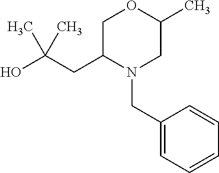 [SMILES: CC1CN(Cc2ccccc2)C(CC(C)(C)O)CO1]

544 mg (2.20 mmol) of 1-(4-benzyl-6-methylmorpholin-3-yl)acetone [diastereomer mixture, 4 isomers] were initially charged in tetrahydrofuran (9.42 ml), and 2.86 ml (2.86 mmol) of a 1M solution of methylmagnesium bromide in tetrahydrofuran were added at −78° C. The mixture was stirred at −78° C. for 15 min and then allowed to warm to RT. Saturated aqueous ammonium chloride solution (70 ml) was then added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The residue was purified by preparative RP-HPLC (acetonitrile/water, isocratic) and separated into the two diastereomers in the process. Yield: 109 mg (19% of theory, diastereomer 1, 2 isomers), 109 mg (19% of theory, diastereomer 2, 2 isomers).

LC-MS (Method 1A): Rₜ=0.49 min (diastereomer 1, 2 isomers), Rₜ=0.54 min (diastereomer 2, 2 isomers); MS (ESIpos): m/z=264 [M+H]⁺.

### Example 154A

### 2-Methyl-1-(6-methylmorpholin-3-yl)propan-2-ol [diastereomer 2, 2 isomers]

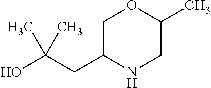

110 mg (0.416 mmol) of 1-(4-benzyl-6-methylmorpholin-3-yl)-2-methylpropan-2-ol [diastereomer 2, 2 isomers, Example 153A] were initially charged in ethanol (4.2 ml), 10.4 mg of palladium on carbon (10%) and 5.2 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 74.4 mg (quant.).

MS (Method 1C): m/z=174 [M+H]⁺.

### Example 155A

### N-Benzyl-2-chloro-N-[(2R)-1,4-dihydroxybutan-2-yl]propanamide [diastereomer mixture, 2 isomers]

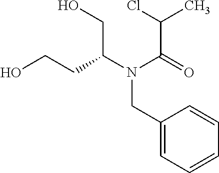 [SMILES: CC(Cl)C(=O)N(Cc1ccccc1)[C@@H](CO)CCO]

45.1 g (55.3 mmol, purity: 72%) of (2R)-2-(benzylamino)butane-1,4-diol [B. L. Feringa, Tetrahedron 1989, 45, 6799-6818] were initially charged in isopropanol (239 ml), the mixture was cooled to 0° C. and 11.2 g (15.4 ml, 111 mmol) of triethylamine were added. 10.5 g (8.23 ml, 83.0 mmol) of 2-chloropropionyl chloride [racemate] were then added dropwise. After 10 min of stirring, the reaction solution was concentrated under reduced pressure and the residue was taken up in ethyl acetate and washed with water. The organic phase was dried over sodium sulphate, filtered and concentrated under reduced pressure. The crude product was used without further purification in the next step. Yield: 21.4 g (quant., purity: 82%, diastereomer ratio about 3:2).

LC-MS (Method 1A): Rₜ=0.65 min (enantiomerically pure isomer 1), Rₜ=0.67 min (enantiomerically pure isomer 2);

MS (ESIpos): m/z=286 [M+H]⁺.

### Example 156A

### (5R)-4-Benzyl-5-(2-hydroxyethyl)-2-methylmorpholin-3-one [diastereomer mixture, 2 isomers]

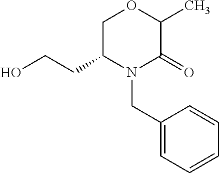 [SMILES: CC1OC[C@@H](CCO)N(Cc2ccccc2)C1=O]

21.4 g (62.1 mmol, purity: 82%) of N-benzyl-2-chloro-N-[(2R)-1,4-dihydroxybutan-2-yl]propanamide [diastereomer mixture, 2 isomers] were initially charged in isopropanol (335 ml), the mixture was cooled to 0° C. and 27.9 g (249 mmol) of potassium tert-butoxide were then added in one portion. The reaction was stirred overnight and allowed to warm to RT during this time.

Most of the isopropanol was removed under reduced pressure, and the residue was taken up in water (300 ml) and extracted with ethyl acetate. The organic phases were dried over sodium sulphate, filtered and concentrated under reduced pressure. The crude product was used without further purification in the next step. Yield: 13.3 g (69% of theory, purity: 81%, diastereomer ratio about 3:2).

LC-MS (Method 7A): Rₜ=3.23 min (enantiomerically pure isomer 1), Rₜ=3.34 min (enantiomerically pure isomer 2);

MS (ESIpos): m/z=250 [M+H]⁺.

### Example 157A

### (5R)-4-Benzyl-5-(2-{[tert-butyl(dimethyl)silyl]oxy}ethyl)-2-methylmorpholin-3-one [diastereomer mixture, 2 isomers]

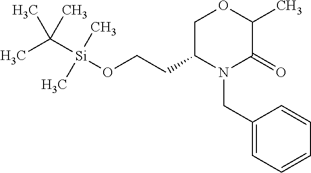 [SMILES: CC1OC[C@@H](CCO[Si](C)(C)C(C)(C)C)N(Cc2ccccc2)C1=O]

13.3 g (43.3 mmol) of (5R)-4-benzyl-5-(2-hydroxyethyl)-2-methylmorpholin-3-one [diastereomer mixture, 2 isomers] were initially charged in N,N-dimethylformamide (60.0 ml), and 8.85 g (130 mmol) of imidazole were added at RT. At 0° C., 9.80 g (65.0 mmol) of tert-butyldimethylsilyl chloride were then added and the reaction solution was stirred overnight and allowed to warm to RT during this time. The mixture was subsequently concentrated under reduced pressure, taken up in ethyl acetate and washed repeatedly with water and once with saturated aqueous sodium chloride solution. The organic phase was dried over magnesium sulphate, filtered and concentrated under reduced pressure. The crude product was then purified by chromatography on silica gel (cyclohexane/ethyl acetate 6:1, then cyclohexane/ethyl acetate 5:1). Yield: 8.03 g (49% of theory, diastereomer ratio: about 2.3:1).

LC-MS (Method 1A): Rₜ=1.41 min; MS (ESIpos): m/z=364 [M+H]⁺;

¹H-NMR (400 MHz, DMSO-d₆): δ[ppm]=7.40-7.18 (m, 5H), 5.12-5.03 (m, 1H), 4.33-4.21 (m, 1H), 4.14 (d, 0.3H), 4.05 (m, 0.7H), 3.95-3.84 (m, 1H), 3.74-3.56 (m, 3H), 3.39 (dd, 0.3H), 3.28 (d, 0.7H), 1.98-1.70 (m, 2H), 1.39 (d, 0.9H), 1.35 (d, 2.1H), 0.82 (s, 9H), 0.02 (s, 1.8H), 0.00 (s, 4.2H).

### Example 158A

### (5R)-4-Benzyl-5-(2-{[tert-butyl(dimethyl)silyl]oxy}ethyl)-2,2-dimethylmorpholin-3-one [enantiomerically pure isomer]

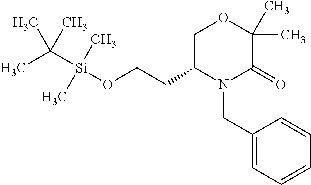 [SMILES: CC1(C)OC[C@@H](CCO[Si](C)(C)C(C)(C)C)N(Cc2ccccc2)C1=O]

7.00 g (18.6 mmol) of (5R)-4-benzyl-5-(2-{[tert-butyl(dimethyl)silyl]oxy}ethyl)-2-methylmorpholin-3-one [diastereomer mixture, 2 isomers] were initially charged in tetrahydrofuran (233 ml), and 13.0 ml (26.1 mmol) of lithium diisopropylamide solution (2.0M in tetrahydrofuran/n-heptane/ethylbenzene) were added dropwise at −78° C. The mixture was stirred for 15 min, and 3.17 g (1.39 ml, 22.4 mmol) of iodomethane were then added. The reaction solution was allowed to warm to RT and stirred for 2 h. The reaction was terminated by addition of saturated aqueous ammonium chloride solution and the mixture was extracted with ethyl acetate. The organic phase was washed with saturated aqueous sodium chloride solution, dried over magnesium sulphate, filtered and concentrated under reduced pressure. The crude product was used without further purification in the next step. Yield: 8.36 g (70% of theory, purity: 59%).

LC-MS (Method 1A): Rₜ=1.47 min; MS (ESIpos): m/z=378 [M+H]⁺.

### Example 159A

### (5R)-4-Benzyl-5-(2-{[tert-butyl(dimethyl)silyl]oxy}ethyl)-2,2-dimethylmorpholine [enantiomerically pure isomer]

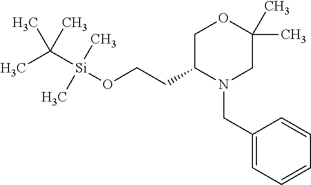 [SMILES: CC1(C)CN(Cc2ccccc2)[C@H](CCO[Si](C)(C)C(C)(C)C)CO1]

8.36 g (13.1 mmol, purity: 59%) of (5R)-4-benzyl-5-(2-{[tert-butyl(dimethyl)silyl]oxy}ethyl)-2,2-dimethylmorpholin-3-one [enantiomerically pure isomer] were initially charged in tetrahydrofuran (133 ml), 26.2 ml (52.3 mmol) of 2M borane/dimethyl sulphide complex solution in tetrahydrofuran were added under argon and the mixture was stirred under reflux for 4 h. The mixture was subsequently cooled to 0° C., methanol (30 ml) was added carefully and the mixture was stirred under reflux for 30 min and then concentrated under reduced pressure. The crude product was used without further purification in the next step. Yield: 8.39 g (96% of theory, purity: 55%).

LC-MS (Method 1A): Rₜ=1.15 min; MS (ESIpos): m/z=364 [M+H]⁺.

### Example 160A

### 2-[(3R)-4-Benzyl-6,6-dimethylmorpholin-3-yl]ethanol [enantiomer mixture, 2 isomers]

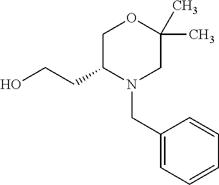 [SMILES: CC1(C)CN(Cc2ccccc2)[C@H](CCO)CO1]

7.39 g (11.2 mmol, purity: 55%) of (5R)-4-benzyl-5-(2-{[tert-butyl(dimethyl)silyl]oxy}ethyl)-2,2-dimethylmorpholine [enantiomerically pure isomer] were initially charged in tetrahydrofuran (148 ml), and 30.5 ml (30.5 mmol) of tetra-n-butylammonium fluoride solution (1.0M in tetrahydrofuran) were added at RT. The reaction solution was stirred at RT for 1 h and then concentrated under reduced pressure. The residue was purified by preparative RP-HPLC (acetonitrile/water, isocratic). Yield: 1.97 g (38% of theory, enantiomer ratio: about 85:15); at this stage, a proportional isomerization of the stereocentre to one of the earlier precursors was noticed.

HPLC (Method 7E): Rₜ=4.41 min, 85:15 R:S enantiomer ratio;

LC-MS (Method 1A): Rₜ=0.35 min; MS (ESIpos): m/z=250 [M+H]⁺;

¹H-NMR (400 MHz, DMSO-d₆): δ[ppm]=7.31 (d, 4H), 7.22 (m_{c}, 1H), 4.45 (t, 1H), 3.93 (d, 1H), 3.60 (dd, 1H), 3.54-3.40 (m, 3H), 3.10 (d, 1H), 2.40-2.29 (m, 2H), 1.85 (d, 1H), 1.79-1.69 (m, 1H), 1.59 (m_{c}, 1H), 1.14 (s, 3H), 1.04 (s, 3H).

### Example 161A

### 2-[(3R)-6,6-Dimethylmorpholin-3-yl]ethanol [enantiomer mixture, 2 isomers]

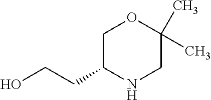

1.00 g (4.01 mmol) of 2-[(3R)-4-benzyl-6,6-dimethylmorpholin-3-yl]ethanol [enantiomer mixture, enantiomer ratio: about 85:15] were initially charged in ethanol (40.0 ml), 150 mg of palladium on carbon (10%) and 150 mg of palladium hydroxide on carbon (20%) were added under argon and the mixture was then stirred under an atmosphere of hydrogen at standard pressure for 4 h. The reaction solution was filtered through kieselguhr and concentrated under reduced pressure. Yield: 680 mg (quant.).

GC-MS (Method 2B): Rₜ=3.71 min; MS (EIpos): m/z=159 [M]⁺;

¹H-NMR (500 MHz, DMSO-d₆): δ[ppm]=4.32 (br. s., 1H), 3.46 (t, 2H), 3.38 (dd, 1H), 3.21 (t, 1H), 2.64-2.54 (m, 2H), 2.47-2.42 (m, 1H), 1.36 (m_{c}, 2H), 1.18 (s, 3H), 1.02 (s, 3H), one proton obscured.

### Example 162A

### 4-Benzyl-5-[(difluoromethoxy)methyl]-2-methylmorpholin-3-one [diastereomer mixture, 4 isomers]

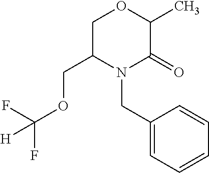 [SMILES: CC1OCC(COC(F)F)N(Cc2ccccc2)C1=O]

4.03 g (17.1 mmol) of 4-benzyl-5-(hydroxymethyl)-2-methylmorpholin-3-one [diastereomer mixture, 4 isomers] were initially charged in acetonitrile (80 ml), and the solution was then degassed by introducing argon for 5 min. Subsequently, 652 mg (3.43 mmol) of copper(I) iodide were added and the reaction mixture was heated to 55° C. A solution of 5.49 g (3.19 ml, 30.8 mmol) of difluoro(fluorosulphonyl)acetic acid in degassed acetonitrile (10 ml) was then added dropwise over 30 min and the mixture was stirred at 55° C. for 3 h. The reaction solution was concentrated under reduced pressure, water was added to the residue and the mixture was extracted with ethyl acetate. The organic phase was dried over sodium sulphate, filtered and concentrated under reduced pressure. The residue was then purified by preparative RP-HPLC (acetonitrile/water, isocratic).

Yield: 3.15 g (64% of theory).

LC-MS (Method 1A): Rₜ=0.92 min; MS (ESIpos): m/z=286 [M+H]⁺.

### Example 163A

### 2-Allyl-4-benzyl-5-[(difluoromethoxy)methyl]-2-methylmorpholin-3-one [diastereomer mixture, 4 isomers]

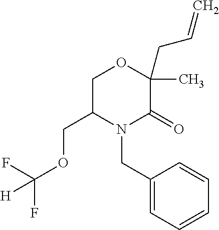 [SMILES: C=CCC1(C)OCC(COC(F)F)N(Cc2ccccc2)C1=O]

8.30 g (29.1 mmol) of 4-benzyl-5-[(difluoromethoxy)methyl]-2-methylmorpholin-3-one [diastereomer mixture, 4 isomers] were initially charged in tetrahydrofuran (364 ml), 69.8 ml (69.8 mmol) of 1M lithium hexamethyldisilazide solution in tetrahydrofuran were added under argon and at −78° C. and the reaction mixture was then stirred for 15 min. Subsequently, at −78° C., 14.7 g (7.98 ml, 87.3 mmol) of allyl iodide were added dropwise, and the reaction mixture was warmed to RT and stirred overnight. The reaction was terminated by addition of saturated aqueous ammonium chloride solution and the mixture was then extracted with ethyl acetate. The organic phase was washed with saturated aqueous sodium chloride solution, dried over magnesium sulphate, filtered and concentrated under reduced pressure. The crude product was used without further purification in the next step. Yield: 9.92 g (94% of theory).

LC-MS (Method 1A): Rₜ=1.09 min; MS (ESIpos): m/z=326 [M+H]⁺.

### Example 164A

### {4-Benzyl-5-[(1,1-difluoroethoxy)methyl]-2-methyl-3-oxomorpholin-2-yl}acetaldehyde [diastereomer mixture, 4 isomers]

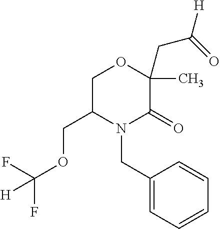 [SMILES: CC1(CC=O)OCC(COC(F)F)N(Cc2ccccc2)C1=O]

10.5 g (30.9 mmol) of 2-allyl-4-benzyl-5-[(difluoromethoxy)methyl]-2-methylmorpholin-3-one [diastereomer mixture, 4 isomers] were initially charged in tetrahydrofuran (250 ml) and water (150 ml), and 1.68 ml (0.618 mmol) of a 2.5% solution of osmium tetroxide in tert-butanol and 19.8 g (92.6 mmol) of sodium periodate were added at 0° C. The mixture was then warmed to RT and stirred overnight. The reaction solution was filtered through kieselguhr and the filter residue was washed with tetrahydrofuran. The reaction solution was taken up in ethyl acetate and diluted with water. After separation of the phases, the organic phase was washed with 1N aqueous sodium sulphite solution (2×800 ml), dried over magnesium sulphate, filtered and concentrated under reduced pressure. The crude product was used without further purification in the next step. Yield: 9.30 g of crude product.

LC-MS (Method 1A): Rₜ=0.96 min; MS (ESIpos): m/z=328 [M+H]⁺.

### Example 165A

### 4-Benzyl-5-[(difluoromethoxy)methyl]-2-(2-hydroxyethyl)-2-methylmorpholin-3-one [diastereomer mixture, 4 isomers]

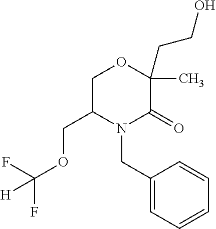 [SMILES: CC1(CCO)OCC(COC(F)F)N(Cc2ccccc2)C1=O]

3.00 g (about 5.96 mmol, crude product) of {4-benzyl-5-[(1,1-difluoroethoxy)methyl]-2-methyl-3-oxomorpholin-2-yl}acetaldehyde [diastereomer mixture, 4 isomers] were initially charged in methanol (44.4 ml), and 676 mg (17.9 mmol) of sodium borohydride were added at 0° C. The mixture was then warmed to RT and stirred for 30 min. Water was added, and the reaction solution was extracted with ethyl acetate. The organic phase was dried over sodium sulphate, filtered and concentrated under reduced pressure. The crude product was used without further purification in the next step. Yield: 2.66 g (83% of theory, purity: 61%).

LC-MS (Method 1A): Rₜ=0.83 min (diastereomer 1, 2 isomers), Rₜ=0.85 min (diastereomer 2, 2 isomers).

MS (ESIpos): m/z=330 [M+H]⁺.

### Example 166A

### 2-{4-Benzyl-5-[(difluoromethoxy)methyl]-2-methylmorpholin-2-yl}ethanol [enantiomerically pure isomer 2]

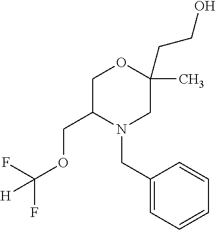 [SMILES: CC1(CCO)CN(Cc2ccccc2)C(COC(F)F)CO1]

2.66 g (4.96 mmol, purity: 61%) of 4-benzyl-5-[(difluoromethoxy)methyl]-2-(2-hydroxyethyl)-2-methylmorpholin-3-one [diastereomer mixture, 4 isomers] were initially charged in tetrahydrofuran (50 ml), 9.91 ml (19.8 mmol) of 2M borane/dimethyl sulphide complex solution in tetrahydrofuran were added under argon and the reaction mixture was then stirred under reflux for 2 h. The mixture was subsequently cooled to 0° C., methanol (45 ml) was added carefully and the mixture was then stirred under reflux for 30 min. The mixture was then concentrated completely under reduced pressure, and the residue was taken up in acetonitrile and purified directly by preparative RP-HPLC (acetonitrile/water). The isomer mixture was separated on a chiral phase according to Methods 43D and 44D. Yield: 316 mg (19% of theory, enantiomerically pure isomer 2).

HPLC (Method 37E): Rₜ=8.60 min, >99.0% ee;

LC-MS (Method 1A): Rₜ=0.80 min; MS (ESIpos): m/z=316 [M+H]⁺;

¹H-NMR (400 MHz, DMSO-d₆): δ[ppm]=7.32 (d, 1H), 7.24 (m_{c}, 1H), 6.67 (t, 1H), 4.22 (t, 1H), 4.06 (dd, 1H), 3.96 (dd, 1H), 3.90 (d, 1H), 3.69 (dd, 1H), 3.54 (dd, 1H), 3.47-3.32 (m, 3H), 2.63-2.55 (m, 1H), 2.36 (d, 1H), 2.04 (d, 1H), 1.80 (m_{c}, 1H), 1.49 (m_{c}, 1H), 1.12 (s, 3H).

### Example 167A

### 2-{5-[(Difluoromethoxy)methyl]-2-methylmorpholin-2-yl}ethanol [enantiomerically pure isomer 2]

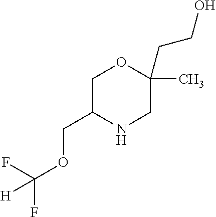

310 mg (0.983 mmol) of 2-{4-benzyl-5-[(difluoromethoxy)methyl]-2-methylmorpholin-2-yl}ethanol [enantiomerically pure isomer 2] were initially charged in ethanol (20.0 ml), 50 mg of palladium on carbon (10%) and 25 mg of palladium hydroxide on carbon (20%) were added under argon and the mixture was then stirred under an atmosphere of hydrogen at standard pressure overnight. The reaction solution was filtered through kieselguhr and concentrated under reduced pressure. Yield: 198 mg (85% of theory).

MS (Method 1C): m/z=226 [M]⁺;

¹H-NMR (400 MHz, DMSO-d₆): δ [ppm]=6.66 (t, 1H), 4.25 (t, 1H), 3.71 (dd, 2H), 3.53-3.43 (m, 3H), 2.84-2.74 (m, 1H), 2.60 (d, 1H), 2.29 (br. s., 1H), 1.57 (m_{c}, 2H), 1.18 (s, 3H), two protons obscured.

### Example 168A

### 2-Allyl-4-benzyl-2-methylmorpholin-3-one [racemate]

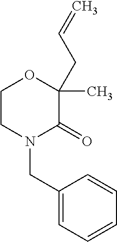 [SMILES: C=CCC1(C)OCCN(Cc2ccccc2)C1=O]

20.0 g (97.4 mmol) of 4-benzyl-2-methylmorpholin-3-one [racemate][R. Perrone et al., Synthesis 1976, 9, 598-600] were initially charged in tetrahydrofuran (500 ml), 136 ml (136 mmol) of 1M lithium hexamethyldisilazide solution in tetrahydrofuran were added under argon and at −78° C. and the reaction mixture was then stirred for 15 min. Subsequently, at −78° C., 19.6 g (10.7 ml, 117 mmol) of allyl iodide were added, and the reaction mixture was warmed to RT and stirred overnight. The mixture was then cooled to 0° C., another 68 ml (68 mmol) of 1M lithiumhexamethyldisilazide solution in tetrahydrofuran and 9.80 g (5.35 ml, 56.5 mmol) were added and the mixture was stirred at RT for 3 h. The reaction was terminated by addition of saturated aqueous ammonium chloride solution and then extracted with ethyl acetate. The organic phase was washed with saturated aqueous sodium chloride solution, dried over sodium sulphate, filtered and concentrated under reduced pressure. The crude product was used without further purification in the next step. Yield: 25.8 g (73% of theory, purity: 67%).

LC-MS (Method 1A): Rₜ=0.97 min; MS (ESIpos): m/z=246 [M+H]⁺.

### Example 169A

### (4-Benzyl-2-methyl-3-oxomorpholin-2-yl)acetaldehyde [racemate]

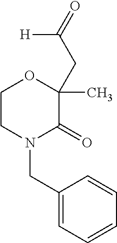 [SMILES: CC1(CC=O)OCCN(Cc2ccccc2)C1=O]

6.40 g (26.1 mmol) of 2-allyl-4-benzyl-2-methylmorpholin-3-one [racemate] were initially charged in tetrahydrofuran (400 ml) and water (250 ml), and 6.55 ml (2.41 mmol) of a 2.5% solution of osmium tetroxide in tert-butanol and 16.7 g (78.3 mmol) of sodium periodate were added at 0° C. The mixture was then warmed to RT and stirred for 20 h. The reaction solution was filtered through kieselguhr and the filter residue was washed with tetrahydrofuran. The reaction solution was taken up in ethyl acetate, washed with saturated aqueous sodium chloride solution, dried over sodium sulphate, filtered and concentrated under reduced pressure. The crude product was used without further purification in the next step. Yield: 6.99 g of crude product.

LC-MS (Method 1A): Rₜ=0.78 min; MS (ESIpos): m/z=248 [M+H]⁺.

### Example 170A

### 4-Benzyl-2-(2-hydroxyethyl)-2-methylmorpholin-3-one [racemate]

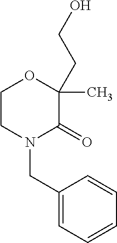 [SMILES: CC1(CCO)OCCN(Cc2ccccc2)C1=O]

6.99 g (about 28.3 mmol, crude product) of (4-benzyl-2-methyl-3-oxomorpholin-2-yl)acetaldehyde [racemate] were initially charged in methanol (120 ml), and 3.04 g (80.4 mmol) of sodium borohydride were added at 0° C. The mixture was then warmed to RT and stirred for 30 min. Water was added to the reaction solution, most of the methanol was removed under reduced pressure and the residue was extracted with ethyl acetate. The organic phase was dried over sodium sulphate, filtered and concentrated under reduced pressure. The crude product was used without further purification in the next step. Yield: 5.80 g (82% of theory, crude product).

LC-MS (Method 2A): Rₜ=0.67 min; MS (ESIpos): m/z=250 [M+H]⁺.

### Example 171A

### 2-(4-Benzyl-2-methylmorpholin-2-yl)ethanol [racemate]

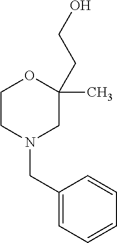 [SMILES: CC1(CCO)CN(Cc2ccccc2)CCO1]

5.80 g (about 23.3 mmol, crude product) of 4-benzyl-2-(2-hydroxyethyl)-2-methylmorpholin-3-one [racemate] were initially charged in tetrahydrofuran (230 ml), 116 ml (233 mmol) of 2M borane/dimethyl sulphide complex solution in tetrahydrofuran were added under argon and the reaction mixture was then stirred under reflux for 1 h. The mixture was subsequently cooled to RT, ethanol (90 ml) was added carefully and the mixture was then stirred under reflux for 1 h. The mixture was then concentrated completely under reduced pressure, and the residue was taken up in acetonitrile and purified directly by preparative RP-HPLC (acetonitrile/water, isocratic). Yield: 4.58 g (83% of theory).

LC-MS (Method 4A): Rₜ=2.42 min; MS (ESIpos): m/z=236 [M+H]⁺;

¹H-NMR (400 MHz, DMSO-d₆): δ[ppm]=7.36-7.21 (m, 5H), 4.27 (t, 1H), 3.59 (t, 2H), 3.48-3.39 (m, 4H), 2.29 (br. d., 2H), 2.18 (d, 1H), 2.09 (d, 1H), 1.91 (dt, 1H), 1.56 (dt, 1H), 1.13 (s, 3H).

### Example 172A

### 2-(2-Methylmorpholin-2-yl)ethanol [racemate]

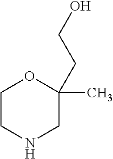

4.65 g (19.8 mmol) of 2-(4-benzyl-2-methylmorpholin-2-yl)ethanol [racemate] were initially charged in ethanol (200 ml), 465 mg of palladium on carbon (10%) and 235 mg of palladium hydroxide on carbon (20%) were added under argon, and the mixture was then stirred under an atmosphere of hydrogen at standard pressure for 36 h. The reaction solution was filtered through kieselguhr and the filter residue was washed with methanol. The filtrate was concentrated under reduced pressure and the product was dried under high vacuum. Yield: 2.66 g (90% of theory).

MS (Method 2C): m/z=146 [M+H]⁺;

¹H-NMR (400 MHz, DMSO-d₆): δ[ppm]=3.53-3.38 (m, 4H), 2.58 (dd, 1H), 2.52 (d, 1H), 2.43 (d, 1H), 1.84 (dt, 1H), 1.55 (dt, 1H), 1.09 (s, 3H), two protons not visible.

### Example 173A

### Benzyl 2-(2-hydroxyethyl)-2-methylmorpholine-4-carboxylate [racemate]

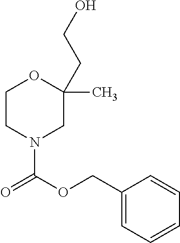 [SMILES: CC1(CCO)CN(C(=O)OCc2ccccc2)CCO1]

1.85 g (12.7 mmol) of 2-(2-methylmorpholin-2-yl)ethanol [racemate] were initially charged in dichloromethane (25.5 ml), and 2.13 g (2.93 ml, 21.0 mmol) of triethylamine and then dropwise 3.57 g (3.00 ml, 21.0 mmol) of benzyl chloroformate were added at 0° C. The reaction solution was warmed to RT and stirred overnight. The reaction solution was diluted with dichloromethane and washed in each case once with 1N aqueous hydrogen chloride solution, saturated aqueous sodium bicarbonate solution and saturated aqueous sodium chloride solution, dried over sodium sulphate, filtered and concentrated under reduced pressure. The residue was taken up in acetonitrile and purified directly by preparative RP-HPLC (acetonitrile/water). Yield: 2.61 g (73% of theory).

LC-MS (Method 1A): Rₜ=0.83 min; MS (ESIpos): m/z=280 [M+H]⁺.

### Example 174A

### Benzyl 2-methyl-2-(2-oxoethyl)morpholine-4-carboxylate [racemate]

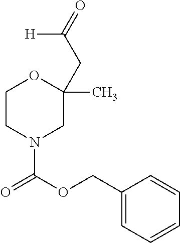 [SMILES: CC1(CC=O)CN(C(=O)OCc2ccccc2)CCO1]

3.61 g (2.48 ml, 28.5 mmol) of oxalyl chloride were initially charged in dichloromethane (63 ml), and 2.15 g (1.95 ml, 27.5 mmol) of dimethyl sulphoxide in dichloromethane (11 ml) were then added slowly at −78° C. After 30 min, 2.65 g (9.49 mmol) of benzyl 2-(2-hydroxyethyl)-2-methylmorpholine-4-carboxylate [racemate] in dichloromethane (11 ml) were added. After 30 min, 5.76 g (7.93 ml, 56.9 mmol) of triethylamine were added slowly (20 min) to the cold reaction mixture, and the reaction mixture was then warmed to RT and stirred overnight. The reaction mixture was poured into water, and after separation of the phases the organic phase was washed with water. The organic phase was dried over sodium sulphate, filtered and concentrated under reduced pressure. The crude product obtained was purified by means of flash chromatography on silica gel (cyclohexane/ethyl acetate 10:1-1:1). Yield: 2.99 g (96% of theory, purity: 85%).

LC-MS (Method 1A): Rₜ=0.90 min; MS (ESIpos): m/z=278 [M+H]⁺.

### Example 175A

### Benzyl 2-(2-hydroxypropyl)-2-methylmorpholine-4-carboxylate [enantiomerically pure isomer 2]

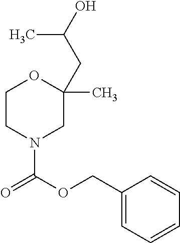 [SMILES: CC(O)CC1(C)CN(C(=O)OCc2ccccc2)CCO1]

2.99 g (9.16 mmol, purity: 85%) of benzyl 2-methyl-2-(2-oxoethyl)morpholine-4-carboxylate [racemate] were initially charged in tetrahydrofuran (39.2 ml), and 11.0 ml (11.0 mmol) of a 1M solution of methylmagnesium bromide in tetrahydrofuran were added at −78° C. The mixture was stirred at −78° C. for 15 min and then warmed to RT. Saturated aqueous ammonium chloride solution (70 ml) was then added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The residue was taken up in acetonitrile and purified directly by preparative RP-HPLC (acetonitrile/water). The isomer mixture was separated repeatedly on a chiral phase according to Method 45D. Yield: 362 mg (14% of theory, enantiomerically pure isomer 2).

HPLC (Method 39E): Rₜ=6.84 min, >95% ee;

LC-MS (Method 1A): Rₜ=0.91 min; MS (ESIpos): m/z=294 [M+H]⁺.

### Example 177A

### 1-(2-Methylmorpholin-2-yl)propan-2-ol [enantiomerically pure isomer 2]

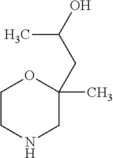

362 mg (1.23 mmol) of benzyl 2-(2-hydroxypropyl)-2-methylmorpholine-4-carboxylate [enantiomerically pure isomer 2] were initially charged in ethanol (12.4 ml), 36.0 mg of palladium on carbon (10%) and 18.0 mg of palladium hydroxide on carbon (20%) were added under argon and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 198 mg (100% of theory).

MS (Method 2C): m/z=160 [M+H]⁺.

### Example 178A

### [4-Benzyl-6,6-dimethylmorpholin-3-yl]acetaldehyde [enantiomer mixture, 2 isomers]

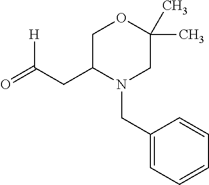 [SMILES: CC1(C)CN(Cc2ccccc2)C(CC=O)CO1]

800 mg (550 μl, 6.30 mmol) of oxalyl chloride were initially charged in dichloromethane (20 ml), and 861 mg (782 μl, 11.0 mmol) of dimethyl sulphoxide in dichloromethane (5.0 ml) were then added slowly at −78° C. 947 mg (3.80 mmol) of 2-[(3R)-4-benzyl-6,6-dimethylmorpholin-3-yl]ethanol [enantiomer mixture, 2 isomers] in dichloromethane (6.0 ml) were then added and the reaction mixture was stirred at −78° C. for 1 h. Over 20 min, 2.11 g (2.91 ml, 20.9 mmol) of triethylamine were then slowly added to the cold reaction mixture, and the reaction mixture was then warmed to RT. The reaction mixture was poured into water, and after separation of the phases the organic phase was washed with water. The organic phase was dried over sodium sulphate, filtered and concentrated under reduced pressure. The crude product was then purified by flash chromatography on silica gel (cyclohexane/ethyl acetate 10:1). Yield: 860 mg (91% of theory); proportional isomerization.

LC-MS (Method 1A): Rₜ=0.49 min; MS (ESIpos): m/z=249 [M+H]⁺;

¹H-NMR (400 MHz, DMSO-d₆): δ[ppm]=9.78 (t, 1H), 7.37-7.16 (m, 5H), 3.81 (d, 1H), 3.67 (dd, 1H), 3.52 (dd, 1H), 3.16 (d, 1H), 2.89-2.80 (m, 1H), 2.72-2.64 (m, 2H), 2.32 (d, 1H), 1.92 (d, 1H), 1.13 (s, 3H), 1.08 (s, 3H).

### Example 179A

### 1-[4-Benzyl-6,6-dimethylmorpholin-3-yl]propan-2-ol [diastereomer mixture, 4 isomers]

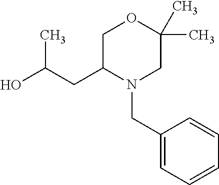 [SMILES: CC(O)CC1COC(C)(C)CN1Cc1ccccc1]

860 mg (3.48 mmol) of [4-benzyl-6,6-dimethylmorpholin-3-yl]acetaldehyde [enantiomer mixture, 2 isomers] were initially charged in tetrahydrofuran (14.9 ml), and 4.17 ml (4.17 mmol) of a 1M solution of methylmagnesium bromide in tetrahydrofuran were added at −78° C. The mixture was stirred at −78° C. for 15 min and then warmed to RT. Saturated aqueous ammonium chloride solution (50 ml) was then added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The residue was directly purified by preparative RP-HPLC (acetonitrile/water, isocratic). Yield: 571 mg (62% of theory, diastereomer ratio: about 1:1).

LC-MS (Method 1A): Rₜ=0.47 min (diastereomer 1, 2 isomers), Rₜ=0.50 min (diastereomer 2, 2 isomers).

MS (ESIpos): m/z=264 [M+H]⁺.

### Example 180A

### 1-[6,6-Dimethylmorpholin-3-yl]propan-2-ol [diastereomer mixture, 4 isomers]

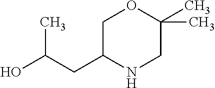

565 mg (2.15 mmol) of 1-[4-benzyl-6,6-dimethylmorpholin-3-yl]propan-2-ol [diastereomer mixture, 4 isomers] were initially charged in ethanol (21.6 ml), 53.9 mg of palladium on carbon (10%) and 27.0 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 366 mg (98% of theory).

MS (Method 2C): m/z=174 [M+H]⁺.

### Example 181A

### N-Benzyl-2-chloro-N-[(2S)-1,4-dihydroxybutan-2-yl]propanamide [diastereomer mixture, 2 isomers]

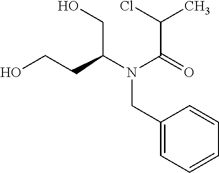 [SMILES: CC(Cl)C(=O)N(Cc1ccccc1)[C@H](CO)CCO]

45.1 g (199 mmol, purity: 86%) of (2S)-2-(benzylamino)butane-1,4-diol [F. Horiuchi, M. Matsui, Agr. Biol. Chem. 1973, 37, 1713-1716] were initially charged in isopropanol (1.00 l), the mixture was cooled to 0° C. and 40.2 g (55.4 ml, 397 mmol) of triethylamine were added. 37.8 g (29.6 ml, 298 mmol) of 2-chloropropionyl chloride [racemate] were then added dropwise. After 30 min of stirring, a further 18.9 g (14.8 ml, 149 mmol) of 2-chloropropionyl chloride [racemate] were added dropwise, and the reaction solution was allowed to warm to RT and then concentrated under reduced pressure. The residue was taken up in ethyl acetate (1.00 l) and washed with water. The organic phase was dried over sodium sulphate, filtered and concentrated under reduced pressure. The crude product was used without further purification in the next step. Yield: 71.8 g (quant., purity: 82%, diastereomer ratio about 1:1).

LC-MS (Method 1A): Rₜ=0.65 min (enantiomerically pure isomer 1), Rₜ=0.67 min (enantiomerically pure isomer 2);

MS (ESIpos): m/z=286 [M+H]⁺.

### Example 182A

### (5S)-4-Benzyl-5-(2-hydroxyethyl)-2-methylmorpholin-3-one [diastereomer mixture, 2 isomers]

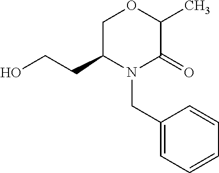 [SMILES: CC1OC[C@H](CCO)N(Cc2ccccc2)C1=O]

71.8 g (206 mmol, purity: 82%) of N-benzyl-2-chloro-N-[(2S)-1,4-dihydroxybutan-2-yl]propanamide [diastereomer mixture, 2 isomers] were initially charged in isopropanol (1.30 l), and the mixture was cooled to 0° C. 92.4 g (824 mmol) of potassium tert-butoxide were then added in one portion, and the mixture was stirred at 0° C. for 30 min. The reaction solution was allowed to warm to RT and the isopropanol was removed under reduced pressure. The residue was taken up in ethyl acetate (500 ml). Water (600 ml) was added, the mixture was extracted and, after phase separation, the aqueous phase was extracted with ethyl acetate (2×300 ml). The organic phases were dried over sodium sulphate, filtered and concentrated under reduced pressure. The crude product was used without further purification in the next step. Yield: 58.6 g (quant., purity: 90%, diastereomer ratio about 3:2).

LC-MS (Method 3A): Rₜ=1.51 min (enantiomerically pure isomer 1), Rₜ=1.53 min (enantiomerically pure isomer 2);

MS (ESIpos): m/z=250 [M+H]⁺.

### Example 183A

### 2-[(3S)-4-Benzyl-6-methylmorpholin-3-yl]ethanol [enantiomerically pure isomer]

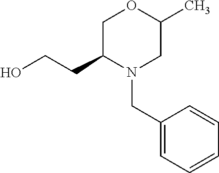 [SMILES: CC1CN(Cc2ccccc2)[C@@H](CCO)CO1]

30.0 g (108 mmol) of (5S)-4-benzyl-5-(2-hydroxyethyl)-2-methylmorpholin-3-one [diastereomer mixture, 2 isomers] were initially charged in tetrahydrofuran (1.10 l), 217 ml (433 mmol) of 2M borane/dimethyl sulphide complex solution in tetrahydrofuran were added under argon and the mixture was stirred under reflux for 2 h. The mixture was subsequently cooled to 0° C., methanol (200 ml) was added carefully and the mixture was stirred under reflux for 30 min. The mixture was subsequently concentrated completely under reduced pressure, and the residue was taken up in acetonitrile and subjected to purification and diastereomer separation by preparative RP-HPLC (acetonitrile/water, isocratic). Here, the target compound eluted as second component (enantiomerically pure isomer 2). Yield: enantiomerically pure isomer 2: 12.1 g (47% of theory); enantiomerically pure isomer 1: 6.23 g (24% of theory).

### Enantiomerically Pure Isomer 2:

LC-MS (Method 4A): Rₜ=2.33 min; MS (ESIpos): m/z=236 [M+H]⁺;

¹H-NMR (400 MHz, DMSO-d₆): δ[ppm]=7.36-7.18 (m, 5H), 4.42 (t, 1H), 3.69-3.35 (m, 7H), 2.65-2.56 (m, 1H), 2.36-2.29 (m, 1H), 2.26-2.16 (m, 1H), 1.81-1.65 (m, 2H), 1.00 (d, 3H).

### Enantiomerically Pure Isomer 1:

LC-MS (Method 4A): Rₜ=2.23 min; MS (ESIpos): m/z=236 [M+H]⁺;

¹H-NMR (400 MHz, DMSO-d₆): δ[ppm]=7.37-7.19 (m, 5H), 4.49 (t, 1H), 4.10 (d, 1H), 3.76 (dd, 1H), 3.58-3.38 (m, 3H), 3.33-3.20 (m, 1H), 2.95 (d, 1H), 2.27 (m_{c}, 1H), 1.80 (m_{c}, 1H), 1.68 (dd, 1H), 1.48 (m_{c}, 1H), 0.94 (d, 3H).

### Example 184A

### (4-Benzyl-6-methylmorpholin-3-yl)acetaldehyde [diastereomer mixture, 2 isomers]

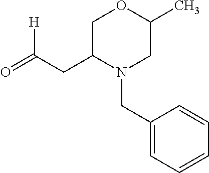 [SMILES: CC1CN(Cc2ccccc2)C(CC=O)CO1]

2.24 g (1.54 ml, 17.6 mmol) of oxalyl chloride were initially charged in dichloromethane (70.5 ml), and 2.41 g (2.19 ml, 30.8 mmol) of dimethyl sulphoxide in dichloromethane (12.5 ml) were then added slowly at −78° C. 2.50 g (10.6 mmol) of 2-[(3S)-4-benzyl-6-methylmorpholin-3-yl]ethanol [enantiomerically pure diastereomer] in dichloromethane (12.5 ml) were then added. Subsequently, 5.91 g (8.14 ml, 58.4 mmol) of triethylamine were added slowly (20 min) to the cold reaction mixture, and the reaction mixture was then warmed to RT and stirred overnight. The reaction mixture was poured into water, and after separation of the phases the organic phase was washed with water. The organic phase was dried over sodium sulphate, filtered and concentrated under reduced pressure. The crude product obtained was purified by means of flash chromatography on silica gel (cyclohexane/ethyl acetate 10:1-1:1). Yield: 2.23 g (89% of theory); proportional isomerization.

LC-MS (Method 4A): Rₜ=2.58 min (enantiomerically pure isomer 1), Rₜ=2.60 min (enantiomerically pure isomer 2)

MS (ESIpos): m/z=234 [M+H]⁺.

### Example 185A

### 1-(4-Benzyl-6-methylmorpholin-3-yl)propan-2-ol [diastereomer mixture, 2 isomers]

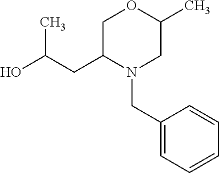 [SMILES: CC(O)CC1COC(C)CN1Cc1ccccc1]

2.20 g (9.43 mmol) of (4-benzyl-6-methylmorpholin-3-yl)acetaldehyde [diastereomer mixture, 2 isomers] were initially charged in tetrahydrofuran (40.4 ml), and 11.3 ml (11.3 mmol) of a 1M solution of methylmagnesium bromide in tetrahydrofuran were added at −78° C. The mixture was stirred at −78° C. for 15 min and then warmed to RT. Saturated aqueous ammonium chloride solution (70 ml) was then added carefully to the reaction solution, most of the tetrahydrofuran was removed under reduced pressure and the residue was taken up in dichloromethane. After phase separation, the organic phase was washed with water, dried over sodium sulphate, filtered, concentrated under reduced pressure and subjected directly to purification and diastereomer separation by preparative RP-HPLC (acetonitrile/water, isocratic). Here, the target compound eluted as third component as a diastereomer mixture (2 isomers). Yield: Target compound: 627 mg (25% of theory, 2 isomers, diastereomers 3+4); diastereomer 1: 476 mg (20% of theory); diastereomer 2: 424 mg (17% of theory).

### Diastereomers 3+4 (2 Isomers, Target Compound):

LC-MS (Method 4A): Rₜ=2.46 min; MS (ESIpos): m/z=250 [M+H]⁺;

### Diastereomer 1:

LC-MS (Method 4A): Rₜ=2.32 min; MS (ESIpos): m/z=250 [M+H]⁺;

### Diastereomer 2:

LC-MS (Method 4A): Rₜ=2.39 min; MS (ESIpos): m/z=250 [M+H]⁺.

### Example 186A

### 1-(6-Methylmorpholin-3-yl)propan-2-ol [diastereomer mixture, 2 isomers]

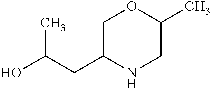

627 mg (2.51 mmol) of 1-(4-benzyl-6-methylmorpholin-3-yl)propan-2-ol [diastereomer mixture, 2 isomers, diastereomers 3+4, Example 186A] were initially charged in ethanol (25.3 ml), 63.2 mg of palladium on carbon (10%) and 31.6 mg of palladium hydroxide on carbon (20%) were added under argon, and the mixture was then stirred under an atmosphere of hydrogen at standard pressure overnight. A further 63.2 mg of palladium on carbon (10%) and 31.6 mg of palladium hydroxide on carbon (20%) were added, and the mixture was once more stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 411 mg (quant.).

MS (Method 1C): m/z=160 [M+H]⁺.

### Example 187A

### N-Benzyl-N-[(2R,3R)-3-{[tert-butyl(dimethyl)silyl]oxy}-1-hydroxybutan-2-yl]-2-chloropropanamide [diastereomer mixture, 2 isomers]

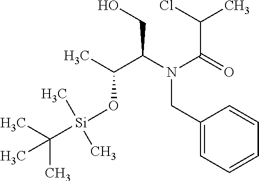 [SMILES: CC(Cl)C(=O)N(Cc1ccccc1)[C@H](CO)[C@@H](C)O[Si](C)(C)C(C)(C)C]

4.10 g (13.3 mmol) of (2R,3R)-2-(benzylamino)-3-{[tert-butyl(dimethyl)silyl]oxy}butan-1-ol [lit.: D. Tanner et al., J. Org. Chem. 1997, 62, 7364-7375] were initially charged in isopropanol (36.9 ml), the mixture was cooled to 0° C. and 2.01 g (2.77 ml, 19.9 mmol) of triethylamine were added. 2.02 g (1.54 ml, 15.9 mmol) of 2-chloropropionyl chloride [racemate] were then added dropwise and the reaction was warmed to RT and stirred for 3 h. The reaction solution was concentrated under reduced pressure, and the residue was taken up in 0.5N aqueous hydrogen chloride solution and extracted repeatedly with dichloromethane. The organic phases were dried over sodium sulphate, filtered and concentrated under reduced pressure. The crude product was used without further purification in the next step. Yield: 5.45 g (76% of theory, purity: 74%).

LC-MS (Method 1A): Rₜ=1.38 min (enantiomerically pure isomer 1), Rₜ=1.41 min (enantiomerically pure isomer 2)

MS (ESIpos): m/z=400 [M+H]⁺.

### Example 188A

### (5R)-4-Benzyl-5-[(1R)-1-{[tert-butyl(dimethyl)silyl]oxy}ethyl]-2-methylmorpholin-3-one [diastereomer mixture, 2 isomers]

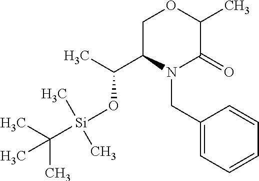

5.45 g (206 mmol, 74% pure) of N-benzyl-N-[(2R,3R)-3-{[tert-butyl(dimethyl)silyl]oxy}-1-hydroxybutan-2-yl]-2-chloropropanamide [diastereomer mixture, 2 isomers] were initially charged in isopropanol (25.9 ml), the mixture was cooled to 0° C. and 3.42 g (30.5 mmol) of potassium tert-butoxide were then added in one portion. The mixture was warmed to RT and stirred for 1 h. Most of the isopropanol was removed under reduced pressure, and 1N aqueous hydrogen chloride solution was added to the residue. The mixture was extracted with dichloromethane, and the organic phases were dried over sodium sulphate, filtered and concentrated under reduced pressure. The crude product was used without further purification in the next step. Yield: 4.88 g (99% of theory, purity: 75%, diastereomer ratio about 1:2).

LC-MS (Method 1A): Rₜ=1.46 min (enantiomerically pure isomer 1), Rₜ=1.47 min (enantiomerically pure isomer 2);

MS (ESIpos): m/z=364 [M+H]⁺.

### Example 189A

### (5R)-4-Benzyl-5-[(1R)-1-hydroxyethyl]-2-methylmorpholin-3-one [diastereomer mixture, 2 isomers]

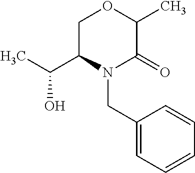

4.55 g (9.41 mmol, purity: 75%) of (5R)-4-benzyl-5-[(1R)-1-{[tert-butyl(dimethyl)-silyl]oxy}ethyl]-2-methylmorpholin-3-one [diastereomer mixture, 2 isomers] were initially charged in tetrahydrofuran (47.5 ml), and 23.5 ml (23.5 mmol) of tetra-n-butylammonium fluoride solution (1.0M in tetrahydrofuran) were added at RT. The reaction solution was stirred at RT for 2 h and the reaction solution was then concentrated under reduced pressure. The residue was taken up in ethyl acetate and washed with water and the organic phase was then dried over sodium sulphate, filtered and concentrated under reduced pressure. The crude product was then purified by flash chromatography on silica gel (dichloromethane, dichloromethane/methanol 50:1). Yield: 2.29 g (81% of theory, purity: 83%).

LC-MS (Method 1A): Rₜ=0.73 min; MS (ESIpos): m/z=250 [M+H]⁺.

### Example 190A

### (1R)-1-[(3R)-4-Benzyl-6-methylmorpholin-3-yl]ethanol [diastereomer mixture, 2 isomers]

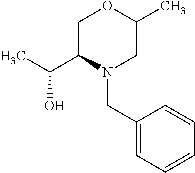

2.29 g (9.19 mmol) of (5R)-4-benzyl-5-[(1R)-1-hydroxyethyl]-2-methylmorpholin-3-one [diastereomer mixture, 2 isomers] were initially charged in tetrahydrofuran (90.4 ml), 18.4 ml (36.4 mmol) of 2M borane/dimethyl sulphide complex solution in tetrahydrofuran were added under argon and the reaction mixture was then stirred under reflux for 2 h. The mixture was subsequently cooled to 0° C., methanol (50 ml) was added carefully and the mixture was then stirred under reflux for 30 min. The mixture was then concentrated completely under reduced pressure, and the residue was taken up in acetonitrile and purified directly by preparative RP-HPLC (acetonitrile/water). The two isomers were isolated but subsequently recombined. Yield: 891 mg (41% of theory).

LC-MS (Method 5A): Rₜ=2.05 min (enantiomerically pure isomer 1), Rₜ=2.20 min (enantiomerically pure isomer 2);

MS (ESIpos): m/z=236 [M+H]⁺.

### Example 191A

### (1R)-1-[(3R)-6-Methylmorpholin-3-yl]ethanol [diastereomer mixture, 2 isomers]

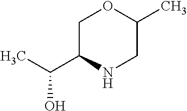

891 mg (3.79 mmol) of (1R)-1-[(3R)-4-benzyl-6-methylmorpholin-3-yl]ethanol [diastereomer mixture, 2 isomers] were initially charged in ethanol (30.4 ml), 90.0 mg of palladium on carbon (10%) and 45.0 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 599 mg (quant.).

MS (Method 5A): m/z=146 [M+H]⁺.

### Example 192A

### 4-Benzyl-3,6,6-trimethyl-5-oxomorpholine-3-carbaldehyde [racemate]

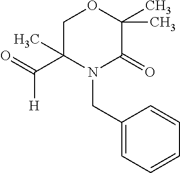 [SMILES: CC1(C)OCC(C)(C=O)N(Cc2ccccc2)C1=O]

17.6 g (12.1 ml, 139 mmol) of oxalyl chloride were initially charged in dichloromethane (324 ml), and 10.5 g (9.54 ml, 134 mmol) of dimethyl sulphoxide in dichloromethane (58 ml) were then added slowly at −78° C. 12.2 g (46.3 mmol) of 4-benzyl-5-(hydroxymethyl)-2,2,5-trimethylmorpholin-3-one [racemate] in dichloromethane (58 ml) were then added and the mixture was then stirred for 30 min. Subsequently, 28.1 g (38.7 ml, 278 mmol) of triethylamine were added slowly (20 min) to the cold reaction mixture, and the reaction mixture was then warmed to RT and stirred overnight. The reaction mixture was poured into water, and after separation of the phases the organic phase was washed with water. The organic phase was dried over sodium sulphate, filtered and concentrated under reduced pressure. The crude product obtained was purified by means of flash chromatography on silica gel (cyclohexane/ethyl acetate 10:1-2:1). Yield: 9.00 g (57% of theory, purity: 76%).

LC-MS (Method 1A): Rₜ=0.88 min; MS (ESIpos): m/z=262 [M+H]⁺.

### Example 193A

### 4-Benzyl-5-(1-hydroxyethyl)-2,2,5-trimethylmorpholin-3-one [diastereomer mixture, 4 isomers]

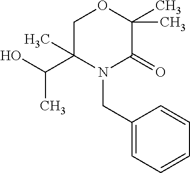 [SMILES: CC(O)C1(C)COC(C)(C)C(=O)N1Cc1ccccc1]

9.00 g (26.4 mmol, purity: 76%) of 4-benzyl-3,6,6-trimethyl-5-oxomorpholine-3-carbaldehyde [racemate] were initially charged in tetrahydrofuran (107 ml), and 39.6 ml (39.6 mmol) of a 1M solution of methylmagnesium bromide in tetrahydrofuran were added at −78° C. The mixture was stirred at −78° C. for 15 min and then warmed to RT. Saturated aqueous ammonium chloride solution (70 ml) was then added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The crude product was used without further purification in the next step. Yield: 9.00 g (94% of theory, purity: 74%).

LC-MS (Method 1A): Rₜ=0.83 min; MS (ESIpos): m/z=278 [M+H]⁺.

### Example 194A

### 1-(4-Benzyl-3,6,6-trimethylmorpholin-3-yl)ethanol [diastereomer, 2 isomers]

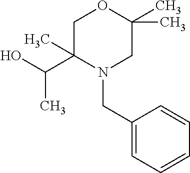 [SMILES: CC(O)C1(C)COC(C)(C)CN1Cc1ccccc1]

9.33 g (33.6 mmol) of 4-benzyl-5-(1-hydroxyethyl)-2,2,5-trimethylmorpholin-3-one [diastereomer mixture, 4 isomers] were initially charged in tetrahydrofuran (331 ml), 67.3 ml (135 mmol) of 2M borane/dimethyl sulphide complex solution in tetrahydrofuran were added under argon and the reaction mixture was then stirred under reflux for 2 h. The mixture was subsequently cooled to 0° C., methanol (78 ml) was added carefully and the mixture was then stirred under reflux for 30 min. The mixture was subsequently concentrated completely under reduced pressure, and the residue was taken up in acetonitrile and subjected directly to purification and diastereomer separation by preparative RP-HPLC (acetonitrile/water, isocratic). Here, the target compound eluted as second component. Yield: Target compound: 832 mg (9% of theory; diastereomer 2, 2 isomers); minor isomer: 3.30 g (37% of theory, diastereomer 1, 2 isomers).

### Diastereomer 2, 2 Isomers (Target Compound):

LC-MS (Method 1A): Rₜ=0.58 min; MS (ESIpos): m/z=264 [M+H]⁺;

¹H-NMR (400 MHz, DMSO-d₆): δ[ppm]=7.39-7.15 (m, 5H), 4.49 (d, 1H), 4.17 (d, 1H), 3.79-3.65 (m, 2H), 3.18-3.07 (m, 2H), 2.23-2.13 (m, 2H), 1.17-1.13 (m, 6H), 1.02 (d, 6H).

### Diastereomer 1 (2 Isomers):

LC-MS (Method 1A): Rₜ=0.63 min; MS (ESIpos): m/z=264 [M+H]⁺;

¹H-NMR (400 MHz, DMSO-d₆): δ [ppm]=7.36-7.15 (m, 5H), 4.58 (d, 1H), 4.07 (quin, 1H), 3.80 (d, 1H), 3.63 (d, 1H), 3.32-3.24 (m, 2H), 2.20 (m, 1H), 2.12 (d, 1H), 1.19 (d, 3H), 1.14 (s, 3H), 1.08 (s, 3H), 1.02 (s, 3H).

### Example 195A

### 1-(3,6,6-Trimethylmorpholin-3-yl)ethanol [diastereomer 2, 2 isomers]

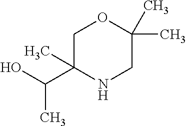

832 mg (3.16 mmol) of 1-(4-benzyl-3,6,6-trimethylmorpholin-3-yl)ethanol [racemate] were initially charged in ethanol (31.8 ml), 91.0 mg of palladium on carbon (10%) and 45.0 mg of palladium hydroxide on carbon (20%) were added under argon and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 549 mg (100% of theory).

MS (Method 6A): m/z=174 [M+H]⁺.

### Example 196A

### 2-Chloro-N-(1,4-dihydroxybutan-2-yl)propanamide [diastereomer mixture, 4 isomers]

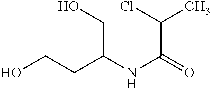

5.50 g (52.3 mmol) of 2-aminobutane-1,4-diol [racemate][lit.: A. S. Jogalekar et al., WO 2008151304, 2008] were initially charged in isopropanol (184 ml), and 5.29 g (7.29 ml, 52.3 mmol) of triethylamine were added. 7.31 g (5.59 ml, 57.5 mmol) of 2-chloropropionyl chloride [racemate] were then added dropwise, and the mixture was stirred at RT overnight. The reaction solution was concentrated under reduced pressure and the crude product obtained was used directly in the next step.

MS (Method 1B): m/z=195 [M+H]⁺.

### Example 197A

### 5-(2-Hydroxyethyl)-2-methylmorpholin-3-one [diastereomer mixture, 4 isomers]

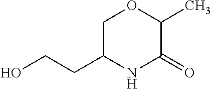

10.2 g (about 52.3 mmol, crude product) of 2-chloro-N-(1,4-dihydroxybutan-2-yl)propanamide [diastereomer mixture, 4 isomers] were initially charged in isopropanol (221 ml), the mixture was cooled to 0° C. and 29.3 g (261 mmol) of potassium tert-butoxide were then added in one portion. The mixture was warmed to RT and stirred for 60 h. The reaction solution was concentrated under reduced pressure and the crude product obtained was used directly in the next step.

LC-MS (Method 3A): Rₜ=0.34 min; MS (ESIpos): m/z=160 [M+H]⁺.

### Example 198A

### 5-(2-{[tert-Butyl(diphenyl)silyl]oxy}ethyl)-2-methylmorpholin-3-one [diastereomer, 2 isomers]

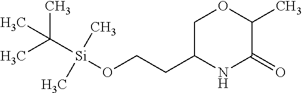 [SMILES: CC1OCC(CCO[Si](C)(C)C(C)(C)C)NC1=O]

3.93 g (about 24.7 mmol, crude product) of 5-(2-hydroxyethyl)-2-methylmorpholin-3-one [diastereomer mixture, 4 isomers] were initially charged in N,N-dimethylformamide (50 ml), and then 5.05 g (74.1 mmol) of imidazole and 10.2 g (37.1 mmol) of tert-butyldiphenylsilyl chloride were added at 0° C. The mixture was stirred for 24 h and warmed to RT during this time. Saturated aqueous ammonium chloride solution was then added and the reaction solution was extracted with ethyl acetate. The combined organic phases were washed with saturated aqueous sodium chloride solution, dried over sodium sulphate, filtered and concentrated under reduced pressure. The crude product obtained was purified by preparative RP-HPLC (acetonitrile/water), giving only the main diastereomer. Yield: 2.05 g (20% of theory).

LC-MS (Method 1A): Rₜ=1.31 min; MS (ESIpos): m/z=398 [M+H]⁺.

### Example 199A

### 5-(2-{[tert-Butyl(diphenyl)silyl]oxy}ethyl)-2-methylmorpholine [diastereomer, 2 isomers]

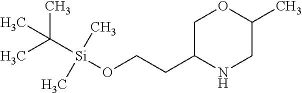 [SMILES: CC1CNC(CCO[Si](C)(C)C(C)(C)C)CO1]

1.20 g (3.02 mmol) of 5-(2-{[tert-butyl(diphenyl)silyl]oxy}ethyl)-2-methylmorpholin-3-one [diastereomer, 2 isomers] were initially charged in tetrahydrofuran (70.6 ml), 7.55 ml (15.1 mmol) of 2M borane/dimethyl sulphide complex solution in tetrahydrofuran were added and the reaction mixture was then stirred at RT for 60 h. The reaction was then concentrated completely under reduced pressure and the residue was taken up in ethanol and stirred under reflux overnight. The reaction mixture was then concentrated completely under reduced pressure and the crude product obtained was used directly in the next step. Yield: 1.22 g (quant.).

LC-MS (Method 1A): Rₜ=1.08 min; MS (ESIpos): m/z=383 [M+H]⁺.

### Example 200A

### [5-(2-{[tert-Butyl(diphenyl)silyl]oxy}ethyl)-2-methylmorpholin-4-yl](2-{[(4-chloropyridin-2-yl)methyl]amino}-7-methoxy-1,3-benzoxazol-5-yl)methanone [diastereomer, 2 isomers]

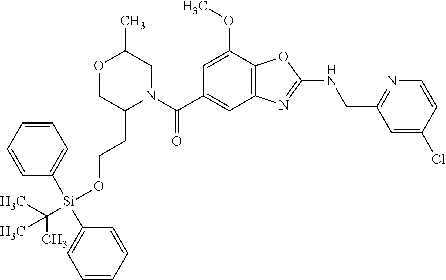 [SMILES: COc1cc(C(=O)N2CC(C)OCC2CCO[Si](c2ccccc2)(c2ccccc2)C(C)(C)C)cc2nc(NCc3cc(Cl)ccn3)oc12]

600 mg (1.80 mmol) of 2-{[(4-chloropyridin-2-yl)methyl]amino}-7-methoxy-1,3-benzoxazole-5-carboxylic acid and 759 mg (1.98 mmol) of 5-(2-{[tert-butyl(diphenyl)silyl]oxy}ethyl)-2-methylmorpholine [racemate] were initially charged in N,N-dimethylformamide (4.90 ml), and 744 mg (1.00 ml, 5.75 mmol) of N,N-diisopropylethylamine were added. 820 mg (2.16 mmol) of HATU were then added at RT, and the mixture was stirred for 1 h. Without further work-up, the reaction solution was then purified directly by preparative RP-HPLC (acetonitrile/water+0.1% formic acid). Yield: 845 mg (67% of theory).

LC-MS (Method 3A): Rₜ=2.94 min; MS (ESIpos): m/z=699 [M+H]⁺.

### Example 201A

### tert-Butyl 2-{[(1-methoxy-1-oxobutan-2-yl)amino]methyl}azetidine-1-carboxylate [diastereomer mixture, 4 isomers]

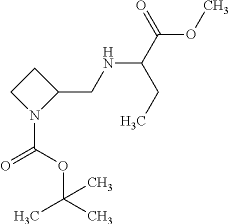 [SMILES: CCC(NCC1CCN1C(=O)OC(C)(C)C)C(=O)OC]

1.80 g (9.66 mmol) of tert-butyl 2-(aminomethyl)azetidine-1-carboxylate [racemate] were dissolved in 25 ml of dichloromethane, 1.62 ml (1.17 g, 11.6 mmol) of triethylamine and 1.11 ml (1.75 g, 9.66 mmol) of methyl 2-bromobutanoate [racemate] were added and the mixture was stirred under reflux overnight. 1.35 ml (0.98 g, 9.66 mmol) of triethylamine and 0.89 ml (1.40 g, 7.73 mmol) of methyl 2-bromobutanoate [racemate] were then added, and the mixture was stirred under reflux overnight. After cooling to room temperature, water was added and the phases were separated. The aqueous phase was extracted twice with dichloromethane and the combined organic phases were washed with saturated aqueous sodium chloride solution, dried over sodium sulphate, filtered and then freed of the solvent under reduced pressure. This gave 2.64 g (83% of theory, purity: 87%) of the desired product.

LC-MS (Method 6A): Rₜ=2.16 min (diastereomer 1, 2 isomers), Rₜ=2.22 min (diastereomer 2, 2 isomers);

MS (ESIpos): m/z=287 [M+H]⁺

### Example 202A

### tert-Butyl 2-({[(benzyloxy)carbonyl](1-methoxy-1-oxobutan-2-yl)amino}methyl)azetidine-1-carboxylate [diastereomer mixture, 4 isomers]

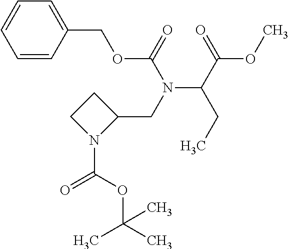 [SMILES: CCC(C(=O)OC)N(CC1CCN1C(=O)OC(C)(C)C)C(=O)OCc1ccccc1]

At 0° C., a solution of 1.88 ml (2.25 g, 13.2 mmol) of benzyl chloroformate in 7 ml of toluene was slowly added dropwise to 3.75 g (8.77 mmol) of tert-butyl 2-{[(1-methoxy-1-oxobutan-2-yl)amino]methyl}azetidine-1-carboxylate [diastereomer mixture, 4 isomers] in 100 ml of THF. A solution of 2.20 ml (1.59 g, 15.8 mmol) of triethylamine in 10 ml of THF was then slowly added dropwise and the mixture was stirred at room temperature overnight. The mixture was concentrated under reduced pressure, and water and ethyl acetate were added to the residue. After phase separation, the aqueous phase was extracted twice with ethyl acetate, and the combined organic phases were washed with saturated aqueous sodium chloride solution. The mixture was dried over sodium sulphate and filtered, the filtrate was concentrated under reduced pressure and the residue was dried under high vacuum. Cyclohexane and ethyl acetate were then added to the residue and the product was purified by silica gel chromatography (cyclohexane/ethyl acetate 10:3). This gave 2.31 g (38% of theory, purity: 62%) of the desired product.

### Example 203A

### Methyl 2-{(azetidin-2-ylmethyl)[(benzyloxy)carbonyl]amino}butanoate trifluoroacetate [diastereomer mixture, 4 isomers]

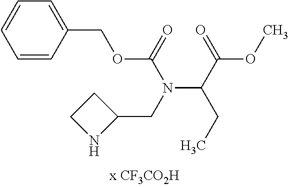 [SMILES: CCC(C(=O)OC)N(CC1CCN1)C(=O)OCc1ccccc1]

2.62 ml (3.88 g, 34.1 mmol) of trifluoroacetic acid were added to 2.31 g (3.41 mmol, purity: 62%) of tert-butyl 2-({[(benzyloxy)carbonyl](1-methoxy-1-oxobutan-2-yl)amino}methyl)azetidine-1-carboxylate [diastereomer mixture, 4 isomers] in 45 ml of dichloromethane, and the mixture was stirred at room temperature overnight. The mixture was then concentrated under reduced pressure, dichloromethane and water were added to the residue and the phases were separated. The aqueous phase was extracted twice with dichloromethane and the combined organic phases were washed with saturated aqueous sodium chloride solution. After drying over sodium sulphate, the mixture was concentrated under reduced pressure and the residue was dried under high vacuum. This gave 1.41 g (28% of theory, purity: 29%) of the desired product.

LC-MS (Method 1A): Rₜ=0.72 min; MS (ESIpos): m/z=320 [M+H-TFA]⁺

### Example 204A

### Benzyl 3-ethyl-2-oxo-1,4-diazabicyclo[4.2.0]octane-4-carboxylate [diastereomer mixture, 4 isomers]

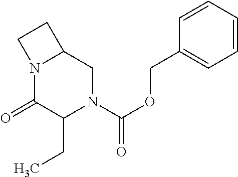 [SMILES: CCC1C(=O)N2CCC2CN1C(=O)OCc1ccccc1]

0.65 g (4.71 mmol) of potassium carbonate was added to 1.41 g (0.94 mmol, purity: 29%) of methyl 2-{(azetidin-2-ylmethyl)[(benzyloxy)carbonyl]amino}butanoate trifluoroacetate [diastereomer mixture, 4 isomers] in 30 ml of methanol, and the mixture was stirred at room temperature overnight. The mixture was then concentrated under reduced pressure, water and ethyl acetate were added to the residue and the phases were separated. The aqueous phase was extracted twice with ethyl acetate and the combined organic phases were washed with saturated aqueous sodium chloride solution. After drying over sodium sulphate, the mixture was concentrated under reduced pressure and the residue was dried under high vacuum. The residue was dissolved in methanol and water and purified by preparative HPLC (RP18 column, mobile phase: acetonitrile/water gradient). This gave 383 mg (quant.) of the desired product.

LC-MS (Method 1A): Rₜ=0.89 min; MS (ESIpos): m/z=289 [M+H]⁺

### Example 205A

### 3-Ethyl-1,4-diazabicyclo[4.2.0]octan-2-one [diastereomer mixture, 4 isomers]

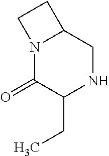

Under argon, 424 mg (0.39 mmol) of 10% palladium on activated carbon were added to 383 mg (0.78 mmol, purity: 60%) of benzyl 3-ethyl-2-oxo-1,4-diazabicyclo[4.2.0]-octane-4-carboxylate [diastereomer mixture, 4 isomers, Example 204A] in 30 ml of methanol, and the mixture was hydrogenated at RT and standard pressure for 4 h. The mixture was then filtered, the filtrate was concentrated under reduced pressure and the residue was dried under high vacuum. This gave 74.8 mg (61% of theory) of the desired product.

MS (Method 1C): m/z=155 [M+H]⁺

### Example 206A

### tert-Butyl 2-{[(1,3-dimethoxy-1-oxopropan-2-yl)amino]methyl}azetidine-1-carboxylate [diastereomer mixture, 4 isomers]

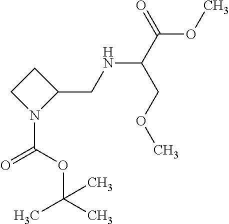 [SMILES: COCC(NCC1CCN1C(=O)OC(C)(C)C)C(=O)OC]

3.00 g (16.1 mmol) of tert-butyl 2-(aminomethyl)azetidine-1-carboxylate [racemate] were dissolved in 40 ml of dichloromethane, 2.69 ml (1.96 g, 19.3 mmol) of triethylamine and 3.17 g (16.1 mmol) of methyl 2-bromo-3-methoxypropanoate [racemate] were added and the mixture was stirred at room temperature overnight. 1.12 ml (0.82 g, 8.05 mmol) of triethylamine and 0.90 g (4.59 mmol) of methyl 2-bromo-3-methoxypropanoate [racemate] were added and the mixture was stirred under reflux overnight. 2.69 ml (1.96 g, 19.3 mmol) of triethylamine and 3.17 g (16.1 mmol) of methyl 2-bromo-3-methoxypropanoate [racemate] were then added and the mixture was stirred under reflux overnight. After cooling, the precipitate was filtered off, water was added to the filtrate and the phases were separated. The aqueous phase was extracted twice with dichloromethane and the combined organic phases were washed with saturated aqueous sodium chloride solution. After drying over sodium sulphate, the mixture was freed of the solvent under reduced pressure and the residue was dried under high vacuum. This gave 6.89 g (94% of theory, purity: 67%) of the desired product.

LC-MS (Method 6A): 1.91 min (diastereomer 1, 2 isomers), Rₜ=1.96 min (diastereomer 2, 2 isomers);

MS (ESIpos): m/z=303 [M+H]⁺

### Example 207A

### Methyl N-(azetidin-2-ylmethyl)-N-[(benzyloxy)carbonyl]-O-methylserinate trifluoroacetate [diastereomer mixture, 4 isomers]

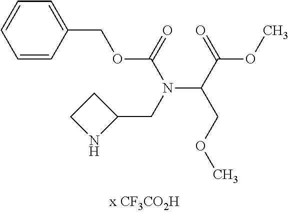 [SMILES: COCC(C(=O)OC)N(CC1CCN1)C(=O)OCc1ccccc1]

17.6 ml (25.9 g, 227 mmol) of trifluoroacetic acid were added to 6.89 g (15.26 mmol, purity: 67%) of tert-butyl 2-{[(1,3-dimethoxy-1-oxopropan-2-yl)amino]methyl}azetidine-1-carboxylate [diastereomer mixture, 4 isomers] in 150 ml of dichloromethane, and the mixture was stirred at room temperature overnight. After the addition of 8.8 ml (12.9 g, 113 mmol) of trifluoroacetic acid, the mixture was stirred overnight at room temperature and then concentrated under reduced pressure and the residue was dissolved in dichloromethane. The solution was concentrated under reduced pressure and the residue obtained was redissolved in dichloromethane and then freed of the solvent under reduced pressure. After drying under high vacuum, the crude product obtained was, without purification, used further in Example 208A.

### Example 208A

### 3-(Methoxymethyl)-1,4-diazabicyclo[4.2.0]octan-2-one [diastereomer mixture, 4 isomers]

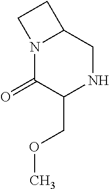

11.4 g (82.7 mmol) of potassium carbonate were added to 10.9 g (20.6 mmol, purity: 60%) of methyl N-(azetidin-2-ylmethyl)-N-[(benzyloxy)carbonyl]-O-methylserinate trifluoroacetate [diastereomer mixture, 4 isomers] in 150 ml of methanol, and the mixture was stirred at room temperature overnight. The mixture was then concentrated under reduced pressure and the residue was dried under high vacuum. This gave 22.5 g of crude product which was used in Example 209A without further purification.

MS (Method 1C): m/z=171 [M+H]⁺

### Example 209A

### Benzyl 3-(methoxymethyl)-2-oxo-1,4-diazabicyclo[4.2.0]octane-4-carboxylate [diastereomer mixture, 4 isomers]

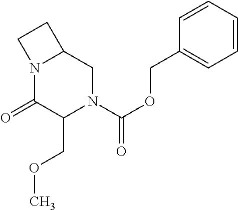 [SMILES: COCC1C(=O)N2CCC2CN1C(=O)OCc1ccccc1]

At 0° C., a solution of 2.82 ml (3.38 g, 19.8 mmol) of benzyl chloroformate in 6.5 ml of toluene was added dropwise to 22.48 g (19.8 mmol, purity: 19%) of 3-(methoxymethyl)-1,4-diazabicyclo[4.2.0]octan-2-one [diastereomer mixture, 4 isomers] in 200 ml of THF. A solution of 3.13 ml (2.41 g, 23.8 mmol) of triethylamine in 10 ml of THF was then slowly added dropwise and the mixture was stirred at room temperature overnight. The mixture was then cooled to 0° C. and first a solution of 1.69 ml (2.03 g, 11.9 mmol) of benzyl chloroformate in 4 ml of toluene and then, slowly, a solution of 1.93 ml (1.40 g, 13.9 mmol) of triethylamine in 10 ml of THF were added dropwise and the mixture was stirred at room temperature overnight. After filtration, the filtrate was concentrated under reduced pressure, and water and ethyl acetate were then added. After phase separation, the aqueous phase was extracted twice with ethyl acetate, and the combined organic phases were washed with saturated aqueous sodium chloride solution. The mixture was dried over sodium sulphate and filtered, the filtrate was concentrated under reduced pressure and the residue was dried under high vacuum. The residue was dissolved in acetonitrile and water and purified by preparative HPLC (RP18 column, mobile phase: acetonitrile/water gradient). This gave 1.08 g (18% of theory) of the desired product.

LC-MS (Method 1A): Rₜ=0.83 min; MS (ESIpos): m/z=305 [M+H]⁺

### Example 210A

### Benzyl 3-(methoxymethyl)-2-oxo-1,4-diazabicyclo[4.2.0]octane-4-carboxylate [enantiomerically pure isomer 3]

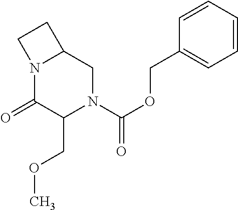 [SMILES: COCC1C(=O)N2CCC2CN1C(=O)OCc1ccccc1]

1.08 g of benzyl 3-(methoxymethyl)-2-oxo-1,4-diazabicyclo[4.2.0]octane-4-carboxylate [diastereomer mixture](Example 209A) were separated into the enantiomers on a chiral phase [Method 49D].

Yield: enantiomerically pure isomer 3: 266.5 mg (99.8% ee)

enantiomerically pure isomer 3: Rₜ=9.74 min [Method 42E].

LC-MS (Method 1A): Rₜ=0.79 min; MS (ESIpos): m/z=305 [M+H]⁺

### Example 211A

### 3-(Methoxymethyl)-1,4-diazabicyclo[4.2.0]octan-2-one [enantiomerically pure isomer 3]

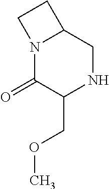

Under argon, 465 mg (0.44 mmol) of 10% palladium on activated carbon were added to 266.5 mg of benzyl 3-(methoxymethyl)-2-oxo-1,4-diazabicyclo[4.2.0]octane-4-carboxylate [enantiomerically pure isomer 3, Example 210A] in 25 ml of methanol, and the mixture was hydrogenated at RT and standard pressure overnight. The mixture was then filtered, the filtrate was concentrated under reduced pressure and the residue was dried under high vacuum. This gave 138 mg (92% of theory) of the desired product.

MS (Method 1C): m/z=171 [M+H]⁺

### Example 212A

### tert-Butyl 2-[(benzylamino)methyl]azetidine-1-carboxylate [racemate]

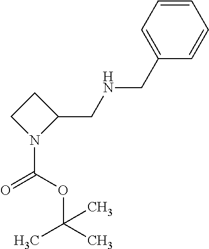 [SMILES: CC(C)(C)OC(=O)N1CCC1CNCc1ccccc1]

10.0 g (53.7 mmol) of tert-butyl 2-(aminomethyl)azetidine-1-carboxylate and 2.03 g (37.8 mmol) of benzaldehyde in 100 ml of methanol were heated under reflux for 2.5 h. The mixture was then cooled to 0° C., and sodium borohydride was added slowly at this temperature over a period of 15 min. The mixture was stirred at RT overnight. The mixture was then concentrated under reduced pressure, dichloromethane and water were added to the residue, the phases were separated and the aqueous phase was extracted twice with dichloromethane. The combined organic phases were washed with saturated aqueous sodium chloride solution, dried over sodium sulphate and filtered, and the filtrate was concentrated under reduced pressure. Dichloromethane was added to the residue obtained, and the product was purified by silica gel chromatography (dichloromethane, then dichloromethane/methanol=100:4). Yield: 7.43 g (50% of theory).

LC-MS (Method 6A): Rₜ=2.41 min; MS (ESIpos): m/z=277 [M+H]⁺.

### Example 213A

### tert-Butyl 2-{[benzyl(1-methoxy-1-oxopropan-2-yl)amino]methyl}azetidine-1-carboxylate [diastereomer mixture, 4 isomers]

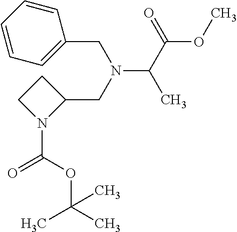 [SMILES: COC(=O)C(C)N(Cc1ccccc1)CC1CCN1C(=O)OC(C)(C)C]

2.50 g (9.05 mmol) of tert-butyl 2-[(benzylamino)methyl]azetidine-1-carboxylate [racemate] were dissolved in dichloromethane (150 ml), 5.55 ml (4.03 g, 39.8 mmol) of triethylamine and 3.04 ml (4.53 g, 27.1 mmol) of methyl 2-bromopropanoate [racemate] were added and the mixture was stirred at RT overnight. 5.55 ml (4.03 g, 39.8 mmol) of triethylamine and 3.04 ml (4.53 g, 27.1 mmol) of methyl 2-bromopropanoate [racemate] were then added, and the mixture was stirred at 40° C. overnight. A further 5.55 ml (4.03 g, 39.8 mmol) of triethylamine and 3.04 ml (4.53 g, 27.1 mmol) of methyl 2-bromopropanoate [racemate] were then added, and the mixture was stirred at 40° C. overnight. After cooling to room temperature, the mixture was diluted with water and dichloromethane, and the phases were separated. The aqueous phase was extracted twice with dichloromethane and the combined organic phases were washed with saturated aqueous sodium chloride solution, dried over sodium sulphate, filtered and then freed of the solvent under reduced pressure. The crude product obtained was purified by silica gel chromatography (dichloromethane, then dichloromethane/methanol=100:1). Yield: 3.22 g (94% of theory).

LC-MS (Method 1A): Rₜ=1.00 min (diastereomer 1), Rₜ=1.13 min (diastereomer 2);

MS (ESIpos): m/z=363 [M+H]⁺;

¹H-NMR (400 MHz, DMSO-d₆): δ[ppm]=7.35-7.28 (m, 4H), 7.27-7.20 (m, 1H), 4.18-3.98 (m, 1H), 3.85-3.73 (m, 1H), 3.71-3.51 (m, 6H), 3.51-3.38 (m, 1H), 3.04-2.88 (m, 1H), 2.85-2.69 (m, 1H), 2.15-1.96 (m, 1H), 1.93-1.65 (m, 1H), 1.34 (d, 9H), 1.26-1.15 (m, 3H).

### Example 214A

### Methyl N-(azetidin-2-ylmethyl)-N-benzylalaninate hydrochloride [diastereomer mixture, 4 isomers]

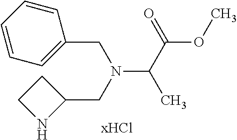 [SMILES: COC(=O)C(C)N(Cc1ccccc1)CC1CCN1]

14.9 ml (59.7 mmol) of a 4N solution of hydrogen chloride in 1,4-dioxane were added to 3.2 g (8.5 mmol) of tert-butyl 2-{[benzyl(1-methoxy-1-oxopropan-2-yl)amino]methyl}azetidine-1-carboxylate [diastereomer mixture, 4 isomers] in dioxane (74 ml), and the mixture was stirred at room temperature overnight. A further 14 ml (59.7 mmol) of the 4N solution of hydrogen chloride in 1,4-dioxane were then added, and the mixture was stirred at RT overnight. The mixture was then concentrated under reduced pressure and the product was dried under high vacuum. Yield: 3.13 g (98% of theory, purity: 80%).

LC-MS (Method 1A): Rₜ=0.68 min (diastereomer 1, 2 isomers), Rₜ=0.70 min (diastereomer 2, 2 isomers);

MS (ESIpos): m/z=263 [M+H-HCl]⁺.

### Example 215A

### 4-Benzyl-3-methyl-1,4-diazabicyclo[4.2.0]octan-2-one [enantiomerically pure isomer 3]

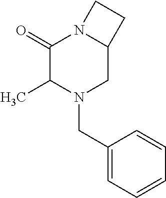 [SMILES: CC1C(=O)N2CCC2CN1Cc1ccccc1]

21.8 g (51.0 mmol, purity: 70%) of methyl N-(azetidin-2-ylmethyl)-N-benzylalaninate [diastereomer mixture, 4 isomers] were initially charged in methanol (562 ml), 28.2 g (204 mmol) of potassium carbonate were added and the mixture was then stirred at RT for 2.5 d. The reaction solution was filtered and most of the solvent was removed at 20° C. under reduced pressure. The residue was taken up in water and extracted repeatedly with dichloromethane and chloroform/isopropanol (7:3). The collected organic phases were dried over sodium sulphate, filtered and concentrated under reduced pressure. Using Method 7D, the crude product (12.1 g) was separated into the corresponding isomers. Here, the target compound eluted as third component.

Yield: 2.47 g (21% of theory).

HPLC (Method 6E): Rₜ=7.49 min, 99.0% ee;

LC-MS (Method 1A): Rₜ=0.50 min; MS (ESIpos): m/z=231 [M+H]⁺.

### Example 216A

### 3-Methyl-1,4-diazabicyclo[4.2.0]octan-2-one [enantiomerically pure isomer 3]

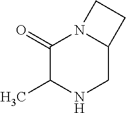

2.40 g (10.4 mmol) of 4-benzyl-3-methyl-1,4-diazabicyclo[4.2.0]octan-2-one [enantiomerically pure isomer 3] were initially charged in ethanol (85 ml), 250 mg of palladium on carbon (10%) and 130 mg of palladium hydroxide on carbon (20%) were added under argon and the mixture was then stirred under an atmosphere of hydrogen at standard pressure overnight. The reaction solution was filtered through kieselguhr and the filter residue was washed with hot ethanol (100 ml). The filtrate was concentrated under reduced pressure and the product was dried under high vacuum. Yield: 1.56 g (quant.).

GC-MS (Method 2B): Rₜ=4.50 min; MS (EIpos): m/z=140 [M]⁺;

MS (Method 1C): m/z=141 [M+H]⁺;

¹H-NMR (400 MHz, DMSO-d₆): δ[ppm]=4.59 (m_{c}, 1H), 4.09-3.89 (m, 2H), 3.27 (q, 1H), 2.95 (dd, 1H), 2.58-2.53 (m, 2H), 2.33-2.04 (m, 2H), 1.12 (d, 3H).

### Example 217A

### tert-Butyl 4-benzyl-5-oxo-4,7-diazaspiro[2.5]octane-7-carboxylate

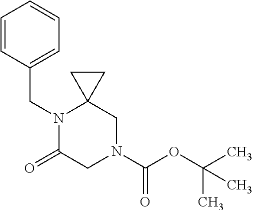 [SMILES: CC(C)(C)OC(=O)N1CC(=O)N(Cc2ccccc2)C2(CC2)C1]

Under argon and at 0° C., 2.47 g (61.9 mmol) of sodium hydride were added a little at a time to 2.50 g (8.84 mmol) of tert-butyl 5-oxo-4,7-diazaspiro[2.5]octane-7-carboxylate in 80 ml of THF, and the mixture was stirred at 0° C. for 30 min. 1.26 ml (1.81 g, 10.6 mmol) of benzyl bromide were then added dropwise, and the mixture was stirred at room temperature overnight. The mixture was then cooled to 0° C., 1.24 g (30.9 mmol) of sodium hydride were added and the mixture was stirred at 0° C. for 30 min. 0.63 ml (0.91 g, 5.3 mmol) of benzyl bromide was added dropwise, and the mixture was stirred at room temperature overnight. At 0° C., first ethanol and then water and ethyl acetate were subsequently added. After phase separation, the aqueous phase was extracted twice with ethyl acetate and the combined organic phases were dried over sodium sulphate After filtration, the filtrate was concentrated under reduced pressure and the residue was dried under high vacuum and purified by silica gel chromatography (cyclohexane/ethyl acetate 10:1) and then by preparative HPLC (RP18 column, mobile phase: acetonitrile/water gradient). This gave 1.98 g (71% of theory) of the desired product.

LC-MS (Method 1A): Rₜ=1.09 min; MS (ESIpos): m/z=317 [M+H]⁺

¹H-NMR (400 MHz, DMSO-d₆): δ[ppm]=7.38-7.14 (m, 5H), 4.41 (s, 2H), 4.16 (br. s., 2H), 1.40 (br. s., 9H), 0.98-0.89 (m, 2H), 0.79-0.72 (m, 2H).

### Example 218A

### tert-Butyl 4-benzyl-6-methyl-5-oxo-4,7-diazaspiro[2.5]octane-7-carboxylate [racemate]

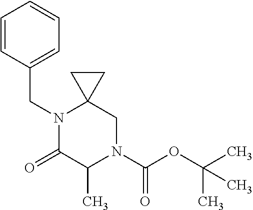 [SMILES: CC1C(=O)N(Cc2ccccc2)C2(CC2)CN1C(=O)OC(C)(C)C]

At −78° C. and under argon, 11.38 ml (11.38 mmol) of a 1M solution of lithium hexamethyldisilazide in THF were added dropwise to 1.20 g (3.79 mmol) of tert-butyl 4-benzyl-5-oxo-4,7-diazaspiro[2.5]octane-7-carboxylate in 48 ml of THF, and the mixture was stirred at −78° C. for 30 min. 0.47 ml (7.59 mmol) of methyl iodide was then added dropwise, and the mixture was stirred for 1.5 h. At 0° C., first saturated aqueous ammonium chloride solution and then ethyl acetate were subsequently added. After phase separation, the aqueous phase was extracted twice with ethyl acetate, and the combined organic phases were washed with saturated aqueous sodium chloride solution and then dried over sodium sulphate. After filtration, the filtrate was concentrated under reduced pressure and the residue was dried under high vacuum, dissolved in acetonitrile and water and purified by preparative HPLC (RP18 column, mobile phase: acetonitrile/water gradient). This gave 0.54 g (41% of theory) of the desired product.

### Example 219A

### tert-Butyl 6-methyl-5-oxo-4,7-diazaspiro[2.5]octane-7-carboxylate [racemate]

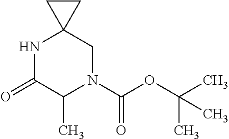 [SMILES: CC1C(=O)NC2(CC2)CN1C(=O)OC(C)(C)C]

At −78° C., 107 mg (15.5 mmol) of lithium were added to 10 ml (7.70 g, 452 mmol) of ammonia, and the mixture was stirred for a few minutes. 540 mg (1.55 mmol) of tert-butyl 4-benzyl-6-methyl-5-oxo-4,7-diazaspiro[2.5]octane-7-carboxylate [racemate] in 5 ml of THF were then added dropwise, and the mixture was slowly warmed to room temperature and then stirred at room temperature overnight. At 0° C., first saturated aqueous ammonium chloride solution and then ethyl acetate were subsequently added. After phase separation, the aqueous phase was extracted twice with ethyl acetate, and the combined organic phases were washed with saturated aqueous sodium chloride solution and dried over sodium sulphate. After filtration, the filtrate was concentrated under reduced pressure and the residue was dried under high vacuum. This gave 353 mg of the crude product which was used without further purification.

MS (Method 1C): m/z=241 [M+H]⁺.

### Example 220A

### 6-Methyl-4,7-diazaspiro[2.5]octan-5-one trifluoroacetate [racemate]

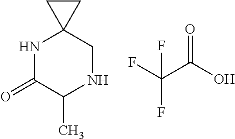

1.06 ml (1.57 g, 13.8 mmol) of trifluoroacetic acid were added to 331 mg (1.38 mmol) of tert-butyl 6-methyl-5-oxo-4,7-diazaspiro[2.5]octane-7-carboxylate [racemate] in 10 ml of dichloromethane, and the mixture was stirred at room temperature for 2 h. The mixture was then concentrated under reduced pressure and the residue was dissolved in dichloromethane. The solution was concentrated under reduced pressure and the residue obtained was re-dissolved in dichloromethane, freed of the solvent under reduced pressure and dried under high vacuum. The crude product obtained (605 mg) was used further without purification.

MS (Method 1C): m/z=141 [M+H]⁺.

### Example 221A

### tert-Butyl 4-benzyl-6-ethyl-5-oxo-4,7-diazaspiro[2.5]octane-7-carboxylate [racemate]

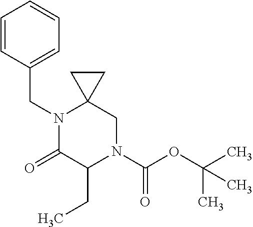 [SMILES: CCC1C(=O)N(Cc2ccccc2)C2(CC2)CN1C(=O)OC(C)(C)C]

At −78° C. and under argon, 5.69 ml (5.69 mmol) of a 1M solution of lithium hexamethyldisilazide in THF were added dropwise to 600 mg (1.89 mmol) of tert-butyl 4-benzyl-5-oxo-4,7-diazaspiro[2.5]octane-7-carboxylate in 24 ml of THF, and the mixture was stirred at −78° C. for 30 min. 491 μl (675 mg. 3.79 mmol) of ethyl trifluoromethanesulphonate were then added dropwise, and the mixture was stirred for 2 h. At 0° C., first saturated aqueous ammonium chloride solution and then ethyl acetate were subsequently added. After phase separation, the aqueous phase was extracted twice with ethyl acetate, and the combined organic phases were washed with saturated aqueous sodium chloride solution and then dried over sodium sulphate. After filtration, the filtrate was concentrated under reduced pressure and the residue was dried under high vacuum. This gave 684 mg (97% of theory; purity: 93%) of the desired product.

### Example 222A

### tert-Butyl 6-ethyl-5-oxo-4,7-diazaspiro[2.5]octane-7-carboxylate [racemate]

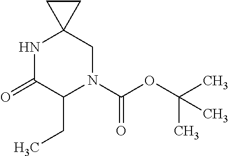 [SMILES: CCC1C(=O)NC2(CC2)CN1C(=O)OC(C)(C)C]

At −78° C., 128 mg (18.4 mmol) of lithium were added to 13 ml of ammonia, and the mixture was stirred for 10 minutes. Then, 684 mg (1.84 mmol, purity: 93%) of tert-butyl 4-benzyl-6-ethyl-5-oxo-4,7-diazaspiro[2.5]octane-7-carboxylate [racemate] in 3 ml of THF were added dropwise, and the mixture was slowly warmed to room temperature and then stirred at room temperature overnight. At 0° C., first saturated aqueous ammonium chloride solution and then water and ethyl acetate were subsequently added. After phase separation, the aqueous phase was extracted twice with ethyl acetate, and the combined organic phases were washed with saturated aqueous sodium chloride solution and then dried over sodium sulphate. After filtration, the filtrate was concentrated under reduced pressure and the residue was dried under high vacuum. This gave 325 mg of the crude product which was used without further purification.

MS (Method 1C): m/z=255 [M+H]⁺

### Example 223A

### 6-Ethyl-4,7-diazaspiro[2.5]octan-5-one trifluoroacetate [racemate]

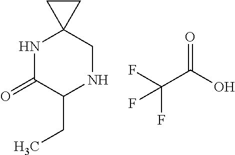

0.985 ml (1.46 g, 12.8 mmol) of trifluoroacetic acid was added to 325 mg (1.28 mmol) of tert-butyl 6-ethyl-5-oxo-4,7-diazaspiro[2.5]octane-7-carboxylate [racemate] in 12 ml of dichloromethane, and the mixture was stirred at room temperature overnight. The mixture was then concentrated under reduced pressure and the residue was dried under high vacuum. The crude product obtained (534 mg) was used further without purification.

MS (Method 1C): m/z=155 [M+H]⁺

### Example 224A

### Benzyl 6-ethyl-5-oxo-4,7-diazaspiro[2.5]octane-7-carboxylate [racemate]

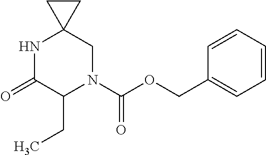 [SMILES: CCC1C(=O)NC2(CC2)CN1C(=O)OCc1ccccc1]

At 0° C., a solution of 0.34 ml (0.40 g, 2.37 mmol) of benzyl chloroformate in 0.7 ml of toluene was added dropwise to 1.33 g (2.37 mmol, purity: 75%) of 6-ethyl-4,7-diazaspiro[2.5]octan-5-one trifluoroacetate [racemate] in 19 ml of THF. A solution of 0.39 ml (0.28 g, 2.84 mmol) of triethylamine in 10 ml of THF was then slowly added dropwise, the mixture was stirred at room temperature overnight and water and ethyl acetate were then added. After phase separation, the aqueous phase was extracted twice with ethyl acetate, and the combined organic phases were washed with saturated aqueous sodium chloride solution. The mixture was dried over sodium sulphate and filtered, the filtrate was concentrated under reduced pressure and the residue was dried under high vacuum, dissolved in methanol and water and purified by preparative HPLC (RP18 column, mobile phase: acetonitrile/water gradient). This gave 120 mg (18% of theory) of the desired product.

MS (Method 1C): m/z=289 [M+H]⁺

### Example 225A

### 6-Ethyl-4,7-diazaspiro[2.5]octan-5-one [racemate]

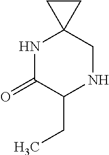

Under argon, 224 mg (0.21 mmol) of 10% palladium on activated carbon were added to 124 mg of benzyl 6-ethyl-5-oxo-4,7-diazaspiro[2.5]octane-7-carboxylate [racemate, Example 224A] in 8 ml of methanol, and the mixture was hydrogenated at RT and standard pressure for 4 h. The mixture was then filtered, the filtrate was concentrated under reduced pressure and the residue was dried under high vacuum. This gave 63 mg (96% of theory) of the desired product.

### Example 226A

### [(tert-Butoxycarbonyl){2-[methoxy(methyl)amino]-2-oxoethyl}amino]acetic acid

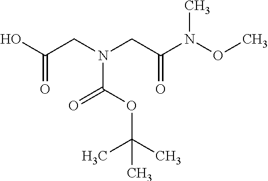 [SMILES: CON(C)C(=O)CN(CC(=O)O)C(=O)OC(C)(C)C]

At 0° C., 33.9 g (177 mmol) of N-ethyl-N′-(3-dimethylaminopropyl)carbodiimide hydrochloride were added a little at a time to 35 g (150 mmol) of 2,2′-[(tert-butoxycarbonyl)imino]diacetic acid in N,N-dimethylformamide (250 ml), and the mixture was stirred at room temperature for 1 h. The mixture was cooled to 0° C., and a solution of 17.3 g (177 mmol) of N,O-dimethylhydroxylamine hydrochloride and 22.8 g (30.8 ml, 177 mmol) of N,N-diisopropylethylamine in 150 ml of dimethylformamide was added dropwise at from 0° C. to 5° C. The mixture was stirred at room temperature overnight. The mixture was then added to a mixture of ice and aqueous 1M hydrogen chloride solution, and ethyl acetate was then added. After phase separation, the aqueous phase was extracted twice with ethyl acetate and the combined organic phases were washed twice with aqueous 1M hydrogen chloride solution and once with saturated aqueous sodium chloride solution and dried over sodium sulphate. After filtration, the filtrate was concentrated under reduced pressure and the residue was dried under high vacuum. Diethyl ether was added to the residue and the mixture was treated in an ultrasonic bath for 20 min. The solid formed was filtered off and dried under high vacuum. This gave 27.8 g (64% of theory) of the desired product.

LC-MS (Method 1A): Rₜ=0.64 min; MS (ESIpos): m/z=277 [M+H]⁺

¹H-NMR (400 MHz, DMSO-d₆): δ[ppm]=12.62 (br. s., 1H), 4.19-4.11 (m, 2H), 3.88 (d, 2H), 3.68 (d, 3H), 3.10 (d, 3H), 1.35 (d, 9H).

### Example 227A

### N-(tert-Butoxycarbonyl)-N-(2-oxopropyl)glycine

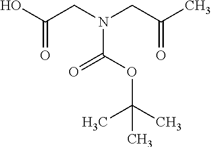 [SMILES: CC(=O)CN(CC(=O)O)C(=O)OC(C)(C)C]

At 0° C., 75.2 ml (26.9 g, 225 mmol) of a 3-molar solution of methylmagnesium bromide in diethyl ether were slowly added dropwise to 13 g (45 mmol) of [(tert-butoxycarbonyl){2-[methoxy(methyl)amino]-2-oxoethyl}amino]acetic acid in 260 ml of THF. The mixture was stirred at room temperature for 2 h. At 0° C., 78 ml of saturated aqueous ammonium chloride solution and 78 ml of water were then added dropwise, and the mixture was allowed to warm slowly to room temperature. Diethyl ether was added, and after phase separation the organic phase was washed with 1N aqueous sodium hydroxide solution. The aqueous phase was cooled to 0° C., acidified with concentrated hydrogen chloride solution and diluted with diethyl ether. The phases were separated and the aqueous phase was washed twice with ethyl acetate. The combined organic phases were washed with saturated aqueous sodium chloride solution and dried over sodium sulphate. After filtration, the filtrate was concentrated under reduced pressure and the residue was dried under high vacuum. This gave 8.85 g (64% of theory, purity: 75%) of the crude product which was used further without purification.

### Example 228A

### tert-Butyl 4-benzyl-3-methyl-5-oxopiperazine-1-carboxylate [racemate]

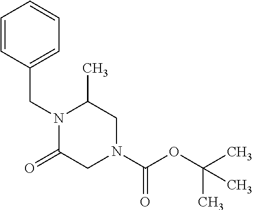 [SMILES: CC1CN(C(=O)OC(C)(C)C)CC(=O)N1Cc1ccccc1]

### i) N-[2-(Benzylamino)propyl]-N-(tert-butoxycarbonyl)glycine

1.76 g (1.81 ml, 16.5 mmol) of benzylamine, 1.53 g (1.46 ml, 25 mmol) of concentrated acetic acid and 7.02 g (16.6 mmol) of sodium triacetoxyborohydride were added to 8.8 g (25 mmol, purity: 67%) of N-(tert-butoxycarbonyl)-N-(2-oxopropyl)glycine in 160 ml of 1,2-dichloroethane, and the mixture was stirred at room temperature overnight. The mixture was then concentrated under reduced pressure.

LC-MS (Method 1A): Rₜ=0.71 min; MS (ESIpos): m/z=323 [M+H]⁺

### ii) tert-Butyl 4-benzyl-3-methyl-5-oxopiperazine-1-carboxylate

The crude product N-[2-(benzylamino)propyl]-N-(tert-butoxycarbonyl)glycine from i) was dissolved in 132 ml of DMF, and 4.88 g (25 mmol) of 1-(3-dimethylaminopropyl)-3-ethylcarbodiimide hydrochloride were added. The mixture was stirred at room temperature overnight, 2.44 g (12.7 mmol) of 1-(3-dimethylaminopropyl)-3-ethylcarbodiimide hydrochloride were added and the mixture was once more stirred overnight. The mixture was washed with saturated aqueous sodium bicarbonate solution and saturated aqueous sodium chloride solution and dried over sodium sulphate. After filtration, the filtrate was concentrated under reduced pressure, and the residue was dried under high vacuum and purified by silica gel chromatography (dichloromethane, then dichloromethane/methanol 100:2). This gave 6.64 g (79% of theory) of the desired product.

LC-MS (Method 1A): Rₜ=1.02 min; MS (ESIpos): m/z=305 [M+H]⁺

### Example 229A

### tert-Butyl 4-benzyl-2-ethyl-5-methyl-3-oxopiperazine-1-carboxylate [diastereomer mixture, 4 isomers]

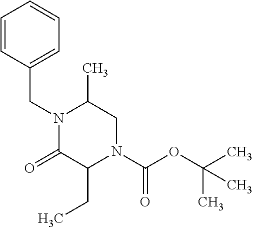 [SMILES: CCC1C(=O)N(Cc2ccccc2)C(C)CN1C(=O)OC(C)(C)C]

At −78° C. and under argon, 5.91 ml (5.91 mmol) of a 1M solution of lithium hexamethyldisilazide in THF were added dropwise to 600 mg (1.97 mmol) of tert-butyl 4-benzyl-3-methyl-5-oxopiperazine-1-carboxylate [racemate] in 24 ml of THF, and the mixture was stirred at −78° C. for 30 min. 0.51 ml (3.94 mmol) of ethyl trifluoromethanesulphonate was then added dropwise, and the mixture was stirred for 2 h. The mixture was then added to saturated aqueous ammonium chloride solution, and ethyl acetate was added. After phase separation, the aqueous phase was extracted twice with ethyl acetate, and the combined organic phases were washed with saturated aqueous sodium chloride solution and dried over sodium sulphate. After filtration, the filtrate was concentrated under reduced pressure and the residue was dried under high vacuum. This gave 821 mg (87% of theory, purity: 70%) of the desired crude product.

LC-MS (Method 1A): Rₜ=1.18 min; MS (ESIpos): m/z=333 [M+H]⁺

### Example 230A

### tert-Butyl 2-ethyl-5-methyl-3-oxopiperazine-1-carboxylate [diastereomer mixture, 4 isomers]

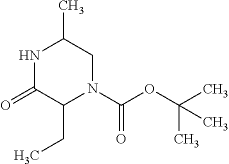 [SMILES: CCC1C(=O)NC(C)CN1C(=O)OC(C)(C)C]

At −78° C., 120 mg (17.3 mmol) of lithium were added to 18 ml of ammonia, and the mixture was stirred for a few minutes. 821 mg (1.72 mmol) of tert-butyl 4-benzyl-2-ethyl-5-methyl-3-oxopiperazine-1-carboxylate [diastereomer mixture, 4 isomers, Example 229A] in 6 ml of THF were then added dropwise, and the mixture was slowly warmed to room temperature and stirred at room temperature overnight. Subsequently, first saturated aqueous ammonium chloride solution and then ethyl acetate were added. After phase separation, the aqueous phase was extracted twice with ethyl acetate, and the combined organic phases were washed with saturated aqueous sodium chloride solution and dried over sodium sulphate. After filtration, the filtrate was concentrated under reduced pressure and the residue was dried under high vacuum. This gave 240 mg of the crude product which was used without further purification.

MS (Method 1C): m/z=243 [M+H]⁺

### Example 231A

### 3-Ethyl-6-methylpiperazin-2-one trifluoroacetate [diastereomer mixture, 4 isomers]

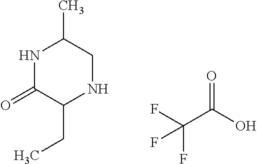

1.46 ml (2.16 g, 18.9 mmol) of trifluoroacetic acid were added to 657 mg (2.71 mmol) of tert-butyl 2-ethyl-5-methyl-3-oxopiperazine-1-carboxylate [diastereomer mixture, 4 isomers, Example 230A] in 22 ml of dichloromethane, and the mixture was stirred at room temperature overnight. The mixture was then concentrated under reduced pressure and the residue was dissolved in dichloromethane. The solution was concentrated under reduced pressure and the residue obtained was re-dissolved in dichloromethane and then freed of the solvent under reduced pressure and dried under high vacuum. The crude product obtained (1.00 g) was used further without purification.

MS (Method 1C): m/z=142 [M+H]⁺

### Example 232A

### Benzyl 2-ethyl-5-methyl-3-oxopiperazine-1-carboxylate [diastereomer mixture, 4 isomers]

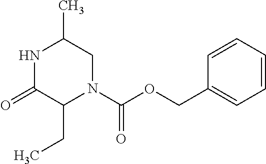 [SMILES: CCC1C(=O)NC(C)CN1C(=O)OCc1ccccc1]

At 0° C., a solution of 0.31 ml (0.37 g, 2.18 mmol) of benzyl chloroformate in 1.2 ml of toluene was added dropwise to 1.55 g (2.18 mmol, purity: 20%) of 3-ethyl-6-methylpiperazin-2-one trifluoroacetate [diastereomer mixture, 4 isomers] in 1 ml of THF. A solution of 0.36 ml (0.26 g, 2.62 mmol) of triethylamine in 0.5 ml of THF was then slowly added dropwise, the mixture was stirred at room temperature overnight and water and ethyl acetate were then added. After phase separation, the aqueous phase was extracted twice with ethyl acetate, and the combined organic phases were washed with saturated aqueous sodium chloride solution. The mixture was dried over sodium sulphate and filtered, the filtrate was concentrated under reduced pressure and the residue was dried under high vacuum, dissolved in methanol and water and purified by preparative HPLC (RP18 column, mobile phase: acetonitrile/water gradient). This gave 84 mg (14% of theory) of the desired product.

LC-MS (Method 1A): Rₜ=0.83 min; MS (ESIpos): m/z=277 [M+H]⁺

### Example 233A

### 3-Ethyl-6-methylpiperazin-2-one [diastereomer mixture, 4 isomers]

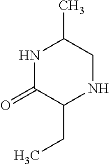

Under argon, 97 mg (0.09 mmol) of 10% palladium on activated carbon were added to 84 mg of benzyl 2-ethyl-5-methyl-3-oxopiperazine-1-carboxylate [diastereomer mixture, 4 isomers, Example 232A] in 10 ml of methanol, and the mixture was hydrogenated at RT and standard pressure for 4 h. The mixture was then filtered, the filtrate was concentrated under reduced pressure and the residue was dried under high vacuum. This gave 41 mg (95% of theory) of the desired product.

MS (Method 1C): m/z=143 [M+H]⁺

### Example 234A

### Benzyl 2-ethyl-5-methyl-3-oxopiperazine-1-carboxylate [enantiomerically pure isomer 3]

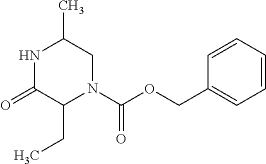 [SMILES: CCC1C(=O)NC(C)CN1C(=O)OCc1ccccc1]

The diastereomer mixture from Example 232A was separated into the enantiomers on a chiral phase [Method 50D]. Yield: enantiomerically pure isomer 3: 50 mg (99% ee)

enantiomerically pure isomer 3: Rₜ=6.72 min [Method 43E].

LC-MS (Method 1A): Rₜ=0.87 min; MS (ESIpos): m/z=277 [M+H]⁺

### Example 235A

### 3-Ethyl-6-methylpiperazin-2-one [enantiomerically pure isomer 3]

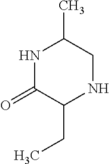

Under argon, 96 mg (0.09 mmol) of 10% palladium on activated carbon were added to 50 mg of benzyl 2-ethyl-5-methyl-3-oxopiperazine-1-carboxylate [enantiomerically pure isomer 3, Example 234A] in 7 ml of ethanol, and the mixture was hydrogenated at RT and standard pressure overnight. The mixture was then filtered, the filtrate was concentrated under reduced pressure and the residue was dried under high vacuum. This gave 28 mg (quant.) of the desired crude product.

MS (Method 1C): m/z=143 [M+H]⁺

### Example 236A

### 2-(5,5-Dimethyl-3-oxopiperazin-2-yl)acetamide [racemate]

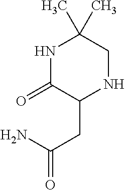

At room temperature, 6.60 g (68 mmol) of 1H-pyrrole-2,5-dione were added to 7.06 ml (6.0 g, 68 mmol) of 2-methylpropane-1,2-diamine in 150 ml of ethanol, and the mixture was stirred at room temperature for 1 h. The mixture was then concentrated under reduced pressure and the residue was dried under high vacuum. This gave 12.3 g (97% of theory) of the desired product.

LC-MS (Method 5A): Rₜ=0.26 min; MS (ESIpos): m/z=186 [M+H]⁺

### Example 237A

### Ethyl (5-methylpyridin-2-yl)acetate

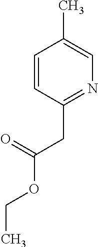

At −50° C., 179 ml (287 mmol) of a 1.6-molar solution of n-butyllithium in hexane were added dropwise to 40.2 ml (29.1 g, 287 mmol) of N,N-diisopropylethylamine and 14.0 ml (10.8 g, 92.8 mmol) of N,N,N,N-tetramethylethylenediamine in 115 ml of THF, and the mixture was stirred at −50° C. for 1 h. 15.1 ml (14.0 g, 130 mmol) of 2,5-dimethylpyridine were then added dropwise, and the mixture was stirred at 0° C. for 1 h. 12.5 ml (14.2 g, 131 mmol) of ethyl chloroformate were then added dropwise at −78° C., and the mixture was stirred at room temperature overnight. At 0° C., first 40 ml of saturated aqueous ammonium chloride solution and then 30 ml of saturated aqueous sodium chloride solution were then added dropwise. At room temperature, ethyl acetate was added, the phases were separated, the aqueous phase was extracted twice with ethyl acetate and the combined organic phases were washed with saturated aqueous sodium chloride solution and dried over sodium sulphate. After filtration, the filtrate was concentrated under reduced pressure and the residue was dried under high vacuum, dissolved in cyclohexane/ethyl acetate and purified by silica gel chromatography (cyclohexane/ethyl acetate 10:1-10:5). This gave 6.12 g (26% of theory) of the desired product.

LC-MS (Method 1A): Rₜ=0.83 min; MS (ESIpos): m/z=180 [M+H]⁺

### Example 238A

### Ethyl (5-methylpiperidin-2-yl)acetate hydrochloride [diastereomer mixture, 4 isomers]

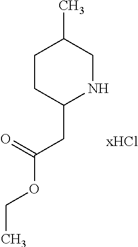

Under argon, 51 mg (0.21 mmol) of platinum(IV) oxide hydrate were added to 2.56 g (13.9 mmol) of ethyl (5-methylpyridin-2-yl)acetate (Example 237A) in 66 ml of acetic acid, and the mixture was hydrogenated at room temperature and standard pressure overnight. The mixture was then filtered through silica gel, the filtrate was concentrated under reduced pressure and 100 ml of 1N aqueous hydrogen chloride solution were added to the residue. The mixture was concentrated under reduced pressure and the residue was dried under high vacuum. This gave 2.44 g (78% of theory) of the desired product.

MS (Method 1C): m/z=223 [M+H]⁺

### Example 239A

### 2-(5-Methylpiperidin-2-yl)ethanol [diastereomer mixture, 4 isomers]

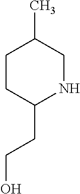

At 0° C., 2.71 ml (2.71 mmol) of a 1.0M solution of lithium aluminium hydride in THF were added dropwise to 400 mg (1.80 mmol) of ethyl (5-methylpiperidin-2-yl)acetate hydrochloride [diastereomer mixture, 4 isomers] in 16 ml of THF, and the mixture was stirred at room temperature overnight. 1.44 ml (1.44 mmol) of a 1.0 molar solution of lithium aluminium hydride in THF were added dropwise at 0° C., and the mixture was stirred at room temperature overnight. At 0° C., 144 μl of water, 156 μl of 3M aqueous sodium hydroxide solution and another 372 μl of water were then added and the precipitate formed was filtered off. The filtrate was concentrated under reduced pressure and the residue was dried under high vacuum. This gave 275 mg (quant.) of the desired product.

MS (Method 1C): m/z=144 [M+H]⁺

### Example 240A

### Ethyl {1-[(2-{[(4-chloropyridin-2-yl)methyl]amino}-7-methoxy-1,3-benzoxazol-5-yl)carbonyl]-5-methylpiperidin-2-yl}acetate [diastereomer mixture, 4 isomers]

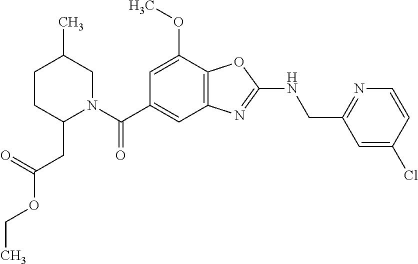 [SMILES: CCOC(=O)CC1CCC(C)CN1C(=O)c1cc(OC)c2oc(NCc3cc(Cl)ccn3)nc2c1]

1.36 g (1.82 ml, 10.5 mmol) of N,N-diisopropylethylamine and 398 mg (1.79 mmol) of ethyl (5-methylpiperidin-2-yl)acetate [diastereomer mixture, 4 isomers] were added to 500 mg (1.49 mmol) of 2-{[(4-chloropyridin-2-yl)methyl]amino}-7-methoxy-1,3-benzoxazole-5-carboxylic acid in N,N-dimethylformamide (15 ml). 684 mg (1.79 mmol) of HATU were then added at 0° C., and the mixture was stirred at room temperature for 2 h. 199 mg (0.899 mmol) of ethyl (5-methylpiperidin-2-yl)acetate [diastereomer mixture, 4 isomers] were then added and the mixture was stirred at room temperature for 1 h. Water was added and the product was purified by preparative HPLC (acetonitrile/water). This gave 110 mg (15% of theory) of the target compound as a diastereomer mixture.

LC-MS (Method 1A): Rₜ=0.98 min (diastereomer 1, 2 isomers), Rₜ=1.00 min (diastereomer 2, 2 isomers);

MS (ESIpos): m/z=501 [M+H]⁺

### Example 241A

### Ethyl (5-methylpiperidin-2-yl)acetate acetate [diastereomer mixture, 4 isomers]

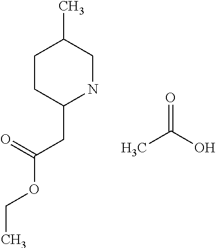

Under argon, 251 mg (1.02 mmol) of platinum(IV) oxide hydrate and 1.09 g (1.02 mmol) of 10% palladium on activated carbon were added to 6.12 g (34.1 mmol) of ethyl (5-methylpyridin-2-yl)acetate (Example 237A) in 250 ml of acetic acid, and the mixture was hydrogenated at room temperature and standard pressure overnight. 125 mg (0.51 mmol) of platinum(IV) oxide hydrate and 545 mg (0.51 mmol) of 10% palladium on activated carbon were added and the mixture was hydrogenated at room temperature and standard pressure overnight. The mixture was then filtered through silica gel, 125 mg (0.51 mmol) of platinum(IV) oxide hydrate and 545 mg (0.51 mmol) of 10% palladium on activated carbon were added to the filtrate and the mixture was hydrogenated at room temperature and standard pressure overnight. The mixture was filtered through silica gel, the filtrate was concentrated under reduced pressure and the residue was dried under high vacuum. This gave 12.4 g of the desired crude product.

GC-MS (Method 2B): Rₜ=3.69 min; MS (ESIpos): m/z=185 [M+H]⁺

### Example 242A

### tert-Butyl 2-(2-ethoxy-2-oxoethyl)-5-methylpiperidine-1-carboxylate [diastereomer mixture, 4 isomers]

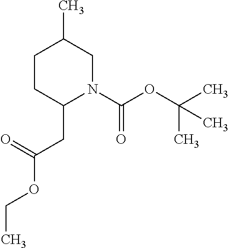 [SMILES: CCOC(=O)CC1CCC(C)CN1C(=O)OC(C)(C)C]

5.96 ml (4.33 g, 42.8 mmol) of triethylamine and 4.00 g (18.3 mmol) of di-tert-butyl dicarbonate were added to 3.00 g (12.2 mmol) of ethyl (5-methylpiperidin-2-yl)acetate acetate [diastereomer mixture, 4 isomers, Example 241A] in 120 ml of dichloromethane. The mixture was stirred at room temperature overnight and then allowed to stand at room temperature for 5 d. The mixture was then concentrated under reduced pressure, ethyl acetate and water were added to the residue, the phases were separated, the aqueous phase was extracted twice with ethyl acetate and the combined organic phases were washed with saturated aqueous sodium chloride solution and then dried over sodium sulphate. After filtration, the filtrate was concentrated under reduced pressure and the residue was dried under high vacuum. This gave 2.11 g (39% of theory, purity: 64%) of the desired product.

LC-MS (Method 4A): Rₜ=3.09 min; MS (ESIpos): m/z=285 [M+H]⁺

### Example 243A

### tert-Butyl 2-[(1-hydroxycyclopropyl)methyl]-5-methylpiperidine-1-carboxylate [diastereomer mixture, 4 isomers]

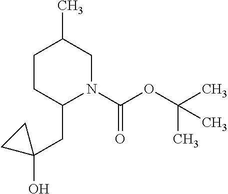 [SMILES: CC1CCC(CC2(O)CC2)N(C(=O)OC(C)(C)C)C1]

Under argon, 99 μl (96 mg, 0.34 mmol) of titanium(IV) tetraprop-2-oxide were added to 1.50 g (3.36 mmol, purity: 64%) of tert-butyl-2-(2-ethoxy-2-oxoethyl)-5-methylpiperidine-1-carboxylate [diastereomer mixture, 4 isomers, Example 242A] in 10.8 ml of diethyl ether. 2.37 ml (7.13 mmol) of a 3M solution of ethylmagnesium bromide in diethyl ether were then slowly added dropwise, and the mixture was stirred at room temperature overnight. 10 ml of diethyl ether were then added, and 99 μl (96 mg, 0.34 mmol) of titanium(IV) tetraprop-2-oxide and 2.37 ml (7.13 mmol) of a 3M solution of ethylmagnesium bromide in diethyl ether were subsequently added dropwise with cooling. The mixture was stirred at room temperature for 2 h and then added to cooled 10% strength aqueous sulphuric acid. After addition of diethyl ether, the phases were separated and the aqueous phase was washed twice with diethyl ether. The combined organic phases were washed with saturated aqueous sodium chloride solution and then dried over sodium sulphate. After filtration, the filtrate was concentrated under reduced pressure and the residue was dried under high vacuum. This gave 930 mg (quant.) of the desired product.

MS (Method 1C): m/z=270 [M+H]⁺

### Example 244A

### 1-[(5-Methylpiperidin-2-yl)methyl]cyclopropanol trifluoroacetate [diastereomer mixture, 4 isomers]

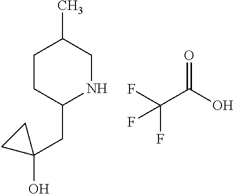

2.65 ml (3.92 g, 34.4 mmol) of trifluoroacetic acid were added to 927 mg (3.44 mmol) of tert-butyl 2-[(1-hydroxycyclopropyl)methyl]-5-methylpiperidine-1-carboxylate [diastereomer mixture, 4 isomers, Example 243A] in 35 ml of dichloromethane, and the mixture was stirred at room temperature overnight. The mixture was then concentrated under reduced pressure and dried under high vacuum. The crude product obtained (1.45 g) was used further without purification.

MS (Method 1C): m/z=170 [M+H-TFA]⁺

### Example 245A

### Methyl 5-methylpyridine-2-carboxylate

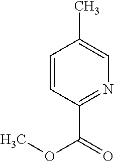

15 ml (60 mmol) of a 4M solution of hydrogen chloride in dioxane were added dropwise to 3.00 g (21.9 mmol) of 5-methylpyridine-2-carboxylic acid in 40 ml of methanol, and the mixture was stirred at room temperature overnight. 10 ml (40 mmol) of a 4M solution of hydrogen chloride in dioxane were then added dropwise, and the mixture was initially stirred at room temperature overnight and then at 70° C. overnight. Methanol and a further 20 ml (80 mmol) of a 4M solution of hydrogen chloride in dioxane were added and the mixture was stirred at reflux overnight. The mixture was then concentrated under reduced pressure, and water and dichloromethane were added to the residue. After phase separation, the aqueous phase was washed twice with dichloromethane and the combined organic phases were washed with sodium chloride solution and then dried over sodium sulphate. After filtration, the filtrate was concentrated under reduced pressure and the residue was dried under high vacuum. This gave 2.08 g (59% of theory) of the desired product.

LC-MS (Method 1A): Rₜ=0.55 min; MS (ESIpos): m/z=152 [M+H]⁺

### Example 246A

### Methyl 5-methylpiperidine-2-carboxylate acetate [diastereomer mixture, 4 isomers]

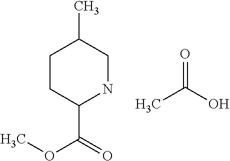

Under argon, 96 mg (0.39 mmol) of platinum(IV) oxide hydrate and 417 mg (0.39 mmol) of 10% palladium on activated carbon were added to 2.08 g (13.1 mmol) of methyl 5-methylpyridine-2-carboxylate (Example 245A) in 90 ml of acetic acid, and the mixture was hydrogenated at room temperature and standard pressure overnight. 48 mg (0.20 mmol) of platinum(IV) oxide hydrate and 208 mg (0.20 mmol) of 10% palladium on activated carbon were then added and the mixture was hydrogenated at room temperature and standard pressure overnight. The mixture was filtered through silica gel, the filtrate was concentrated under reduced pressure and the residue was dried under high vacuum. This gave 3.10 g of the desired crude product.

LC-MS (Method 2B): Rₜ=2.94 min; MS (ESIpos): m/z=158 [M+H]⁺

### Example 247A

### 1-tert-Butyl 2-methyl 5-methylpiperidine-1,2-dicarboxylate [diastereomer mixture, 4 isomers]

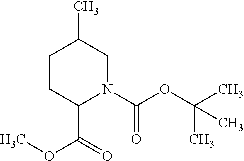 [SMILES: COC(=O)C1CCC(C)CN1C(=O)OC(C)(C)C]

4.81 ml (3.49 g, 34.5 mmol) of triethylamine and 3.01 g (13.8 mmol) of di-tert-butyl dicarbonate were added to 1.50 g (6.90 mmol) of methyl 5-methylpiperidine-2-carboxylate acetate [diastereomer mixture, 4 isomers, Example 246A] in 70 ml of dichloromethane. The mixture was stirred at room temperature overnight. The mixture was then concentrated under reduced pressure, ethyl acetate and water were added to the residue, the phases were separated, the aqueous phase was extracted twice with ethyl acetate and the combined organic phases were washed with saturated aqueous sodium chloride solution and then dried over sodium sulphate. After filtration, the filtrate was concentrated under reduced pressure and the residue was dried under high vacuum. This gave 1.43 g (39% of theory, purity: 62%) of the desired product.

LC-MS (Method 4A): Rₜ=2.948 min (diastereomer 1, 2 isomers), Rₜ=2.99 min (diastereomer 2, 2 isomers);

MS (ESIpos): m/z=158 [M-Boc+H]⁺

### Example 248A

### tert-Butyl 2-(1-hydroxycyclopropyl)-5-methylpiperidine-1-carboxylate [diastereomer mixture, 4 isomers]

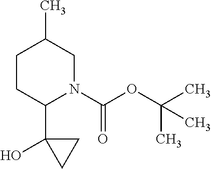 [SMILES: CC1CCC(C2(O)CC2)N(C(=O)OC(C)(C)C)C1]

Under argon, 115 μl (110 mg, 0,389 mmol) of titanium(IV) tetraprop-2-oxide were added to 1.00 g (3.39 mmol, purity: 62%) of 1-tert-butyl 2-methyl 5-methylpiperidine-1,2-dicarboxylate (diastereomer mixture, 4 isomers, Example 247A) in 12.5 ml of diethyl ether. 2.75 ml (8.25 mmol) of a 3 molar solution of ethylmagnesium bromide in diethyl ether were then slowly added dropwise, and the mixture was stirred at room temperature overnight. 10 ml of diethyl ether were added, and 115 μl (110 mg, 0.389 mmol) of titanium(IV) tetraprop-2-oxide and 2.75 ml (8.25 mmol) of a 3M solution of ethylmagnesium bromide in diethyl ether were subsequently added dropwise with cooling. The mixture was stirred at room temperature for 2 h and then added to cooled 10% strength aqueous sulphuric acid. After addition of diethyl ether, the phases were separated and the aqueous phase was washed twice with diethyl ether. The combined organic phases were washed with saturated aqueous sodium chloride solution and then dried over sodium sulphate. After filtration, the filtrate was concentrated under reduced pressure and the residue was dried under high vacuum. This gave 410 mg (41% of theory) of the desired product.

MS (Method 1C): m/z=256 [M+H]⁺

### Example 249A

### 1-(5-Methylpiperidin-2-yl)cyclopropanol trifluoroacetate [diastereomer mixture, 4 isomers]

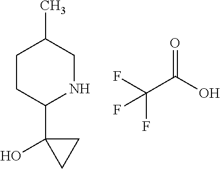

1.24 ml (1.83 g, 16.1 mmol) of trifluoroacetic acid were added to 410 mg (1.06 mmol) of tert-butyl 2-(1-hydroxycyclopropyl)-5-methylpiperidine-1-carboxylate [diastereomer mixture, 4 isomers, Example 248A] in 16 ml of dichloromethane, and the mixture was stirred at room temperature overnight. The mixture was then concentrated under reduced pressure and dried under high vacuum. The crude product obtained (645 mg) was used further without purification.

MS (Method 1C): m/z=156 [M+H-TFA]⁺

### Example 250A

### 2-(5-Methyl-3-oxopiperazin-2-yl)acetamide [diastereomer mixture, 4 isomers]

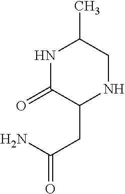

At room temperature, 6.55 g (67 mmol) of 1H-pyrrole-2,5-dione were added to 5.74 ml (5.0 g, 67 mmol) of propane-1,2-diamine in 160 ml of ethanol, and the mixture was stirred at room temperature overnight. The mixture was then concentrated under reduced pressure and the residue was dried under high vacuum. This gave 12.6 g of the desired crude product.

MS (Method 1C): m/z=172 [M+H]⁺

### Example 251A

### 2-{1-[(2-{[(4-Chloropyridin-2-yl)methyl]amino}-7-methoxy-1,3-benzoxazol-5-yl)carbonyl]-5-methyl-3-oxopiperazin-2-yl}acetamide [diastereomer mixture, 4 isomers]

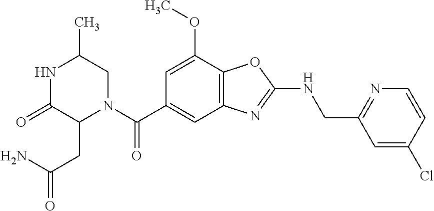 [SMILES: COc1cc(C(=O)N2CC(C)NC(=O)C2CC(N)=O)cc2nc(NCc3cc(Cl)ccn3)oc12]

2.06 g (2.78 ml, 15.9 mmol) of N,N-diisopropylethylamine, 3.31 g (8.70 mmol) of HATU and 1.49 g (8.70 mmol) of 2-(5-methyl-3-oxopiperazin-2-yl)acetamide [diastereomer mixture, 4 isomers] were added to 2.42 g (7.25 mmol) of 2-{[(4-chloropyridin-2-yl)methyl]amino}-7-methoxy-1,3-benzoxazole-5-carboxylic acid in N,N-dimethylformamide (70 ml), and the mixture was stirred at room temperature for 2.5 h. After addition of dichloromethane and water, the phases were separated and the aqueous phase was washed twice with dichloromethane. The combined organic phases were washed with saturated aqueous sodium chloride solution and then dried over sodium sulphate. After filtration, the filtrate was concentrated under reduced pressure and the residue was dried under high vacuum. Methanol and water were added to the residue, and the product was purified by preparative HPLC (acetonitrile/water). This gave 480 mg (14% of theory) of the target compound. The aqueous phase from the extraction was also concentrated under reduced pressure and methanol and water were added to the residue, and the product was purified by preparative HPLC (acetonitrile water). This gave another 850 mg (24% of theory) of the target compound.

LC-MS (Method 1A): Rₜ=0.63 min; MS (ESIpos): m/z=487 [M+H]⁺

### Example 252A

### 4-Benzyl-5-({[(2,2-dimethylpropyl)(dimethyl)silyl]oxy}methyl)-2,2-dimethylmorpholin-3-one [racemate]

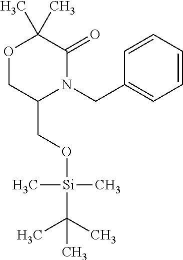 [SMILES: CC1(C)OCC(CO[Si](C)(C)C(C)(C)C)N(Cc2ccccc2)C1=O]

At −78° C. and under argon, 32.4 ml (58.4 mmol) of a 1.8M solution of lithium diisopropylamide in THF/heptane were added dropwise to 17.0 g (48.6 mmol) of 4-benzyl-5-({[tert-butyl(dimethyl)silyl]oxy}methyl)-2-methylmorpholin-3-one [diastereomer mixture, 4 isomers] in 340 ml of THF. Over 20 min, the mixture was warmed to 0° C., 3.94 ml (8.97 g, 63.2 mmol) of methyl iodide were added and the mixture was stirred for 1.5 h. The mixture was cooled to −78° C., 5.40 ml (9.72 mmol) of a 1.8M solution of lithium diisopropylamide in THF/heptane were added, the mixture was warmed to 0° C. over 20 min and 0.91 ml (2.07 g, 14.6 mmol) of methyl iodide was added. The mixture was stirred at room temperature for 1 h and water was then added carefully with ice cooling. The mixture was then concentrated under reduced pressure, ethyl acetate was added to the residue and the mixture was washed with water and saturated aqueous sodium chloride solution and dried over sodium sulphate. After filtration, the filtrate was concentrated under reduced pressure and the residue was dried under high vacuum. This gave 19.8 g (98% of theory) of the desired crude product.

LC-MS (Method 1A): Rₜ=1.45 min; MS (ESIpos): m/z=364 [M+H]⁺

### Example 253A

### 4-Benzyl-5-(hydroxymethyl)-2,2-dimethylmorpholin-3-one [racemate]

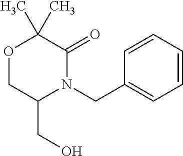 [SMILES: CC1(C)OCC(CO)N(Cc2ccccc2)C1=O]

At room temperature, 109.5 ml (109.5 mmol) of a 1M solution of tetra-n-butylammonium fluoride in THF were added to 18.1 g (43.8 mmol) of 4-benzyl-5-({[(2,2-dimethylpropyl)(dimethyl)silyl]oxy}methyl)-2,2-dimethylmorpholin-3-one [racemate] in 330 ml of THF, and the mixture was stirred overnight. The mixture was concentrated under reduced pressure, ethyl acetate was added to the residue and the mixture was washed with water. After separation of the phases, the organic phase was dried over sodium sulphate. After filtration, the filtrate was concentrated under reduced pressure and the residue was purified by silica gel chromatography (dichloromethane—dichloromethane/methanol 100:3). This gave 9.99 g (89% of theory) of the desired product.

LC-MS (Method 1A): Rₜ=0.73 min; MS (ESIpos): m/z=250 [M+H]⁺

¹H-NMR (400 MHz, DMSO-d₆): δ[ppm]=7.43-7.20 (m, 5H), 5.11-4.92 (m, 2H), 4.23 (d, 1H), 3.93-3.71 (m, 2H), 3.66-3.54 (m, 2H), 3.18-3.10 (m, 1H), 1.42 (s, 3H), 1.39 (s, 3H).

### Example 254A

### 4-Benzyl-6,6-dimethyl-5-oxomorpholine-3-carboxylic acid [racemate]

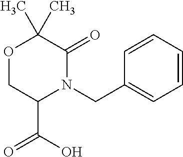 [SMILES: CC1(C)OCC(C(=O)O)N(Cc2ccccc2)C1=O]

At room temperature, 37.65 g (165 mmol) of periodic acid were added to 19.5 g (43.8 mmol) of 4-benzyl-5-(hydroxymethyl)-2,2-dimethylmorpholin-3-one [racemate] in 1200 ml of acetonitrile, and the mixture was stirred for 15 min. At 0° C., 647 mg (3.00 mmol) of pyridinium chlorochromate in 45 ml acetonitrile were then added and the mixture was stirred at 0° C. for 2 h. The mixture was concentrated under reduced pressure, water was then added to the residue and the mixture was washed with ethyl acetate. After separation of the phases, the organic phase was dried over sodium sulphate. After filtration, the filtrate was concentrated under reduced pressure and the residue was dried under high vacuum. This gave 18.4 g (56% of theory, purity: 60%) of the desired crude product.

LC-MS (Method 1A): Rₜ=0.69 min; MS (ESIpos): m/z=264 [M+H]⁺

### Example 255A

### Methyl 4-benzyl-6,6-dimethyl-5-oxomorpholine-3-carboxylate [racemate]

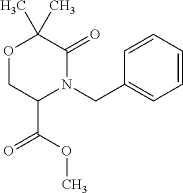 [SMILES: COC(=O)C1COC(C)(C)C(=O)N1Cc1ccccc1]

At 0° C., 12.2 ml (16.7 g, 141 mmol) of thionyl chloride were slowly added dropwise to 18.5 g (70.4 mmol, purity: 60%) of 4-benzyl-6,6-dimethyl-5-oxomorpholine-3-carboxylic acid [racemate] in 232 ml of methanol. With stirring, the mixture was then heated at reflux for 2 h. The mixture was concentrated under reduced pressure and the residue was dried under high vacuum. This gave 18.4 g (80% of theory, purity: 85%) of the desired crude product.

LC-MS (Method 1A): Rₜ=0.87 min; MS (ESIpos): m/z=278 [M+H]⁺

### Example 256A

### 4-Benzyl-5-(2-hydroxypropan-2-yl)-2,2-dimethylmorpholin-3-one [racemate]

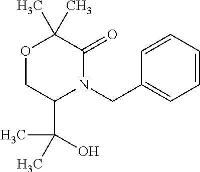 [SMILES: CC1(C)OCC(C(C)(C)O)N(Cc2ccccc2)C1=O]

At 0° C., 32.5 ml (97.6 mmol) of a 3M solution of methylmagnesium bromide in diethyl ether were slowly added dropwise to 9.1 g (27.9 mmol, purity: 85%) of methyl 4-benzyl-6,6-dimethyl-5-oxomorpholine-3-carboxylate [racemate] in 507 ml of THF. Subsequently, the mixture was stirred at 0° C. for 1 h and then at room temperature overnight. At 0° C., 16.7 ml (50.2 mmol) of a 3M solution of methylmagnesium bromide in diethyl ether were added and the mixture was stirred at room temperature overnight. At 0° C., saturated aqueous ammonium chloride solution was then added and the mixture was freed from THF under reduced pressure. Dichloromethane and water were added to the residue, the phases were separated and the organic phase was washed twice with water. The aqueous phase was then washed with dichloromethane. The combined organic phases were washed with saturated aqueous sodium chloride solution and then dried over sodium sulphate.

After filtration, the filtrate was concentrated under reduced pressure and the residue was dried under high vacuum and purified by preparative HPLC (RP18 column, mobile phase: acetonitrile/water gradient). This gave 3.36 g (43% of theory) of the desired product.

LC-MS (Method 1A): Rₜ=0.85 min; MS (ESIpos): m/z=278 [M+H]⁺

¹H-NMR (400 MHz, DMSO-d₆): δ[ppm]=7.41-6.99 (m, 5H), 5.35-5.06 (m, 1H), 4.76 (s, 1H), 4.52 (d, 1H), 4.25-3.95 (m, 1H), 3.89-3.57 (m, 1H), 3.12-2.97 (m, 1H), 1.38-1.33 (m, 6H), 1.28-1.23 (m, 3H), 1.20 (s, 3H).

### Example 257A

### 2-(4-Benzyl-6,6-dimethylmorpholin-3-yl)propan-2-ol [racemate]

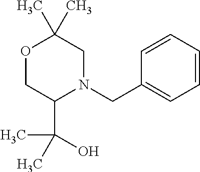 [SMILES: CC1(C)CN(Cc2ccccc2)C(C(C)(C)O)CO1]

At room temperature, 59.3 ml (118 mmol) of a 2M solution of dimethyl sulphide/borane complex in THF were slowly added dropwise to 3.36 g (11.8 mmol) of 4-benzyl-5-(2-hydroxypropan-2-yl)-2,2-dimethylmorpholin-3-one [racemate] in 421 ml of methanol. The mixture was stirred at room temperature overnight and then under reflux for 7 h. Subsequently, 300 ml of methanol were added slowly at room temperature and the mixture was, with stirring, heated at reflux for 4 h. Subsequently, the mixture was concentrated and the residue was dried under high vacuum and purified by preparative HPLC (RP18 column, mobile phase: acetonitrile/water gradient). This gave 1.51 g (48% of theory) of the desired product.

LC-MS (Method 1A): Rₜ=0.58 min; MS (ESIpos): m/z=264 [M+H]⁺

¹H-NMR (400 MHz, DMSO-d₆): δ[ppm]=7.40-7.15 (m, 5H), 5.04 (d, 1H), 4.59 (s, 1H), 3.56 (dd, 1H), 3.41 (t, 1H), 3.02-2.89 (m, 1H), 2.41-2.26 (m, 2H), 1.83 (d, 1H), 1.24 (s, 3H), 1.15 (s, 3H), 1.09 (s, 3H), 0.96 (s, 3H).

### Example 258A

### 2-(4-Benzyl-6,6-dimethylmorpholin-3-yl)propan-2-ol [enantiomerically pure isomer 1]

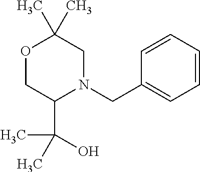 [SMILES: CC1(C)CN(Cc2ccccc2)C(C(C)(C)O)CO1]

1.51 g of 2-(4-benzyl-6,6-dimethylmorpholin-3-yl)propan-2-ol [racemate, Example 257A] were separated into the enantiomers on a chiral phase [Method 51D].

Yield: enantiomerically pure isomer 1: 448 mg (100% ee)

enantiomerically pure isomer 1: Rₜ=5.40 min [Method 44E].

LC-MS (Method 1A): Rₜ=0.66 min; MS (ESIpos): m/z=264 [M+H]⁺

¹H-NMR (400 MHz, DMSO-d₆): δ[ppm]=7.39-7.11 (m, 5H), 5.04 (d, 1H), 4.59 (s, 1H), 3.56 (dd, 1H), 3.46-3.35 (m, 1H), 3.30 (s, 1H), 2.96 (d, 1H), 2.40-2.26 (m, 2H), 1.24 (s, 3H), 1.15 (s, 3H), 1.09 (s, 3H), 0.96 (s, 3H).

### Example 259A

### 2-(6,6-Dimethylmorpholin-3-yl)propan-2-ol [enantiomerically pure isomer 1]

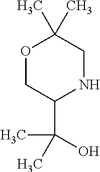

Under argon, 60 mg (0.56 mmol) of 10% palladium on activated carbon and 30 mg (0.21 mmol) of palladium(II) hydroxide were added to 477 mg (1.81 mmol) of 2-(4-benzyl-6,6-dimethylmorpholin-3-yl)propan-2-ol][enantiomerically pure isomer 1] in 20 ml of ethanol, and the mixture was hydrogenated at RT and standard pressure overnight. The mixture was then filtered through silica gel, the filtrate was concentrated under reduced pressure and the residue was dried under high vacuum. This gave 307 mg (98% of theory) of the desired product.

MS (Method 1C): m/z=174 [M+H]⁺

¹H-NMR (400 MHz, DMSO-d₆): δ[ppm]=4.41-4.23 (m, 1H), 3.50-3.26 (m, 3H), 2.72-2.60 (m, 1H), 2.37 (dd, 1H), 1.18 (s, 3H), 1.10-0.97 (m, 9H).

### Example 260A

### 4-Benzyl-5-(1-hydroxycyclopropyl)-2,2-dimethylmorpholin-3-one [racemate]

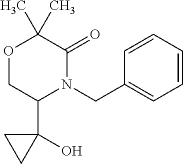 [SMILES: CC1(C)OCC(C2(O)CC2)N(Cc2ccccc2)C1=O]

At room temperature, 0.83 ml (0.79 g, 2.79 mmol) of titanium(IV) tetraprop-2-oxide and then 19.7 ml (59.1 mmol) of a 3M solution of ethylmagnesium bromide in diethyl ether were slowly added dropwise to 9.1 g (27.9 mmol, purity: 85%) of methyl 4-benzyl-6,6-dimethyl-5-oxomorpholine-3-carboxylate [racemate] in 350 ml of diethyl ether. The mixture was stirred at room temperature overnight, and 0.83 ml (0.79 g, 2.79 mmol) of titanium(IV) tetraprop-2-oxide and 19.7 ml (59.1 mmol) of a 3M solution of ethylmagnesium bromide in diethyl ether were then slowly added dropwise, and the mixture was stirred overnight. 0.21 ml (0.19 g, 0.69 mmol) of titanium(IV) tetraprop-2-oxide and 4.92 ml (14.7 mmol) of a 3M solution of ethylmagnesium bromide in diethyl ether were then slowly added dropwise, and the mixture was stirred at room temperature for 4 h. The mixture was added to cooled 10% strength aqueous sulphuric acid and diluted with diethyl ether. The phases were separated, the aqueous phase was washed twice with diethyl ether and the combined organic phases were washed with saturated aqueous sodium chloride solution and then dried over sodium sulphate. After filtration, the filtrate was concentrated under reduced pressure and the residue was dried under high vacuum and purified by preparative HPLC (RP18 column, mobile phase: acetonitrile/water gradient). This gave 1.67 g (20% of theory) of the desired product.

LC-MS (Method 1A): Rₜ=0.81 min; MS (ESIpos): m/z=276 [M+H]⁺

¹H-NMR (400 MHz, DMSO-d₆): δ[ppm]=7.42-7.07 (m, 5H), 5.48 (s, 1H), 5.38 (d, 1H), 4.29-4.13 (m, 1H), 4.08-3.94 (m, 1H), 3.83 (dd, 1H), 2.79 (dd, 1H), 1.47-1.25 (m, 6H), 0.80-0.66 (m, 1H), 0.62-0.45 (m, 1H), 0.42-0.21 (m, 2H).

### Example 261A

### 1-(4-Benzyl-6,6-dimethylmorpholin-3-yl)cyclopropanol [racemate]

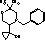 [SMILES: CC1(C)CN(Cc2ccccc2)C(C2(O)CC2)CO1]

At room temperature, 26.8 ml (53.6 mmol) of a 2M solution of dimethyl sulphide/borane complex in THF were slowly added dropwise to 1.57 g (5.36 mmol) of 4-benzyl-5-(1-hydroxycyclopropyl)-2,2-dimethylmorpholin-3-one [racemate] in 300 ml of methanol. The mixture was stirred at room temperature overnight and then under reflux for 2 h. Subsequently, 300 ml of methanol were added slowly at room temperature and the mixture was heated at reflux for 4 h. The mixture was then concentrated and the residue was purified by preparative HPLC (RP18 column, mobile phase: acetonitrile/water gradient). This gave 1.08 g (77% of theory) of the desired product.

LC-MS (Method 1A): Rₜ=0.49 min; MS (ESIpos): m/z=262 [M+H]⁺

¹H-NMR (400 MHz, DMSO-d₆): δ[ppm]=7.47-7.15 (m, 5H), 4.96 (s, 1H), 4.68 (d, 1H), 3.91 (t, 1H), 3.53 (dd, 1H), 2.80 (d, 1H), 2.32 (d, 1H), 1.70 (d, 1H), 1.53 (dd, 1H), 1.26-1.14 (m, 3H), 0.95 (s, 3H), 0.82-0.70 (m, 1H), 0.58-0.45 (m, 2H), 0.40-0.30 (m, 1H)

### Example 262A

### 1-(4-Benzyl-6,6-dimethylmorpholin-3-yl)cyclopropanol [enantiomerically pure isomer 1]

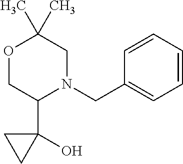 [SMILES: CC1(C)CN(Cc2ccccc2)C(C2(O)CC2)CO1]

1.08 g of 1-(4-benzyl-6,6-dimethylmorpholin-3-yl)cyclopropanol [racemate, Example 261A] were separated into the enantiomers on a chiral phase [Method 52D].

Yield: enantiomerically pure isomer 1: 340 mg (100% ee)

enantiomerically pure isomer 1: Rₜ=4.53 min [Method 44E].

LC-MS (Method 1A): Rₜ=0.55 min; MS (ESIpos): m/z=262 [M+H]⁺

¹H-NMR (400 MHz, DMSO-d₆): δ[ppm]=7.47-7.15 (m, 5H), 4.96 (s, 1H), 4.68 (d, 1H), 3.91 (dd, 1H), 3.53 (dd, 1H), 2.80 (d, 1H), 2.32 (d, 1H), 1.70 (d, 1H), 1.53 (dd, 1H), 1.26-1.14 (m, 3H), 0.95 (s, 3H), 0.82-0.70 (m, 1H), 0.58-0.45 (m, 2H), 0.40-0.30 (m, 1H).

### Example 263A

### 1-(6,6-Dimethylmorpholin-3-yl)cyclopropanol [enantiomerically pure isomer 1]

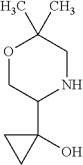

Under argon, 43 mg (0.40 mmol) of 10% palladium on activated carbon and 21 mg (0.15 mmol) of palladium(II) hydroxide were added to 339 mg (1.29 mmol) of 1-(4-benzyl-6,6-dimethylmorpholin-3-yl)cyclopropanol [enantiomerically pure isomer 1] in 15 ml of ethanol, and the mixture was hydrogenated at RT and standard pressure overnight. The mixture was then filtered through silica gel, the filtrate was concentrated under reduced pressure and the residue was dried under high vacuum. This gave 217 mg (98% of theory) of the desired product.

MS (Method 1C): m/z=172 [M+H]⁺

### Example 264A

### 4-Benzyl-6,6-dimethyl-5-oxomorpholine-3-carbaldehyde [racemate]

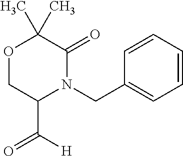 [SMILES: CC1(C)OCC(C=O)N(Cc2ccccc2)C1=O]

At −50° C., a solution of 2.42 ml (2.67 g, 34.2 mmol) of DMSO in 35 ml of dichloromethane was slowly added dropwise to 1.67 ml (2.43 g, 19 mmol) of ethanedioyl dichloride in 195 ml of dichloromethane, and the mixture was stirred at −50° C. for 10 min. A solution of 3.48 g (13.7 mmol) of 4-benzyl-5-(hydroxymethyl)-2,2-dimethylmorpholin-3-one [racemate] in 45 ml of dichloromethane was then slowly added dropwise, and the mixture was stirred at −50° C. for 10 min. At −78° C., a solution of 9.53 ml (6.92 g, 68.3 mmol) of triethylamine in 25 ml of dichloromethane was added dropwise. The mixture was stirred at −78° C. for 2 h and then allowed to slowly warm to room temperature. Saturated aqueous sodium bicarbonate solution and dichloromethane were added and the phases were separated. The aqueous phase was washed twice with dichloromethane. The combined organic phases were washed with saturated aqueous sodium chloride solution and then dried over sodium sulphate. After filtration, the filtrate was concentrated under reduced pressure and the residue was dried under high vacuum. This gave 3.86 g (purity: 47%) of the desired crude product.

LC-MS (Method 1A): Rₜ=0.84 min; MS (ESIpos): m/z=248 [M+H]⁺

### Example 265A

### 4-Benzyl-5-(1-hydroxyethyl)-2,2-dimethylmorpholin-3-one [diastereomer mixture, 4 isomers]

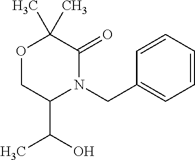 [SMILES: CC(O)C1COC(C)(C)C(=O)N1Cc1ccccc1]

At 0° C., 15.6 ml (46.8 mmol) of a 3M solution of methylmagnesium iodide in diethyl ether were slowly added dropwise to 3.86 g (15.6 mmol) of 4-benzyl-6,6-dimethyl-5-oxomorpholine-3-carbaldehyde [racemate] in 50 ml of THF, and the mixture was stirred at room temperature for 1.5 h. Saturated aqueous ammonium chloride solution was added and the mixture was freed from THF under reduced pressure. Dichloromethane and water were added to the residue and the phases were separated. The organic phase was washed with water and dried over sodium sulphate. After filtration, the filtrate was concentrated under reduced pressure and the residue was dried under high vacuum. This gave 3.65 g (82% of theory, purity: 92%) of the desired product.

LC-MS (Method 1A): Rₜ=0.80 min; MS (ESIpos): m/z=264 [M+H]⁺

¹H-NMR (400 MHz, DMSO-d₆): δ[ppm]=7.40-7.12 (m, 5H), 5.17-4.91 (m, 2H), 4.31-4.20 (m, 1H), 4.10-3.95 (m, 1H), 3.89-3.66 (m, 2H), 3.09-2.93 (m, 1H), 1.41-1.27 (m, 6H), 1.11 (d, 3H).

### Example 266A

### 1-(4-Benzyl-6,6-dimethylmorpholin-3-yl)ethanol [diastereomer mixture, 4 isomers]

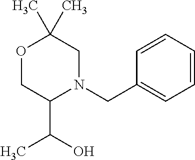 [SMILES: CC(O)C1COC(C)(C)CN1Cc1ccccc1]

At room temperature, 64.5 ml (129 mmol) of a 2M solution of dimethyl sulphide/borane complex in THF were slowly added dropwise to 3.65 g (12.9 mmol, purity: 92%) of 4-benzyl-5-(1-hydroxyethyl)-2,2-dimethylmorpholin-3-one [diastereomer mixture, 4 isomers] in 400 ml of methanol. The mixture was stirred at room temperature overnight and then under reflux for 2 h. Subsequently, 400 ml of methanol were added slowly at room temperature and the mixture was then heated at reflux for 4 h. After concentration, the residue was purified by silica gel chromatography (dichloromethane/methanol 100:2-100:3). This gave 2.11 g (65% of theory) of the desired product.

LC-MS (Method 1A): Rₜ=0.50 min; MS (ESIpos): m/z=250 [M+H]⁺

### Example 267A

### 1-(4-Benzyl-6,6-dimethylmorpholin-3-yl)ethanol [enantiomerically pure isomer 2]

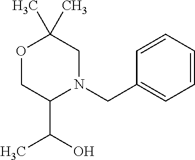 [SMILES: CC(O)C1COC(C)(C)CN1Cc1ccccc1]

2.11 g of 1-(4-benzyl-6,6-dimethylmorpholin-3-yl)ethanol [diastereomer mixture, 4 isomers, Example 266A] were separated into the enantiomers on a chiral phase [Method 53D].

Yield: enantiomerically pure isomer 2: 577 mg (100% ee)

enantiomerically pure isomer 2: Rₜ=6.55 min [Method 45E].

LC-MS (Method 1A): Rₜ=0.52 min; MS (ESIpos): m/z=250 [M+H]⁺

¹H-NMR (400 MHz, DMSO-d₆): δ[ppm]=7.40-7.16 (m, 5H), 4.66 (d, 1H), 4.31-4.19 (m, 1H), 4.05 (d, 1H), 3.72-3.48 (m, 2H), 3.00 (d, 1H), 2.40-2.23 (m, 2H), 1.79 (d, 1H), 1.20-1.06 (m, 6H), 0.98 (s, 3H).

### Example 268A

### 1-(4-Benzyl-6,6-dimethylmorpholin-3-yl)ethanol [enantiomerically pure isomer 1]

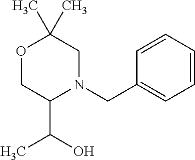 [SMILES: CC(O)C1COC(C)(C)CN1Cc1ccccc1]

2.11 g of 1-(4-benzyl-6,6-dimethylmorpholin-3-yl)ethanol [diastereomer mixture, 4 isomers, Example 266A] were separated into the enantiomers on a chiral phase [Method 53D].

Yield: enantiomerically pure isomer 1: 65 mg (100% ee)

enantiomerically pure isomer 1: Rₜ=5.75 min [Method 45E].

LC-MS (Method 1A): Rₜ=0.50 min; MS (ESIpos): m/z=250 [M+H]⁺

¹H-NMR (400 MHz, DMSO-d₆): δ[ppm]=7.41-7.17 (m, 5H), 4.63 (d, 1H), 4.48 (d, 1H), 3.91-3.80 (m, 1H), 3.57-3.43 (m, 2H), 2.96 (d, 1H), 2.40-2.20 (m, 2H), 1.79 (d, 1H), 1.22-1.09 (m, 6H), 1.01-0.90 (m, 3H).

### Example 269A

### 1-(6,6-Dimethylmorpholin-3-yl)ethanol [enantiomerically pure isomer 2]

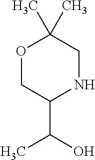

Under argon, 76 mg (0.71 mmol) of 10% palladium on activated carbon and 38 mg (0.27 mmol) of palladium(II) hydroxide were added to 575 mg (2.31 mmol) of 1-(4-benzyl-6,6-dimethylmorpholin-3-yl)ethanol [enantiomerically pure isomer 2] in 26 ml of ethanol, and the mixture was hydrogenated at RT and standard pressure overnight. The mixture was then filtered through silica gel, the filtrate was concentrated under reduced pressure and the residue was dried under high vacuum. This gave 217 mg (98% of theory) of the desired product.

MS (Method 1C): m/z=160 [M+H]⁺

¹H-NMR (400 MHz, DMSO-d₆): δ[ppm]=6.93-6.58 (m, 1H), 4.70-4.48 (m, 2H), 4.43-4.31 (m, 1H), 2.64 (d, 2H), 2.33 (ddd, 2H), 1.36 (s, 3H), 1.25-1.12 (m, 6H).

### Example 270A

### 1-(6,6-Dimethylmorpholin-3-yl)ethanol [enantiomerically pure isomer 1]

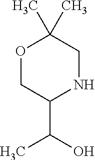

Under argon, 8.6 mg (0.07 mmol) of 10% palladium on activated carbon and 4.3 mg (0.03 mmol) of palladium(II) hydroxide were added to 64 mg (0.26 mmol) of 1-(4-benzyl-6,6-dimethylmorpholin-3-yl)ethanol [enantiomerically pure isomer 1] in 10 ml of ethanol, and the mixture was hydrogenated at room temperature and standard pressure overnight. The mixture was then filtered through silica gel, the filtrate was concentrated under reduced pressure and the residue was dried under high vacuum. This gave 41 mg (100% of theory) of the desired product.

MS (Method 1C): m/z=160 [M+H]⁺

### WORKING EXAMPLES

### Example 1

### (2-{[(4-Chloropyridin-2-yl)methyl]amino}-7-methoxy-1,3-benzoxazol-5-yl)[(4R)-4-ethoxy-2-(hydroxymethyl)pyrrolidin-1-yl]methanone [diastereomer mixture, 2 isomers]

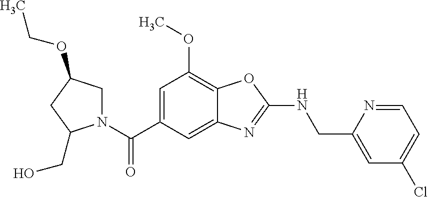 [SMILES: CCO[C@@H]1CC(CO)N(C(=O)c2cc(OC)c3oc(NCc4cc(Cl)ccn4)nc3c2)C1]

120 mg (0.291 mmol, purity: 81%) of 2-{[(4-chloropyridin-2-yl)methyl]amino}-7-methoxy-1,3-benzoxazole-5-carboxylic acid and 63.4 mg (0.437 mmol) of [(4R)-4-ethoxypyrrolidin-2-yl]methanol [diastereomer mixture, 2 isomers] were initially charged in N,N-dimethylformamide (1.34 ml), and 132 mg (178 μl, 0.510 mmol) of N,N-diisopropylethylamine were added. 133 mg (0.350 mmol) of HATU were then added at RT, and the mixture was stirred for 1 h. Without further work-up, the reaction solution was then purified directly by preparative RP-HPLC (acetonitrile/water). Yield: 87.4 mg (63% of theory).

LC-MS (Method 2A): Rₜ=0.76 min (enantiomerically pure isomer 1), Rₜ=0.78 min (enantiomerically pure isomer 2);

MS (ESIpos): m/z=461 [M+H]⁺.

### Example 2

### (2-{[(4-Chloropyridin-2-yl)methyl]amino}-7-methoxy-1,3-benzoxazol-5-yl)[(4R)-4-ethoxy-2-(hydroxymethyl)pyrrolidin-1-yl]methanone [enantiomerically pure isomer 1]

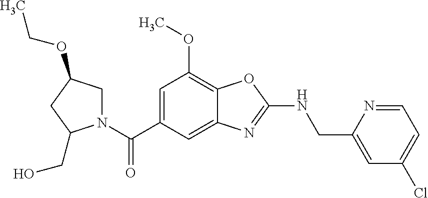 [SMILES: CCO[C@@H]1CC(CO)N(C(=O)c2cc(OC)c3oc(NCc4cc(Cl)ccn4)nc3c2)C1]

Diastereomer separation on a chiral phase of 83.0 mg of the compound from Example 1 according to Method 28D gave 46.0 mg of Example 2 (enantiomerically pure isomer 1) and 21.0 mg of Example 3 (enantiomerically pure isomer 2).

HPLC (Method 26E): Rₜ=5.10 min, >99.9% ee;

LC-MS (Method 1A): Rₜ=0.80 min; MS (ESIpos): m/z=461 [M+H]⁺;

¹H-NMR (400 MHz, DMSO-d₆): δ[ppm]=8.70 (t, 1H), 8.52 (d, 1H), 7.53 (d, 1H), 7.46 (dd, 1H), 6.98 (s, 1H), 6.84 (s, 1H), 4.77 (t, 1H), 4.63 (d, 2H), 4.20 (br. s., 1H), 3.95 (br. s., 1H), 3.92 (s, 3H), 3.71-3.62 (m, 1H), 3.59-3.48 (m, 2H), 3.25-3.16 (m, 1H), 2.02 (d, 2H), 1.00 (t, 3H), two protons obscured.

### Example 3

### (2-{[(4-Chloropyridin-2-yl)methyl]amino}-7-methoxy-1,3-benzoxazol-5-yl)[(4R)-4-ethoxy-2-(hydroxymethyl)pyrrolidin-1-yl]methanone [enantiomerically pure isomer 2]

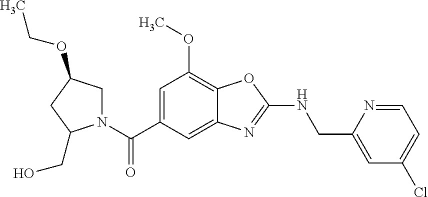 [SMILES: CCO[C@@H]1CC(CO)N(C(=O)c2cc(OC)c3oc(NCc4cc(Cl)ccn4)nc3c2)C1]

Diastereomer separation on a chiral phase of 83.0 mg of the compound from Example 1 according to Method 28D gave 46.0 mg of Example 2 (enantiomerically pure isomer 1) and 21.0 mg of Example 3 (enantiomerically pure isomer 2).

HPLC (Method 26E): Rₜ=10.9 min, >99.9% ee;

LC-MS (Method 1A): Rₜ=0.82 min; MS (ESIpos): m/z=461 [M+H]⁺;

¹H-NMR (400 MHz, DMSO-d₆): δ[ppm]=8.69 (t, 1H), 8.52 (d, 1H), 7.53 (d, 1H), 7.45 (dd, 1H), 6.96 (s, 1H), 6.81 (s, 1H), 4.80 (br. s., 1H), 4.63 (d, 2H), 4.23-4.06 (m, 1H), 3.91 (s, 4H), 3.58 (br. s., 2H), 2.25-1.88 (m, 2H), 1.06 (br. s., 3H), four protons obscured.

### Example 4

### (2-{[1-(4-Chloropyridin-2-yl)ethyl]amino}-7-methoxy-1,3-benzoxazol-5-yl)[(4R)-4-ethoxy-2-(hydroxymethyl)pyrrolidin-1-yl]methanone [diastereomer mixture, 2 isomers]

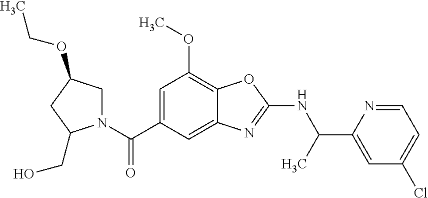 [SMILES: CCO[C@@H]1CC(CO)N(C(=O)c2cc(OC)c3oc(NC(C)c4cc(Cl)ccn4)nc3c2)C1]

200 mg (0.454 mmol, purity: 79%) of 2-{[1-(4-chloropyridin-2-yl)ethyl]amino}-7-methoxy-1,3-benzoxazole-5-carboxylic acid [racemate] and 99.0 mg (0.682 mmol) of [(4R)-4-ethoxypyrrolidin-2-yl]methanol [diastereomer mixture, 2 isomers] were initially charged in N,N-dimethylformamide (2.09 ml), and 206 mg (277 μl, 1.59 mmol) of N,N-diisopropylethylamine were added. 207 mg (0.545 mmol) of HATU were then added at RT, and the mixture was stirred for 1 h. Without further work-up, the reaction solution was then purified directly by preparative RP-HPLC (acetonitrile/water). Yield: 141 mg (65% of theory).

LC-MS (Method 1A): Rₜ=0.85 min (enantiomerically pure isomer 1), Rₜ=0.87 min (enantiomerically pure isomer 2);

MS (ESIpos): m/z=475 [M+H]⁺;

LC-MS (Method 2A): Rₜ=0.82 min; MS (ESIpos): m/z=475 [M+H]⁺.

### Example 5

### (2-{[1-(4-Chloropyridin-2-yl)ethyl]amino}-7-methoxy-1,3-benzoxazol-5-yl)[(4R)-4-ethoxy-2-(hydroxymethyl)pyrrolidin-1-yl]methanone [enantiomerically pure isomer 1]

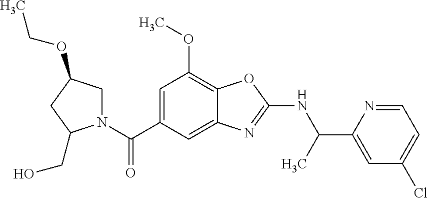 [SMILES: CCO[C@@H]1CC(CO)N(C(=O)c2cc(OC)c3oc(NC(C)c4cc(Cl)ccn4)nc3c2)C1]

Diastereomer separation on a chiral phase of 137 mg of the compound from Example 4 according to Method 29D gave 46.0 mg of Example 5 (enantiomerically pure isomer 1) and 48.0 mg of Example 6 (enantiomerically pure isomer 2); it was only possible to isolate the two isomers of the main pyrrolidine isomer from Example 4.

HPLC (Method 27E): Rₜ=6.39 min, >99.9% ee;

LC-MS (Method 1A): Rₜ=0.85 min; MS (ESIpos): m/z=475 [M+H]⁺;

¹H-NMR (400 MHz, DMSO-d₆): δ[ppm]=8.68 (d, 1H), 8.52 (d, 1H), 7.58 (d, 1H), 7.44 (dd, 1H), 6.97 (s, 1H), 6.82 (s, 1H), 4.98 (quin, 1H), 4.76 (t, 1H), 4.19 (br. s., 1H), 3.95 (br. s., 1H), 3.91 (s, 3H), 3.72-3.60 (m, 1H), 3.58-3.45 (m, 2H), 3.25-3.12 (m, 1H), 2.01 (br. d., 2H), 1.52 (d, 3H), 0.99 (t, 3H), two protons obscured.

### Example 6

### (2-{[(4-Chloropyridin-2-yl)methyl]amino}-7-methoxy-1,3-benzoxazol-5-yl)[(4R)-4-ethoxy-2-(hydroxymethyl)pyrrolidin-1-yl]methanone [enantiomerically pure isomer 2]

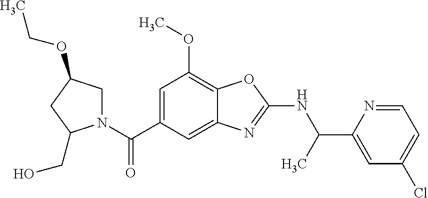 [SMILES: CCO[C@@H]1CC(CO)N(C(=O)c2cc(OC)c3oc(NC(C)c4cc(Cl)ccn4)nc3c2)C1]

Diastereomer separation on a chiral phase of 137 mg of the compound from Example 4 according to Method 29D gave 46.0 mg of Example 5 (enantiomerically pure isomer 1) and 48.0 mg of Example 6 (enantiomerically pure isomer 2); it was only possible to isolate the two isomers of the main pyrrolidine isomer from Example 4.

HPLC (Method 27E): Rₜ=8.23 min, >97% ee;

LC-MS (Method 1A): Rₜ=0.85 min; MS (ESIpos): m/z=475 [M+H]⁺;

¹H-NMR (400 MHz, DMSO-d₆): δ[ppm]=8.69 (d, 1H), 8.53 (d, 1H), 7.58 (d, 1H), 7.44 (dd, 1H), 6.96 (s, 1H), 6.82 (s, 1H), 4.99 (quin, 1H), 4.81-4.69 (m, 1H), 4.19 (br. s., 1H), 3.99-3.85 (m, 4H), 3.70-3.60 (m, 1H), 3.56-3.48 (d, 2H), 3.24-3.13 (m, 1H), 2.01 (br. d., 2H), 1.51 (d, 3H), 0.99 (t, 3H), two protons obscured.

### Example 7

### (2-{[(4-Chloropyridin-2-yl)methyl]amino}-7-methoxy-1,3-benzoxazol-5-yl)[(4R)-4-(2,2-difluoroethoxy)-2-(hydroxymethyl)pyrrolidin-1-yl]methanone [enantiomerically pure isomer 2]

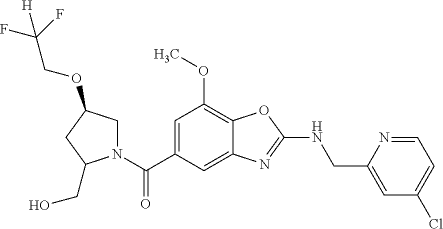 [SMILES: COc1cc(C(=O)N2C[C@H](OCC(F)F)CC2CO)cc2nc(NCc3cc(Cl)ccn3)oc12]

80.0 mg (0.194 mmol, purity: 81%) of 2-{[(4-chloropyridin-2-yl)methyl]amino}-7-methoxy-1,3-benzoxazole-5-carboxylic acid and 38.7 mg (0.214 mmol) of [(4R)-4-(2,2-difluoroethoxy)pyrrolidin-2-yl]methanol [enantiomerically pure isomer 2] were initially charged in N,N-dimethylformamide (0.84 ml), and 55.2 mg (74 μl, 0.427 mmol) of N,N-diisopropylethylamine were added. Subsequently, 88.6 mg (0.233 mmol) of HATU were added at RT and the mixture was stirred overnight. Without further work-up, the reaction solution was then purified directly by preparative RP-HPLC (acetonitrile/water). Yield: 46.1 mg (47% of theory).

¹H-NMR (400 MHz, DMSO-d₆): δ[ppm]=8.69 (t, 1H), 8.52 (d, 1H), 7.53 (s, 1H), 7.45 (dd, 1H), 6.99 (s, 1H), 6.84 (s, 1H), 6.03 (tt, 1H), 4.78 (t, 1H), 4.63 (d, 2H), 4.21 (br. s., 1H), 4.09 (br. s., 1H), 3.92 (s, 3H), 3.76-3.36 (m, 6H), 2.12-2.01 (m, 2H).

### Example 8

### (2-{[1-(4-Chloropyridin-2-yl)ethyl]amino}-7-methoxy-1,3-benzoxazol-5-yl)[(4R)-4-[(1,1-²H₂)ethyloxy]-2-(hydroxymethyl)pyrrolidin-1-yl]methanone [enantiomerically pure isomer 2]

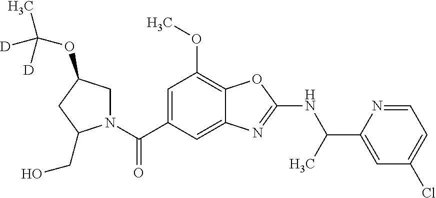 [SMILES: [2H]C([2H])(C)O[C@@H]1CC(CO)N(C(=O)c2cc(OC)c3oc(NC(C)c4cc(Cl)ccn4)nc3c2)C1]

Diastereomer separation on achiral phases of 247 mg of the compound from Example 97 according to Method 2F and further diastereomer separation on a chiral phase according to Method 29D gave 30.0 mg of Example 8 (enantiomerically pure isomer 2).

HPLC (Method 27E): Rₜ=10.1 min, >99.9% ee;

LC-MS (Method 1A): Rₜ=0.86 min; MS (ESIpos): m/z=477 [M+H]⁺;

¹H-NMR (400 MHz, DMSO-d₆): δ[ppm]=8.69 (d, 1H), 8.53 (d, 1H), 7.58 (s, 1H), 7.44 (dd, 1H), 6.96 (s, 1H), 6.82 (s, 1H), 4.99 (quin, 1H), 4.77 (br. s., 1H), 4.19 (br. s., 1H), 3.99-3.85 (m, 4H), 3.74-3.59 (m, 1H), 3.53 (d, 2H), 2.01 (d, 2H), 1.51 (d, 3H), 0.98 (s, 3H), one proton obscured.

### Example 9

### {(4R)-1-[(2-{[(4-Chloropyridin-2-yl)methyl]amino}-7-methoxy-1,3-benzoxazol-5-yl)carbonyl]-4-[(1,1-²H₂)ethyloxy]pyrrolidin-2-yl}acetonitrile [enantiomerically pure isomer]

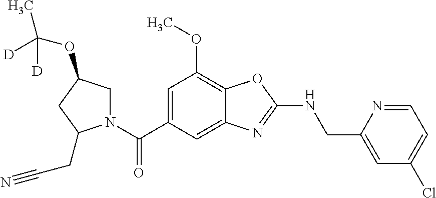 [SMILES: [2H]C([2H])(C)O[C@@H]1CC(CC#N)N(C(=O)c2cc(OC)c3oc(NCc4cc(Cl)ccn4)nc3c2)C1]

200 mg (0.599 mmol) of 2-{[(4-chloropyridin-2-yl)methyl]amino}-7-methoxy-1,3-benzoxazole-5-carboxylic acid and 85.1 mg (0.545 mmol) of {(4R)-4-[(1,1-²H₂)ethyloxy]pyrrolidin-2-yl}acetonitrile [diastereomer mixture, 2 isomers] were initially charged in N,N-dimethylformamide (2.51 ml), and 246 mg (332 μl, 1.91 mmol) of N,N-diisopropylethylamine were added. Subsequently, 249 mg (0.654 mmol) of HATU were added at RT and the mixture was stirred overnight. Without further work-up, the reaction solution was then purified directly by preparative RP-HPLC (acetonitrile/water). The crude product was then purified further according to Method 30D and again by preparative RP-HPLC (acetonitrile/water). Yield: 15.5 mg (5% of theory), it was only possible to isolate one isomer.

HPLC (Method 28E): Rₜ=16.8 min, >99.9% ee;

LC-MS (Method 2A): Rₜ=0.91 min; MS (ESIpos): m/z=472 [M+H]⁺;

¹H-NMR (400 MHz, DMSO-d₆): δ[ppm]=8.75 (t, 1H), 8.52 (d, 1H), 7.53 (d, 1H), 7.46 (dd, 1H), 6.97 (s, 1H), 6.84 (s, 1H), 4.63 (d, 2H), 4.28 (m_{c}, 1H), 4.02 (br. s., 1H), 3.93 (s, 3H), 3.69 (dd, 1H), 3.40 (d, 1H), 3.10 (dd, 1H), 2.98 (d, 1H), 2.34-2.23 (m, 1H), 1.90 (m_{c}, 1H), 0.99 (m, 3H).

### Example 10

### (2-{[(4-Chloropyridin-2-yl)methyl]amino}-7-methoxy-1,3-benzoxazol-5-yl){(2S,4R)-4-(cyclopropyloxy)-2-[(3,3-difluoroazetidin-1-yl)carbonyl]pyrrolidin-1-yl}methanone [enantiomerically pure isomer]

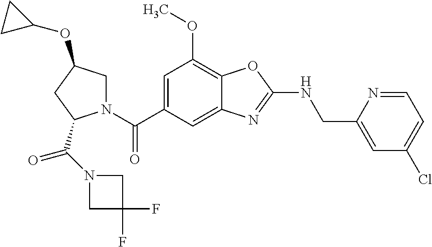 [SMILES: COc1cc(C(=O)N2C[C@H](OC3CC3)C[C@H]2C(=O)N2CC(F)(F)C2)cc2nc(NCc3cc(Cl)ccn3)oc12]

292 mg (crude product, purity: 26%) of (4R)-1-[(2-{[(4-chloropyridin-2-yl)methyl]amino}-7-methoxy-1,3-benzoxazol-5-yl)carbonyl]-4-(cyclopropyloxy)-L-proline [enantiomerically pure isomer, Example 26A] and 85.6 mg (0.661 mmol) of 3,3-difluoroazetidine hydrochloride were initially charged in N,N-dimethylformamide (4.90 ml), and 311 mg (419 μl, 2.40 mmol) of N,N-diisopropylethylamine were added. 274 mg (0.721 mmol) of HATU were then added at RT, and the mixture was stirred for 2 h. Without further work-up, the reaction solution was initially purified directly by preparative RP-HPLC (acetonitrile/water) and then once more by Method 3F. Yield: 33.8 mg (9% of theory).

LC-MS (Method 1A): Rₜ=0.86 min; MS (ESIpos): m/z=562 [M+H]⁺;

¹H-NMR (400 MHz, DMSO-d₆): δ[ppm]=8.73 (t, 1H), 8.52 (d, 1H), 7.53 (d, 1H), 7.46 (dd, 1H), 7.00 (d, 1H), 6.84 (d, 1H), 4.96 (m_{c}, 1H), 4.75 (m_{c}, 1H), 4.64 (d, 2H), 4.48-4.23 (m, 3H), 4.19 (br. s., 1H), 3.93 (m, 3H), 3.82 (dd, 1H), 3.55 (d, 1H), 3.24-3.13 (m, 1H), 2.38-2.29 (m, 1H), 2.02 (m_{c}, 1H), 0.59-0.29 (m, 4H).

### Example 11

### (2-{[(4-Chloropyridin-2-yl)methyl]amino}-7-methoxy-1,3-benzoxazol-5-yl){(2S,4R)-4-(cyclopropyloxy)-2-[(3-fluoroazetidin-1-yl)carbonyl]pyrrolidin-1-yl}methanone [enantiomerically pure isomer]

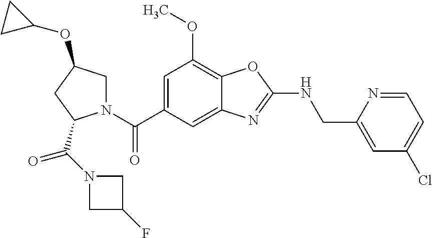 [SMILES: COc1cc(C(=O)N2C[C@H](OC3CC3)C[C@H]2C(=O)N2CC(F)C2)cc2nc(NCc3cc(Cl)ccn3)oc12]

292 mg (crude product, purity: 26%) of (4R)-1-[(2-{[(4-chloropyridin-2-yl)methyl]amino}-7-methoxy-1,3-benzoxazol-5-yl)carbonyl]-4-(cyclopropyloxy)-L-proline [enantiomerically pure isomer, Example 26A] and 73.7 mg (0.661 mmol) of 3-fluoroazetidine hydrochloride were initially charged in N,N-dimethylformamide (4.90 ml), and 311 mg (419 μl, 2.40 mmol) of N,N-diisopropylethylamine were added. 274 mg (0.721 mmol) of HATU were then added at RT, and the mixture was stirred for 2 h. Without further work-up, the reaction solution was initially purified directly by preparative RP-HPLC (acetonitrile/water) and then once more by Method 3F. Yield: 26.4 mg (8% of theory).

LC-MS (Method 1A): Rₜ=0.80 min; MS (ESIpos): m/z=544 [M+H]⁺;

¹H-NMR (400 MHz, DMSO-d₆): δ[ppm]=8.73 (t, 1H), 8.52 (d, 1H), 7.53 (s, 1H), 7.46 (dd, 1H), 6.99 (s, 1H), 6.83 (d, 1H), 5.61-5.29 (m, 1H), 4.92-4.07 (m, 7H), 3.98-3.88 (m, 4H), 3.79 (d, 1H), 3.54 (d, 1H), 3.20 (br. s., 1H), 2.35-2.22 (m, 1H), 1.99 (d, 1H), 0.57-0.27 (m, 4H).

### Example 12

### (2-{[(4-Chloropyridin-2-yl)methyl]amino}-7-methoxy-1,3-benzoxazol-5-yl)[(2S,4R)-4-(cyclopropyloxy)-2-(hydroxymethyl)pyrrolidin-1-yl]methanone [enantiomerically pure isomer]

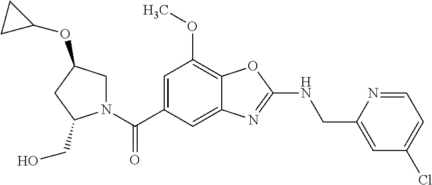 [SMILES: COc1cc(C(=O)N2C[C@H](OC3CC3)C[C@H]2CO)cc2nc(NCc3cc(Cl)ccn3)oc12]

60.0 mg (0.146 mmol, purity: 81%) of 2-{[(4-chloropyridin-2-yl)methyl]amino}-7-methoxy-1,3-benzoxazole-5-carboxylic acid and 26.5 mg (0.160 mmol) of [(2S,4R)-4-(cyclopropyloxy)pyrrolidin-2-yl]methanol [enantiomerically pure isomer] were initially charged in N,N-dimethylformamide (1.20 ml), and 75 mg (101 μl, 0.58 mmol) of N,N-diisopropylethylamine were added. Subsequently, 66 mg (0.18 mmol) of HATU were added at RT and the mixture was stirred overnight. Without further work-up, the reaction solution was then purified directly by preparative RP-HPLC (acetonitrile/water). Yield: 27.0 mg (37% of theory).

LC-MS (Method 1A): Rₜ=0.78 min; MS (ESIpos): m/z=473 [M+H]⁺;

¹H-NMR (400 MHz, DMSO-d₆): δ[ppm]=8.69 (t, 1H), 8.52 (d, 1H), 7.53 (s, 1H), 7.45 (dd, 1H), 7.00 (s, 1H), 6.86 (s, 1H), 4.77 (t, 1H), 4.63 (d, 2H), 4.18 (br. s., 1H), 4.06 (br. s., 1H), 3.92 (s, 3H), 3.73-3.41 (m, 4H), 3.09 (m_{c}, 1H), 2.07-2.03 (m, 2H), 0.47-0.23 (m, 4H).

### Example 13

### (2-{[1-(4-Chloropyridin-2-yl)ethyl]amino}-7-methoxy-1,3-benzoxazol-5-yl)[(2S,4R)-4-(cyclopropyloxy)-2-(hydroxymethyl)pyrrolidin-1-yl]methanone [diastereomer mixture, 2 isomers]

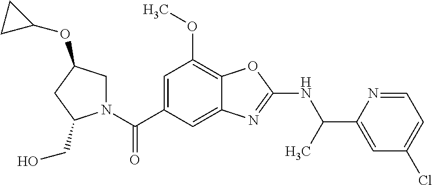 [SMILES: COc1cc(C(=O)N2C[C@H](OC3CC3)C[C@H]2CO)cc2nc(NC(C)c3cc(Cl)ccn3)oc12]

150 mg (0.341 mmol, purity: 79%) of 2-{[1-(4-chloropyridin-2-yl)ethyl]amino}-7-methoxy-1,3-benzoxazole-5-carboxylic acid [racemate] and 62.0 mg (0.375 mmol) of [(2S,4R)-4-(cyclopropyloxy)pyrrolidin-2-yl]methanol [enantiomerically pure isomer] were initially charged in N,N-dimethylformamide (1.48 ml), and 96.9 mg (131 μl, 0.750 mmol) of N,N-diisopropylethylamine were added. 155 mg (0.409 mmol) of HATU were then added at RT, and the mixture was stirred for 2 h. Without further work-up, the reaction solution was then purified directly by preparative RP-HPLC (acetonitrile/water). Yield: 70 mg (41% of theory).

LC-MS (Method 1A): Rₜ=0.88 min; MS (ESIpos): m/z=487 [M+H]⁺.

### Example 14

### (2-{[1-(4-Chloropyridin-2-yl)ethyl]amino}-7-methoxy-1,3-benzoxazol-5-yl)[(2S,4R)-4-(cyclopropyloxy)-2-(hydroxymethyl)pyrrolidin-1-yl]methanone [enantiomerically pure isomer 1]

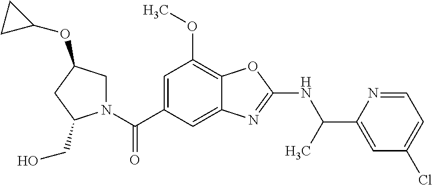 [SMILES: COc1cc(C(=O)N2C[C@H](OC3CC3)C[C@H]2CO)cc2nc(NC(C)c3cc(Cl)ccn3)oc12]

Diastereomer separation on a chiral phase of 55.5 mg of the compound from Example 13 according to Method 31D gave 16.3 mg of Example 14 (enantiomerically pure isomer 1) and 18.3 mg of Example 15 (enantiomerically pure isomer 2).

HPLC (Method 4E): Rₜ=5.81 min, >99.0% ee;

LC-MS (Method 2A): Rₜ=0.85 min; MS (ESIpos): m/z=487 [M+H]⁺;

¹H-NMR (400 MHz, DMSO-d₆): δ[ppm]=8.68 (d, 1H), 8.52 (d, 1H), 7.57 (d, 1H), 7.43 (dd, 1H), 6.99 (s, 1H), 6.84 (s, 1H), 4.98 (quin., 1H), 4.76 (t, 1H), 4.17 (br. s., 1H), 4.06 (br. s., 1H), 3.91 (s, 3H), 3.75-3.38 (m, 4H), 3.09 (br. s., 1H), 2.05 (d, 2H), 1.52 (d, 3H), 0.43-0.22 (m, 4H).

### Example 15

### (2-{[1-(4-Chloropyridin-2-yl)ethyl]amino}-7-methoxy-1,3-benzoxazol-5-yl)[(2S,4R)-4-(cyclopropyloxy)-2-(hydroxymethyl)pyrrolidin-1-yl]methanone [enantiomerically pure isomer 2]

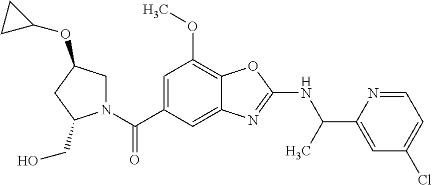 [SMILES: COc1cc(C(=O)N2C[C@H](OC3CC3)C[C@H]2CO)cc2nc(NC(C)c3cc(Cl)ccn3)oc12]

Diastereomer separation on a chiral phase of 55.5 mg of the compound from Example 13 according to Method 31D gave 16.3 mg of Example 14 (enantiomerically pure isomer 1) and 18.3 mg of Example 15 (enantiomerically pure isomer 2).

HPLC (Method 4E): Rₜ=7.08 min, >99.0% ee;

LC-MS (Method 2A): Rₜ=0.85 min; MS (ESIpos): m/z=487 [M+H]⁺;

¹H-NMR (400 MHz, DMSO-d₆): δ[ppm]=8.69 (d, 1H), 8.53 (d, 1H), 7.58 (d, 1H), 7.44 (dd, 1H), 6.97 (s, 1H), 6.84 (s, 1H), 4.99 (quin., 1H), 4.77 (t, 1H), 4.17 (br. s., 1H), 4.04 (br. s., 1H), 3.91 (s, 4H), 3.76-3.38 (m, 4H), 3.08 (br. s., 1H), 2.05 (d, 2H), 1.51 (d, 3H), 0.44-0.22 (m, 4H).

### Example 16

### (2-{[(4-Chloropyridin-2-yl)methyl]amino}-7-methoxy-1,3-benzoxazol-5-yl){2-[(3,3-difluoroazetidin-1-yl)carbonyl]-2-methylpyrrolidin-1-yl}methanone [enantiomerically pure isomer]

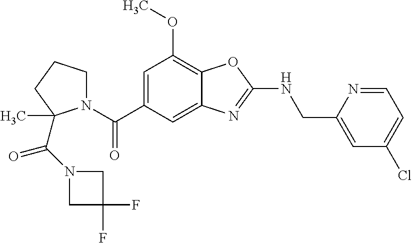 [SMILES: COc1cc(C(=O)N2CCCC2(C)C(=O)N2CC(F)(F)C2)cc2nc(NCc3cc(Cl)ccn3)oc12]

96.0 mg (crude product, purity: 71%) of 1-[(2-{[(4-chloropyridin-2-yl)methyl]amino}-7-methoxy-1,3-benzoxazol-5-yl)carbonyl]-2-methylproline [enantiomerically pure isomer] and 22.0 mg (0.170 mmol) of 3,3-difluoroazetidine hydrochloride were initially charged in N,N-dimethylformamide (2.36 ml), and 79.9 mg (108 μl, 618 mmol) of N,N-diisopropylethylamine were added. Subsequently, 70.5 mg (0.186 mmol) of HATU were added at RT and the mixture was stirred overnight. Without further work-up, the reaction solution was purified directly by preparative RP-HPLC (acetonitrile/water). Yield: 43.2 mg (53% of theory).

LC-MS (Method 3A): Rₜ=1.95 min; MS (ESIpos): m/z=520 [M+H]⁺;

¹H-NMR (400 MHz, DMSO-d₆): δ[ppm]=8.74 (t, 1H), 8.52 (d, 1H), 7.52 (d, 1H), 7.45 (dd, 1H), 7.07 (d, 1H), 6.80 (d, 1H), 4.63 (d, 2H), 4.35 (q, 2H), 3.93 (s, 3H), 3.73-3.60 (m, 1H), 3.58-3.48 (m, 1H), 2.19-2.08 (m, 1H), 1.97-1.78 (m, 3H), 1.53 (s, 3H), two protons obscured.

### Example 17

### (2-{[(4-Chloropyridin-2-yl)methyl]amino}-7-methoxy-1,3-benzoxazol-5-yl){2-[(3-fluoroazetidin-1-yl)carbonyl]-2-methylpyrrolidin-1-yl}methanone [enantiomerically pure isomer]

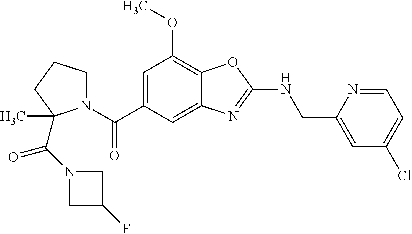 [SMILES: COc1cc(C(=O)N2CCCC2(C)C(=O)N2CC(F)C2)cc2nc(NCc3cc(Cl)ccn3)oc12]

96.0 mg (crude product, purity: 71%) of 1-[(2-{[(4-chloropyridin-2-yl)methyl]amino}-7-methoxy-1,3-benzoxazol-5-yl)carbonyl]-2-methylproline [enantiomerically pure isomer] and 19.0 mg (0.170 mmol) of 3-fluoroazetidine hydrochloride were initially charged in N,N-dimethylformamide (1.29 ml), and 79.9 mg (108 μl, 618 mmol) of N,N-diisopropylethylamine were added. Subsequently, 70.5 mg (0.186 mmol) of HATU were added at RT and the mixture was stirred overnight. Without further work-up, the reaction solution was purified directly by preparative RP-HPLC (acetonitrile/water). Yield: 34.7 mg (44% of theory).

LC-MS (Method 1A): Rₜ=0.82 min; MS (ESIpos): m/z=502 [M+H]⁺;

¹H-NMR (400 MHz, DMSO-d₆): δ[ppm]=8.73 (t, 1H), 8.52 (d, 1H), 7.52 (d, 1H), 7.45 (dd, 1H), 7.05 (d, 1H), 6.80 (d, 1H), 5.37 (dm_{c}, 1H), 4.63 (d, 2H), 4.42-3.85 (m, 7H), 3.68-3.58 (m, 1H), 3.57-3.49 (m, 1H), 2.15-2.04 (m, 1H), 1.98-1.80 (m, 3H), 1.51 (s, 3H).

### Example 18

### (2-{[(4-Chloropyridin-2-yl)methyl]amino}-7-methoxy-1,3-benzoxazol-5-yl) [2-methyl-2-(morpholin-4-ylcarbonyl)pyrrolidin-1-yl]methanone [enantiomerically pure isomer]

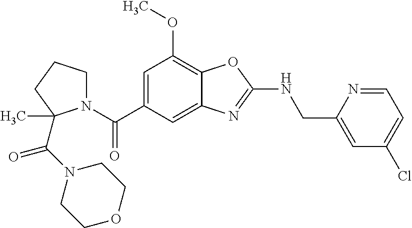 [SMILES: COc1cc(C(=O)N2CCCC2(C)C(=O)N2CCOCC2)cc2nc(NCc3cc(Cl)ccn3)oc12]

96.0 mg (crude product, purity: 71%) of 1-[(2-{[(4-chloropyridin-2-yl)methyl]amino}-7-methoxy-1,3-benzoxazol-5-yl)carbonyl]-2-methylproline [enantiomerically pure isomer] and 14.8 mg (0.170 mmol) of morpholine were initially charged in N,N-dimethylformamide (2.36 ml), and 79.9 mg (108 μl, 618 mmol) of N,N-diisopropylethylamine were added. Subsequently, 70.5 mg (0.186 mmol) of HATU were added at RT and the mixture was stirred overnight. Without further work-up, the reaction solution was purified directly by preparative RP-HPLC (acetonitrile/water).

Yield: 41.9 mg (52% of theory).

LC-MS (Method 1A): Rₜ=0.82 min; MS (ESIpos): m/z=513 [M+H]⁺;

¹H-NMR (400 MHz, DMSO-d₆): δ[ppm]=8.72 (t, 1H), 8.52 (d, 1H), 7.52 (d, 1H), 7.45 (dd, 1H), 6.97 (d, 1H), 6.71 (d, 1H), 4.62 (d, 2H), 3.92 (s, 3H), 3.81-3.67 (m, 1H), 3.61-3.37 (m, 9H), 2.19-1.73 (m, 4H), 1.52 (s, 3H).

### Example 19

### (2-{[(4-Chloropyridin-2-yl)methyl]amino}-7-methoxy-1,3-benzoxazol-5-yl) [5-(hydroxymethyl)-2,5-dimethylmorpholin-4-yl]methanone [enantiomerically pure isomer 1]

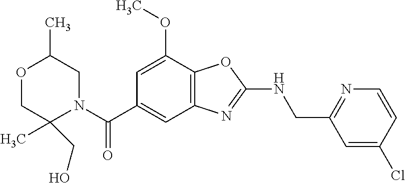 [SMILES: COc1cc(C(=O)N2CC(C)OCC2(C)CO)cc2nc(NCc3cc(Cl)ccn3)oc12]

60.0 mg (0.169 mmol) of 2-{[(4-chloropyridin-2-yl)methyl]amino}-7-methoxy-1,3-benzoxazole-5-carboxylic acid and 29.4 mg (0.203 mmol) of (3,6-dimethylmorpholin-3-yl)methanol [enantiomerically pure isomer 1, Example 40A] were initially charged in N,N-dimethylformamide (0.78 ml), and 76.4 mg (103 μl, 0.591 mmol) of N,N-diisopropylethylamine were added. Subsequently, 77.1 mg (0.203 mmol) of HATU were added at RT and the mixture was stirred overnight. Without further work-up, the reaction solution was then purified directly by preparative RP-HPLC (acetonitrile/water). Yield: 22.2 mg (28% of theory).

LC-MS (Method 1A): Rₜ=0.83 min; MS (ESIpos): m/z=461 [M+H]⁺;

¹H-NMR (400 MHz, DMSO-d₆): δ [ppm]=8.69 (t, 1H), 8.51 (d, 1H), 7.52 (d, 1H), 7.45 (dd, 1H), 6.86 (d, 1H), 6.71 (d, 1H), 4.87 (t, 1H), 4.62 (d, 2H), 3.94-3.85 (m, 4H), 3.81-3.64 (m, 3H), 3.38 (dd, 1H), 3.21 (d, 1H), 2.87 (dd, 1H), 1.34 (s, 3H), 0.97 (d, 3H).

### Example 20

### (2-{[(4-Chloropyridin-2-yl)methyl]amino}-7-methoxy-1,3-benzoxazol-5-yl) [5-(hydroxymethyl)-2,5-dimethylmorpholin-4-yl]methanone [enantiomerically pure isomer 4]

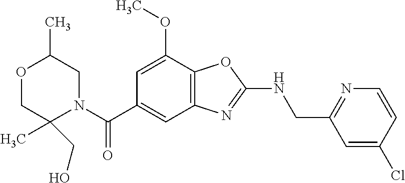 [SMILES: COc1cc(C(=O)N2CC(C)OCC2(C)CO)cc2nc(NCc3cc(Cl)ccn3)oc12]

60.0 mg (0.169 mmol) of 2-{[(4-chloropyridin-2-yl)methyl]amino}-7-methoxy-1,3-benzoxazole-5-carboxylic acid and 29.4 mg (0.203 mmol) of (3,6-dimethylmorpholin-3-yl)methanol [enantiomerically pure isomer 4, Example 41A] were initially charged in N,N-dimethylformamide (0.78 ml), and 76.4 mg (103 μl, 0.591 mmol) of N,N-diisopropylethylamine were added. Subsequently, 77.1 mg (0.203 mmol) of HATU were added at RT and the mixture was stirred overnight. Without further work-up, the reaction solution was then purified directly by preparative RP-HPLC (acetonitrile/water). Yield: 28.1 mg (36% of theory).

LC-MS (Method 1A): Rₜ=0.87 min; MS (ESIpos): m/z=461 [M+H]⁺;

¹H-NMR (400 MHz, DMSO-d₆): 8 [ppm]=8.70 (t, 1H), 8.52 (d, 1H), 7.52 (d, 1H), 7.46 (dd, 1H), 6.86 (d, 1H), 6.71 (s, 1H), 4.81 (t, 1H), 4.63 (d, 2H), 3.91 (s, 3H), 3.79 (d, 2H), 3.65 (d, 2H), 3.53 (d, 1H), 2.91 (dd, 1H), 1.32 (s, 3H), 0.98 (d, 3H), one proton obscured.

### Example 21

### (2-{[(4-Chloropyridin-2-yl)methyl]amino}-7-methoxy-1,3-benzoxazol-5-yl) [5-(fluoromethyl)-2-(2-hydroxyethyl)-2-methylmorpholin-4-yl]methanone [diastereomer mixture, 4 isomers]

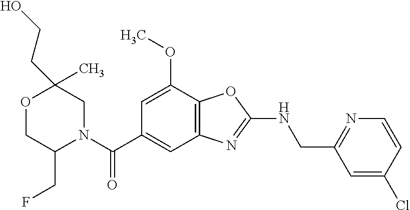 [SMILES: COc1cc(C(=O)N2CC(C)(CCO)OCC2CF)cc2nc(NCc3cc(Cl)ccn3)oc12]

200 mg (0.563 mmol) of 2-{[(4-chloropyridin-2-yl)methyl]amino}-7-methoxy-1,3-benzoxazole-5-carboxylic acid and 120 mg (0.676 mmol) of 2-[5-(fluoromethyl)-2-methylmorpholin-2-yl]ethanol [diastereomer mixture, 4 isomers] were initially charged in N,N-dimethylformamide (2.67 ml), and 255 mg (343 μl, 1.97 mmol) of N,N-diisopropylethylamine were added. Subsequently, 257 mg (0.676 mmol) of HATU were added at RT and the mixture was stirred overnight. Without further work-up, the reaction solution was then purified directly by preparative RP-HPLC (acetonitrile/water). Yield: 138 mg (49% of theory).

LC-MS (Method 1A): Rₜ=0.80 min; MS (ESIpos): m/z=493 [M+H]⁺.

### Example 22

### (2-{[(4-Chloropyridin-2-yl)methyl]amino}-7-methoxy-1,3-benzoxazol-5-yl) [5-(fluoromethyl)-2-(2-hydroxypropyl)-2-methylmorpholin-4-yl]methanone [racemate]

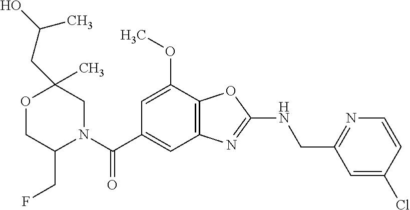 [SMILES: COc1cc(C(=O)N2CC(C)(CC(C)O)OCC2CF)cc2nc(NCc3cc(Cl)ccn3)oc12]

92.1 mg (0.276 mmol) of 2-{[(4-chloropyridin-2-yl)methyl]amino}-7-methoxy-1,3-benzoxazole-5-carboxylic acid and 101 mg (0.331 mmol) of 1-[5-(fluoromethyl)-2-methylmorpholin-2-yl]propan-2-ol trifluoroacetate [racemate] were initially charged in N,N-dimethylformamide (1.27 ml), and 125 mg (168 μl, 0,965 mmol) of N,N-diisopropylethylamine were added. Subsequently, 126 mg (0.331 mmol) of HATU were added at RT and the mixture was stirred overnight. Without further work-up, the reaction solution was then purified directly by preparative RP-HPLC (acetonitrile/water). Yield: 57.6 mg (41% of theory).

LC-MS (Method 1A): Rₜ=0.84 min; MS (ESIpos): m/z=507 [M+H]⁺.

### Example 23

### (2-{[(4-Chloropyridin-2-yl)methyl]amino}-7-methoxy-1,3-benzoxazol-5-yl) [5-(fluoromethyl)-2-(2-hydroxypropyl)-2-methylmorpholin-4-yl]methanone [enantiomerically pure isomer 1]

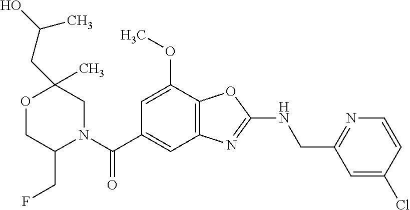 [SMILES: COc1cc(C(=O)N2CC(C)(CC(C)O)OCC2CF)cc2nc(NCc3cc(Cl)ccn3)oc12]

Enantiomer separation on a chiral phase of 52 mg of the compound from Example 22 according to Method 33D gave 19.6 mg of Example 23 (enantiomerically pure isomer 1).

HPLC (Method 30E): Rₜ=9.64 min, >99.0% ee;

LC-MS (Method 1A): Rₜ=0.84 min; MS (ESIpos): m/z=507 [M+H]⁺.

### Example 24

### (2-{[(4-Chloropyridin-2-yl)methyl]amino}-7-methoxy-1,3-benzoxazol-5-yl)[(5R)-2-(2-hydroxyethyl)-2,5-dimethylmorpholin-4-yl]methanone [enantiomerically pure isomer]

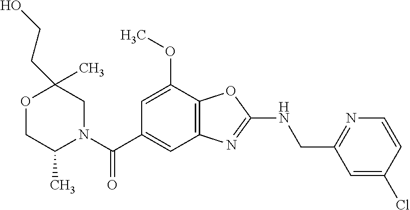 [SMILES: COc1cc(C(=O)N2CC(C)(CCO)OC[C@H]2C)cc2nc(NCc3cc(Cl)ccn3)oc12]

100 mg (0.282 mmol) of 2-{[(4-chloropyridin-2-yl)methyl]amino}-7-methoxy-1,3-benzoxazole-5-carboxylic acid and 53.8 mg (0.338 mmol) of 2-[(5R)-2,5-dimethylmorpholin-2-yl]ethanol [enantiomerically pure isomer] were initially charged in N,N-dimethylformamide (1.30 ml), and 127 mg (172 μl, 0.986 mmol) of N,N-diisopropylethylamine were added. Subsequently, 129 mg (0.338 mmol) of HATU were added at RT and the mixture was stirred overnight. Without further work-up, the reaction solution was then purified directly by preparative RP-HPLC (acetonitrile/water). Yield: 96.6 mg (72% of theory).

LC-MS (Method 1A): Rₜ=0.77 min; MS (ESIpos): m/z=475 [M+H]⁺;

¹H-NMR (400 MHz, DMSO-d₆): δ[ppm]=8.69 (t, 1H), 8.52 (d, 1H), 7.53 (d, 1H), 7.45 (dd, 1H), 6.82 (s, 1H), 6.67 (s, 1H), 4.63 (d, 2H), 4.30 (t, 1H), 3.92 (s, 3H), 3.74 (dd, 1H), 3.38 (br. s., 2H), 2.96 (br. s., 1H), 2.02 (m_{c}, 1H), 1.45 (br. s., 1H), 1.22 (d, 3H), 1.08 (br. s., 3H), three protons obscured.

### Example 25

### (2-{[(4-Chloropyridin-2-yl)methyl]amino}-7-methoxy-1,3-benzoxazol-5-yl)[(5R)-2-(2-hydroxypropyl)-2,5-dimethylmorpholin-4-yl]methanone [enantiomerically pure isomer]

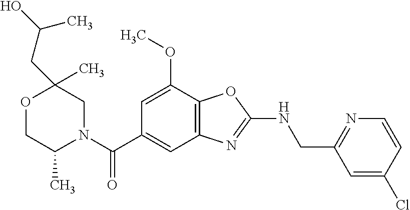 [SMILES: COc1cc(C(=O)N2CC(C)(CC(C)O)OC[C@H]2C)cc2nc(NCc3cc(Cl)ccn3)oc12]

80.0 mg (0.225 mmol) of 2-{[(4-chloropyridin-2-yl)methyl]amino}-7-methoxy-1,3-benzoxazole-5-carboxylic acid and 46.8 mg (0.270 mmol) of 1-[(5R)-2,5-dimethylmorpholin-2-yl]propan-2-ol [enantiomerically pure isomer] were initially charged in N,N-dimethylformamide (1.27 ml), and 102 mg (137 μl, 0.789 mmol) of N,N-diisopropylethylamine were added. Subsequently, 103 mg (0.270 mmol) of HATU were added at RT and the mixture was stirred overnight. Without further work-up, the reaction solution was then purified directly by preparative RP-HPLC (acetonitrile/water). Yield: 80.5 mg (71% of theory).

LC-MS (Method 1A): Rₜ=0.82 min; MS (ESIpos): m/z=489 [M+H]⁺;

¹H-NMR (400 MHz, DMSO-d₆): δ[ppm]=8.68 (t, 1H), 8.51 (d, 1H), 7.52 (d, 1H), 7.45 (dd, 1H), 6.81 (s, 1H), 6.66 (d, 1H), 4.63 (d, 2H), 4.23 (d, 1H), 3.92 (s, 3H), 3.78-3.67 (m, 2H), 3.29 (br. s., 1H), 2.96 (br. d., 1H), 1.91 (dd, 1H), 1.35-1.25 (m, 1H), 1.21 (d, 3H), 1.16 (br. s., 3H), 1.10 (d, 3H), one proton obscured.

### Example 26

### (2-{[1-(4-Chloropyridin-2-yl)ethyl]amino}-7-methoxy-1,3-benzoxazol-5-yl)[(5R)-2-(2-hydroxypropyl)-2,5-dimethylmorpholin-4-yl]methanone [diastereomer mixture, 2 isomers]

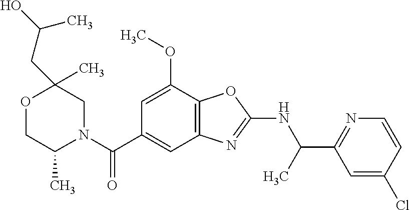 [SMILES: COc1cc(C(=O)N2CC(C)(CC(C)O)OC[C@H]2C)cc2nc(NC(C)c3cc(Cl)ccn3)oc12]

120 mg (0.276 mmol, purity: 80%) of 2-{[1-(4-chloropyridin-2-yl)ethyl]amino}-7-methoxy-1,3-benzoxazole-5-carboxylic acid [racemate] and 57.4 mg (0.331 mmol) of 1-[(5R)-2,5-dimethylmorpholin-2-yl]propan-2-ol [enantiomerically pure isomer] were initially charged in N,N-dimethylformamide (1.27 ml), and 125 mg (168 μl, 0.966 mmol) of N,N-diisopropylethylamine were added. Subsequently, 126 mg (0.331 mmol) of HATU were added at RT and the mixture was stirred overnight. Without further work-up, the reaction solution was then purified directly by preparative RP-HPLC (acetonitrile/water) and subsequently by Method 4F. Yield: 30.2 mg (19% of theory).

LC-MS (Method 1A): Rₜ=0.89 min; MS (ESIpos): m/z=503 [M+H]⁺.

### Example 27

### (2-{[1-(4-Chloropyridin-2-yl)ethyl]amino}-7-methoxy-1,3-benzoxazol-5-yl)[(5R)-2-(2-hydroxypropyl)-2,5-dimethylmorpholin-4-yl]methanone [enantiomerically pure isomer 2]

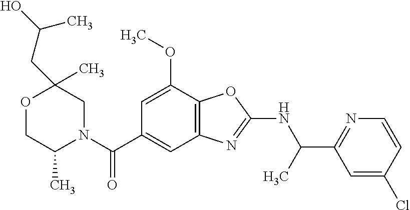 [SMILES: COc1cc(C(=O)N2CC(C)(CC(C)O)OC[C@H]2C)cc2nc(NC(C)c3cc(Cl)ccn3)oc12]

Diastereomer separation on a chiral phase of 25 mg of the compound from Example 26 according to Method 34D and further purification according to Method 5F gave 5.3 mg of Example 27 (enantiomerically pure isomer 2).

HPLC (Method 31E): Rₜ=8.00 min, >99.0% ee;

LC-MS (Method 1A): Rₜ=0.91 min; MS (ESIpos): m/z=503 [M+H]⁺;

¹H-NMR (400 MHz, DMSO-d₆): δ[ppm]=8.70 (d, 1H), 8.52 (d, 1H), 7.57 (d, 1H), 7.44 (dd, 1H), 6.78 (s, 1H), 6.65 (s, 1H), 4.98 (quin, 1H), 3.91 (s, 3H), 3.78-3.63 (m, 2H), 2.95 (br. s., 1H), 1.91 (dd, 1H), 1.51 (d, 3H), 1.35-1.12 (m, 7H), 1.09 (d, 3H), four protons obscured.

### Example 28

### (2-{[1-(4-Chloropyridin-2-yl)ethyl]amino}-7-methoxy-1,3-benzoxazol-5-yl)[(5R)-5-ethyl-2-(2-hydroxyethyl)-2-methylmorpholin-4-yl]methanone [diastereomer mixture, 2 isomers]

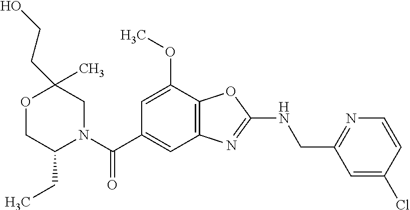 [SMILES: CC[C@@H]1COC(C)(CCO)CN1C(=O)c1cc(OC)c2oc(NCc3cc(Cl)ccn3)nc2c1]

200 mg (0.599 mmol) of 2-{[(4-chloropyridin-2-yl)methyl]amino}-7-methoxy-1,3-benzoxazole-5-carboxylic acid and 145 mg (0.840 mmol) of 2-[(5R)-5-ethyl-2-methylmorpholin-2-yl]ethanol [diastereomer mixture, 2 isomers] were initially charged in N,N-dimethylformamide (2.76 ml), and 271 mg (365 μl, 2.10 mmol) of N,N-diisopropylethylamine were added. Subsequently, 273 mg (0.719 mmol) of HATU were added at RT and the mixture was stirred overnight. Without further work-up, the reaction solution was then purified directly by preparative RP-HPLC (acetonitrile/water). Yield: 196 mg (64% of theory).

LC-MS (Method 1A): Rₜ=0.83 min (enantiomerically pure isomer 1), Rₜ=0.85 min (enantiomerically pure isomer 2);

MS (ESIpos): m/z=489 [M+H]⁺.

### Example 29

### (2-{[1-(4-Chloropyridin-2-yl)ethyl]amino}-7-methoxy-1,3-benzoxazol-5-yl)[(5R)-5-ethyl-2-(2-hydroxyethyl)-2-methylmorpholin-4-yl]methanone [enantiomerically pure isomer 1]

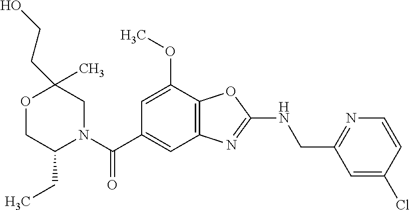 [SMILES: CC[C@@H]1COC(C)(CCO)CN1C(=O)c1cc(OC)c2oc(NCc3cc(Cl)ccn3)nc2c1]

Diastereomer separation on a chiral phase of 195 mg of the compound from Example 28 according to Method 35D and further purification by preparative RP-HPLC (acetonitrile/water) gave 21 mg of Example 29 (enantiomerically pure isomer 1).

HPLC (Method 1E): Rₜ=9.78 min, >99.9% ee;

LC-MS (Method 1A): Rₜ=0.79 min; MS (ESIpos): m/z=489 [M+H]⁺;

¹H-NMR (400 MHz, DMSO-d₆): δ[ppm]=8.72 (t, 1H), 8.52 (d, 1H), 7.53 (d, 1H), 7.46 (dd, 1H), 6.81 (br. s., 1H), 6.65 (s, 1H), 4.63 (d, 2H), 4.33 (br. s., 1H), 3.91 (s, 3H), 3.82 (d, 1H), 3.49 (br. s., 3H), 1.79 (m_{c}, 1H), 1.60 (br. s., 3H), 1.13 (br. s., 3H), 0.83 (br. s., 3H), three protons obscured.

### Example 30

### ((2-{[(4-Chloropyridin-2-yl)methyl]amino}-7-methoxy-1,3-benzoxazol-5-yl)[2-(2-hydroxyethyl)-5-(methoxymethyl)-2-methylmorpholin-4-yl]methanone [diastereomer mixture, 4 isomers]

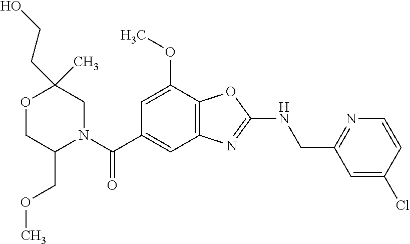 [SMILES: COCC1COC(C)(CCO)CN1C(=O)c1cc(OC)c2oc(NCc3cc(Cl)ccn3)nc2c1]

300 mg (0.899 mmol) of 2-{[(4-chloropyridin-2-yl)methyl]amino}-7-methoxy-1,3-benzoxazole-5-carboxylic acid and 221 mg (1.17 mmol) of 2-[5-(methoxymethyl)-2-methylmorpholin-2-yl]ethanol [diastereomer mixture, 4 isomers] were initially charged in N,N-dimethylformamide (4.14 ml), and 407 mg (548 μl, 3.15 mmol) of N,N-diisopropylethylamine were added. Subsequently, 410 mg (1.08 mmol) of HATU were added at RT and the mixture was stirred overnight. Without further work-up, the reaction solution was then purified directly by preparative RP-HPLC (acetonitrile/water). Yield: 277 mg (61% of theory).

LC-MS (Method 1A): Rₜ=0.80 min; MS (ESIpos): m/z=505 [M+H]⁺.

### Example 31

### ((2-{[(4-Chloropyridin-2-yl)methyl]amino}-7-methoxy-1,3-benzoxazol-5-yl)[2-(2-hydroxyethyl)-5-(methoxymethyl)-2-methylmorpholin-4-yl]methanone [enantiomerically pure isomer 1]

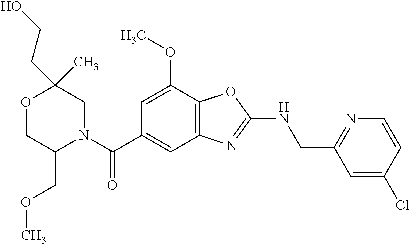 [SMILES: COCC1COC(C)(CCO)CN1C(=O)c1cc(OC)c2oc(NCc3cc(Cl)ccn3)nc2c1]

Diastereomer separation on a chiral phase of 270 mg of the compound from Example 30 according to Method 36D gave 21 mg of Example 31 (enantiomerically pure isomer 1).

HPLC (Method 32E): Rₜ=4.68 min, >99.9% ee;

LC-MS (Method 1A): Rₜ=0.73 min; MS (ESIpos): m/z=505 [M+H]⁺.

### Example 32

### (2-{[(4-Chloropyridin-2-yl)methyl]amino}-7-methoxy-1,3-benzoxazol-5-yl) [2-(2-hydroxyethyl)-2,5,5-trimethylmorpholin-4-yl]methanone [racemate]

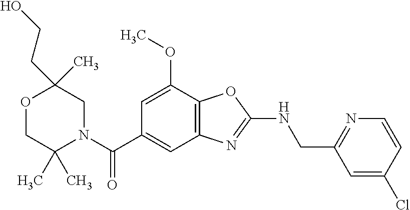 [SMILES: COc1cc(C(=O)N2CC(C)(CCO)OCC2(C)C)cc2nc(NCc3cc(Cl)ccn3)oc12]

120 mg (0.338 mmol) of 2-{[(4-chloropyridin-2-yl)methyl]amino}-7-methoxy-1,3-benzoxazole-5-carboxylic acid and 70.2 mg (0.406 mmol) of 2-(2,5,5-trimethylmorpholin-2-yl)ethanol [racemate] were initially charged in N,N-dimethylformamide (1.56 ml), and 153 mg (206 μl, 1.18 mmol) of N,N-diisopropylethylamine were added. Subsequently, 154 mg (0.406 mmol) of HATU were added at RT and the mixture was stirred overnight. Without further work-up, the reaction solution was then purified directly by preparative RP-HPLC (acetonitrile/water). Yield: 60.6 mg (34% of theory).

LC-MS (Method 1A): Rₜ=0.85 min; MS (ESIpos): m/z=489 [M+H]⁺;

¹H-NMR (400 MHz, DMSO-d₆): δ[ppm]=8.66 (t, 1H), 8.51 (d, 1H), 7.53 (d, 1H), 7.45 (dd, 1H), 6.75 (d, 1H), 6.61 (d, 1H), 4.63 (d, 2H), 4.26 (t, 1H), 3.90 (s, 3H), 3.45-3.34 (s, 4H), 3.22 (d, 1H), 3.12 (d, 1H), 1.74 (m_{c}, 1H), 1.54-1.37 (m, 7H), 1.08 (s, 3H).

### Example 33

### (2-{[(4-Chloropyridin-2-yl)methyl]amino}-7-methoxy-1,3-benzoxazol-5-yl) [2-(2-hydroxyethyl)-2,5,5-trimethylmorpholin-4-yl]methanone [enantiomerically pure isomer 2]

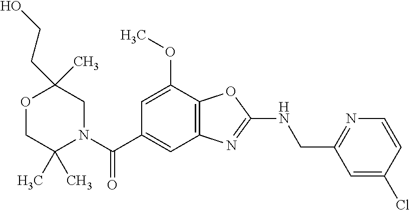 [SMILES: COc1cc(C(=O)N2CC(C)(CCO)OCC2(C)C)cc2nc(NCc3cc(Cl)ccn3)oc12]

Enantiomer separation on a chiral phase of 55.0 mg of the compound from Example 32 according to Method 37D gave 24.4 mg of Example 33 (enantiomerically pure isomer 2).

HPLC (Method 33E): Rₜ=6.74 min, >99.0% ee;

LC-MS (Method 1A): Rₜ=0.81 min; MS (ESIpos): m/z=489 [M+H]⁺;

¹H-NMR (400 MHz, DMSO-d₆): δ[ppm]=8.66 (t, 1H), 8.51 (d, 1H), 7.53 (d, 1H), 7.45 (dd, 1H), 6.75 (d, 1H), 6.61 (d, 1H), 4.63 (d, 2H), 4.29 (t, 1H), 3.90 (s, 3H), 3.45-3.34 (s, 4H), 3.22 (d, 1H), 3.12 (d, 1H), 1.74 (m_{c}, 1H), 1.54-1.37 (m, 7H), 1.08 (s, 3H).

### Example 34

### (2-{[(4-Chloropyridin-2-yl)methyl]amino}-7-methoxy-1,3-benzoxazol-5-yl) [2-(2-hydroxypropyl)-2,5,5-trimethylmorpholin-4-yl]methanone [racemate]

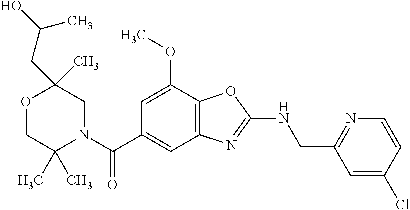 [SMILES: COc1cc(C(=O)N2CC(C)(CC(C)O)OCC2(C)C)cc2nc(NCc3cc(Cl)ccn3)oc12]

120 mg (0.338 mmol) of 2-{[(4-chloropyridin-2-yl)methyl]amino}-7-methoxy-1,3-benzoxazole-5-carboxylic acid and 76.0 mg (0.406 mmol) of 1-[6,6-dimethylmorpholin-3-yl]propan-2-ol [diastereomer mixture, 4 isomers] were initially charged in N,N-dimethylformamide (1.56 ml), and 153 mg (206 μl, 1.18 mmol) of N,N-diisopropylethylamine were added. Subsequently, 154 mg (0.406 mmol) of HATU were added at RT and the mixture was stirred overnight. Without further work-up, the reaction solution was then purified directly by preparative RP-HPLC (acetonitrile/water). Yield: 53.9 mg (31% of theory).

LC-MS (Method 1A): Rₜ=0.91 min; MS (ESIpos): m/z=503 [M+H]⁺;

¹H-NMR (400 MHz, DMSO-d₆): δ[ppm]=8.64 (t, 1H), 8.51 (d, 1H), 7.53 (d, 1H), 7.45 (dd, 1H), 6.74 (d, 1H), 6.61 (d, 1H), 4.62 (d, 2H), 4.13 (d, 1H), 3.90 (s, 3H), 3.71 (br. s., 1H), 3.40 (m_{c}, 2H), 3.22 (m_{c}, 2H), 1.68-1.32 (m, 8H), 1.16 (s, 3H), 1.03 (d, 3H).

### Example 35

### (2-{[(4-Chloropyridin-2-yl)methyl]amino}-7-methoxy-1,3-benzoxazol-5-yl) [2-(2-hydroxypropyl)-2,5,5-trimethylmorpholin-4-yl]methanone [enantiomerically pure isomer 1]

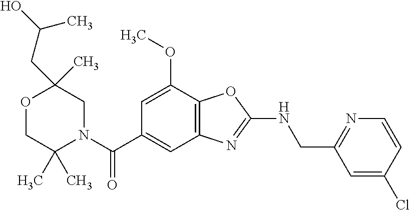 [SMILES: COc1cc(C(=O)N2CC(C)(CC(C)O)OCC2(C)C)cc2nc(NCc3cc(Cl)ccn3)oc12]

Enantiomer separation on a chiral phase of 48.0 mg of the compound from Example 34 according to Method 31D gave 24.4 mg of Example 35 (enantiomerically pure isomer 1).

HPLC (Method 4E): Rₜ=5.96 min, >99.9% ee;

LC-MS (Method 1A): Rₜ=0.91 min; MS (ESIpos): m/z=503 [M+H]⁺;

¹H-NMR (400 MHz, DMSO-d₆): δ[ppm]=8.67 (t, 1H), 8.51 (d, 1H), 7.53 (d, 1H), 7.45 (dd, 1H), 6.74 (d, 1H), 6.61 (d, 1H), 4.62 (d, 2H), 4.15 (d, 1H), 3.90 (s, 3H), 3.71 (br. s., 1H), 3.40 (m_{c}, 2H), 3.22 (m_{c}, 2H), 1.68-1.32 (m, 8H), 1.16 (s, 3H), 1.03 (d, 3H).

### Example 36

### (2-{[(4-Chloropyridin-2-yl)methyl]amino}-7-methoxy-1,3-benzoxazol-5-yl) [5-(2-hydroxypropan-2-yl)-2-methylmorpholin-4-yl]methanone [enantiomerically pure isomer 1]

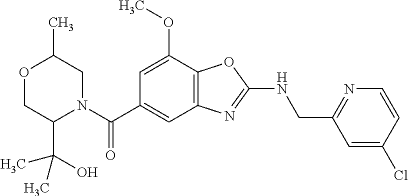 [SMILES: COc1cc(C(=O)N2CC(C)OCC2C(C)(C)O)cc2nc(NCc3cc(Cl)ccn3)oc12]

87.3 mg (0.246 mmol) of 2-{[(4-chloropyridin-2-yl)methyl]amino}-7-methoxy-1,3-benzoxazole-5-carboxylic acid and 47.0 mg (0.295 mmol) of 2-(6-methylmorpholin-3-yl)propan-2-ol [enantiomerically pure isomer 1, Example 99A] were initially charged in N,N-dimethylformamide (1.13 ml), and 111 mg (150 μl, 0.861 mmol) of N,N-diisopropylethylamine were added. Subsequently, 112 mg (0.295 mmol) of HATU were added at RT and the mixture was stirred overnight. Without further work-up, the reaction solution was then purified directly by preparative RP-HPLC (acetonitrile/water). Yield: 88.4 mg (73% of theory).

LC-MS (Method 1A): Rₜ=0.81 min; MS (ESIpos): m/z=475 [M+H]⁺.

### Example 37

### (2-{[(4-Chloropyridin-2-yl)methyl]amino}-7-methoxy-1,3-benzoxazol-5-yl) [5-(2-hydroxypropan-2-yl)-2-methylmorpholin-4-yl]methanone [enantiomerically pure isomer 2]

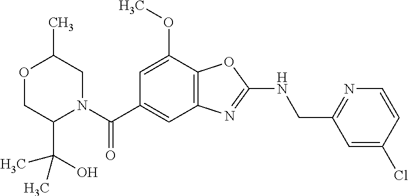 [SMILES: COc1cc(C(=O)N2CC(C)OCC2C(C)(C)O)cc2nc(NCc3cc(Cl)ccn3)oc12]

108 mg (0.304 mmol) of 2-{[(4-chloropyridin-2-yl)methyl]amino}-7-methoxy-1,3-benzoxazole-5-carboxylic acid and 58.0 mg (0.364 mmol) of 2-(6-methylmorpholin-3-yl)propan-2-ol [enantiomerically pure isomer 2, Example 100A] were initially charged in N,N-dimethylformamide (1.40 ml), and 137 mg (185 μl, 1.06 mmol) of N,N-diisopropylethylamine were added. Subsequently, 139 mg (0.364 mmol) of HATU were added at RT and the mixture was stirred overnight. Without further work-up, the reaction solution was then purified directly by preparative RP-HPLC (acetonitrile/water). Yield: 94.6 mg (65% of theory).

LC-MS (Method 1A): Rₜ=0.81 min; MS (ESIpos): m/z=475 [M+H]⁺.

### Example 38

### (2-{[(4-Chloropyridin-2-yl)methyl]amino}-7-methoxy-1,3-benzoxazol-5-yl) [5-(hydroxymethyl)-2,2,5-trimethylmorpholin-4-yl]methanone [racemate]

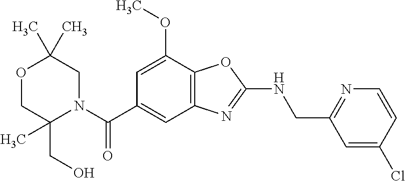 [SMILES: COc1cc(C(=O)N2CC(C)(C)OCC2(C)CO)cc2nc(NCc3cc(Cl)ccn3)oc12]

150 mg (0.449 mmol) of 2-{[(4-chloropyridin-2-yl)methyl]amino}-7-methoxy-1,3-benzoxazole-5-carboxylic acid and 85.9 mg (0.539 mmol) of (3,6,6-trimethylmorpholin-3-yl)methanol [racemate] were initially charged in N,N-dimethylformamide (2.07 ml), and 203 mg (274 μl, 1.57 mmol) of N,N-diisopropylethylamine were added. Subsequently, 205 mg (0.539 mmol) of HATU were added at RT and the mixture was stirred overnight. Without further work-up, the reaction solution was then purified directly by preparative RP-HPLC (acetonitrile/water). Yield: 45.0 mg (21% of theory).

LC-MS (Method 1A): Rₜ=0.88 min; MS (ESIpos): m/z=475 [M+H]⁺.

### Example 39

### (2-{[(4-Chloropyridin-2-yl)methyl]amino}-7-methoxy-1,3-benzoxazol-5-yl) [5-(hydroxymethyl)-2,2,5-trimethylmorpholin-4-yl]methanone [enantiomerically pure isomer 1]

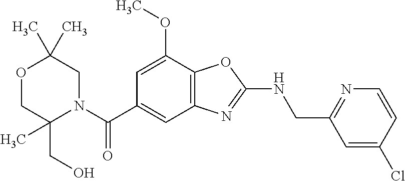 [SMILES: COc1cc(C(=O)N2CC(C)(C)OCC2(C)CO)cc2nc(NCc3cc(Cl)ccn3)oc12]

Enantiomer separation on a chiral phase of 64.9 mg of the compound from Example 38 according to Method 38D gave 27.0 mg of Example 39 (enantiomerically pure isomer 1) and 23.0 mg of Example 40 (enantiomerically pure isomer 2).

HPLC (Method 34E): Rₜ=10.9 min, >99.0% ee;

LC-MS (Method 1A): Rₜ=0.83 min; MS (ESIpos): m/z=475 [M+H]⁺.

### Example 40

### (2-{[(4-Chloropyridin-2-yl)methyl]amino}-7-methoxy-1,3-benzoxazol-5-yl) [5-(hydroxymethyl)-2,2,5-trimethylmorpholin-4-yl]methanone [enantiomerically pure isomer 2]

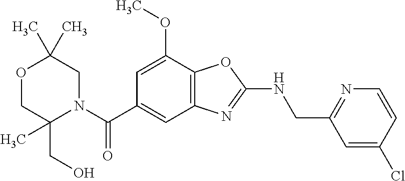 [SMILES: COc1cc(C(=O)N2CC(C)(C)OCC2(C)CO)cc2nc(NCc3cc(Cl)ccn3)oc12]

Enantiomer separation on a chiral phase of 64.9 mg of the compound from Example 38 according to Method 38D gave 27.0 mg of Example 39 (enantiomerically pure isomer 1) and 23.0 mg of Example 40 (enantiomerically pure isomer 2).

HPLC (Method 34E): Rₜ=13.6 min, >99.0% ee;

LC-MS (Method 1A): Rₜ=0.83 min; MS (ESIpos): m/z=475 [M+H]⁺.

### Example 41

### (2-{[(4-Chloropyridin-2-yl)methyl]amino}-7-methoxy-1,3-benzoxazol-5-yl) [5-(methoxymethyl)-2,2-dimethylmorpholin-4-yl]methanone [racemate]

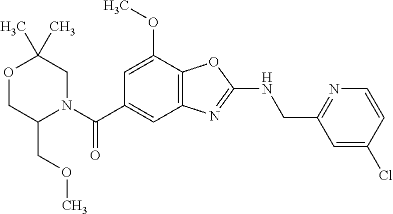 [SMILES: COCC1COC(C)(C)CN1C(=O)c1cc(OC)c2oc(NCc3cc(Cl)ccn3)nc2c1]

120 mg (0.360 mmol) of 2-{[(4-chloropyridin-2-yl)methyl]amino}-7-methoxy-1,3-benzoxazole-5-carboxylic acid and 68.7 mg (0.431 mmol) of 5-(methoxymethyl)-2,2-dimethylmorpholine [racemate] were initially charged in N,N-dimethylformamide (1.66 ml), and 163 mg (219 μl, 1.26 mmol) of N,N-diisopropylethylamine were added. Subsequently, 164 mg (0.431 mmol) of HATU were added at RT and the mixture was stirred overnight. Without further work-up, the reaction solution was then purified directly by preparative RP-HPLC (acetonitrile/water). Yield: 146 mg (84% of theory).

LC-MS (Method 1A): Rₜ=0.90 min; MS (ESIpos): m/z=475 [M+H]⁺;

¹H-NMR (400 MHz, DMSO-d₆): δ[ppm]=8.71 (t, 1H), 8.52 (d, 1H), 7.52 (d, 1H), 7.46 (dd, 1H), 6.86 (br. s., 1H), 6.72 (br. s., 1H), 4.63 (d, 2H), 3.91 (s, 3H), 3.78 (dd, 1H), 3.65-3.12 (m, 32H), 1.26-0.94 (m, 6H), nine protons, very broad and obscured by the water signal.

### Example 42

### (2-{[(4-Chloropyridin-2-yl)methyl]amino}-7-methoxy-1,3-benzoxazol-5-yl) [5-(fluoromethyl)-2,2-dimethylmorpholin-4-yl]methanone [racemate]

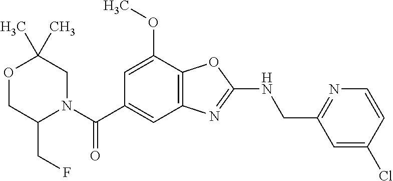 [SMILES: COc1cc(C(=O)N2CC(C)(C)OCC2CF)cc2nc(NCc3cc(Cl)ccn3)oc12]

80.0 mg (0.240 mmol) of 2-{[(4-chloropyridin-2-yl)methyl]amino}-7-methoxy-1,3-benzoxazole-5-carboxylic acid and 42.3 mg (0.288 mmol) of 5-(fluoromethyl)-2,2-dimethylmorpholine [racemate] were initially charged in N,N-dimethylformamide (1.10 ml), and 108 mg (146 μl, 0,839 mmol) of N,N-diisopropylethylamine were added. Subsequently, 109 mg (0.288 mmol) of HATU were added at RT and the mixture was stirred overnight. Without further work-up, the reaction solution was then purified directly by preparative RP-HPLC (acetonitrile/water). Yield: 54.8 mg (45% of theory).

LC-MS (Method 1A): Rₜ=1.94 min; MS (ESIpos): m/z=462 [M+H]⁺;

¹H-NMR (400 MHz, DMSO-d₆): δ[ppm]=8.72 (t, 1H), 8.52 (d, 1H), 7.52 (d, 1H), 7.45 (dd, 1H), 6.85 (br. s., 1H), 6.68 (s, 1H), 4.83-4.57 (m, 4H), 3.91 (s, 3H), 3.88-3.80 (m, 1H), 1.17 (br. s., 6H), four protons obscured or very broad.

### Example 43

### (2-{[(4-Chloropyridin-2-yl)methyl]amino}-7-methoxy-1,3-benzoxazol-5-yl) [5-(fluoromethyl)-2,2-dimethylmorpholin-4-yl]methanone [enantiomerically pure isomer 1]

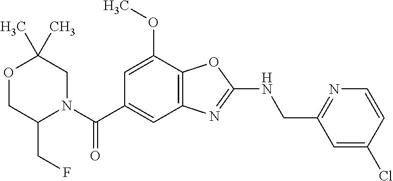 [SMILES: COc1cc(C(=O)N2CC(C)(C)OCC2CF)cc2nc(NCc3cc(Cl)ccn3)oc12]

Enantiomer separation on a chiral phase of 50 mg of the compound from Example 42 according to Method 39D gave 16.4 mg of Example 43 (enantiomerically pure isomer 1).

HPLC (Method 35E): Rₜ=5.31 min, >99.0% ee;

LC-MS (Method 1A): Rₜ=0.91 min; MS (ESIpos): m/z=463 [M+H]⁺;

¹H-NMR (400 MHz, DMSO-d₆): δ[ppm]=8.72 (t, 1H), 8.52 (d, 1H), 7.52 (d, 1H), 7.45 (dd, 1H), 6.85 (br. s., 1H), 6.68 (s, 1H), 4.83-4.57 (m, 4H), 3.91 (s, 3H), 3.88-3.80 (m, 1H), 1.17 (br. s., 6H), four protons obscured very broad.

### Example 44

### (2-{[(4-Chloropyridin-2-yl)methyl]amino}-7-methoxy-1,3-benzoxazol-5-yl)(2,5,5-trimethylmorpholin-4-yl)methanone [racemate]

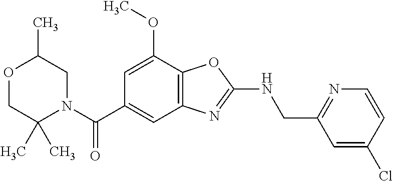 [SMILES: COc1cc(C(=O)N2CC(C)OCC2(C)C)cc2nc(NCc3cc(Cl)ccn3)oc12]

150 mg (0.422 mmol) of 2-{[(4-chloropyridin-2-yl)methyl]amino}-7-methoxy-1,3-benzoxazole-5-carboxylic acid and 65.5 mg (0.507 mmol) of 2,5,5-trimethylmorpholine [racemate] were initially charged in N,N-dimethylformamide (2.01 ml), and 191 mg (258 μl, 0,507 mmol) of N,N-diisopropylethylamine were added. 193 mg (0.507 mmol) of HATU were then added at RT, and the mixture was stirred for 1 h. Without further work-up, the reaction solution was then purified directly by preparative RP-HPLC (acetonitrile/water). Yield: 126 mg (67% of theory).

LC-MS (Method 1A): Rₜ=0.95 min; MS (ESIpos): m/z=445 [M+H]⁺;

¹H-NMR (400 MHz, DMSO-d₆): δ[ppm]=8.68 (t, 1H), 8.51 (d, 1H), 7.52 (d, 1H), 7.45 (dd, 1H), 6.88 (d, 1H), 6.73 (d, 1H), 4.63 (d, 2H), 3.91 (s, 3H), 3.69 (m_{c}, 1H), 3.48 (m_{c}, 1H), 3.43-3.34 (m, 2H), 2.80 (dd, 1H), 1.40 (s, 3H), 1.36 (s, 3H), 0.99 (d, 3H).

### Example 45

### (2-{[(4-Chloropyridin-2-yl)methyl]amino}-7-methoxy-1,3-benzoxazol-5-yl)(2,5,5-trimethylmorpholin-4-yl)methanone [enantiomerically pure isomer 1]

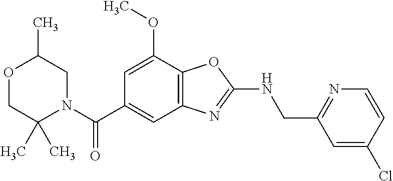 [SMILES: COc1cc(C(=O)N2CC(C)OCC2(C)C)cc2nc(NCc3cc(Cl)ccn3)oc12]

Enantiomer separation on a chiral phase of 118 mg of the compound from Example 44 according to Method 40D gave 53.4 mg of Example 45 (enantiomerically pure isomer 1).

HPLC (Method 36E): Rₜ=8.18 min, >99.9% ee;

LC-MS (Method 1A): Rₜ=0.95 min; MS (ESIpos): m/z=445 [M+H]⁺;

¹H-NMR (400 MHz, DMSO-d₆): δ [ppm]=8.68 (t, 1H), 8.51 (d, 1H), 7.52 (d, 1H), 7.45 (dd, 1H), 6.88 (d, 1H), 6.73 (d, 1H), 4.62 (d, 2H), 3.91 (s, 3H), 3.69 (m_{c}, 1H), 3.48 (d, 1H), 3.42-3.34 (m, 2H), 2.80 (dd, 1H), 1.40 (s, 3H), 1.36 (s, 3H), 0.99 (d, 3H).

### Example 46

### (2-{[(4-Chloropyridin-2-yl)methyl]amino}-7-methoxy-1,3-benzoxazol-5-yl) [5-(3-hydroxycyclobutyl)-2-methylmorpholin-4-yl]methanone [diastereomer mixture, 4 isomers]

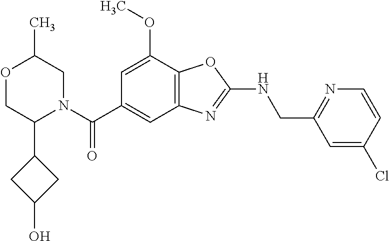 [SMILES: COc1cc(C(=O)N2CC(C)OCC2C2CC(O)C2)cc2nc(NCc3cc(Cl)ccn3)oc12]

100 mg (0.282 mmol) of 2-{[(4-chloropyridin-2-yl)methyl]amino}-7-methoxy-1,3-benzoxazole-5-carboxylic acid and 57.9 mg (0.338 mmol) of 3-(6-methylmorpholin-3-yl)cyclobutanol [diastereomer mixture, 4 isomers] were initially charged in N,N-dimethylformamide (1.30 ml), and 127 mg (172 μl, 0,986 mmol) of N,N-diisopropylethylamine were added. Subsequently, 129 mg (0.338 mmol) of HATU were added at RT and the mixture was stirred overnight. Without further work-up, the reaction solution was then purified directly by preparative RP-HPLC (acetonitrile/water). Yield: 91.8 mg (67% of theory).

LC-MS (Method 1A): Rₜ=0.78 min (diastereomer 1, 2 isomers), Rₜ=0.79 min (diastereomer 2, 2 isomers);

MS (ESIpos): m/z=487 [M+H]⁺.

### Example 47

### (2-{[(4-Chloropyridin-2-yl)methyl]amino}-7-methoxy-1,3-benzoxazol-5-yl) [5-(3-hydroxycyclobutyl)-2-methylmorpholin-4-yl]methanone [diastereomer 1, 2 isomers]

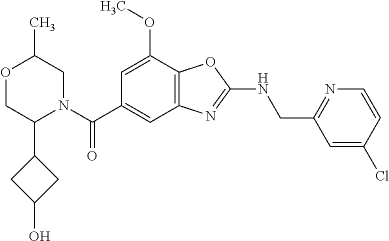 [SMILES: COc1cc(C(=O)N2CC(C)OCC2C2CC(O)C2)cc2nc(NCc3cc(Cl)ccn3)oc12]

Diastereomer separation on an achiral phase of 86.0 mg of the compound from Example 46 according to Method 1F gave 18.1 mg of Example 47 (diastereomer 1, 2 isomers) and 49.3 mg of Example 48 (diastereomer 2, 2 isomers).

LC-MS (Method 1A): Rₜ=0.81 min (diastereomer 1, 2 isomers), MS (ESIpos): m/z=487 [M+H]⁺.

### Example 48

### (2-{[(4-Chloropyridin-2-yl)methyl]amino}-7-methoxy-1,3-benzoxazol-5-yl) [5-(3-hydroxycyclobutyl)-2-methylmorpholin-4-yl]methanone [diastereomer 2, 2 isomers]

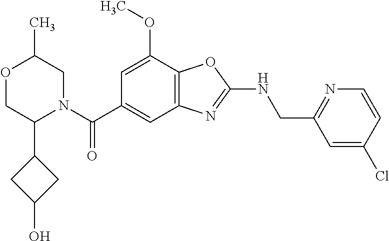 [SMILES: COc1cc(C(=O)N2CC(C)OCC2C2CC(O)C2)cc2nc(NCc3cc(Cl)ccn3)oc12]

Diastereomer separation on an achiral phase of 86.0 mg of the compound from Example 46 according to Method 1F gave 18.1 mg of Example 47 (diastereomer 1, 2 isomers) and 49.3 mg of Example 48 (diastereomer 2, 2 isomers).

LC-MS (Method 1A): Rₜ=0.82 min (diastereomer 2, 2 isomers), MS (ESIpos): m/z=487 [M+H]⁺.

### Example 49

### (2-{[(4-Chloropyridin-2-yl)methyl]amino}-7-methoxy-1,3-benzoxazol-5-yl) [5-(3-hydroxycyclobutyl)-2-methylmorpholin-4-yl]methanone [enantiomerically pure isomer 1]

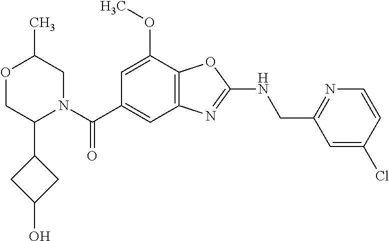 [SMILES: COc1cc(C(=O)N2CC(C)OCC2C2CC(O)C2)cc2nc(NCc3cc(Cl)ccn3)oc12]

Enantiomer separation on a chiral phase of 15.0 mg of the compound from Example 47 according to Method 41D gave 6.2 mg of Example 49 (enantiomerically pure isomer 1) and 7.2 mg of Example 50 (enantiomerically pure isomer 2).

HPLC (Method 20E): Rₜ=6.63 min, >99.9% ee;

LC-MS (Method 1A): Rₜ=0.81 min; MS (ESIpos): m/z=487 [M+H]⁺.

### Example 50

### (2-{[(4-Chloropyridin-2-yl)methyl]amino}-7-methoxy-1,3-benzoxazol-5-yl) [5-(3-hydroxycyclobutyl)-2-methylmorpholin-4-yl]methanone [enantiomerically pure isomer 2]

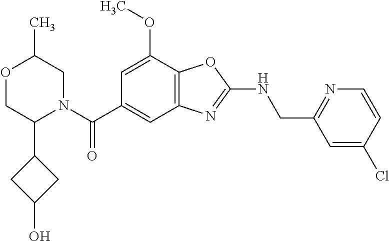 [SMILES: COc1cc(C(=O)N2CC(C)OCC2C2CC(O)C2)cc2nc(NCc3cc(Cl)ccn3)oc12]

Enantiomer separation on a chiral phase of 15.0 mg of the compound from Example 47 according to Method 41D gave 6.2 mg of Example 49 (enantiomerically pure isomer 1) and 7.2 mg of Example 50 (enantiomerically pure isomer 2).

HPLC (Method 20E): Rₜ=15.9 min, >99.9% ee;

LC-MS (Method 1A): Rₜ=0.81 min; MS (ESIpos): m/z=487 [M+H]⁺.

### Example 51

### (2-{[(4-Chloropyridin-2-yl)methyl]amino}-7-methoxy-1,3-benzoxazol-5-yl) [5-(3-hydroxycyclobutyl)-2-methylmorpholin-4-yl]methanone [enantiomerically pure isomer 3]

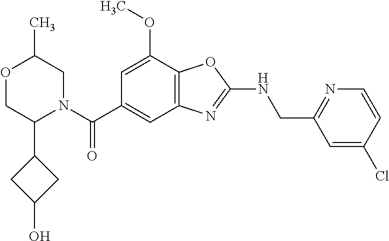 [SMILES: COc1cc(C(=O)N2CC(C)OCC2C2CC(O)C2)cc2nc(NCc3cc(Cl)ccn3)oc12]

Enantiomer separation on a chiral phase of 45.0 mg of the compound from Example 48 according to Method 42D gave 14.2 mg of Example 51 (enantiomerically pure isomer 3) and 19.8 mg of Example 52 (enantiomerically pure isomer 4).

HPLC (Method 27E): Rₜ=5.23 min, >99.9% ee;

LC-MS (Method 1A): Rₜ=0.78 min; MS (ESIpos): m/z=487 [M+H]⁺.

### Example 52

### (2-{[(4-Chloropyridin-2-yl)methyl]amino}-7-methoxy-1,3-benzoxazol-5-yl) [5-(3-hydroxycyclobutyl)-2-methylmorpholin-4-yl]methanone [enantiomerically pure isomer 4]

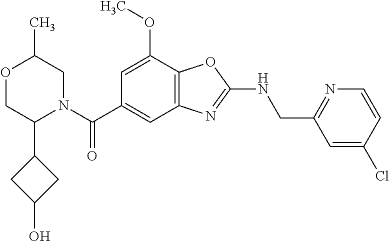 [SMILES: COc1cc(C(=O)N2CC(C)OCC2C2CC(O)C2)cc2nc(NCc3cc(Cl)ccn3)oc12]

Enantiomer separation on a chiral phase of 45.0 mg of the compound from Example 48 according to Method 42D gave 14.2 mg of Example 51 (enantiomerically pure isomer 3) and 19.8 mg of Example 52 (enantiomerically pure isomer 4).

HPLC (Method 27E): Rₜ=10.4 min, >99.9% ee;

LC-MS (Method 1A): Rₜ=0.78 min; MS (ESIpos): m/z=487 [M+H]⁺.

### Example 53

### (2-{[(4-Chloropyridin-2-yl)methyl]amino}-7-methoxy-1,3-benzoxazol-5-yl)(2-hydroxy-7-methyl-8-oxa-5-azaspiro[3.5]non-5-yl)methanone [enantiomerically pure cis-isomer 1]

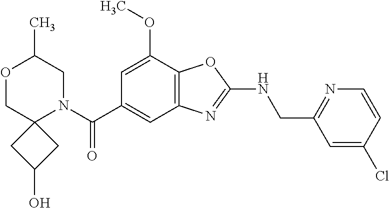 [SMILES: COc1cc(C(=O)N2CC(C)OCC23CC(O)C3)cc2nc(NCc3cc(Cl)ccn3)oc12]

60.0 mg (0.169 mmol) of 2-{[(4-chloropyridin-2-yl)methyl]amino}-7-methoxy-1,3-benzoxazole-5-carboxylic acid and 39.3 mg (0.203 mmol) of cis-7-methyl-8-oxa-5-azaspiro[3.5]nonan-2-ol hydrochloride [enantiomerically pure isomer 1] were initially charged in N,N-dimethylformamide (0.78 ml), and 76.4 mg (103 μl, 0.591 mmol) of N,N-diisopropylethylamine were added. 77.1 mg (0,203 mmol) of HATU were then added at RT, and the mixture was stirred for 1 h. A further 39.3 mg (0.203 mmol) of cis-7-methyl-8-oxa-5-azaspiro[3.5]nonan-2-ol hydrochloride [enantiomerically pure isomer 1] and 76.4 mg (103 μl, 0.591 mmol) of N,N-diisopropylethylamine were then added, and the mixture was stirred at RT for 1 h. Without further work-up, the reaction solution was then purified directly by preparative RP-HPLC (acetonitrile/water). Yield: 49.0 mg (60% of theory).

LC-MS (Method 1A): Rₜ=0.76; MS (ESIpos): m/z=473 [M+H]⁺;

¹H-NMR (400 MHz, DMSO-d₆): δ[ppm]=8.71 (t, 1H), 8.52 (d, 1H), 7.53 (d, 1H), 7.46 (dd, 1H), 6.92 (d, 1H), 6.79 (d, 1H), 5.09 (d, 1H), 4.63 (d, 2H), 3.93 (s, 3H), 3.86 (q, 1H), 3.62 (dd, 1H), 3.49 (d, 2H), 2.91 (dd, 1H), 2.69 (m_{c}, 1H), 2.39-2.28 (m, 1H), 2.21-2.12 (m, 1H), 1.85 (t, 1H), 0.91 (d, 3H), one proton obscured.

### Example 54

### (2-{[(4-Chloropyridin-2-yl)methyl]amino}-7-methoxy-1,3-benzoxazol-5-yl) [5-(difluormethyl)-2-(2-hydroxyethyl)-2-methylmorpholin-4-yl]methanone [racemate]

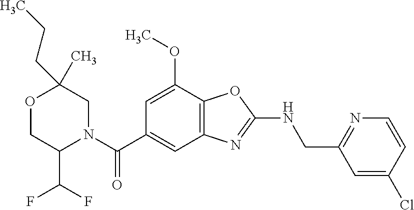 [SMILES: CCCC1(C)CN(C(=O)c2cc(OC)c3oc(NCc4cc(Cl)ccn4)nc3c2)C(C(F)F)CO1]

100 mg (0.282 mmol) of 2-{[(4-chloropyridin-2-yl)methyl]amino}-7-methoxy-1,3-benzoxazole-5-carboxylic acid and 70.2 mg (0.338 mmol) of 2-[5-(difluoromethyl)-2-methylmorpholin-2-yl]ethanol [diastereomer 1, 2 isomers] were initially charged in N,N-dimethylformamide (1.34 ml), and 127 mg (171 μl, 0.986 mmol) of N,N-diisopropylethylamine were added. Subsequently, 129 mg (0.338 mmol) of HATU were added at RT and the mixture was stirred overnight. Without further work-up, the reaction solution was then purified directly by preparative RP-HPLC (acetonitrile/water) and subsequently by Method 6F. Yield: 5.4 mg (3% of theory).

LC-MS (Method 1A): Rₜ=0.85 min; MS (ESIpos): m/z=511 [M+H]⁺.

### Example 55

### (2-{[(4-Chloropyridin-2-yl)methyl]amino}-7-methoxy-1,3-benzoxazol-5-yl) [5-(2-hydroxy-2-methylpropyl)-2-methylmorpholin-4-yl]methanone [racemate]

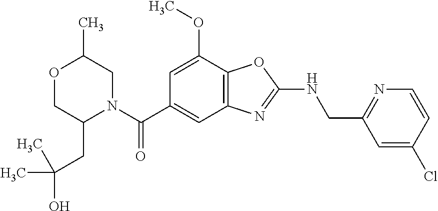 [SMILES: COc1cc(C(=O)N2CC(C)OCC2CC(C)(C)O)cc2nc(NCc3cc(Cl)ccn3)oc12]

63.2 mg (0.178 mmol) of 2-{[(4-chloropyridin-2-yl)methyl]amino}-7-methoxy-1,3-benzoxazole-5-carboxylic acid and 37.0 mg (0.214 mmol) of 2-methyl-1-(6-methylmorpholin-3-yl)propan-2-ol [diastereomer 2, 2 isomers] were initially charged in N,N-dimethylformamide (0.82 ml), and 80.5 mg (108 μl, 0.623 mmol) of N,N-diisopropylethylamine were added. Subsequently, 81.2 mg (0.214 mmol) of HATU were added at RT and the mixture was stirred overnight. Without further work-up, the reaction solution was then purified directly by preparative RP-HPLC (acetonitrile/water).

Yield: 66.4 mg (76% of theory).

LC-MS (Method 1A): Rₜ=0.86 min; MS (ESIpos): m/z=489 [M+H]⁺.

### Example 56

### (2-{[(4-Chloropyridin-2-yl)methyl]amino}-7-methoxy-1,3-benzoxazol-5-yl) [5-(2-hydroxy-2-methylpropyl)-2-methylmorpholin-4-yl]methanone [enantiomerically pure isomer 1]

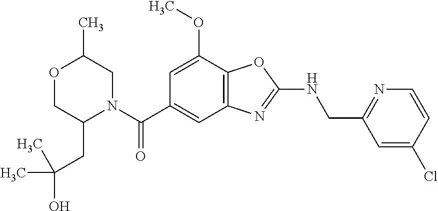 [SMILES: COc1cc(C(=O)N2CC(C)OCC2CC(C)(C)O)cc2nc(NCc3cc(Cl)ccn3)oc12]

Enantiomer separation on a chiral phase of 60.0 mg of the compound from Example 55 according to Method 23D gave 28.0 mg of Example 56 (enantiomerically pure isomer 1) and 29.0 mg of Example 57 (enantiomerically pure isomer 2).

HPLC (Method 31E): Rₜ=7.06 min, >99.9% ee;

LC-MS (Method 1A): Rₜ=0.83 min; MS (ESIpos): m/z=489 [M+H]⁺.

### Example 57

### (2-{[(4-Chloropyridin-2-yl)methyl]amino}-7-methoxy-1,3-benzoxazol-5-yl) [5-(2-hydroxy-2-methylpropyl)-2-methylmorpholin-4-yl]methanone [enantiomerically pure isomer 2]

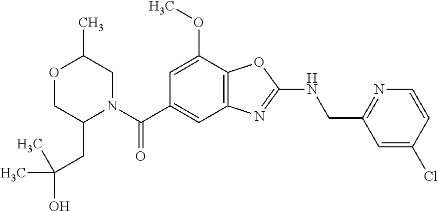 [SMILES: COc1cc(C(=O)N2CC(C)OCC2CC(C)(C)O)cc2nc(NCc3cc(Cl)ccn3)oc12]

Enantiomer separation on a chiral phase of 60.0 mg of the compound from Example 55 according to Method 23D gave 28.0 mg of Example 56 (enantiomerically pure isomer 1) and 29.0 mg of Example 57 (enantiomerically pure isomer 2).

HPLC (Method 31E): Rₜ=9.33 min, >99.9% ee;

LC-MS (Method 1A): Rₜ=0.83 min; MS (ESIpos): m/z=489 [M+H]⁺.

### Example 58

### (2-{[(4-Chloropyridin-2-yl)methyl]amino}-7-methoxy-1,3-benzoxazol-5-yl)[(5R)-5-(2-hydroxyethyl)-2,2-dimethylmorpholin-4-yl]methanone [enantiomer mixture, 2 isomers]

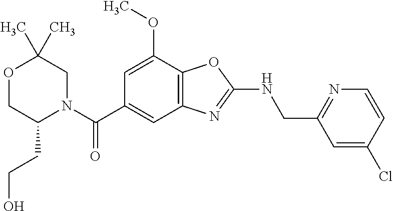 [SMILES: COc1cc(C(=O)N2CC(C)(C)OC[C@H]2CCO)cc2nc(NCc3cc(Cl)ccn3)oc12]

70.0 mg (0.210 mmol) of 2-{[(4-chloropyridin-2-yl)methyl]amino}-7-methoxy-1,3-benzoxazole-5-carboxylic acid and 41.1 mg (0.252 mmol) of 2-[(3R)-6,6-dimethylmorpholin-3-yl]ethanol [enantiomer mixture, 2 isomers, enantiomer ratio: about 85:15] were initially charged in N,N-dimethylformamide (1.00 ml), and 94.9 mg (128 μl, 0.734 mmol) of N,N-diisopropylethylamine were added. 95.7 mg (0.252 mmol) of HATU were then added at RT, and the mixture was stirred for 1 h. Without further work-up, the reaction solution was then purified directly by preparative RP-HPLC (acetonitrile/water). Yield: 64.0 mg (61% of theory; enantiomer mixture, 2 isomers, enantiomer ratio: about 85:15).

LC-MS (Method 1A): Rₜ=0.79 min; MS (ESIpos): m/z=475 [M+H]⁺;

¹H-NMR (500 MHz, DMSO-d₆): δ[ppm]=8.66 (br. s., 1H), 8.51 (d, 1H), 7.52 (d, 1H), 7.45 (dd, 1H), 6.82 (br. s., 1H), 6.67 (s, 1H), 4.63 (s, 2H), 4.37 (br. s., 1H), 3.91 (s, 3H), 3.79 (dd, 1H), 3.51-3.34 (m., 3H), 1.99-1.76 (m, 2H), 1.27-0.96 (m, 6H), three protons very broad or obscured.

### Example 59

### (2-{[1-(4-Chloropyridin-2-yl)ethyl]amino}-7-methoxy-1,3-benzoxazol-5-yl)[(5R)-5-(2-hydroxyethyl)-2,2-dimethylmorpholin-4-yl]methanone [diastereomer mixture, 4 isomers]

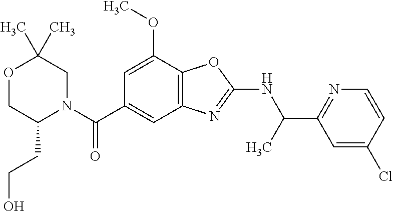 [SMILES: COc1cc(C(=O)N2CC(C)(C)OC[C@H]2CCO)cc2nc(NC(C)c3cc(Cl)ccn3)oc12]

70.0 mg (0.159 mmol, purity: 79%) of 2-{[1-(4-chloropyridin-2-yl)ethyl]amino}-7-methoxy-1,3-benzoxazole-5-carboxylic acid [racemate] and 30.4 mg (0.191 mmol) of 2-[(3R)-6,6-dimethylmorpholin-3-yl]ethanol [enantiomer mixture, 2 isomers, enantiomer ratio: about 85:15] were initially charged in N,N-imethylformamide (0.73 ml), and 71.9 mg (97 μl, 0.557 mmol) of N,N-diisopropylethylamine were added. 72.6 mg (0.191 mmol) of HATU were then added at RT, and the mixture was stirred for 1 h. Without further work-up, the reaction solution was then purified directly by preparative RP-HPLC (acetonitrile/water) and by Method F. Yield: 44.0 mg (51% of theory; diasteromer mixture, 4 isomers, diasteromer ratio: about 91:9).

LC-MS (Method 1A): Rₜ=0.75 min; 0.84 min; MS (ESIpos): m/z=489 [M+H]⁺.

### Example 60

### (2-{[(4-Chloropyridin-2-yl)methyl]amino}-7-methoxy-1,3-benzoxazol-5-yl){5-[(difluoromethoxy)methyl]-2-(2-hydroxyethyl)-2-methylmorpholin-4-yl}methanone [enantiomerically pure isomer]

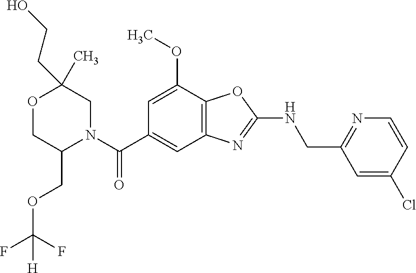 [SMILES: COc1cc(C(=O)N2CC(C)(CCO)OCC2COC(F)F)cc2nc(NCc3cc(Cl)ccn3)oc12]

50.0 mg (0.141 mmol) of 2-{[(4-chloropyridin-2-yl)methyl]amino}-7-methoxy-1,3-benzoxazole-5-carboxylic acid and 38.1 mg (0.169 mmol) of 2-{5-[(difluoromethoxy)methyl]-2-methylmorpholin-²-yl}ethanol [enantiomerically pure isomer] were initially charged in N,N-dimethylformamide (0.671 ml), and 63.7 mg (85.9 μl, 0.493 mmol) of N,N-diisopropylethylamine were added. Subsequently, 64.3 mg (0.169 mmol) of HATU were added at RT and the mixture was stirred overnight. Without further work-up, the reaction solution was then purified directly by preparative RP-HPLC (acetonitrile/water). Yield: 18.6 mg (23% of theory).

LC-MS (Method 1A): Rₜ=0.87 min; MS (ESIpos): m/z=541 [M+H]⁺.

### Example 61

### (2-{[(4-Chloropyridin-2-yl)methyl]amino}-7-methoxy-1,3-benzoxazol-5-yl) [2-(2-hydroxypropyl)-2-methylmorpholin-4-yl]methanone [enantiomerically pure isomer]

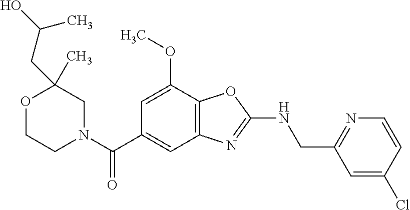 [SMILES: COc1cc(C(=O)N2CCOC(C)(CC(C)O)C2)cc2nc(NCc3cc(Cl)ccn3)oc12]

80.0 mg (0.225 mmol) of 2-{[(4-chloropyridin-2-yl)methyl]amino}-7-methoxy-1,3-benzoxazole-5-carboxylic acid and 43.1 mg (0.270 mmol) of 1-(2-methylmorpholin-2-yl)propan-2-ol [enantiomerically pure isomer] were initially charged in N,N-dimethylformamide (1.04 ml), and 102 mg (137 μl, 0,789 mmol) of N,N-diisopropylethylamine were added. Subsequently, 103 mg (0.270 mmol) of HATU were added at RT and the mixture was stirred overnight. Without further work-up, the reaction solution was then purified directly by preparative RP-HPLC (acetonitrile/water). Yield: 81.5 mg (76% of theory).

LC-MS (Method 1A): Rₜ=0.78 min; MS (ESIpos): m/z=475 [M+H]⁺;

¹H-NMR (400 MHz, DMSO-d₆): δ[ppm]=8.71 (t, 1H), 8.52 (d, 1H), 7.52 (d, 1H), 7.46 (dd, 1H), 6.85 (br. s., 1H), 6.71 (s, 1H), 4.63 (d, 2H), 4.40-4.14 (br. m., 1H), 3.91 (s, 3H), 3.86-3.45 (br. m., 4H), 3.18 (d, 1H), 1.68-0.93 (m, 8H), two protons obscured.

### Example 62

### (2-{[(4-Chloropyridin-2-yl)methyl]amino}-7-methoxy-1,3-benzoxazol-5-yl) [5-(2-hydroxypropyl)-2,2-dimethylmorpholin-4-yl]methanone [enantiomerically pure isomer 1]

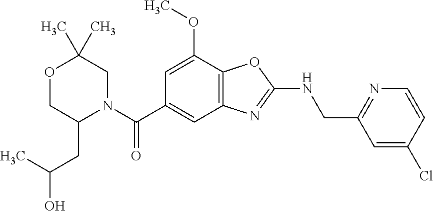 [SMILES: COc1cc(C(=O)N2CC(C)(C)OCC2CC(C)O)cc2nc(NCc3cc(Cl)ccn3)oc12]

125 mg (0.376 mmol) of 2-{[(4-chloropyridin-2-yl)methyl]amino}-7-methoxy-1,3-benzoxazole-5-carboxylic acid and 78.1 mg (0.451 mmol) of 1-[6,6-dimethylmorpholin-3-yl]propan-2-ol [diastereomer mixture, 4 isomers] were initially charged in N,N-dimethylformamide (1.79 ml), and 170 mg (229 μl, 1.32 mmol) of N,N-diisopropylethylamine were added. 171 mg (0.451 mmol) of HATU were then added at RT, and the mixture was stirred for 1 h. Without further work-up, the reaction solution was then purified directly by preparative RP-HPLC (acetonitrile/water) and separated into the two diastereomers. Yield: 82.9 mg (45% of theory, diastereomer 1), 73.1 mg (39% of theory, diastereomer 2). Enantiomer separation on a chiral phase of 73.1 mg of diastereomer 1 according to Method 46D gave 31.0 mg of Example 62 (enantiomerically pure isomer 1) and 44.0 mg of Example 63 (enantiomerically pure isomer 2).

HPLC (Method 40E): Rₜ=4.78 min, >99.0% ee;

LC-MS (Method 1A): Rₜ=0.85 min; MS (ESIpos): m/z=489 [M+H]⁺.

### Example 63

### (2-{[(4-Chloropyridin-2-yl)methyl]amino}-7-methoxy-1,3-benzoxazol-5-yl) [5-(2-hydroxypropyl)-2,2-dimethylmorpholin-4-yl]methanone [enantiomerically pure isomer 2]

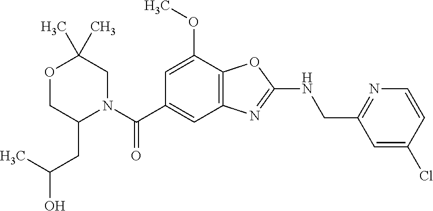 [SMILES: COc1cc(C(=O)N2CC(C)(C)OCC2CC(C)O)cc2nc(NCc3cc(Cl)ccn3)oc12]

125 mg (0.376 mmol) of 2-{[(4-chloropyridin-2-yl)methyl]amino}-7-methoxy-1,3-benzoxazole-5-carboxylic acid and 78.1 mg (0.451 mmol) of 1-[6,6-dimethylmorpholin-3-yl]propan-2-ol [diastereomer mixture, 4 isomers] were initially charged in N,N-dimethylformamide (1.79 ml), and 170 mg (229 μl, 1.32 mmol) of N,N-diisopropylethylamine were added. 171 mg (0.451 mmol) of HATU were then added at RT, and the mixture was stirred for 1 h. Without further work-up, the reaction solution was then purified directly by preparative RP-HPLC (acetonitrile/water) and separated into the two diastereomers. Yield: 82.9 mg (45% of theory, diastereomer 1), 73.1 mg (39% of theory, diastereomer 2). Enantiomer separation on a chiral phase of 73.1 mg of diastereomer 1 according to Method 46D gave 31.0 mg of Example 62 (enantiomerically pure isomer 1) and 44.0 mg of Example 63 (enantiomerically pure isomer 2).

HPLC (Method 40E): Rₜ=6.32 min, >99.0% ee;

LC-MS (Method 1A): Rₜ=0.82 min; MS (ESIpos): m/z=489 [M+H]⁺.

### Example 64

### (2-{[(4-Chloropyridin-2-yl)methyl]amino}-7-methoxy-1,3-benzoxazol-5-yl) [5-(2-hydroxypropyl)-2,2-dimethylmorpholin-4-yl]methanone [enantiomerically pure isomer 3]

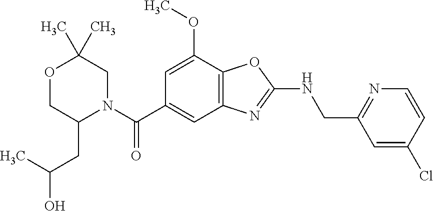 [SMILES: COc1cc(C(=O)N2CC(C)(C)OCC2CC(C)O)cc2nc(NCc3cc(Cl)ccn3)oc12]

125 mg (0.376 mmol) of 2-{[(4-chloropyridin-2-yl)methyl]amino}-7-methoxy-1,3-benzoxazole-5-carboxylic acid and 78.1 mg (0.451 mmol) of 1-[6,6-dimethylmorpholin-3-yl]propan-2-ol [diastereomer mixture, 4 isomers] were initially charged in N,N-dimethylformamide (1.79 ml), and 170 mg (229 μl, 1.32 mmol) of N,N-diisopropylethylamine were added. 171 mg (0.451 mmol) of HATU were then added at RT, and the mixture was stirred for 1 h. Without further work-up, the reaction solution was then purified directly by preparative RP-HPLC (acetonitrile/water) and separated into the two diastereomers. Yield: 82.9 mg (45% of theory, diastereomer 1), 73.1 mg (39% of theory, diastereomer 2). Enantiomer separation on a chiral phase of 73.0 mg of diastereomer 2 according to Method 46D gave 18.5 mg of Example 64 (enantiomerically pure isomer 3) and 24.0 mg of Example 65 (enantiomerically pure isomer 4).

HPLC (Method 41E): Rₜ=5.13 min, >99.0% ee;

LC-MS (Method 1A): Rₜ=0.84 min; MS (ESIpos): m/z=488 [M+H]⁺.

### Example 65

### (2-{[(4-Chloropyridin-2-yl)methyl]amino}-7-methoxy-1,3-benzoxazol-5-yl) [5-(2-hydroxypropyl)-2,2-dimethylmorpholin-4-yl]methanone [enantiomerically pure isomer 4]

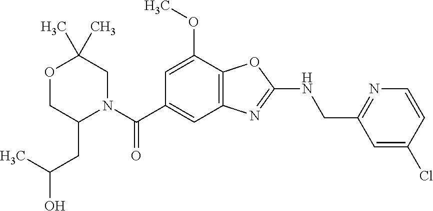 [SMILES: COc1cc(C(=O)N2CC(C)(C)OCC2CC(C)O)cc2nc(NCc3cc(Cl)ccn3)oc12]

125 mg (0.376 mmol) of 2-{[(4-chloropyridin-2-yl)methyl]amino}-7-methoxy-1,3-benzoxazole-5-carboxylic acid and 78.1 mg (0.451 mmol) of 1-[6,6-dimethylmorpholin-3-yl]propan-2-ol [diastereomer mixture, 4 isomers] were initially charged in N,N-dimethylformamide (1.79 ml), and 170 mg (229 μl, 1.32 mmol) of N,N-diisopropylethylamine were added. 171 mg (0.451 mmol) of HATU were then added at RT, and the mixture was stirred for 1 h. Without further work-up, the reaction solution was then purified directly by preparative RP-HPLC (acetonitrile/water) and separated into the two diastereomers. Yield: 82.9 mg (45% of theory, diastereomer 1), 73.1 mg (39% of theory, diastereomer 2). Enantiomer separation on a chiral phase of 73.0 mg of diastereomer 2 according to Method 46D gave 18.5 mg of Example 64 (enantiomerically pure isomer 3) and 24.0 mg of Example 65 (enantiomerically pure isomer 4).

HPLC (Method 41E): Rₜ=5.13 min, >99.0% ee;

LC-MS (Method 1A): Rₜ=0.84 min; MS (ESIpos): m/z=489 [M+H]⁺.

### Example 66

### (2-{[(4-Chloropyridin-2-yl)methyl]amino}-7-methoxy-1,3-benzoxazol-5-yl) [5-(2-hydroxypropyl)-2-methylmorpholin-4-yl]methanone [enantiomerically pure isomer 1]

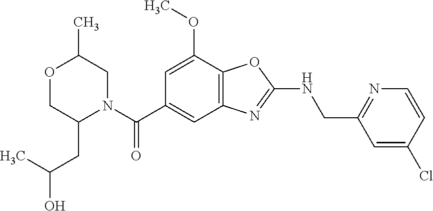 [SMILES: COc1cc(C(=O)N2CC(C)OCC2CC(C)O)cc2nc(NCc3cc(Cl)ccn3)oc12]

150 mg (0.422 mmol) of 2-{[(4-chloropyridin-2-yl)methyl]amino}-7-methoxy-1,3-benzoxazole-5-carboxylic acid and 80.7 mg (0.406 mmol) of 1-(6-methylmorpholin-3-yl)propan-2-ol [diastereomer mixture, 2 isomers] were initially charged in N,N-dimethylformamide (2.01 ml), and 191 mg (258 μl, 1.48 mmol) of N,N-diisopropylethylamine were added. Subsequently, 193 mg (0.507 mmol) of HATU were added at RT and the mixture was stirred overnight. Without further work-up, the reaction solution was then purified directly by preparative RP-HPLC (acetonitrile/water). Diastereomer separation on a chiral phase of 100 mg according to Method 47D gave 44.4 mg of Example 66 (enantiomerically pure isomer 1) and 43.1 mg of Example 67 (enantiomerically pure isomer 2).

HPLC (Method 14E): Rₜ=4.19 min, >99.0% ee;

LC-MS (Method 1A): Rₜ=0.79 min; MS (ESIpos): m/z=475 [M+H]⁺.

### Example 67

### (2-{[(4-Chloropyridin-2-yl)methyl]amino}-7-methoxy-1,3-benzoxazol-5-yl) [5-(2-hydroxypropyl)-2-methylmorpholin-4-yl]methanone [enantiomerically pure isomer 2]

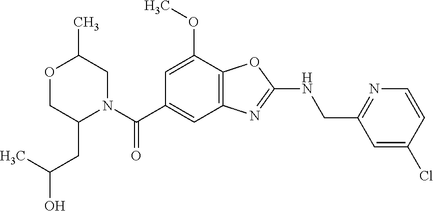 [SMILES: COc1cc(C(=O)N2CC(C)OCC2CC(C)O)cc2nc(NCc3cc(Cl)ccn3)oc12]

150 mg (0.422 mmol) of 2-{[(4-chloropyridin-2-yl)methyl]amino}-7-methoxy-1,3-benzoxazole-5-carboxylic acid and 80.7 mg (0.406 mmol) of 1-(6-methylmorpholin-3-yl)propan-2-ol [diastereomer mixture, 2 isomers] were initially charged in N,N-dimethylformamide (2.01 ml), and 191 mg (258 μl, 1.48 mmol) of N,N-diisopropylethylamine were added. Subsequently, 193 mg (0.507 mmol) of HATU were added at RT and the mixture was stirred overnight. Without further work-up, the reaction solution was then purified directly by preparative RP-HPLC (acetonitrile/water). Diastereomer separation on a chiral phase of 100 mg according to Method 47D gave 44.4 mg of Example 66 (enantiomerically pure isomer 1) and 43.1 mg of Example 67 (enantiomerically pure isomer 2).

HPLC (Method 14E): Rₜ=5.87 min, >99.0% ee;

LC-MS (Method 1A): Rₜ=0.82 min; MS (ESIpos): m/z=475 [M+H]⁺.

### Example 68

### (2-{[(4-Chloropyridin-2-yl)methyl]amino}-7-methoxy-1,3-benzoxazol-5-yl){(5R)-5-[(1R)-1-hydroxyethyl]-2-methylmorpholin-4-yl}methanone [diastereomer mixture, 2 isomers]

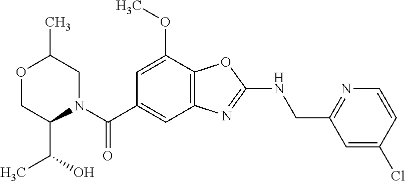

120 mg (0.360 mmol) of 2-{[(4-chloropyridin-2-yl)methyl]amino}-7-methoxy-1,3-benzoxazole-5-carboxylic acid and 62.7 mg (0.431 mmol) of (1R)-1-[(3R)-6-methylmorpholin-3-yl]ethanol [diastereomer mixture, 2 isomers] were initially charged in N,N-dimethylformamide (1.66 ml), and 163 mg (219 μl, 1.26 mmol) of N,N-diisopropylethylamine were added. Subsequently, 164 mg (0.431 mmol) of HATU were added at RT and the mixture was stirred overnight. Without further work-up, the reaction solution was then purified directly by preparative RP-HPLC (acetonitrile/water). Yield: 112 mg (67% of theory).

LC-MS (Method 7A): Rₜ=0.79 min (diastereomer 1), Rₜ=0.80 min (diastereomer 2);

MS (ESIpos): m/z=461 [M+H]⁺.

### Example 69

### (2-{[(4-Chloropyridin-2-yl)methyl]amino}-7-methoxy-1,3-benzoxazol-5-yl) [5-(1-hydroxyethyl)-2,2,5-trimethylmorpholin-4-yl]methanone [diastereomer mixture, 2 isomers]

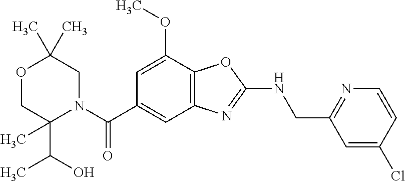 [SMILES: COc1cc(C(=O)N2CC(C)(C)OCC2(C)C(C)O)cc2nc(NCc3cc(Cl)ccn3)oc12]

120 mg (0.360 mmol) of 2-{[(4-chloropyridin-2-yl)methyl]amino}-7-methoxy-1,3-benzoxazole-5-carboxylic acid and 74.8 mg (0.431 mmol) of 1-(3,6,6-trimethylmorpholin-3-yl)ethanol [diastereomer 2, 2 isomers] were initially charged in N,N-dimethylformamide (1.66 ml), and 163 mg (219 μl, 1.26 mmol) of N,N-diisopropylethylamine were added. Subsequently, 164 mg (0.431 mmol) of HATU were added at RT and the mixture was stirred overnight. Without further work-up, the reaction solution was then purified directly by preparative RP-HPLC (acetonitrile/water).

Yield: 13 mg (7% of theory).

LC-MS (Method 1A): Rₜ=0.79 min; MS (ESIpos): m/z=489 [M+H]⁺;

¹H-NMR (400 MHz, DMSO-d₆): δ[ppm]=8.67 (t, 1H), 8.52 (d, 1H), 7.53 (d, 1H), 7.46 (dd, 1H), 6.77 (d, 1H), 6.63 (d, 1H), 5.08 (d, 1H), 4.62 (d, 2H), 4.41 (quin, 1H), 3.98 (d, 1H), 3.89 (s, 3H), 3.67-3.44 (m, 1H), 3.25 (d, 1H), 3.09 (d, 1H), 1.56 (s, 3H), 1.10 (s, 3H), 1.06 (d, 3H), 0.98 (s, 3H).

### Example 70

### (2-{[(4-Chloropyridin-2-yl)methyl]amino}-7-methoxy-1,3-benzoxazol-5-yl) [5-(2-hydroxyethyl)-2-methylmorpholin-4-yl]methanone [racemate]

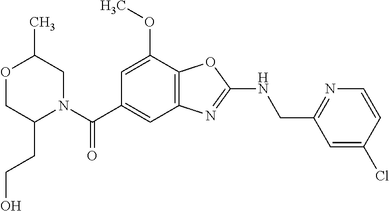 [SMILES: COc1cc(C(=O)N2CC(C)OCC2CCO)cc2nc(NCc3cc(Cl)ccn3)oc12]

840 mg (1.20 mmol) of [5-(2-{[tert-butyl(diphenyl)silyl]oxy}ethyl)-2-methylmorpholin-4-yl](2-{[(4-chloropyridin-2-yl)methyl]amino}-7-methoxy-1,3-benzoxazol-5-yl)methanone [racemate] were initially charged in tetrahydrofuran (25.2 ml), 628 mg (2.40 mmol) of tetra-n-butylammonium fluoride were added at RT and the mixture was stirred for 30 min. The reaction solution was then concentrated under reduced pressure and the residue was purified directly by preparative RP-HPLC (acetonitrile/water). Yield: 525 mg (91% of theory).

LC-MS (Method 2A): Rₜ=0.73 min; MS (ESIpos): m/z=461 [M+H]⁺.

### Example 71

### (2-{[(4-Chloropyridin-2-yl)methyl]amino}-7-methoxy-1,3-benzoxazol-5-yl) [5-(2-hydroxyethyl)-2-methylmorpholin-4-yl]methanone [enantiomerically pure isomer 1]

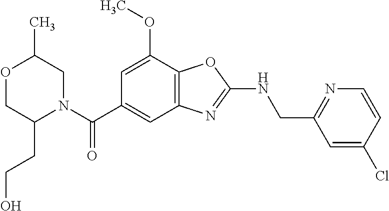 [SMILES: COc1cc(C(=O)N2CC(C)OCC2CCO)cc2nc(NCc3cc(Cl)ccn3)oc12]

Enantiomer separation on a chiral phase of 512 mg of the compound from Example 70 according to Method 48D gave, after re-purification by preparative RP-HPLC (acetonitrile/water), 178 mg of Example 71 (enantiomerically pure isomer 1) and 168 mg of Example 72 (enantiomerically pure isomer 2).

HPLC (Method 19E): Rₜ=4.65 min, >99.0% ee;

LC-MS (Method 2A): Rₜ=0.74 min; MS (ESIpos): m/z=461 [M+H]⁺;

¹H-NMR (400 MHz, DMSO-d₆): δ[ppm]=8.70 (d, 1H), 8.52 (d, 1H), 7.53 (d, 1H), 7.46 (dd, 1H), 6.85 (d, 1H), 6.71 (br. s., 1H), 4.63 (d, 2H), 4.52 (br. s., 1H), 4.19 (d, 1H), 3.92 (s, 3H), 3.84-3.37 (m, 5H), 3.09-2.59 (m, 1H), 2.03-1.71 (m, 2H), 1.23-0.91 (m, 3H), one proton obscured.

### Example 72

### (2-{[(4-Chloropyridin-2-yl)methyl]amino}-7-methoxy-1,3-benzoxazol-5-yl) [5-(2-hydroxyethyl)-2-methylmorpholin-4-yl]methanone [enantiomerically pure isomer 2]

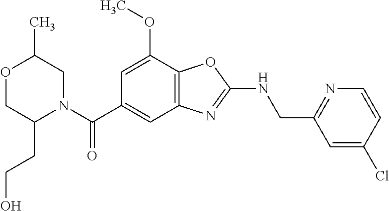 [SMILES: COc1cc(C(=O)N2CC(C)OCC2CCO)cc2nc(NCc3cc(Cl)ccn3)oc12]

Enantiomer separation on a chiral phase of 512 mg of the compound from Example 70 according to Method 48D gave, after re-purification by preparative RP-HPLC (acetonitrile/water), 178 mg of Example 71 (enantiomerically pure isomer 1) and 168 mg of Example 72 (enantiomerically pure isomer 2).

HPLC (Method 19E): Rₜ=6.21 min, >99.0% ee;

LC-MS (Method 2A): Rₜ=0.74 min; MS (ESIpos): m/z=461 [M+H]⁺;

¹H-NMR (400 MHz, DMSO-d₆): δ[ppm]=8.70 (d, 1H), 8.52 (d, 1H), 7.52 (d, 1H), 7.45 (dd, 1H), 6.84 (br. s., 1H), 6.70 (br. s., 1H), 4.62 (d, 2H), 4.56-4.14 (m, 2H), 3.91 (s, 3H), 3.84-3.37 (m, 5H), 3.07-2.60 (m, 1H), 2.01-1.75 (m, 2H), 1.21-0.87 (m, 3H), one proton obscured.

### Example 73

### 4-[(2-{[(4-Chloropyridin-2-yl)methyl]amino}-7-methoxy-1,3-benzoxazol-5-yl)carbonyl]-3-ethyl-1,4-diazabicyclo[4.2.0]octan-2-one [diastereomer mixture, 4 isomers]

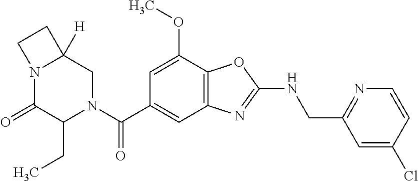 [SMILES: CCC1C(=O)N2CCC2CN1C(=O)c1cc(OC)c2oc(NCc3cc(Cl)ccn3)nc2c1]

60 mg (0.39 mmol) of 3-ethyl-1,4-diazabicyclo[4.2.0]octan-2-one [diastereomer mixture, 4 isomers], 23.8 mg (0.19 mmol) of 4-dimethylaminopyridine and 95.9 mg (0.500 mmol) of N-[3-(dimethylamino)propyl]-N′-ethylcarbodiimide were added to 92.7 mg (0.278 mmol) of 2-{[(4-chloropyridin-2-yl)methyl]amino}-7-methoxy-1,3-benzoxazole-5-carboxylic acid in N,N-dimethylformamide (2.8 ml), and the mixture was stirred at room temperature for 1.5 h.

Acetonitrile and water were then added and the product was purified by preparative HPLC (acetonitrile/water). This gave 62.8 mg (48% of theory) of the target compound.

LC-MS (Method 1A): Rₜ=0.84 min; MS (ESIpos): m/z=471 [M+H]⁺

¹H-NMR (400 MHz, DMSO-d₆): δ[ppm]=8.71 (t, 1H), 8.52 (d, 1H), 7.52 (s, 1H), 7.46 (d, 1H), 6.92-6.86 (m, 1H), 6.77-6.70 (m, 1H), 4.63 (d, 3H), 4.17-4.00 (m, 1H), 3.98-3.87 (m, 4H), 3.71 (dd, 1H), 3.02-2.90 (m, 1H), 2.08-2.00 (m, 1H), 1.71-1.42 (m, 1H), 0.97 (t, 1H), 0.84 (br. s., 3H); 2H presumably hidden under DMSO signal.

### Example 74

### 4-[(2-{[(4-Chloropyridin-2-yl)methyl]amino}-7-methoxy-1,3-benzoxazol-5-yl)carbonyl]-3-(methoxymethyl)-1,4-diazabicyclo[4.2.0]octan-2-one [enantiomerically pure isomer 3]

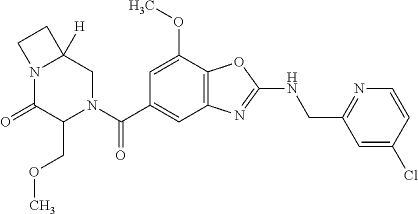 [SMILES: COCC1C(=O)N2CCC2CN1C(=O)c1cc(OC)c2oc(NCc3cc(Cl)ccn3)nc2c1]

69 mg (0.40 mmol) of 3-(methoxymethyl)-1,4-diazabicyclo[4.2.0]octan-2-one [enantiomerically pure isomer 3], 24 mg (0.20 mmol) of 4-dimethylaminopyridine and 99 mg (0.52 mmol) of N-[3-(dimethylamino)propyl]-N′-ethylcarbodiimide were added to 96 mg (0.29 mmol) of 2-{[(4-chloropyridin-2-yl)methyl]amino}-7-methoxy-1,3-benzoxazole-5-carboxylic acid in N,N-dimethylformamide (2.9 ml), and the mixture was stirred at room temperature for 1 h. Acetonitrile and water were then added and the product was purified by preparative HPLC (acetonitrile/water). This gave 56.7 mg (40% of theory) of the target compound.

LC-MS (Method 1A): Rₜ=0.74 min; MS (ESIpos): m/z=486 [M+H]⁺

¹H-NMR (400 MHz, DMSO-d₆): δ[ppm]=8.72 (t, 1H), 8.52 (d, 1H), 7.52 (s, 2H), 6.90 (s, 1H), 6.77 (s, 1H), 4.69-4.51 (m, 3H), 4.40 (br. s., 1H), 4.12-3.98 (m, 1H), 3.96-3.86 (m, 4H), 3.74-3.54 (m, 3H), 3.41 (t, 1H), 2.89-2.73 (s, 1H), 2.45-2.37 (m, 1H), 2.14-1.98 (m, 2H); 1H presumably hidden under DMSO signal.

### Example 75

### 4-[(2-{[(4-Chloropyridin-2-yl)methyl]amino}-7-methoxy-1,3-benzoxazol-5-yl)carbonyl]-3-methyl-1,4-diazabicyclo[4.2.0]octan-2-one [enantiomerically pure isomer 1]

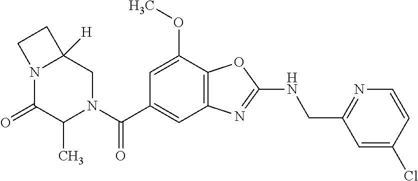 [SMILES: COc1cc(C(=O)N2CC3CCN3C(=O)C2C)cc2nc(NCc3cc(Cl)ccn3)oc12]

120 mg (162 μl, 0.929 mmol) of N,N-diisopropylethylamine, 121 mg (0.32 mmol) of HATU and 74 mg (0.53 mmol) of methyl 1,4-diazabicyclo[4.2.0]octan-2-one [diastereomer mixture, 4 isomers] were added to 88 mg (0.27 mmol) of 2-{[(4-chloropyridin-2-yl)methyl]amino}-7-methoxy-1,3-benzoxazole-5-carboxylic acid in N,N-dimethylformamide (2 ml), and the mixture was stirred at room temperature for 2.5 h. The product was then purified by preparative HPLC (acetonitrile/water). This gave 41 mg (33% of theory) of the target compound as a diastereomer mixture, 4 isomers.

LC-MS (Method 1A): Rₜ=0.75 min; MS (ESIpos): m/z=456 [M+H]⁺

¹H-NMR (400 MHz, DMSO-d₆): δ[ppm]=8.78-8.66 (m, 1H), 8.52 (d, 1H), 7.58-7.42 (m, 2H), 6.92-6.83 (m, 1H), 6.79-6.72 (m, 1H), 4.63 (d, 4H), 4.23 (d, 1H), 4.11-3.85 (m, 6H), 3.79-3.65 (m, 1H), 2.08 (d, 1H), 1.44-1.32 (m, 3H).

The diastereomer mixture was separated into the enantiomers on a chiral phase [Method 54D].

enantiomerically pure isomer 1: Yield: 7 mg (99% ee)

enantiomerically pure isomer 1: Rₜ=10.86 min [Method 46E].

LC-MS (Method 1A): Rₜ=0.73 min; MS (ESIpos): m/z=456 [M+H]⁺

¹H-NMR (400 MHz, DMSO-d₆): δ[ppm]=8.72 (t, 1H), 8.52 (d, 1H), 7.52 (s, 1H), 7.44-7.40 (m, 1H), 6.87 (s, 1H), 6.72 (s, 1H), 4.77-4.56 (m, 4H), 4.12-3.97 (m, 2H), 3.96-3.93 (m, 1H), 3.92 (s, 3H), 3.74-3.63 (m, 1H), 2.31-2.11 (m, 1H), 1.44-1.30 (m, 3H).

### Example 76

### 7-[(2-{[(4-Chloropyridin-2-yl)methyl]amino}-7-methoxy-1,3-benzoxazol-5-yl)carbonyl]-6-methyl-4,7-diazaspiro[2.5]octan-5-one [racemate]

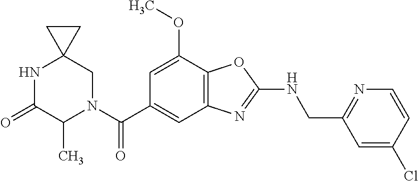 [SMILES: COc1cc(C(=O)N2CC3(CC3)NC(=O)C2C)cc2nc(NCc3cc(Cl)ccn3)oc12]

130 mg (0.927 mmol) of 6-methyl-4,7-diazaspiro[2.5]octan-5-one trifluoroacetate [racemate], 57 mg (0.46 mmol) of 4-dimethylaminopyridine and 228 mg (1.19 mmol) of N-[3-(dimethylamino)propyl]-N′-ethylcarbodiimide were added to 221 mg (0.662 mmol) of 2-{[(4-chloropyridin-5-yl)methyl]amino}-7-methoxy-1,3-benzoxazole-5-carboxylic acid in N,N-dimethylformamide (6 ml), and the mixture was stirred at room temperature for 1 h. Acetonitrile and water were then added and the product was purified by preparative HPLC (acetonitrile/water). This gave 174 mg (58% of theory) of the target compound as a racemate.

LC-MS (Method 1A): Rₜ=0.78 min; MS (ESIpos): m/z=455 [M+H]⁺

¹H-NMR (400 MHz, DMSO-d₆): δ[ppm]=8.72 (t, 1H), 8.51 (d, 1H), 8.15 (s, 1H), 7.52 (s, 1H), 7.45 (dd, 1H), 6.84 (s, 1H), 6.70 (s, 1H), 4.63 (d, 2H), 3.92 (s, 3H), 1.43 (d, 3H), 0.84-0.50 (m, 4H); 3H presumably hidden under DMSO signal.

### Example 77

### 7-[(2-{[(4-Chloropyridin-2-yl)methyl]amino}-7-methoxy-1,3-benzoxazol-5-yl)carbonyl]-6-methyl-4,7-diazaspiro[2.5]octan-5-one [enantiomerically pure isomer 1]

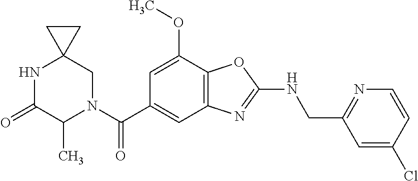 [SMILES: COc1cc(C(=O)N2CC3(CC3)NC(=O)C2C)cc2nc(NCc3cc(Cl)ccn3)oc12]

The racemate from Example 76 was separated into the enantiomers on a chiral phase [Method 55D].

enantiomerically pure isomer 1: Yield: 79 mg (100% ee)

enantiomerically pure isomer 1: Rₜ=4.48 min [Method 47E].

LC-MS (Method 1A): Rₜ=0.77 min; MS (ESIpos): m/z=455 [M+H]⁺

¹H-NMR (400 MHz, DMSO-d₆): δ[ppm]=8.72 (t, 1H), 8.52 (d, 1H), 8.15 (s, 1H), 7.53 (s, 2H), 6.84 (s, 1H), 6.71 (s, 1H), 4.63 (d, 2H), 3.92 (s, 3H), 1.51-1.35 (m, 4H), 0.90-0.54 (m, 4H); 2H presumably hidden under DMSO signal.

### Example 78

### 7-[(2-{[(4-Chloropyridin-2-yl)methyl]amino}-7-methoxy-1,3-benzoxazol-5-yl)carbonyl]-6-ethyl-4,7-diazaspiro[2.5]octan-5-one [racemate]

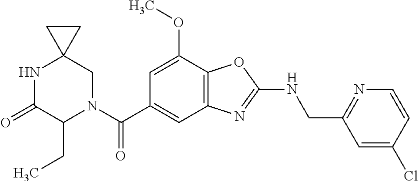 [SMILES: CCC1C(=O)NC2(CC2)CN1C(=O)c1cc(OC)c2oc(NCc3cc(Cl)ccn3)nc2c1]

60 mg (0.39 mmol) of 6-ethyl-4,7-diazaspiro[2.5]octan-5-one [racemate], 24 mg (0.19 mmol) of 4-dimethylaminopyridine and 96 mg (0.50 mmol) of N-[3-(dimethylamino)propyl]-N′-ethylcarbodiimide were added to 92 mg (0.28 mmol) of 2-{[(4-chloropyridin-5-yl)methyl]amino}-7-methoxy-1,3-benzoxazole-5-carboxylic acid in N,N-dimethylformamide (2.8 ml), and the mixture was stirred at room temperature for 1.5 h. Acetonitrile and water were then added and the product was purified by preparative HPLC (acetonitrile/water). This gave 50 mg (38% of theory) of the target compound as a racemate.

LC-MS (Method 1A): Rₜ=0.83 min; MS (ESIpos): m/z=470 [M+H]⁺

¹H-NMR (400 MHz, DMSO-d₆): δ[ppm]=8.73 (t, 1H), 8.52 (d, 1H), 8.10 (s, 1H), 7.58-7.39 (m, 2H), 6.85 (br. s., 1H), 6.70 (s, 1H), 4.87-4.58 (m., 3H), 3.92 (s, 3H), 3.78-3.68 (m, 1H), 3.01-2.89 (m, 1H), 2.02-1.83 (m, 2H), 0.97 (t, 3H), 0.77-0.45 (m, 3H), 0.29 (br. s., 1H).

### Example 79

### 7-[(2-{[(4-Chloropyridin-2-yl)methyl]amino}-7-methoxy-1,3-benzoxazol-5-yl)carbonyl]-6-ethyl-4,7-diazaspiro[2.5]octan-5-one [enantiomerically pure isomer 1]

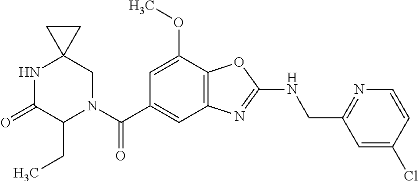 [SMILES: CCC1C(=O)NC2(CC2)CN1C(=O)c1cc(OC)c2oc(NCc3cc(Cl)ccn3)nc2c1]

50 mg of 7-[(2-{[(4-chloropyridin-2-yl)methyl]amino}-7-methoxy-1,3-benzoxazol-5-yl)carbonyl]-6-ethyl-4,7-diazaspiro[2.5]octan-5-one [racemate] Example 78 were separated into the enantiomers on a chiral phase [Method 56D].

enantiomerically pure isomer 1: Yield: 21 mg (100% ee)

enantiomerically pure isomer 1: Rₜ=4.20 min [Method 48E].

LC-MS (Method 1A): Rₜ=0.81 min; MS (ESIpos): m/z=470 [M+H]⁺

¹H-NMR (400 MHz, DMSO-d₆): δ[ppm]=8.73 (t, 1H), 8.51 (d, 1H), 8.09 (s, 1H), 7.52 (s, 1H), 7.48-7.42 (m, 1H), 6.84 (br. s., 1H), 6.70 (s, 1H), 4.88-4.72 (m, 1H), 4.62 (d, 2H), 3.92 (s, 3H), 3.78-3.67 (m, 1H), 3.18-3.09 (m, 1H), 1.98-1.82 (m, 2H), 1.05-0.52 (m, 6H), 0.29 (br. s., 1H).

### Example 80

### 4-[(2-{[(4-Chloropyridin-2-yl)methyl]amino}-7-methoxy-1,3-benzoxazol-5-yl)carbonyl]-3-ethyl-6-methylpiperazin-2-one [diastereomer 2, 2 isomers]

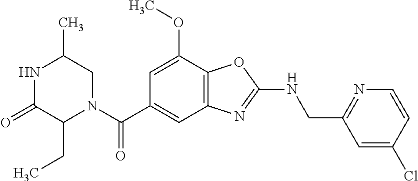 [SMILES: CCC1C(=O)NC(C)CN1C(=O)c1cc(OC)c2oc(NCc3cc(Cl)ccn3)nc2c1]

86 mg (116 μl, 0.67 mmol) of N,N-diisopropylethylamine, 87 mg (0.23 mmol) of HATU and 54 mg (0.38 mmol) of 3-ethyl-6-methylpiperazin-2-one [diastereomer mixture, 4 isomers] were added to 63.7 mg (0.191 mmol) of 2-{[(4-chloropyridin-2-yl)methyl]amino}-7-methoxy-1,3-benzoxazole-5-carboxylic acid in N,N-dimethylformamide (2 ml), and the mixture was stirred at room temperature for 2.5 h. The mixture was then concentrated under reduced pressure, and the residue was dissolved in methanol and water and purified by preparative HPLC (acetonitrile/water). This gave 45 mg (51% of theory) of the target compound as a diastereomer mixture, 4 isomers.

LC-MS (Method 1A): Rₜ=0.74 min (diastereomer 1, 2 isomers), Rₜ=0.77 min (diastereomer 2, 2 isomers);

MS (ESIpos): m/z=458 [M+H]⁺

The diastereomer mixture, 4 isomers, was separated into diastereomer 1, 2 isomers, and diastereomer 2, 2 isomers [Method 2G].

Diastereomer 2 (2 isomer): Rₜ=9.1 min

Diastereomer 2 (2 isomer): Yield: 16 mg

LC-MS (Method 1A): Rₜ=0.77 min; MS (ESIpos): m/z=458 [M+H]⁺

¹H-NMR (400 MHz, DMSO-d₆): δ[ppm]=8.72 (t, 1H), 8.52 (d, 1H), 7.99 (br. s., 1H), 7.53 (s, 1H), 7.48-7.43 (m, 1H), 6.88 (br. s., 1H), 6.73 (s, 1H), 4.73 (br. s., 1H), 4.63 (d, 2H), 3.92 (s, 3H), 3.67-3.40 (m, 2H), 3.03-2.93 (m, 1H), 1.97-1.72 (m, 2H), 0.93 (br. s., 6H).

### Example 81

### 4-[(2-{[(4-Chloropyridin-2-yl)methyl]amino}-7-methoxy-1,3-benzoxazol-5-yl)carbonyl]-3-ethyl-6-methylpiperazin-2-one [enantiomerically pure isomer 3]

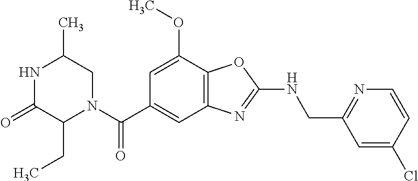 [SMILES: CCC1C(=O)NC(C)CN1C(=O)c1cc(OC)c2oc(NCc3cc(Cl)ccn3)nc2c1]

28.2 mg (0.19 mmol) of 3-ethyl-6-methylpiperazin-2-one [enantiomerically pure isomer 3], 12 mg (0.10 mmol) of 4-dimethylaminopyridine and 48 mg (0.25 mmol) of N-[3-(dimethylamino)propyl]-N′-ethylcarbodiimide were added to 47.3 mg (0.142 mmol) of 2-{[(4-chloropyridin-5-yl)methyl]amino}-7-methoxy-1,3-benzoxazole-5-carboxylic acid in N,N-dimethylformamide (1.4 ml), and the mixture was stirred at room temperature for 1 h. Methanol and water were then added and the product was purified by preparative HPLC (acetonitrile/water). This gave 12 mg (18% of theory) of the target compound.

LC-MS (Method 1A): Rₜ=0.80 min; MS (ESIpos): m/z=458 [M+H]⁺

¹H-NMR (400 MHz, DMSO-d₆): δ[ppm]=8.73 (br. s., 1H), 8.52 (d, 1H), 7.99 (br. s., 1H), 7.53 (s, 1H), 7.46 (dd, 1H), 6.88 (br. s., 1H), 6.73 (s, 1H), 4.78-4.67 m, 1H), 4.62 (br. s., 2H), 3.92 (s, 3H), 3.74-3.41 (m, 2H), 3.04-2.91 (m, 1H), 1.96-1.73 (m, 2H), 1.05-0.83 (m, 6H).

### Example 82

### 2-{1-[(2-{[(4-Chloropyridin-2-yl)methyl]amino}-7-methoxy-1,3-benzoxazol-5-yl)carbonyl]-5,5-dimethyl-3-oxopiperazin-2-yl}acetamide [racemate]

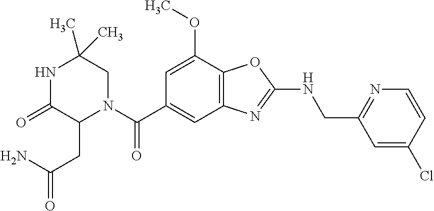 [SMILES: COc1cc(C(=O)N2CC(C)(C)NC(=O)C2CC(N)=O)cc2nc(NCc3cc(Cl)ccn3)oc12]

1.15 g (1.55 ml, 8.92 mmol) of N,N-diisopropylethylamine, 1.85 g (4.87 mmol) of HATU and 902 mg (4.87 mmol) of 2-(5,5-dimethyl-3-oxopiperazin-2-yl)acetamide [racemate] were added to 1.44 g (4.06 mmol) of 2-{[(4-chloropyridin-2-yl)methyl]amino}-7-methoxy-1,3-benzoxazole-5-carboxylic acid in N,N-dimethylformamide (18 ml), and the mixture was stirred at room temperature for 3 h. The mixture was then concentrated under reduced pressure and the residue was purified by preparative RP-HPLC (acetonitrile/water). This gave 390 mg (19% of theory) of the target compound.

LC-MS (Method 1A): Rₜ=0.66 min; MS (ESIpos): m/z=501 [M+H]⁺

### Example 83

### {1-[(2-{[(4-Chloropyridin-2-yl)methyl]amino}-7-methoxy-1,3-benzoxazol-5-yl)carbonyl]-5,5-dimethyl-3-oxopiperazin-2-yl}acetonitrile [racemate]

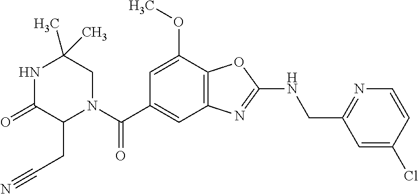 [SMILES: COc1cc(C(=O)N2CC(C)(C)NC(=O)C2CC#N)cc2nc(NCc3cc(Cl)ccn3)oc12]

288 mg (295 μl, 3.64 mmol) of pyridine and 764 mg (514 μl, 3.64 mmol) of trifluoroacetic anhydride were added to 1.44 g (4.06 mmol) of 2-{1-[(2-{[(4-chloropyridin-2-yl)methyl]amino}-7-methoxy-1,3-benzoxazol-5-yl)carbonyl]-5,5-dimethyl-3-oxopiperazin-2-yl}acetamide [racemate] in THF (8 ml), and the mixture was stirred at room temperature for 2 h. The mixture was then added to water, methanol was added and the product was purified by preparative HPLC (acetonitrile/water). This gave 150 mg (33% of theory) of the target compound as a racemate.

LC-MS (Method 1A): Rₜ=0.76 min; MS (ESIpos): m/z=483 [M+H]⁺

### Example 84

### {1-[(2-{[(4-chloropyridin-2-yl)methyl]amino}-7-methoxy-1,3-benzoxazol-5-yl)carbonyl]-5,5-dimethyl-3-oxopiperazin-2-yl}acetonitrile [enantiomerically pure isomer 2]

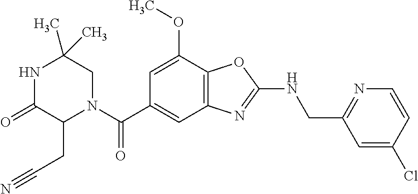 [SMILES: COc1cc(C(=O)N2CC(C)(C)NC(=O)C2CC#N)cc2nc(NCc3cc(Cl)ccn3)oc12]

The racemate from Example 83 was separated into the enantiomers on a chiral phase [Method 57D].

Enantiomerically pure isomer 2: Yield: 23 mg (97% ee)

Enantiomerically pure isomer 2: Rₜ=6.33 min [Method 49E].

LC-MS (Method 1A): Rₜ=0.76 min; MS (ESIpos): m/z=483 [M+H]⁺

¹H-NMR (400 MHz, DMSO-d₆): δ[ppm]=8.74 (t, 1H), 8.52 (d, 1H), 8.41 (s, 1H), 7.53 (d, 1H), 7.45 (dd, 1H), 6.89 (s, 1H), 6.76 (s, 1H), 5.75 (s, 1H), 4.63 (d, 2H), 3.92 (s, 3H), 3.44-3.34 (m, 1H), 3.27-3.18 (m, 1H), 3.15-3.01 (m, 1H), 1.07 (d, 6H); 1H presumably hidden under DMSO signal.

### Example 85

### (2-{[(4-Chloropyridin-2-yl)methyl]amino}-7-methoxy-1,3-benzoxazol-5-yl) [2-(2-hydroxyethyl)-5-methylpiperidin-1-yl]methanone [diastereomer 2, 2 isomers]

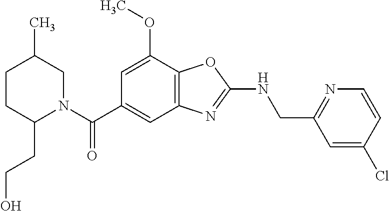 [SMILES: COc1cc(C(=O)N2CC(C)CCC2CCO)cc2nc(NCc3cc(Cl)ccn3)oc12]

195 mg (264 μl, 1.51 mmol) of N,N-diisopropylethylamine and 74.5 mg (0.416 mmol) of 2-(5-methylpiperidin-2-yl)ethanol [diastereomer mixture, 4 isomers] were added to 126 mg (0.378 mmol) of 2-{[(4-chloropyridin-2-yl)methyl]amino}-7-methoxy-1,3-benzoxazole-5-carboxylic acid in N,N-dimethylformamide (1.9 ml). 172 mg (0.454 mmol) of HATU were then added at 0° C., and the mixture was stirred at room temperature for 4 h. Water was added and the product was purified by preparative HPLC (acetonitrile/water). This gave 61 mg (18% of theory) of the target compound as a diastereomer mixture.

LC-MS (Method 1A): Rₜ=0.88 min (diastereomer 1, 2 isomers), Rₜ=0.89 min (diastereomer 2, 2 isomers);

MS (ESIpos): m/z=459 [M+H]⁺

The diastereomer mixture was separated [Method 3G].

Diastereomer 2 (2 isomer): Rₜ=7.1 min

Diastereomer 2 (2 isomer): Yield: 2 mg

LC-MS (Method 1A): Rₜ=0.91 min; MS (ESIpos): m/z=459 [M+H]⁺

### Example 86

### (2-{[(4-Chloropyridin-2-yl)methyl]amino}-7-methoxy-1,3-benzoxazol-5-yl) [2-(2-hydroxy-2-methylpropyl)-5-methylpiperidin-1-yl]methanone [diastereomer 1, 2 isomers]

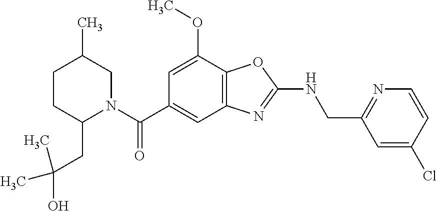 [SMILES: COc1cc(C(=O)N2CC(C)CCC2CC(C)(C)O)cc2nc(NCc3cc(Cl)ccn3)oc12]

At 0° C., 256 μl (0.768 mmol) of a 3M solution of methylmagnesium bromide in diethyl ether were added dropwise to 110 mg (0.22 mmol) of ethyl {1-[(2-{[(4-chloropyridin-2-yl)methyl]amino}-7-methoxy-1,3-benzoxazol-5-yl)carbonyl]-5-methylpiperidin-2-yl}acetate [diastereomer mixture, 4 isomers] in 2.44 ml of THF, and the mixture was stirred at 0° C. for 15 min and then at room temperature overnight. Saturated aqueous ammonium chloride solution was added carefully and the mixture was concentrated under reduced pressure to half its original volume. Water and dichloromethane were added and the phases were separated. The organic phase was twice washed with water and then dried over sodium sulphate. After filtration, the filtrate was concentrated under reduced pressure and the residue was purified by preparative HPLC (acetonitrile/water). This gave 67 mg (62% of theory) of the target compound as a diastereomer mixture, 4 isomers.

LC-MS (Method 1A): Rₜ=0.96 min (diastereomer 1, 2 isomers), Rₜ=0.99 min (diastereomer 2, 2 isomers);

MS (ESIpos): m/z=487 [M+H]⁺

The diastereomer mixture was separated [Method 60D].

Diastereomer 1 (2 isomer): Rₜ=7.1 min

Diastereomer 1 (2 isomer): Yield: 17 mg

Diastereomer 1 (2 isomer): Rₜ=6.42 min [Method 52E].

LC-MS (Method 1A): Rₜ=0.93 min; MS (ESIpos): m/z=487 [M+H]⁺

¹H-NMR (500 MHz, DMSO-d₆): δ[ppm]=8.64 (d, 1H), 8.51 (d, 1H), 7.52 (s, 1H), 7.47-7.41 (m, 1H), 6.83-6.72 (m, 1H), 6.71-6.57 (m, 1H), 4.89 (br. s., 1H), 4.62 (d, 2H), 4.30-4.15 (m, 1H), 3.90 (s, 3H), 2.79 (t, 1H), 1.97-1.21 (m, 6H), 1.19-1.10 (m, 4H), 1.09-0.64 (m, 6H).

### Example 87

### (2-{[(4-Chloropyridin-2-yl)methyl]amino}-7-methoxy-1,3-benzoxazol-5-yl){2-[(1-hydroxycyclopropyl)methyl]-5-methylpiperidin-1-yl}methanone [diastereomer mixture, 4 isomers]

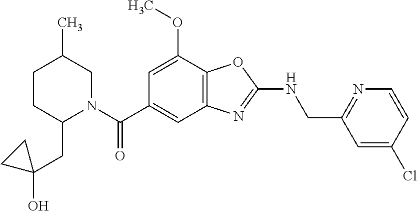 [SMILES: COc1cc(C(=O)N2CC(C)CCC2CC2(O)CC2)cc2nc(NCc3cc(Cl)ccn3)oc12]

365 mg (492 μl, 2.82 mmol) of N,N-diisopropylethylamine, 257 mg (0.678 mmol) of HATU and 320 mg (1.13 mmol) of 1-[(5-methylpiperidin-2-yl)methyl]cyclopropanol [diastereomer mixture, 4 isomers] were added to 188 mg (0.565 mmol) of 2-{[(4-chloropyridin-2-yl)methyl]amino}-7-methoxy-1,3-benzoxazole-5-carboxylic acid in N,N-dimethylformamide (5.5 ml). The mixture was stirred at room temperature for 2 h and purified by preparative HPLC (acetonitrile/water). This gave 56 mg (18% of theory) of the target compound as a diastereomer mixture, 4 isomers.

LC-MS (Method 1A): Rₜ=1.04 min; MS (ESIpos): m/z=485 [M+H]⁺

¹H-NMR (400 MHz, DMSO-d₆): δ[ppm]=8.67 (t, 1H), 8.51 (d, 1H), 7.52 (s, 1H), 7.46-7.40 (m, 1H), 6.83-6.56 (m, 2H), 5.15-5.03 (m, 1H), 4.63 (d, 2H), 3.96-3.81 (m, 3H), 2.86-2.59 (m, 2H), 1.96-1.08 (m, 8H), 0.97-0.65 (m, 6H), 0.61-0.24 (m, 1H).

### Example 88

### (2-{[(4-Chloropyridin-2-yl)methyl]amino}-7-methoxy-1,3-benzoxazol-5-yl){2-[(1-hydroxycyclopropyl)methyl]-5-methylpiperidin-1-yl}methanone [enantiomerically pure isomer 2]

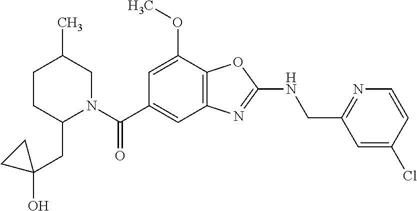 [SMILES: COc1cc(C(=O)N2CC(C)CCC2CC2(O)CC2)cc2nc(NCc3cc(Cl)ccn3)oc12]

The diastereomer mixture from Example 87 was separated [Method 8F].

Enantiomerically pure isomer 2: Yield: 18 mg

LC-MS (Method 7A): Rₜ=3.06 min; MS (ESIpos): m/z=485 [M+H]⁺

### Example 89

### (2-{[(4-Chloropyridin-2-yl)methyl]amino}-7-methoxy-1,3-benzoxazol-5-yl) [2-(1-hydroxycyclopropyl)-5-methylpiperidin-1-yl]methanone [diastereomer 1, 2 isomers]

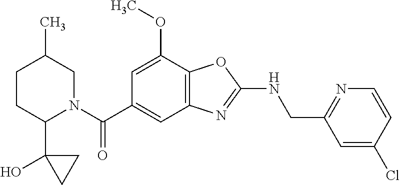 [SMILES: COc1cc(C(=O)N2CC(C)CCC2C2(O)CC2)cc2nc(NCc3cc(Cl)ccn3)oc12]

384 mg (518 μl, 2.97 mmol) of N,N-diisopropylethylamine, 271 mg (0.713 mmol) of HATU and 320 mg (1.19 mmol) of 1-(5-methylpiperidin-2-yl)cyclopropanol trifluoroacetate [diastereomer mixture, 4 isomers] were added to 198 mg (0.594 mmol) of 2-{[(4-chloropyridin-2-yl)methyl]amino}-7-methoxy-1,3-benzoxazole-5-carboxylic acid in N,N-dimethylformamide (6.5 ml). The mixture was stirred at room temperature for 2 h and purified by preparative HPLC (acetonitrile/water). This gave 45 mg (15% of theory) of diastereomer 1, 2 isomers.

LC-MS (Method 1A): Rₜ=0.93 min; MS (ESIpos): m/z=471 [M+H]⁺

¹H-NMR (400 MHz, DMSO-d₆): δ[ppm]=8.66 (t, 1H), 8.52 (d, 1H), 7.53 (s, 1H), 7.47-7.39 (m, 1H), 7.33-7.18 (m, 1H), 6.83 (br. s., 1H), 6.67 (s, 1H), 5.40 (s, 1H), 4.63 (d, 2H), 3.95-3.86 (m, 3H), 1.93-1.87 (m, 1H), 1.78-1.41 (m, 4H), 1.07-0.45 (m, 8H); 2H presumably obscured by DMSO signal.

### Example 90

### (2-{[(4-Chloropyridin-2-yl)methyl]amino}-7-methoxy-1,3-benzoxazol-5-yl) [2-(1-hydroxycyclopropyl)-5-methylpiperidin-1-yl]methanone [diastereomer 2, 2 isomers]

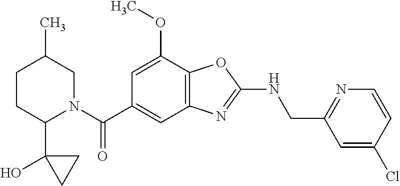 [SMILES: COc1cc(C(=O)N2CC(C)CCC2C2(O)CC2)cc2nc(NCc3cc(Cl)ccn3)oc12]

384 mg (518 μl, 2.97 mmol) of N,N-diisopropylethylamine, 271 mg (0.713 mmol) of HATU and 320 mg (1.19 mmol) of 1-(5-methylpiperidin-2-yl)cyclopropanol trifluoroacetate [diastereomer mixture, 4 isomers] were added to 198 mg (0.594 mmol) of 2-{[(4-chloropyridin-2-yl)methyl]amino}-7-methoxy-1,3-benzoxazole-5-carboxylic acid in N,N-dimethylformamide (6.5 ml). The mixture was stirred at room temperature for 2 h and purified by preparative HPLC (acetonitrile/water). This gave 45 mg (15% of theory) of diastereomer 2, 2 isomers.

LC-MS (Method 1A): Rₜ=1.06 min; MS (ESIpos): m/z=471 [M+H]⁺

¹H-NMR (400 MHz, DMSO-d₆): δ [ppm]=8.74-8.63 (m, 1H), 8.51 (d, 1H), 7.53 (br. s., 1H), 7.48-7.41 (m, 1H), 7.33-7.21 (m, 1H), 6.88-6.55 (m, 2H), 5.30-5.15 (m, 1H), 4.63 (d, 2H), 3.96-3.86 (m, 3H), 3.77-3.42 (m, 1H), 1.98-1.48 (m, 4H), 1.09-0.62 (m, 8H); 2H presumably obscured by DMSO signal.

### Example 91

### (2-{[(4-Chloropyridin-2-yl)methyl]amino}-7-methoxy-1,3-benzoxazol-5-yl) [2-(1-hydroxycyclopropyl)-5-methylpiperidine-1-yl]methanone [enantiomerically pure isomer 2]

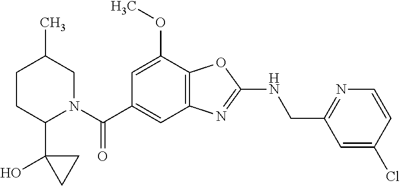 [SMILES: COc1cc(C(=O)N2CC(C)CCC2C2(O)CC2)cc2nc(NCc3cc(Cl)ccn3)oc12]

(2-{[(4-Chloropyridin-2-yl)methyl]amino}-7-methoxy-1,3-benzoxazol-5-yl) [2-(1-hydroxycyclopropyl)-5-methylpiperidin-1-yl]methanone [diastereomer 1, 2 isomers] Example 89 were separated on a chiral phase [Method 58D].

Enantiomerically pure isomer 2: Yield: 14 mg

Enantiomerically pure isomer 2: Rₜ=9.26 min [Method 50E].

LC-MS (Method 1A): Rₜ=0.93 min; MS (ESIpos): m/z=471 [M+H]⁺

¹H-NMR (400 MHz, DMSO-d₆): δ[ppm]=8.69 (t, 1H), 8.52 (d, 1H), 7.53 (s, 1H), 7.47-7.39 (m, 1H), 6.88-6.78 (m, 1H), 6.67 (s, 1H), 5.42 (s, 1H), 4.63 (d, 2H), 4.07 (br. s., 1H), 3.94-3.85 (m, 3H), 3.58-3.50 (m, 2H), 2.10-1.19 (m, 6H), 0.97-0.40 (m, 6H).

### Example 92

### {1-[(2-{[(4-Chloropyridin-2-yl)methyl]amino}-7-methoxy-1,3-benzoxazol-5-yl)carbonyl]-5-methyl-3-oxopiperazin-2-yl}acetonitrile [enantiomerically pure isomer 4]

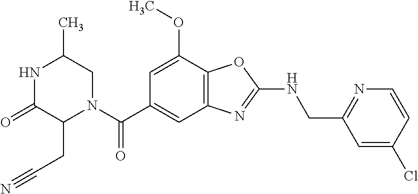 [SMILES: COc1cc(C(=O)N2CC(C)NC(=O)C2CC#N)cc2nc(NCc3cc(Cl)ccn3)oc12]

609 mg (623 μl, 7.70 mmol) of pyridine and 1.62 g (1.08 ml, 7.70 mmol) of trifluoroacetic anhydride were added to 850 mg (1.77 mmol) of 2-{1-[(2-{[(4-chloropyridin-2-yl)methyl]amino}-7-methoxy-1,3-benzoxazol-5-yl)carbonyl]-5-methyl-3-oxopiperazin-2-yl}acetamide [diastereomer mixture, 4 isomers] in THF (17 ml), and the mixture was stirred at room temperature for 1 h. The mixture was then carefully added to ice-water, dichloromethane was added, the phases were separated and the aqueous phase was washed twice with dichloromethane. The combined organic phases were washed with saturated aqueous sodium chloride solution and dried over sodium sulphate. After filtration, the filtrate was concentrated under reduced pressure, acetonitrile and water were added to the residue and the product was purified by silica gel chromatography (dichloromethane/methanol 100:3-100:7). This gave 336 mg (42% of theory) of the target compound as a diastereomer mixture, 4 isomers.

LC-MS (Method 1A): Rₜ=0.72 min; MS (ESIpos): m/z=469 [M+H]⁺

The diastereomer mixture was separated into the enantiomers on a chiral phase [Method 59D].

enantiomerically pure isomer 4: Yield: 18 mg (97% ee)

enantiomerically pure isomer 4: Rₜ=5.86 min [Method 51E].

LC-MS (Method 1A): Rₜ=0.73 min; MS (ESIpos): m/z=469 [M+H]⁺

¹H-NMR (400 MHz, DMSO-d₆): δ[ppm]=8.73 (t, 1H), 8.52 (d, 1H), 8.40 (s, 1H), 7.53 (s, 1H), 7.45 (dd, 1H), 6.96-6.87 (m, 1H), 6.83-6.67 (m, 1H), 4.63 (d, 2H), 3.92 (s, 3H), 3.61-3.54 (m, 1H), 3.24-3.03 (m, 3H), 1.00 (br. s., 3H); 2H presumably hidden under DMSO signal.

### Example 93

### (2-{[(4-Chloropyridin-2-yl)methyl]amino}-7-methoxy-1,3-benzoxazol-5-yl) [5-(2-hydroxypropan-2-yl)-2,2-dimethylmorpholin-4-yl]methanone [enantiomerically pure isomer 1]

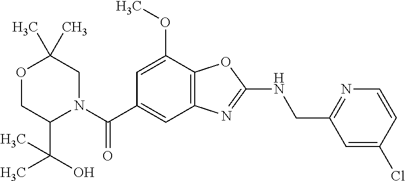 [SMILES: COc1cc(C(=O)N2CC(C)(C)OCC2C(C)(C)O)cc2nc(NCc3cc(Cl)ccn3)oc12]

130 mg (176 μl, 1.01 mmol) of N,N-diisopropylethylamine, 131 mg (0.35 mmol) of HATU and 2-(6,6-dimethylmorpholin-3-yl)propan-2-ol [enantiomerically pure isomer 1, Example 259A] were added to 96 mg (0.29 mmol) of 2-{[(4-chloropyridin-2-yl)methyl]amino}-7-methoxy-1,3-benzoxazole-5-carboxylic acid in N,N-dimethylformamide (3.0 ml), and the mixture was stirred at room temperature for 4.5 h. 56.5 mg (75 μl, 0.43 mmol) of N,N-diisopropylethylamine, 655 mg (1.75 mmol) of HATU and 50 mg (0.288 mmol) of 2-(6,6-dimethylmorpholin-3-yl)propan-2-ol [enantiomerically pure isomer 1, Example 259A] were then added and the mixture was stirred at room temperature for 2 h. The mixture was concentrated under reduced pressure and the residue was purified by preparative HPLC (acetonitrile/water). This gave 8 mg (6% of theory) of the target compound.

LC-MS (Method 1A): Rₜ=0.83 min; MS (ESIpos): m/z=489 [M+H]⁺

### Example 94

### (2-{[(4-Chloropyridin-2-yl)methyl]amino}-7-methoxy-1,3-benzoxazol-5-yl) [5-(1-hydroxycyclopropyl)-2,2-dimethylmorpholin-4-yl]methanone [enantiomerically pure isomer 1]

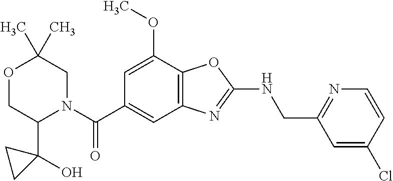 [SMILES: COc1cc(C(=O)N2CC(C)(C)OCC2C2(O)CC2)cc2nc(NCc3cc(Cl)ccn3)oc12]

143 mg (192 μl, 1.10 mmol) of N,N-diisopropylethylamine, 144 mg (0.378 mmol) of HATU and 108 mg (0.631 mmol) of 1-(6,6-dimethylmorpholin-3-yl)cyclopropanol [enantiomerically pure isomer 1, Example 263A] were added to 105 mg (0.315 mmol) of 2-{[(4-chloropyridin-2-yl)methyl]amino}-7-methoxy-1,3-benzoxazole-5-carboxylic acid in N,N-dimethylformamide (3.3 ml), and the mixture was stirred at room temperature for 3 h. The mixture was then concentrated under reduced pressure and the residue was purified by preparative HPLC (acetonitrile/water). This gave 55 mg (36% of theory) of the target compound.

LC-MS (Method 1A): Rₜ=0.83 min; M S (ESIpos): m/z=487 [M+H]⁺

¹H-NMR (400 MHz, DMSO-d₆): δ[ppm]=8.69 (t, 1H), 8.52 (d, 1H), 7.52 (s, 1H), 7.46 (dd, 1H), 6.96 (s, 1H), 6.77 (s, 1H), 5.76 (s, 1H), 5.52 (s, 1H), 4.62 (d, 2H), 3.91 (s, 4H), 3.75 (dd, 1H), 1.31-0.75 (m, 8H), 0.69-0.41 (m, 2H); 2H presumably hidden under DMSO signal.

### Example 95

### (2-{[(4-Chloropyridin-2-yl)methyl]amino}-7-methoxy-1,3-benzoxazol-5-yl) [5-(1-hydroxyethyl)-2,2-dimethylmorpholin-4-yl]methanone [enantiomerically pure isomer 1]

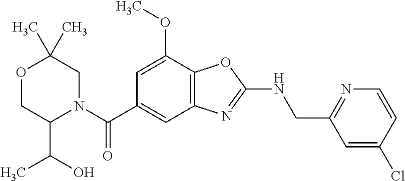 [SMILES: COc1cc(C(=O)N2CC(C)(C)OCC2C(C)O)cc2nc(NCc3cc(Cl)ccn3)oc12]

58 mg (78 μl, 0.45 mmol) of N,N-diisopropylethylamine, 58 mg (0.15 mmol) of HATU and 41 mg (0.26 mmol) of 1-(6,6-dimethylmorpholin-3-yl)ethanol [enantiomerically pure isomer 1, Example 270A] were added to 44 mg (0.13 mmol) of 2-{[(4-chloropyridin-2-yl)methyl]amino}-7-methoxy-1,3-benzoxazole-5-carboxylic acid in N,N-dimethylformamide (1 ml), and the mixture was stirred at room temperature for 2 h. The mixture was concentrated under reduced pressure and the residue was purified by preparative HPLC (acetonitrile/water). This gave 18 mg (29% of theory) of the target compound.

LC-MS (Method 1A): Rₜ=0.78 min; MS (ESIpos): m/z=475 [M+H]⁺

¹H-NMR (400 MHz, DMSO-d₆): δ[ppm]=8.73 (br. s., 1H), 8.52 (d, 1H), 7.52 (s, 1H), 7.46 (dd, 1H), 6.90-6.58 (m, 2H), 5.76 (s, 1H), 4.99-4.78 (m, 1H), 4.63 (d, 2H), 4.17-3.98 (m, 2H), 3.97-3.87 (m, 4H), 3.86-3.58 (m, 1H), 3.22-2.97 (m, 2H), 1.32-0.84 (m, 9H).

### Example 96

### (2-{[(4-Chloropyridin-2-yl)methyl]amino}-7-methoxy-1,3-benzoxazol-5-yl) [5-(1-hydroxyethyl)-2,2-dimethylmorpholin-4-yl]methanone [enantiomerically pure isomer 2]

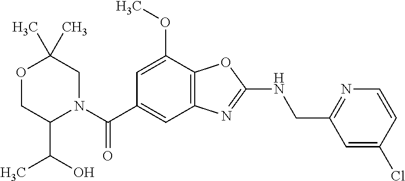 [SMILES: COc1cc(C(=O)N2CC(C)(C)OCC2C(C)O)cc2nc(NCc3cc(Cl)ccn3)oc12]

142 mg (191 μl, 1.11 mmol) of N,N-diisopropylethylamine, 143 mg (0.38 mmol) of HATU and 100 mg (0.628 mmol) of 1-(6,6-dimethylmorpholin-3-yl)ethanol [enantiomerically pure isomer 2, Example 269A] were added to 105 mg (0.31 mmol) of 2-{[(4-chloropyridin-2-yl)methyl]amino}-7-methoxy-1,3-benzoxazole-5-carboxylic acid in N,N-dimethylformamide (3 ml), and the mixture was stirred at room temperature for 3 h. The mixture was concentrated under reduced pressure and the residue was purified by preparative HPLC (acetonitrile/water). This gave 50 mg (32% of theory) of the target compound.

LC-MS (Method 1A): Rₜ=0.80 min; MS (ESIpos): m/z=475 [M+H]⁺

¹H-NMR (400 MHz, DMSO-d₆): δ[ppm]=8.67 (br. s., 1H), 8.52 (d, 1H), 7.53 (d, 1H), 7.45 (dd, 1H), 7.05-6.72 (m, 2H), 5.16-4.94 (m, 1H), 4.62 (d, 2H), 4.21-4.02 (m, 2H), 3.90 (s, 3H), 3.79-3.52 (m, 2H), 1.28-0.86 (m, 9H); 2H presumably hidden under DMSO signal.

### Example 97

### (2-{[1-(4-Chloropyridin-2-yl)ethyl]amino}-7-methoxy-1,3-benzoxazol-5-yl)[(4R)-4-[(1,1-²H₂)ethyloxy]-2-(hydroxymethyl)pyrrolidin-1-yl]methanone [diastereomer mixture, 4 isomers]

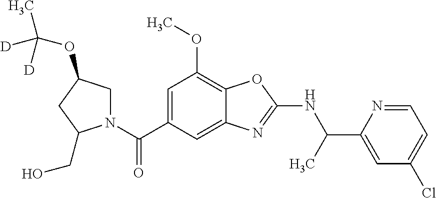 [SMILES: [2H]C([2H])(C)O[C@@H]1CC(CO)N(C(=O)c2cc(OC)c3oc(NC(C)c4cc(Cl)ccn4)nc3c2)C1]

300 mg (0.682 mmol, purity: 79%) of (2-{[1-(4-chloropyridin-2-yl)ethyl]amino}-7-methoxy-1,3-benzoxazole-5-carboxylic acid [racemate] and 120 mg (0.818 mmol) of {(4R)-4-[(1,1-²H₂)ethyloxy]pyrrolidin-2-yl}methanol [diastereomer mixture, 2 isomers] were initially charged in N,N-dimethylformamide (3.14 ml), and 308 mg (415 μl, 2.39 mmol) of N,N-diisopropylethylamine were added. 311 mg (0.818 mmol) of HATU were then added at RT, and the mixture was stirred for 1 h. Without further work-up, the reaction solution was then purified directly by preparative RP-HPLC (acetonitrile/water). Yield: 247 mg (76% of theory).

LC-MS (Method 1A): Rₜ=0.84 min (enantiomerically pure isomer 1), Rₜ=0.85 min (enantiomerically pure isomer 2), Rₜ=0.87 min (enantiomerically pure isomer 3); enantiomerically pure isomer 4 obscured.

MS (ESIpos): m/z=477 [M+H]⁺.

### B) ASSESSMENT OF PHYSIOLOGICAL EFFICACY

The suitability of the compounds according to the invention for treating thromboembolic disorders can be demonstrated in the following assay systems:

### a) Test Descriptions (In Vitro)

### a.1) Measurement of the Thrombin Inhibition in Buffer

To determine the thrombin inhibition of the substances listed above, a biochemical test system is constructed in which the conversion of a thrombin substrate is used for determining the enzymatic activity of human thrombin. Here, thrombin cleaves aminomethylcoumarin, which is measured fluorescently, from the peptic substrate. The determinations are carried out in microtitre plates.

Substances to be tested are dissolved in various concentrations in dimethyl sulphoxide and incubated for 15 min with human thrombin (0.06 nmol/l dissolved in 50 mmol/l of Tris buffer [C,C,C-tris(hydroxymethyl)aminomethane], 100 mmol/l of sodium chloride, 0.1% BSA [bovine serum albumin], pH 7.4) at 22° C. The substrate (5 μmol/l Boc-Asp(OBzl)-Pro-Arg-AMC from Bachem) is then added. After 30 min of incubation, the sample is excited at a wavelength of 360 nm and the emission is measured at 460 nm. The measured emissions of the test mixtures with test substance are compared to the control mixtures without test substance (only dimethyl sulphoxide instead of test substance in dimethyl sulphoxide) and IC₅₀ values are calculated from the concentration/activity relationships. Representative activity data from this test are given in Table 1 below (in some cases as means of individual determinations):

TABLE 1

| Example No. | IC₅₀ [nM] | Example No. | IC₅₀ [nM] |
|---|---|---|---|
| 1 | 15 | 2 | 6.2 |
| 3 | 330 | 4 | 39 |
| 5 | 2800 | 6 | 15 |
| 7 | 37 | 8 | 2600 |
| 9 | 9.7 | 10 | 31 |
| 11 | 31 | 12 | 4.4 |
| 13 | 21 | 14 | 550 |
| 15 | 6.4 | 16 | 30 |
| 17 | 33 | 18 | 17 |
| 19 | 34 | 20 | 36 |
| 21 | 10 | 22 | 1.7 |
| 23 | 0.8 | 24 | 2.5 |
| 25 | 1.0 | 26 | 6.2 |
| 27 | 2.1 | 28 | 3.2 |
| 29 | 1.9 | 30 | 25 |
| 31 | 11 | 32 | 12 |
| 33 | 7.2 | 34 | 7.3 |
| 35 | 5.0 | 36 | 39 |
| 37 | 37 | 38 | 13 |
| 39 | 11 | 40 | 18 |
| 41 | 50 | 42 | 27 |
| 43 | 18 | 44 | 32 |
| 45 | 24 | 46 | 14 |
| 47 | 11 | 48 | 11 |
| 49 | 24 | 50 | 12 |
| 51 | 8.0 | 52 | 28 |
| 53 | 36 | 54 | 11 |
| 55 | 21 | 56 | 25 |
| 57 | 20 | 58 | 17 |
| 59 | 60 | 60 | 5.2 |
| 61 | 24 | 62 | 68 |
| 63 | 11 | 64 | 44 |
| 65 | 24 | 66 | 28 |
| 67 | 16 | 68 | 59 |
| 69 | 17 | 70 | 38 |
| 71 | 97 | 72 | 29 |
| 73 | 51 | 74 | 32 |
| 75 | 32 | 76 | 19 |
| 77 | 14 | 78 | 13 |
| 79 | 4.5 | 80 | 16 |
| 81 | 12 | 82 | 170 |
| 83 | 36 | 84 | 11 |
| 85 | 17 | 87 | 5.3 |
| 88 | 6.6 | 89 | 17 |
| 90 | 28 | 91 | 13 |
| 92 | 96 | 93 | 8.8 |
| 94 | 18 | 95 | 6.8 |
| 96 | 41 | 97 | 17 |

### a.2) Determination of the Selectivity

To demonstrate the selectivity of the substances with respect to thrombin inhibition, the test substances are examined for their inhibition of other human serin proteases, such as factor Xa, factor XIIa, factor XIa, trypsin and plasmin. To determine the enzymatic activity of factor Xa (1.3 nmol/l from Kordia), factor XIIa (10 nmol/l from Kordia), factor XIa (0.4 nmol/l from Kordia), trypsin (83 mU/ml from Sigma) and plasmin (0.1 μg/ml from Kordia), these enzymes are dissolved (50 mmol/l of Tris buffer [C,C,C-tris(hydroxymethyl)aminomethane], 100 mmol/1 of sodium chloride, 0.1% BSA [bovine serum albumin], 5 mmol/1 of calcium chloride, pH 7.4) and incubated for 15 min with test substance in various concentrations in dimethyl sulphoxide and also with dimethyl sulphoxide without test substance. The enzymatic reaction is then started by addition of the appropriate substrates (5 μmol/l Boc-Ile-Glu-Gly-Arg-AMC from Bachem for FXa, 5 μmol/l H-Pro-Phe-Arg-AMC from Bachem for factor XIIa, 5 μmol/l Boc-Ile-Glu-Gly-Arg-AMC from Bachem for trypsin, 5 μmol/l Boc-Glu(OBzl)-Ala-Arg-AMC from Bachem for factor XIa, 50 μmol/l MeOSuc-Ala-Phe-Lys-AMC from Bachem for plasmin). After an incubation time of 30 min at 22° C., fluorescence is measured (excitation: 360 nm, emission: 460 nm). The measured emissions of the test mixtures with test substance are compared to the control mixtures without test substance (only dimethyl sulphoxide instead of test substance in dimethyl sulphoxide) and IC₅₀ values are calculated from the concentration/activity relationships.

### a.3) Determination of the Thrombin-Inhibitory Activity of the Potential Inhibitors in Plasma Samples

To determine the inhibition of thrombin in plasma samples, plasma prothrombinase is activated by ecarin. Thrombin activity and/or its inhibition by potential inhibitors is/are then measured fluorenscently by addition of a substrate.

The substances to be tested are dissolved in various concentrations in dimethyl sulphoxide and diluted with water. In white 96-well flat-bottomed plates, 20 μl of substance dilution are mixed with 20 μl of ecarin solution (ecarin reagent, from Sigma E-0504, final concentration 20 mU per reaction) in Ca buffer (200 mM Hepes+560 mM sodium chloride+10 mM calcium chloride+0.4% PEG) or with 20 μl of Ca buffer (as unstimulated control). Furthermore, 20 μl of fluorogenic thrombin substrate (from Bachem 1-1120, final concentration 50 μmol/1) and 20 μl of citrate plasma (from Octapharma) are added, and the mixture is homogenized well. The plate is measured in a SpectraFluorplus Reader using a 360 nm excitation filter and a 465 nm emission filter every minute over 20 minutes. The IC₅₀ value is determined when about 70% of the maximum signal is reached (about 12 min). Representative activity data from this test are given in Table 2 below (in some cases as means of individual determinations):

TABLE 2

| Example No. | IC₅₀ [nM] | Example No. | IC₅₀ [nM] |
|---|---|---|---|
| 1 | 25 | 2 | 19 |
| 4 | 66 | 7 | 70 |
| 9 | 13 | 10 | 39 |
| 11 | 101 | 12 | 8.9 |
| 13 | 31 | 15 | 21 |
| 16 | 36 | 17 | 52 |
| 18 | 27 | 19 | 53 |
| 20 | 66 | 21 | 55 |
| 22 | 11 | 23 | 6.5 |
| 24 | 12 | 25 | 6.8 |
| 26 | 44 | 27 | 7.6 |
| 28 | 17 | 29 | 8.8 |
| 30 | 65 | 31 | 34 |
| 32 | 36 | 33 | 14 |
| 34 | 37 | 35 | 71 |
| 36 | 57 | 37 | 44 |
| 38 | 22 | 39 | 40 |
| 40 | 33 | 41 | 50 |
| 42 | 34 | 43 | 17 |
| 45 | 41 | 46 | 23 |
| 47 | 24 | 48 | 16 |
| 49 | 53 | 50 | 28 |
| 51 | 17 | 52 | 34 |
| 53 | 49 | 54 | 35 |
| 55 | 33 | 56 | 57 |
| 57 | 30 | 58 | 27 |
| 59 | 90 | 60 | 24 |
| 61 | 100 | 62 | 107 |
| 63 | 20 | 65 | 61 |
| 67 | 38 | 68 | 72 |
| 69 | 27 | 70 | 74 |
| 72 | 37 | 73 | 50 |
| 74 | 56 | 75 | 48 |
| 76 | 36 | 77 | 23 |
| 78 | 19 | 79 | 14 |
| 80 | 16 | 81 | 32 |
| 83 | 62 | 84 | 21 |
| 85 | 47 | 87 | 27 |
| 89 | 35 | 90 | 94 |
| 91 | 24 | 92 | 148 |
| 93 | 17 | 94 | 28 |
| 95 | 13 | 96 | 43 |
| 97 | 27 | | |

### a.4) Thrombin Generation Assay (Thrombogram)

The effect of the test substances on the thrombogram (thrombin generation assay according to Hemker) is determined in vitro in human plasma (Octaplas® from Octapharma). In the thrombin generation assay according to Hemker, the activity of thrombin in coagulating plasma is determined by measuring the fluorescent cleavage products of the substrate I-1140 (Z-Gly-Gly-Arg-AMC, Bachem). To initiate the coagulation reaction, reagents from Thrombinoscope are used (PPP reagent: 30 pM recombinant tissue factor, 24 μM phospholipids in HEPES). The reaction is carried out in the presence of varying concentrations of test substance or the corresponding solvent. Moreover, a thrombin calibrator from Thrombinoscope is used whose amidolytic activity is required for calculating the thrombin activity in a plasma sample.

The test is carried out according to the manufacturer's instructions (Thrombinoscope BV): 4 μl of test substance or of the solvent, 76 μl of plasma and 20 μl of PPP reagent or thrombin calibrator are incubated at 37° C. for 5 min. After addition of 20 μl of 2.5 mM thrombin substrate in 20 mM HEPES, 60 mg/ml of BSA, 102 mM of calcium chloride, the thrombin generation is measured every 20 s over a period of 120 min. Measurement is carried out using a fluorometer (Fluoroskan Ascent) from Thermo Electron fitted with a 390/460 nm filter pair and a dispenser. Using the Thrombinoscope software, the thrombogram is calculated and represented graphically. The following parameters are calculated: lag time, time to peak, peak, ETP (endogenous thrombin potential) and start tail.

### a.5) Determination of the Anticoagulatory Activity

The anticoagulatory activity of the test substances is determined in vitro in human plasma, rabbit plasma and rat plasma. To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 4000 g for 15 minutes. The supernatant is pipetted off.

The prothrombin time (PT, synonyms: thromboplastin time, quick test) is determined in the presence of varying concentrations of test substance or the corresponding solvent using a commercial test kit (Neoplastin® from Boehringer Mannheim or Hemoliance® RecombiPlastin from Instrumentation Laboratory). The test compounds are incubated with the plasma at 37° C. for 3 minutes. Coagulation is then started by addition of thromboplastin, and the time when coagulation occurs is determined. The concentration of test substance which effects a doubling of the prothrombin time is determined. Representative activity data from this test are given in Table 3 below (in some cases as means of individual determinations):

TABLE 3

| Example No. | IC₅₀ [μM] | Example No. | IC₅₀ [μM] |
|---|---|---|---|
| 2 | 2.79 | 6 | 6.2 |
| 9 | 2.74 | 12 | 0.93 |
| 15 | 3.38 | 16 | 3.84 |
| 18 | 2.45 | 20 | 5.75 |
| 21 | 5.65 | 24 | 1.14 |
| 25 | 0.93 | 26 | 4.58 |
| 27 | 2.19 | 29 | 1.0 |
| 30 | 5.8 | 31 | 2.79 |
| 32 | 3.28 | 33 | 2.95 |
| 34 | 6.04 | 37 | 3.48 |
| 38 | 3.81 | 45 | 5.27 |
| 47 | 3.23 | 48 | 2.49 |
| 51 | 2.11 | 52 | 4.24 |
| 58 | 2.85 | 60 | 2.55 |
| 63 | 2.36 | 65 | 5.18 |
| 67 | 3.95 | 69 | 5.05 |
| 72 | 4.73 | 75 | 4.15 |
| 77 | 2.83 | 78 | 2.63 |
| 79 | 1.24 | 80 | 3.27 |
| 81 | 2.46 | 84 | 2.55 |
| 85 | 4.26 | 93 | 2.07 |
| 94 | 2.93 | 95 | 1.61 |
| 97 | 5.49 | | |

The thrombin time (TT) is determined in the presence of varying concentrations of test substance or the corresponding solvent using a commercial test kit (thrombin reagent from Roche). The test compounds are incubated with the plasma at 37° C. for 3 minutes. Coagulation is then started by addition of the thrombin reagent, and the time when coagulation occurs is determined. The concentration of test substance which effects a doubling of the thrombin time is determined.

The activated partial thromboplastin time (APTT) is determined in the presence of varying concentrations of test substance or the corresponding solvent using a commercial test kit (PTT reagent from Roche). The test compounds are incubated with the plasma and the PTT reagent (cephalin, kaolin) at 37° C. for 3 minutes. Coagulation is then started by addition of 25 mM calcium chloride, and the time when coagulation occurs is determined. The concentration of test substance which effects a doubling of the APTT is determined.

### a.6) Thromboelastography (Thromboelastogram)

The thromboelastography is carried out with the aid of the thromboelastograph ROTEM from Pentapharm and its accessories, cup and pin. The measurement is carried out in whole blood drawn off beforehand into sodium citrate monovettes from Sarstedt. The blood in the monovettes is kept in motion using a shaker and preincubated at 37° C. for 30 min.

A 2-molar stock solution of calcium chloride in water is prepared. This is diluted 1:10 with an aqueous 0.9% sodium chloride solution. For the measurement, 20 μl of this 200 mM calcium chloride solution are initially charged into the cups (final concentration 12.5 mM calcium chloride). 3.2 μl of substance or solvent are added. The measurement is started by addition of 300 μl of whole blood. After the addition, using the tip of the pipette, the mixture is briefly drawn into the pipette and released again without generating air bubbles. The measurement is carried out over a period of 2.5 hours or stopped when fibrinolysis sets in. For evaluation, the following parameters are determined: CT(clotting time/[sec.]), CFT (clotting formation time/[sec.]), MCF (maximum clot firmness/[mm]) and the alpha angle [°]. The measurement points are determined every 3 seconds and represented graphically, with the y axis for MCF [mm] and the x axis for time [sec.].

### a.7) Inhibition of the Coagulation Factor Thrombin Bound to the Thrombus

Blood clots formed either prior to the start of a therapy with anticoagulants, during therapy-free periods or in spite of therapy contain large amounts of coagulation factors which may favour progressive thrombus formation. These coagulation factors are tightly bound to the thrombus and can not be washed out. In certain clinical situations, this may result in a risk for the patient. Using the tests listed below, it is possible to demonstrate, in human thrombi, both thrombin and FXa having biological (procoagulatory) activity.

### Thrombi Formed In Vitro

Thrombi are formed in vitro from human plasma and examined for activity of the bound coagulation factors thrombin and FXa. To this end, 300 μl of plasma, 30 μl of lipid vesicles and 30 μl of an aqueous calcium chloride solution are mixed in a 48 MTP plate and incubated for 30 min. This step and the following steps are carried out at 37° C. and with constant agitation (300 rpm). The thrombi formed are transferred to a new 48 MTP plate and twice washed for 10 min in 0.9% sodium chloride solution, the thrombus being dabbed on filter paper between the washing steps. The thrombus is transferred into buffer B (Owren's Veronal buffer, 1% BSA) and incubated for 15 min, dabbed on filter paper and incubated for 30 min in test substance in various concentrations in buffer B. The clots are then washed twice as described above. The thrombi are dabbed and transferred into buffer D: (240 μl Owren's Veronal buffer, 1% BSA and 15.6 mM calcium chloride) and incubated with or without 0.6 μM prothrombin for 45 min. The reaction is stopped with 75 μl of 1% EDTA solution. Thrombin activity is measured separately in the thrombus in buffer A (7.5 mM Na₂EDTA×2H₂O, 175 mM sodium chloride, 1% BSA, pH 8.4) or in the supernatant from the last step. To this end, the thrombin substrate used in a.1) is employed in a final concentration of 50 μM, and the resulting fluorescence is measured in a fluorescence plate reader (360/465 nm).

### a.8) Effect of the Thrombin Inhibitors on Thrombolysis in Platelet-Poor Plasma

The effect of the test substances on in vitro thrombolysis in platelet-poor plasma is tested in the presence of tissue plasminogen activator (tPA). To this end, with monitoring by turbidity measurement (UV absorption at 405 nm), initially a clot is formed in a microtitre plate in human plasma with addition of tissue factor, and the dissolution of the clot is adjusted to a certain time window by simultaneous addition of tissue plasminogen activator (tPA). Simultaneous addition of different amounts of the test substance may result in a shortening of the thrombolysis time (the time it takes from maximum turbidity to getting back to the baseline).

In a 384-well microtitre plate, 0.7 μl of an ethanol/water mixture (1:1) comprising various concentrations of the test substances, 1.7 μl of a solution of human thrombomodulin (final concentration 10 nM) and 1.7 μl of a solution of human tissue plasminogen activator (Actilyse®, final concentration 3 nM) are added to 63 μl of human plasma (German Red Cross, corresponds to 90% plasma in the test). Coagulation is initiated by addition of 3.5 μl of a tissue factor-containing solution (Recombiplastin 2G in a 1:100 dilution in 0.2M calcium chloride solution) at 37° C. Measurement of turbidity (UV absorptions measurement at 405 nm) at one minute intervals is then started immediately. The thrombolysis time is calculated as the time it takes from maximum absorption to getting back to the baseline.

### b) Determination of Antithrombotic Activity (In Vivo)

### b.1) Arteriovenous Shunt and Haemorrhage Model (Combi-Model Rat)

Fasting male rats (strain: HSD CPB:WU) having a weight of 300-350 g are anaesthetized using Inactin (150-180 mg/kg). Thrombus formation is initiated in an arteriovenous shunt in accordance with the method described by Christopher N. Berry et al., Br. J. Pharmacol. (1994), 113, 1209 1214. To this end, the left jugular vein and the right carotid artery are exposed. The two vessels are connected by an extracorporeal shunt using a polyethylene tube (PE 60) having a length of 10 cm. In the middle, this polyethylene tube is attached to a further polyethylene tube (PE 160) having a length of 3 cm which contains a roughened nylon thread arranged to form a loop, to form a thrombogenic surface. The extracorporeal circulation is maintained for 15 minutes. The shunt is then removed and the nylon thread with the thrombus is weighed immediately. The weight of the nylon thread on its own is determined before the experiment is started.

To determine the bleeding time, immediately after opening of the shunt circulation, the tip of the tail of the rats is docked by 3 mm using a razor blade. The tail is then placed into physiological saline kept at a temperature of 37° C., and the bleeding from the cut is observed over a period of 15 minutes. What is determined is the time until bleeding ceases for at least 30 seconds (initial bleeding time), total bleeding time over a period of 15 minutes (cumulative bleeding time) and the quantitative blood loss via photometric determination of the collected haemoglobin.

Before the extracorporeal circulation is set up and the tip of the tail is docked, the test substances are administered to the animals while awake either intravenously via the contralateral jugular vein as a single bole or as a bole with subsequent continuous infusion or orally using a pharyngeal tube.

### b.2) Iron(II) Chloride Damage and Bleeding Model (Combi Model II, Rat)

Male rats (strain: HsdRCCHan:Wist) having a weight of 300 g-325 g are anaesthetized intraperitoneally with Inactin (180 mg/kg). Thrombus formation is triggered using iron(II) chloride in the carotid artery. To this end, the right carotid artery is exposed. A flow probe head is then attached, and the blood flow is recorded for 10 minutes. Artery and surroundings are then drained. Parafilm (10×8 mm) and filter paper (10×6 mm folded) are placed under the carotid artery and wetted with 20 μl iron(II) chloride solution (iron(II) chloride tetrahydrate reagent plus 99%, Sigma, 5% solution in water is prepared). A small piece of filter paper is placed on top of the carotid artery and also wetted with iron(II) chloride solution. The carotid artery prepared in this manner is covered with a moist swab and left for 5 minutes. Parafilm and filter paper are then removed and the artery is rinsed with physiological sodium chloride solution. The flow probe head is reattached and the blood flow is recorded for 30 minutes. The measurement is then stopped and the exposed section of the carotid artery is pinched off with tissue clamps and excised. The thrombus located in the vessel is removed from the vessel with the aid of a pair of tweezers and weighed immediately.

To determine the bleeding time, after injury and re-attachment of the flow probe head the tip of the tail of the rat is docked by 3 mm using a razor blade. The tail is then placed into water kept at a temperature of 37° C., and the bleeding from the cut is observed over a period of 15 minutes. What is determined is the time until bleeding ceases for at least 30 seconds (initial bleeding time), total bleeding time over a period of 15 minutes (cumulative bleeding time) and the quantitative blood loss via photometric determination of the collected haemoglobin.

The test substances are administered either intravenously via the jugular vein as single bole directly before the start of the experiment or as a bole (prior to the start) with subsequent continuous infusion.

### b.3) Rabbit Venous Reperfusion and Bleeding Model (Combi Model Rabbit)

Male New Zealand rabbits having a weight of 2.8-3.4 kg are anaesthetized using an intramuscular ketamine/Rompun bole injection. The animal is then shaved at the places needed for the surgery. A continuous infusion of anaesthetic (ketamine/Rompun) is administered via the left auricular vein using an indwelling catheter. Left and right femoral vein and right femoral artery are catheterized with a polyethylene tube (PE50). The jugular vein is then carefully exposed such that the vessel is stressed and damaged as little as possible and no more fat is present at the vessel. Using a suitable apparatus for measuring flow (Powerlab, Transonic TS420 incl. flow probe head), the flow in the jugular vein is recorded (Lab Chart Software). Prior to the start of the experiment, twice 1.4 ml of citrated blood are removed from the rabbit via the femoral artery, and the basal bleeding time at the rim of the ear is determined. Once there has been a constant flow from the jugular vein for 10 min (complete regeneration of the vessel after preparation), a 2 cm section of the vein is pinched off using small vessel clamps. In a Petri dish, the citrated blood removed earlier (300 μl) is mixed with calcium chloride (0.25M, 90 μl) and thrombin (25 U/ml, 60 μl). 180 μl of the blood/calcium chloride/thrombin mixture are quickly drawn into a 1 ml syringe and, via a 27G cannula, injected into the pinched-off segment of the vessel. The injection site is pinched off with a pair of tweezers for one minute so that no blood can escape. Two minutes after injection of the thrombus, the test substance is administered as bole and infusion via the left femoral vein catheter. 14 minutes after the thrombus injection, tissue plasminogen activator is administered as bole and infusion (Actilyse®, 20 μg/kg bole & 150 μg/kg/h infusion) at the right femoral vein. 15 minutes after thrombus injection, the stasis is opened and the flow probe head is attached. Blood flow in the vessel is recorded for 120 minutes, and the vessel is kept moist with warm 0.9% aqueous sodium chloride solution during this time. After 105 minutes of reperfusion, the ear bleeding time is determined again. At the end of the experiment, after 120 minutes of reperfusion, 1.4 ml of citrated blood are removed, the animal is sacrificed painlessly by a bole injection of 1.5 ml of T61 and the weight of the thrombus in the jugular vein is determined. The blood removed before and after the experiment is used to obtain plasma and to determine the ex vivo coagulation time.

The area under the blood flow/time curve (AUC) is calculated and correlated to the maximum achievable area, which is calculated from the blood flow before the experiment and the time (120 min). The area obtainable with tissue plasminogen activator alone is subtracted from the area achieved using the respective substance or dosage. The resulting area is a measure of the improvement of reperfusion by the test substance.

### c) Determination of Pharmacokinetics

### c.1) Pharmacokinetics Following Intravenous Administration of the Test Substance

Male Wistar rats are anaesthetized, and a catheter is placed in the jugular vein. The next day, a defined dose of the test substance is administered as a solution by injection into the tail vein. Blood samples are collected via the catheter over a period of 7 hours (9 points in time).

A defined dose of the test substance is administered to female Beagles as a solution via the cephalic vein as a 15 min infusion. Blood samples are collected via a catheter in the cephalic vein over a period of 7 hours (12 points in time).

The blood is centrifuged in heparin tubes. To precipitate the protein, acetonitrile is added and the plasma sample is centrifuged. The test substance is quantified in the supernatant by LC/MS-MS. The test substance plasma concentrations determined are used to calculate pharmacokinetic parameters such as AUC (area under the plasma concentration/time curve), Vₛₛ (distribution volume), Cmax (highest concentration the test substance in the plasma after administration), t_{1/2} (half-life) and CL (total clearance of the test substance from the plasma). To calculate the blood clearance, the blood/plasma distribution is determined by incubating the test substance in blood. After removal of the plasma by centrifugation, the concentration of the test substance in the plasma is determined by LC/MS-MS.

### c.2) Pharmacokinetics Following Oral Administration of the Test Substance

Male Wistar rats are anaesthetized, and a catheter is placed in the jugular vein. The next day, a defined dose of the test substance is administered orally. Blood samples are collected via the catheter over a period of 24 hours (9 points in time).

A defined dose of the test substance is administered orally to female Beagles. Blood samples are collected via a catheter in the cephalic vein over a period of 24 hours (9 points in time).

The blood is centrifuged in heparin tubes. To precipitate the protein, acetonitrile is added and the plasma sample is centrifuged. The test substance is quantified in the supernatant by LC/MS-MS. The test substance plasma concentrations determined are used to calculate pharmacokinetic parameters such as AUC (area under the plasma concentration/time curve), Cmax (highest concentration of the test substance in the plasma after administration), t_{1/2} (half-life) and F (bioavailability).

### c.3) Caco-2 Permeability Assay

The in vitro permeability of the test substance through a Caco-2 cell monolayer is determined using an established in vitro system for predicting the permeability through the gastrointestinal tract [1]. Caco-2 cells (ACC No. 169, DSMZ, Deutsche Sammlung von Mikroorganismen und Zellkulturen, Brunswick, Germany) are sown in 24-well plates and cultivated for 14 to 16 days. The test substance is dissolved in DMSO and diluted to a concentration of 2 μM in transport buffer (HBSS, Hanks Buffered Salt Solution, Gibco/Invitrogen, supplemented with glucose (final concentration 19.9 mM) and HEPES (final concentration 9.8 mM)). To determine the permeability from apical to basolateral (Pₐₚₚ A-B), the test substance is added on the apical side and transport buffer is added at the basolateral side of the cell monolayer. To determine the permeability from basolateral to apical (Pₐₚₚ B-A), the test substance is added on the basolateral side and transport buffer is added at the apical side of the cell monolayer. At the start of the experiment, samples are taken from the donor compartment to determine the mass balance. After an incubation time of 2 hours at 37° C., samples were taken from the two compartments. The samples were quantified by LC-MS/MS, and the permeability coefficients were calculated. For each cell monolayer, the permeability of Lucifer Yellow was determined to ensure cell monolayer integrity. In each test run, the permeability of atenolol (marker for low permeability) and sulfasalazine (marker for active excretion) is also determined to check the quality of the cells.

Literature: Artursson, P. and Karlsson, J. (1991). Correlation between oral drug absorption in humans and apparent drug permeability coefficients in human intestinal epithelial (Caco-2) cells. Biochem. Biophys. 175 (3), 880-885.

c.4) In Vitro Clearance Determinations with Hepatocytes

Incubations with fresh primary hepatocytes are carried out at 37° C. in a total volume of 1.5 ml with a modified Janus® robot (Perkin Elmer) while shaking. The incubations typically contain 1 million living liver cells/ml, ˜1 μM substrate and 0.05M potassium phosphate buffer (pH=7.4). The final ACN concentration in the incubation is ≦1%.

Aliquots of 125 μl are withdrawn from the incubations after 2, 10, 20, 30, 50, 70 and 90 min and transferred into 96-well filter plates (0.45 μm low-binding hydrophilic PTFE; Millipore: MultiScreen Solvinert). Each of these contain 250 μl of ACN to stop the reaction. After the centrifugation, the filtrates are analyzed by MS/MS (typically API 3000).

The in vitro clearances are calculated from the half-lives of the substance degradation, using the following equations:

CL′_{intrinsic} [ml/(min·kg)]=(0.693/in vitro t1/2 [min])·(liver weight [g liver/kg body weight])·(cell number [1.1·10̂8]/liver weight [g])/(cell number [1·10̂6]/incubation volume [ml])

CL_{blood} is calculated without taking into account the free fraction (“nonrestricted well stirred model”) by the following equation:

CL_{blood} well-stirred [L/(h·kg)]=(Q_{H} [L/(h·kg)]·CL′_{intrinsic} [L/(h·kg)])/(Q_{H} [L/(h·kg)]+CL′_{intrinsic} [L/(h·kg)])

The species-specific extrapolation factors used for the calculation are summarized in Table 4 below:

TABLE 4

| | male/female | | | | | |
|---|---|---|---|---|---|---|
| | Mouse | | | | | Man |
| | m | Mouse f | Rat m/f | Dog m/f | Cyno f | m/f |
| Cell number/g | 110 | 110 | 110 | 110 | 110 | 110 |
| Liver [Mio | | | | | | |
| cells] | | | | | | |
| Liver [g]/ | 50 | 43 | 32 | 39 | 30 | 21 |
| kg Body Weight | | | | | | |
| Liver Blood | 5.4 | 5.4 | 4.2 | 2.1 | 2.5 | 1.3 |
| Flow [l/(h · kg)] | | | | | | |

Fₘₐₓ values which state the maximum possible bioavailability—based on the hepatic extraction—are calculated as follows:

Fₘₐₓ well-stirred [%]=(1−(CL_{blood} well-stirred [L/(h·kg)]/Q_{H} [L/(h·kg)]))·100

### c.5) CYP Inhibition Test

Inhibitory properties of an active compound on the cytochromes P450 (CYP) of the human body may entail extensive clinical effects (drug interactions) because most prescribed medicaments are degraded (metabolized) by these enzymes. Involved in this in particular are the CYP isoenzymes of the 1A and 2C families, CYP2D6 and, with a proportion of almost 50%, CYP3A4. In order to preclude or minimize these possible drug interactions (Drug-Drug Interactions, DDI), the ability of substances to be able to inhibit CYP1A2, CYP2C8, CYP2C9, CYP2D6 and CYP3A4 in humans is investigated using human liver microsomes (pool from various individuals). This takes place by measuring CYP isoform-specific metabolites formed from standard substrates such as, for example, phenacetin, amodiaquin, diclofenac, dextromethorphan, midazolam and testosterone. The inhibitory effects are investigated at six different concentrations of the test compounds (1.5, 3.1, 6.3, 12.5, 25 and 50 μM as maximum concentration or 0.6, 1.3, 2.5, 5, 10 and 20 μM as maximum concentration), compared with the extent of the CYP isoform-specific metabolite formation of the standard substrates in the absence of the test compounds, and the corresponding IC₅₀ values are calculated. CYP isoform-specific standard inhibitors such as, for example, furafylline, montelukast, sulfaphenazole, fluoxetine and ketoconazole serve as control of the results obtained. In order to obtain indications of the possible mechanism-based inhibitors (MBI) on CYP3A4, the human liver microsomes are incubated in the presence of the inhibitor to be investigated for 30 minutes before the addition of midazolam or testosterone as standard substrates of CYP3A4. A reduction in the IC₅₀ value obtained by comparison with the mixture without preincubation serves as an indicator of a mechanism-based inhibition. Mibefradil serves as positive control.

### Procedure:

The incubations of the standard substrates with human liver microsomes (14-100 μg/ml) in the presence of the test compound (as potential inhibitor) are carried out at 37° C. in 96-well plates on a workstation (Tecan, Genesis; Hamiltion, MICROLAB STARLET). The incubation times are 10-15 minutes. The test compounds are preferably dissolved in acetonitrile (1.0, 2.0 or 2.5, 5.0 mM stock solution). The 96-well plates are prepared by sequential addition of a stock solution of NADP+, EDTA, glucose 6-phosphate and glucose 6-phosphate dehydrogenase in phosphate buffer (pH 7.4), the test compound and a solution of standard substrate and human liver microsomes in phosphate buffer (pH 7.4). The total volume is 200 μl. Also located on the 96-well plate are the corresponding control incubations with and without standard inhibitor. After the respective incubation time, the incubations are stopped by addition of 100 μl of acetonitrile comprising a suitable internal standard. Precipitated proteins are removed by centrifugation (3000 rpm, 10 minutes, 10° C.). The resulting supernatants of the respective plates are combined on a plate and analyzed by LC-MS/MS. From the measurement data obtained, the IC₅₀ values are generated and used to assess the inhibitory potential of the test compound.

### c.6) Cellular In Vitro Test for Determining the Induction of Drug-Degrading Cytochromal Enzymes (CYPs) in Primary Human Hepatocytes

Enzyme induction is an unwanted property of a drug which puts broad and safe use of the active compound into question. A consequence of enzyme induction is an accelerated degradation (metabolization) of drugs in the liver. Combined intake of an enzyme inducer and other medicaments such as, for example, immunosuppressives, coagulants or else contraceptives may lead to complete ineffectiveness of the drugs.

The object of the investigation is to provide substances which do not have this unwanted drug interaction. Enzyme inducers are identified with the aid of primary human hepatocytes in long-term culture. To cultivate the cells, hepatocytes are plated on a collagen I layer (density 100000 cells/cm²), and the grown-on cells are then covered with a second collagen layer (sandwich method). (Kern A, Bader A, Pichlmayr R, and Sewing K F, Biochem Pharmacol., 54, 761-772 (1997). To obtain the effect of the test substances on the regulation of the liver enzymes, the hepatocytes are incubated with the active compounds for several days in long-term culture.

### Assay Procedure:

After a two-day regeneration phase, the cells are treated in Williams Medium E, 10% FCS, prednisolone, insulin, glucagon and L-glutamine, penicillin and streptomycin with the test substances. To this end, stock solutions of the active compounds having a concentration of 1 mg/ml in acetonitrile or methanol are prepared and, in 8 dilution steps (1:3) in cell culture medium, pipetted to the cell cultures, which are then incubated in a cell incubator (96% atmospheric humidity, 5% v/v carbon dioxide, 37° C.) for about 5 days. The cell culture medium is changed daily. After this incubation time, the cell cultures are incubated with cytochrome P450(CYP)-specific substrates to determine the activity of the liver enzymes CYP1A2, CYP3A4, CYP2B6 and CYP2C19. The samples thus stopped are either analyzed directly or stored at −20° C. until analysis.

To this end, the media of the cell cultures are chromatographed using suitable C18-reversed-phase columns and variable mixtures of acetonitrile and 10 mM ammonium formate (HPLC-MS/MS).

The mass spectrometric data serve to quantify the substrate turnover and, derived therefrom, to calculate the liver enzyme activities. Active compounds having unfavourable properties with respect to liver enzyme regulation are not persued any further.

### C) WORKING EXAMPLES OF PHARMACEUTICAL COMPOSITIONS

The substances according to the invention can be converted to pharmaceutical preparations as follows:

### Tablet:

### Composition:

100 mg of the compound of Example 1, 50 mg of lactose (monohydrate), 50 mg of maize starch, 10 mg of polyvinylpyrrolidone (PVP 25) (from BASF, Germany) and 2 mg of magnesium stearate.

Tablet weight 212 mg. Diameter 8 mm, radius of curvature 12 mm.

### Production:

The mixture of the compound of Example 1, lactose and starch is granulated with a 5% strength solution (m/m) of the PVP in water. After drying, the granules are mixed with the magnesium stearate for 5 min. This mixture is compressed in a conventional tabletting press (see above for format of the tablet).

### Oral Suspension:

### Composition:

1000 mg of the compound of Example 1, 1000 mg of ethanol (96%), 400 mg of Rhodigel (xanthan gum) (from FMC, USA) and 99 g of water.

10 ml of oral suspension correspond to a single dose of 100 mg of the compound according to the invention.

### Production:

The Rhodigel is suspended in ethanol, and the compound of Example 1 is added to the suspension. The water is added while stirring. The mixture is stirred for about 6 h until swelling of the Rhodigel is complete.

### Intravenously Administrable Solution:

### Composition:

1 mg of the compound of Example 1, 15 g of polyethylene glycol 400 and 250 g of water for injection purposes.

### Production:

The compound of Example 1 is dissolved together with polyethylene glycol 400 by stirring in the water. The solution is sterilized by filtration (pore diameter 0.22 μm) and dispensed under aseptic conditions into heat-sterilized infusion bottles. The latter are closed with infusion stoppers and crimped caps.

1. Compound of the formula 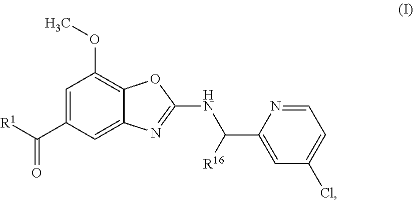
in which
R¹ represents a group of the formula 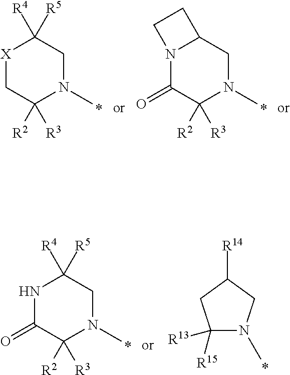
where * is the point of attachment to the carbonyl group,
X represents an oxygen atom, a sulphur atom or CH—R⁶,
where
R⁶ represents hydrogen or hydroxy,
R² represents hydrogen, aminocarbonyl, C₁-C₆-alkyl, C₃-C₆-cycloalkyl or phenyl,
where alkyl and cycloalkyl may be substituted by a substituent selected from the group consisting of hydroxy, methoxy, cyano, hydroxycarbonyl, aminocarbonyl, methylsulphonyl, difluoromethoxy, trifluoromethoxy and cyclopropyl,
where cyclopropyl for its part may be substituted by a hydroxy substituent,
or
where alkyl and cycloalkyl may be substituted by 1 to 3 fluorine substituents,
R³ represents hydrogen or C₁-C₄-alkyl,
or
R² and R³ together with the carbon atom to which they are attached form a cyclopropyl ring, cyclobutyl ring or cyclopentyl ring,
where the cyclobutyl ring and the cyclopentyl ring may be substituted by a hydroxy substituent,
R⁴ represents hydrogen or C₁-C₆-alkyl,
where alkyl may be substituted by a hydroxy substituent,
or
where alkyl may be substituted by 1 to 3 fluorine substituents,
R⁵ represents C₁-C₄-alkyl,
or
R⁴ and R⁵ together with the carbon atom to which they are attached form a cyclopropyl ring, cyclobutyl ring or cyclopentyl ring,
where the cyclobutyl ring and the cyclopentyl ring may be substituted by a hydroxy substituent,
R⁷ represents hydrogen or C₁-C₆-alkyl,
where alkyl may be substituted by one substituent selected from the group consisting of cyano, hydroxy and methoxy,
or
where alkyl may be substituted by 1 to 3 fluorine substituents,
R⁸ represents hydrogen,
R⁹ represents hydrogen or C₁-C₆-alkyl,
where alkyl may be substituted by one substituent selected from the group consisting of hydroxy, cyano and aminocarbonyl,
or
where alkyl may be substituted by 1 to 3 fluorine substituents,
R¹⁰ represents hydrogen,
R¹¹ represents C₁-C₄-alkyl,
where alkyl may be substituted by a hydroxy substituent,
R¹² represents hydrogen or C₁-C₄-alkyl,
or
R¹¹ and R¹² together with the carbon atom to which they are attached form a cyclopropyl ring, cyclobutyl ring or cyclopentyl ring,
where the cyclobutyl ring and the cyclopentyl ring may be substituted by a hydroxy substituent,
R¹³ represents methyl, ethyl, (3-fluoroazetidin-1-yl)carbonyl, (3,3-difluoroazetidin-1-yl)carbonyl or morpholin-4-ylcarbonyl,
where methyl and ethyl are substituted by a substituent selected from the group consisting of cyano and hydroxy,
R¹⁴ represents hydrogen, methoxy, ethoxy or cyclopropyloxy,
where methoxy and ethoxy may be substituted by 1 to 3 substituents selected from the group consisting of deuterium and fluorine,
R¹⁵ represents hydrogen or methyl,
and
R¹⁶ represents hydrogen, methyl or fluoromethyl,
or one of the salts thereof, solvates thereof or solvates of the salts thereof.

2. Compound according to claim 1, characterized in that
R¹ represents a group of the formula 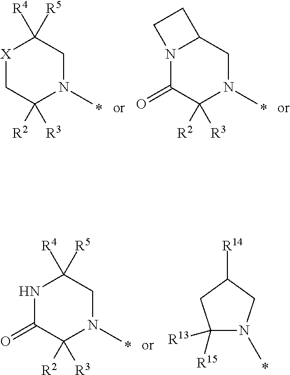
where * is the point of attachment to the carbonyl group,
X represents an oxygen atom or CH—R⁶,
where
R⁶ represents hydrogen,
R² represents hydrogen, C₁-C₄-alkyl or C₃-C₆-cycloalkyl,
where alkyl and cycloalkyl may be substituted by a substituent selected from the group consisting of hydroxy, methoxy, hydroxycarbonyl, difluoromethoxy and cyclopropyl,
where cyclopropyl for its part may be substituted by a hydroxy substituent,
or
where alkyl may be substituted by 1 to 3 fluorine substituents,
R³ represents hydrogen or C₁-C₄-alkyl,
or
R² and R³ together with the carbon atom to which they are attached form a cyclobutyl ring,
where the cyclobutyl ring may be substituted by a hydroxy substituent,
R⁴ represents hydrogen or C₁-C₄-alkyl,
where alkyl may be substituted by a hydroxy substituent,
R⁵ represents C₁-C₄-alkyl,
R⁷ represents C₁-C₄-alkyl,
where alkyl may be substituted by a methoxy substituent,
R⁸ represents hydrogen,
R⁹ represents C₁-C₄-alkyl,
where alkyl may be substituted by one substituent selected from the group consisting of cyano and aminocarbonyl,
R¹⁰ represents hydrogen,
R¹¹ represents C₁-C₄-alkyl,
R¹² represents hydrogen or C₁-C₄-alkyl,
or
R¹¹ and R¹² together with the carbon atom to which they are bonded form a cyclopropyl ring,
R¹³ represents methyl, ethyl, (3-fluoroazetidin-1-yl)carbonyl, (3,3-difluoroazetidin-1-yl)carbonyl or morpholin-4-ylcarbonyl,
where methyl and ethyl are substituted by a substituent selected from the group consisting of cyano and hydroxy,
R¹⁴ represents hydrogen, ethoxy or cyclopropyloxy,
where ethoxy may be substituted by 1 to 3 substituents selected from the group consisting of deuterium and fluorine,
R¹⁵ represents hydrogen or methyl,
and
R¹⁶ represents hydrogen or methyl,
or one of the salts thereof, solvates thereof or solvates of the salts thereof.

3. Compound according to claim 1, characterized in that
R¹ represents a group of the formula 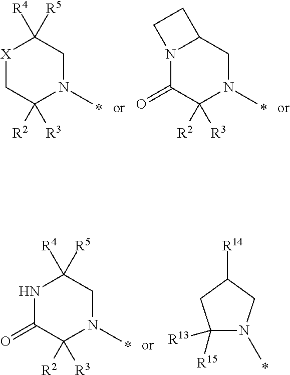
where * is the point of attachment to the carbonyl group,
X represents an oxygen atom,
R² represents C₁-C₄-alkyl or cyclobutyl,
where alkyl is substituted by a hydroxy substituent,
and
where cyclobutyl is substituted by a hydroxy substituent,
R³ represents hydrogen,
R⁴ represents hydrogen or methyl,
and
R⁵ represents methyl,
or
R² represents methyl,
where methyl may be substituted by 1 to 2 fluorine substituents,
R³ represents hydrogen or methyl,
R⁴ represents C₁-C₄-alkyl,
where alkyl is substituted by a hydroxy substituent,
and
R⁵ represents methyl,
or
R² and R³ together with the carbon atom to which they are attached form a cyclobutyl ring,
where the cyclobutyl ring is substituted by a hydroxy substituent,
R⁴ represents hydrogen,
and
R⁵ represents methyl,
R⁷ represents methyl,
R⁸ represents hydrogen,
R⁹ represents methyl or ethyl,
where methyl may be substituted by a cyano substituent,
R¹⁰ represents hydrogen,
R¹¹ represents methyl,
R¹² represents hydrogen,
or
R¹¹ and R¹² together with the carbon atom to which they are bonded form a cyclopropyl ring,
R¹³ represents methyl,
where methyl is substituted by a hydroxy substituent,
R¹⁴ represents ethoxy or cyclopropyloxy,
where ethoxy may be substituted by 1 to 3 substituents selected from the group consisting of deuterium and fluorine,
R¹⁵ represents hydrogen,
and
R¹⁶ represents hydrogen or methyl,
or one of the salts thereof, solvates thereof or solvates of the salts thereof.

4. Compound according to claim 1, characterized in that
R¹ represents a group of the formula 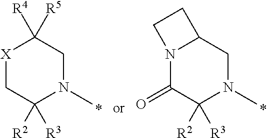
where * is the point of attachment to the carbonyl group,
X represents an oxygen atom,
R² represents C₁-C₄-alkyl or cyclobutyl,
where alkyl is substituted by a hydroxy substituent,
and
where cyclobutyl is substituted by a hydroxy substituent,
R³ represents hydrogen,
R⁴ represents hydrogen or methyl,
and
R⁵ represents methyl,
or
R² represents methyl,
where methyl may be substituted by 1 to 2 fluorine substituents,
R³ represents hydrogen or methyl,
R⁴ represents C₁-C₄-alkyl,
where alkyl is substituted by a hydroxy substituent,
and
R⁵ represents methyl,
or
R² and R³ together with the carbon atom to which they are attached form a cyclobutyl ring,
where the cyclobutyl ring is substituted by a hydroxy substituent,
R⁴ represents hydrogen,
and
R⁵ represents methyl,
R⁷ represents methyl,
R⁸ represents hydrogen,
and
R¹⁶ represents hydrogen or methyl,
or one of the salts thereof, solvates thereof or solvates of the salts thereof.

5. Compound according to claim 1, characterized in that
R¹ represents a group of the formula 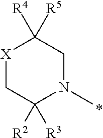
where * is the point of attachment to the carbonyl group,
X represents an oxygen atom,
R² represents C₁-C₄-alkyl or cyclobutyl,
where alkyl is substituted by a hydroxy substituent,
and
where cyclobutyl is substituted by a hydroxy substituent,
R³ represents hydrogen,
R⁴ represents hydrogen or methyl,
and
R⁵ represents methyl,
or
R² represents methyl,
where methyl may be substituted by 1 to 2 fluorine substituents,
R³ represents hydrogen or methyl,
R⁴ represents C₁-C₄-alkyl,
where alkyl is substituted by a hydroxy substituent,
and
R⁵ represents methyl,
or
R² and R³ together with the carbon atom to which they are attached form a cyclopropyl ring,
where the cyclobutyl ring is substituted by a hydroxy substituent,
R⁴ represents hydrogen,
and
R⁵ represents methyl,
and
R¹⁶ represents hydrogen or methyl,
or one of the salts thereof, solvates thereof or solvates of the salts thereof.

6. Compound according to claim 1, characterized in that the compound is
(2-{[(4-chloropyridin-2-yl)methyl]amino}-7-methoxy-1,3-benzoxazol-5-yl)[(5R)-2-(2-hydroxyethyl)-2,5-dimethylmorpholin-4-yl]methanone [enantiomerically pure isomer]
or
(2-{[(4-chloropyridin-2-yl)methyl]amino}-7-methoxy-1,3-benzoxazol-5-yl) [5-(2-hydroxypropan-2-yl)-2-methylmorpholin-4-yl]methanone [enantiomerically pure isomer 2]
or
7-[(2-{[(4-chloropyridin-2-yl)methyl]amino}-7-methoxy-1,3-benzoxazol-5-yl)carbonyl]-6-ethyl-4,7-diazaspiro[2.5]octan-5-one [enantiomerically pure isomer 1]
or
{1-[(2-{[(4-chloropyridin-2-yl)methyl]amino}-7-methoxy-1,3-benzoxazol-5-yl)carbonyl]-5,5-dimethyl-3-oxopiperazin-2-yl}acetonitrile [enantiomerically pure isomer 2]
or
(2-{[(4-chloropyridin-2-yl)methyl]amino}-7-methoxy-1,3-benzoxazol-5-yl) [5-(1-hydroxyethyl)-2,2-dimethylmorpholin-4-yl]methanone [enantiomerically pure isomer 1]
or one of the salts, the solvates or the solvates of the salts of these compounds.

7. Process for preparing a compound of the formula (I) or one of the salts thereof, solvates thereof or solvates of the salts thereof according to claim 1, characterized in that a compound of the formula 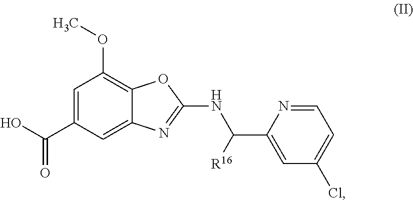
in which
R¹⁶ has the meaning given in claim 1,
is reacted with a compound of the formula
R¹—H (III),
in which
R¹ has the meaning given in claim 1,
with dehydrating agents.

8. Compound according to claim 1, for the treatment and/or prophylaxis of diseases.

9. Use of a compound according to claim 1, for producing a medicament for the treatment and/or prophylaxis of diseases.

10. Use of the compound according to claim 1, for producing a medicament for the treatment and/or prophylaxis of thromboembolic disorders.

11. Use of the compound according to claim 1, for producing a medicament for the treatment and/or prophylaxis of acute coronary syndrome (ACS), venous thromboembolisms, venous thromboses, in particular in deep leg veins and kidney veins, pulmonary embolisms, stroke and/or thrombosis prophylaxis in the context of surgical interventions, in particular in the context of surgical interventions in patients suffering from cancer.

12. Medicament comprising the compound according to claim 1, in combination with an inert, nontoxic, pharmaceutically suitable excipient.

13. Medicament according to claim 12, for the treatment and/or prophylaxis of thromboembolic disorders.

14. Method for combating thromboembolic disorders in humans and animals by administration of a therapeutically effective amount of the compound according to claim 1.
